(19) United States
(12) Patent Application Publication
(10) Pub. No.: US 2026/0000756 A1
(43) Pub. Date: Jan. 1, 2026

(54) COMBINATIONS OF IMMUNOTHERAPIES AND USES THEREOF

(71) Applicant: OncoResponse, Inc., Seattle, WA (US)

(72) Inventors: Siddarth Chandrasekaran, Issaquah, WA (US); Tyrel T. Smith, Seattle, WA (US); Kamal D. Puri, Seattle, WA (US); Randi M. Simmons, Seattle, WA (US); Peter Probst, Seattle, WA (US)

(21) Appl. No.: 19/036,864

(22) Filed: Jan. 24, 2025

Related U.S. Application Data
(63) Continuation of application No. 17/929,655, filed on Sep. 2, 2022, now abandoned.
(60) Provisional application No. 63/260,916, filed on Sep. 3, 2021.
(60) Provisional application No. 63/261,191, filed on Sep. 14, 2021.
(60) Provisional application No. 63/365,858, filed on Jun. 3, 2022.

(51) Int. Cl. A61K 39/395; A61K 9/00; A61K 31/337; A61K 39/00; A61P 11/00; A61P 35/00

(52) U.S. Cl. CPC A61K 39/3955; A61K 9/0019; A61K 31/337; A61P 11/00; A61P 35/00; A61K 2039/507

(57) ABSTRACT

Provided herein are uses of antibodies that bind to CD163 expressed on a human myeloid cell in combination with checkpoint inhibitors. Among other things, these CD163 antibodies can be used with checkpoint inhibitors in methods of treatment of humans, such as methods of treating a cancer or methods of relieving T cell suppression.

### CROSS-REFERENCE

This application is a continuation of U.S. application Ser. No. 17/929,655, filed Sep. 2, 2022, which claims the benefit of and priority to U.S. Provisional Patent Application No. 63/260,916, filed Sep. 3, 2021; U.S. Provisional Patent Application No. 63/261,191, filed Sep. 14, 2021; and U.S. Provisional Patent Application No. 63/365,858, filed Jun. 3, 2022, the disclosures each of which are hereby incorporated by reference in its entirety for all purposes.

### REFERENCE TO AN ELECTRONIC SEQUENCE LISTING XML

This application contains a Sequence Listing which has been submitted electronically in XML format. The Sequence Listing XML, including all contents therein, (File Name: ONR-007_SL.xml; Size: 49,639 bytes; and Date of Creation: Jan. 20, 2023) is herein incorporated by reference in its entirety.

### SUMMARY OF THE DISCLOSURE

Disclosed herein, in certain embodiments, are methods of treating a cancer in a subject in need thereof, comprising administering to the individual (a) a CD163 antibody and (b) an immune checkpoint inhibitor. In some embodiments, the CD163 antibody is an immunomodulatory CD163 antibody. In some embodiments, administering the immunomodulatory CD163 antibody and the immune checkpoint inhibitor to the subject additively or synergistically produces a therapeutic effect. In some embodiments, administering the immunomodulatory CD163 antibody and the immune checkpoint inhibitor to the subject additively or synergistically reduces immunosuppression of T cells and increases T cell activity and proliferation, resulting in a greater immune response to the cancer as compared to the immune response generated when the immunomodulatory CD163 antibody or the immune checkpoint inhibitor is administered alone. In some embodiments, the immunomodulatory CD163 antibody binds to human CD163 (hCD163) (e.g., hCD163 on a myeloid cell).

In some embodiments, the immunomodulatory CD163 antibody comprises: a light chain variable domain (V_{L}) having a sequence at least 80% identical to the amino acid sequence selected from the group consisting of: SEQ ID NO: 28, SEQ ID NO: 30, SEQ ID NO: 32, SEQ ID NO: 34, SEQ ID NO: 36, SEQ ID NO: 38, and SEQ ID NO: 40 and a heavy chain variable domain (V_{H}) having a sequence at least 80% identical to the amino acid sequence selected from the group consisting of: SEQ ID NO: 29, SEQ ID NO: 31, SEQ ID NO: 33, SEQ ID NO: 35, SEQ ID NO: 37, SEQ ID NO: 39, and SEQ ID NO: 41; and b) an immune checkpoint inhibitor.

In some embodiments, the immunomodulatory CD163 antibody comprises: (i) a light chain variable domain (V_{L}) and a heavy chain variable domain (V_{H}) and with amino acid sequences as set forth in SEQ ID NOs: 40 and 41; or (ii) six CDRs with amino acid sequences as set forth in SEQ ID NOS: 1 (CDR L1), 2 (CDR L2), 3 (CDR L3), 4 (CDR H1), 5 (CDR H2), and 6 (CDR H3).

In some embodiments, the immunomodulatory antibody comprises a light chain variable domain (V_{L}) having a sequence 100% identical to the amino acid sequence selected from the group consisting of: SEQ ID NO: 28, SEQ ID NO: 30, SEQ ID NO: 32, SEQ ID NO: 34, SEQ ID NO: 36, SEQ ID NO: 38, and SEQ ID NO: 40. In some embodiments, an immunomodulatory CD163 antibody comprises a heavy chain variable domain (V_{H}) having a sequence 100% identical to the amino acid sequence selected from the group consisting of: SEQ ID NO: 29, SEQ ID NO: 31, SEQ ID NO: 33, SEQ ID NO: 35, SEQ ID NO: 37, SEQ ID NO: 39, and SEQ ID NO: 41. In some embodiments, the immunomodulatory CD163 antibody comprises a CDR H1 that has a sequence as set forth in an amino acid sequence selected from the group consisting of: SEQ ID NO: 4, SEQ ID NO: 16, SEQ ID NO: 19, SEQ ID NO: 22 and SX₁X₂MH, wherein X1=Y, E, Q, D; and X2=A, D, T, V, S, G, E. In some embodiments, the immunomodulatory CD163 antibody comprises a CDR H2 that has a sequence as set forth in an amino acid sequence selected from the group consisting of: SEQ ID NO: 5, SEQ ID NO: 17, SEQ ID NO: 20, SEQ ID NO: 23, and SEQ ID NO: 26. In some embodiments, the immunomodulatory CD163 antibody comprises a CDR H3 that has a sequence as set forth in an amino acid sequence selected from the group consisting of: SEQ ID NO: 6, SEQ ID NO: 18, SEQ ID NO: 21, SEQ ID NO: 24, and SEQ ID NO: 27. In some embodiments, the immunomodulatory CD163 antibody comprises a CDR L1 that has a sequence as set forth in an amino acid sequence selected from the group consisting of: SEQ ID NO: 1, SEQ ID NO: 7, and SEQ ID NO: 13. In some embodiments, the immunomodulatory CD163 antibody comprises a CDR L2 that has a sequence as set forth in an amino acid sequence selected from the group consisting of: SEQ ID NO: 2, SEQ ID NO: 9, and SEQ ID NO: 14. In some embodiments, the immunomodulatory CD163 antibody comprises a CDR L3 that has a sequence as set forth in an amino acid sequence selected from the group consisting of: SEQ ID NO: 3, SEQ ID NO: 8, SEQ ID NO: 10, SEQ ID NO: 11, SEQ ID NO: 12, and SEQ ID NO: 15. In some embodiments, the immunomodulatory CD163 antibody that comprises a heavy chain variable domain and a light chain variable domain, wherein the variable domains comprise amino acid sequences that are 100% identical at each of CDR H1, CDR H2, CDR H2, CDR L1, CDR L2, and CDR L3 as follows: (a) SEQ ID NOs: 1, 2, 3, 4, 5, and 6; (b) SEQ ID NOs: 7, 2, 8, 16, 17, and 18; (c) SEQ ID NOs: 7, 9, 10, 19, 20, and 21; (d) SEQ ID NOs: 7, 2, 11, 22, 23, and 24; (e) SEQ ID NOs: 7, 2, 8, 22, 17, and 18; (f) SEQ ID NOs: 7, 2, 10, 16, 17, and 24; and (g) SEQ ID NOs: 7, 2, 12, 19, 17, and 18. In some embodiments, the immunomodulatory CD163 antibody comprises a pair of variable domains consisting of a light chain variable domain and a heavy chain variable domain, which pair of domains is selected from the group consisting of: (a) SEQ ID NOs: 28 and 29; (b) SEQ ID NOs: 30 and 31; (c) SEQ ID NOs: 32 and 33; (d) SEQ ID NOs: 34 and 35; (e) SEQ ID NOs: 36 and 37; (f) SEQ ID NOs: 38 and 39; and (g) SEQ ID NOs: 40 and 41. In some embodiments, the immunomodulatory CD163 antibody comprises amino acid sequences comprising six CDRs as set forth in Tables 2 and 3, wherein the six CDRs are selected from the group consisting of: (a) SEQ ID NOs: 1, 2, 3, 4, 5, and 6; (b) SEQ ID NOs: 7, 2, 8, 16, 17, and 18; (c) SEQ ID NOs: 7, 9, 10, 19, 20, and 21; (d) SEQ ID NOs: 7, 2, 11, 22, 23, and 24; (e) SEQ ID NOs: 7, 2, 8, 22, 17, and 18; (f) SEQ ID NOs: 7, 2, 10, 16, 17, and 24; and (g) SEQ ID NOs: 7, 2, 12, 19, 17, and 18.

In some embodiments, the immunomodulatory CD163 antibody comprises a heavy chain variable domain and a light chain variable domain, the variable domains together comprising six CDRs set forth as follows:
(a) RASQSISX8YLN (SEQ ID NO: 13), wherein X8=S, R, K, H;
(b) AASSLQX9 (SEQ ID NO: 14), wherein X9=S, N, Q, T;
(c) QQSYSTX10X11GX12 (SEQ ID NO: 15), wherein X10=P, Q, T, S, N, A, G; X11=R, G, A, S; and X12=T, S, A, G, N;
(d) SX₁X₂MH, wherein X1=Y, E, Q, D; and X2=A, D, T, V, S, G, E;
(e) VISX3DGSNKYX4ADSVKG (SEQ ID NO: 26), wherein X3=Y, E, Q, D; and X4=Y, N, H, E, D, K, Q, R; and
(f) ENVRPYYDFWX5GYX6SEYYYYGX7DV (SEQ ID NO: 27), wherein X5=S, R, K, H; X6=Y, S, N, T, A, Q; and X7=M, L, I, V.

Disclosed herein, in certain embodiments, are methods of treating a cancer with an immunotherapy wherein the cancer is associated with the presence of immunosuppressive macrophages (e.g., tumor-associated macrophages, e.g., M2-like macrophages), wherein at least a portion of the immunosuppressive macrophages is located in a tumor, in a subject in need thereof, the method comprising administering to the subject a therapeutically effective amount of (a) an immunomodulatory CD163 antibody that specifically binds to human CD163 (hCD163) and (b) an immune checkpoint inhibitor.

In some embodiments, the immunomodulatory CD163 antibody is an IgG1 antibody or IgG4 antibody.

In some embodiments, the immunomodulatory CD163 antibody specifically binds to hCD163. In some embodiments, a myeloid cell is an immunosuppressive macrophage or a myeloid-derived suppressor cell. In some embodiments, the immunomodulatory CD163 antibody comprises a constant domain that interacts with a macrophage. “Interacts” as described herein can include binding or modulating said macrophage. In some embodiments, the immunomodulatory CD163 antibody comprises a constant domain that interacts with a macrophage via interaction with CD16, CD32, CD64, or combinations thereof.

In some embodiments, the immunomodulatory CD163 antibody binding to the myeloid cell antagonizes an immunosuppressive function mediated by immune cells (i) directly, through cancer cell-immune cell interactions; and/or (ii) through secreted products of the cancer cells or the immune cells. The immune cell may be a myeloid cell, such as a tumor-associated macrophage. In some embodiments, the antagonism of the immunosuppressive function is greater than when the immunomodulatory CD163 antibody is administered in the absence of the immune checkpoint inhibitor.

In some embodiments, the immunomodulatory CD163 antibody binding promotes proliferation of CD3+ T cells in the subject. In some embodiments, the immunomodulatory CD163 antibody binding promotes proliferation of CD4+ T cells in the subject. In some embodiments, the immunomodulatory CD163 antibody binding promotes proliferation of CD8+ T cells in the subject. In some embodiments, the immunomodulatory CD163 antibody binding promotes proliferation of CD4+ and CD8+ T cells in the subject.

In some embodiments, the proliferation of CD3+ T cells in the subject is greater than when the immunomodulatory CD163 antibody binds in the absence of the immune checkpoint inhibitor. In some embodiments, the immunomodulatory CD163 antibody binding promotes increased cytokine and pore-forming protein perforin levels in the subject. In some embodiments, the increased cytokine levels are greater than when the immunomodulatory CD163 antibody binds in the absence of the immune checkpoint inhibitor. In some embodiments, the increased cytokine levels comprise increased levels of one or more of interferon gamma (IFN-γ), TNF-α, and IL-2. In some embodiments, the increased cytokine levels are increased to a greater degree than when the immunomodulatory CD163 antibody binds in the absence of the immune checkpoint inhibitor.

In some embodiments, the binding of the immunomodulatory CD163 antibody to the myeloid cell promotes T-cell-mediated killing of cancer cells in the subject. In some embodiments, the immune checkpoint inhibitor inhibits binding of a receptor on a T cell to its ligand (typically expressed by immune suppressive cells in the tumor microenvironment such as myeloid-derived suppressor cells (MDSC), regulatory T cells (Treg), and tumor-associated macrophages (TAM). In some embodiments, the immune checkpoint inhibitor modulates immunosuppression by cancer cells or other immunosuppressive cells in the subject, wherein at least a portion of the cancer cells or other immunosuppressive cells in the TME express the receptor or its ligand. In some embodiments, the immune checkpoint inhibitor inhibits the inhibitory receptor-mediated or ligand-mediated immunosuppression by the cancer, and the immunomodulatory CD163 antibody promotes an immunostimulatory response against the cancer cell.

In some embodiments, the immune checkpoint inhibitor is an antagonist to an immune checkpoint protein or to a ligand thereof, wherein the immune checkpoint protein is selected from the group consisting of CTLA-4, PD-1, PD-L1, LAG3, TIM3, TIGIT, or any combination thereof.

In some embodiments, the immune checkpoint inhibitor is a PD-1 antagonist.

In some embodiments, the PD-1 antagonist is a PD-1 antibody. In some embodiments, the PD-1 antagonist is a small molecule. In some embodiments, the PD-1 antagonist is a rationally designed peptide antagonist of PD-1. See, e.g., Liu et al., Cancer Cell Int (2021) 21:239.

In some embodiments, the PD-1 antibody is an IgG1 antibody or IgG4 antibody. In some embodiments, the PD-1 antibody is selected from the group consisting of nivolumab, pembrolizumab, cemiplimab, dostarlimab, JTX-4014, spartalizumab, camrelizumab, sintilimab, tislelizumab, toripalimab, INCMGA00012, AMP-224, AMP-514, zimberelimab, and fragments or combinations thereof. In some embodiments, the PD-1 antagonist comprises a PD-1 binding domain comprising CDRs of an antibody selected from the group consisting of nivolumab, pembrolizumab, cemiplimab, dostarlimab, JTX-4014, spartalizumab, camrelizumab, sintilimab, tislelizumab, toripalimab, INCMGA00012, AMP-224, or AMP-514, zimberelimab, and fragments or combinations thereof. In some embodiments, the binding of the immunomodulatory CD163 antibody to the myeloid cell potentiates an immune response disinhibited by the PD-1 antagonist; in some embodiments, the PD-1 antagonist disinhibits immunosuppression by cancer cells in the subject and the immunomodulatory CD163 antibody binding promotes immunostimulatory responses against the cancer cells; and/or in some embodiments, the PD-1 antagonist inhibits PD-1 mediated immunosuppression by cancer cells in the subject and the immunomodulatory CD163 antibody promotes an immunostimulatory response against the cancer cells.

In some embodiments, the immune checkpoint inhibitor is a PD-L1 antagonist. In some embodiments, the PD-L1 antagonist is an antibody. In some embodiments, the PD-L1 antibody is an IgG1 antibody or IgG4 antibody. In some embodiments, the PD-L1 antagonist is selected from the group consisting of avelumab, durvalumab, atezolizumab, envafolimab, cosibelimab (CK-301), LY3300054, CA-170, BMS-936559 and combinations and fragments thereof. In some embodiments, the PD-L1 antagonist comprises a PD-L1 binding domain comprising CDRs of an antibody selected from the group consisting of avelumab, durvalumab, atezolizumab, envafolimab, cosibelimab (CK-301), LY3300054, CA-170, BMS-936559, and combinations and PD-L1-binding fragments thereof. In some embodiments, the PD-L1 antagonist is a peptide (e.g., AUNP-12 or BMS-986189). In some embodiments, the binding of the immunomodulatory CD163 antibody to the myeloid cell potentiates an immune response disinhibited by the PD-L1 antagonist; the PD-L1 antagonist disinhibits immunosuppression by cancer cells in the subject and the immunomodulatory CD163 antibody binding promotes immunostimulatory responses against the cancer cells; and/or the PD-L1 antagonist inhibits PD-L1 mediated immunosuppression by cancer cells in the subject and the immunomodulatory CD163 antibody promotes an immunostimulatory response against the cancer cells.

In some embodiments, the immunomodulatory CD163 antibody and the immune checkpoint inhibitor are administered concomitantly or sequentially. In some embodiments, the sequential administration comprises administering the immunomodulatory CD163 antibody prior to administering the immune checkpoint inhibitor. In some embodiments, the sequential administration comprises administering the immune checkpoint inhibitor prior to administering the immunomodulatory CD163 antibody. In some embodiments, when administration is sequential, the interval between administration of the immunomodulatory CD163 antibody and administration of the immune checkpoint inhibitor is between one hour and 28, 30, 35, 42, 45, 49, 56, or 60 days. In some embodiments, the doses are administered about one week (seven days) apart, i.e., a weekly interval, about two weeks (about 14 days) apart, i.e., a twice-weekly interval, or about three weeks (about 21 days) apart, i.e., a three-week interval or about six weeks (about 42 days) apart, i.e., a six-week interval. In some embodiments, the immunomodulatory CD163 antibody that binds to CD163 on a human myeloid cell is administered at a dose of 150-1200 mg.

In some embodiments, the immunomodulatory CD163 antibody is administered intravenously or subcutaneously. In some embodiments, more than one dose of immunomodulatory CD163 antibody is administered. In some embodiments, the immunomodulatory CD163 antibody may be administered over an extended period, such as a 30-minute period, a 45-minute period, a 60-minute period, a 90-minute period, a 120-minute period, a 180-minute period, or longer. In some embodiments, administration of CD163 antibody occurs once per week. In some embodiments, weekly administration is repeated for two, three, four, five, six, seven, eight, nine, ten, eleven, twelve or more weeks.

In some embodiments, the immune checkpoint inhibitor is administered intravenously or subcutaneously. In some embodiments, more than one dose of checkpoint inhibitor is administered. In some embodiments, the administration of the immune checkpoint inhibitor is over a 30-minute period. In some embodiments, the 30-minute period is the same 30-minute period. In some embodiments, the 30-minute period is a different 30-minute period.

In some embodiments, pembrolizumab is administered at a dose of 200 mg or 400 mg. In some embodiments, the dose of pembrolizumab is administered more than once. In some embodiments, when the dose of pembrolizumab is 200 mg, it is administered once every three weeks, and when the dose of pembrolizumab is 400 mg, it is administered once every six weeks, or in either case until disease progression or unacceptable toxicity. In some cases, administration may be terminated after two years in patients with responses.

In some embodiments, cemiplimab is administered at a dose of 350 mg. In some embodiments, the dose of cemiplimab is administered more than once. In some embodiments, cemiplimab is administered at a dose of 350 mg once every three weeks, or until disease progression or unacceptable toxicity.

In some embodiments, nivolumab is administered at a dose of 240 mg or 480 mg. In some embodiments, the dose of nivolumab is administered more than once. In some embodiments, when the dose of nivolumab is 240 mg it is administered once every two weeks, and when the dose of nivolumab is 480 mg it is administered once every four weeks, or in either case until disease progression or unacceptable toxicity.

In some embodiments, the dose of checkpoint inhibitor is not administered if the disease progresses or unacceptable toxicity occurs.

In some embodiments, the cancer is a solid tumor. In some embodiments of the method, the cancer is or comprises a carcinoma, sarcoma, or melanoma. In some embodiments, the cancer is a lung cancer, skin cancer, head and neck cancer, hematological cancer, breast cancer, pancreatic cancer, colorectal cancer, gastrointestinal cancer, gastric cancer, thyroid cancer, brain cancer, prostate cancer, kidney cancer, uterine cancer, cervical cancer, ovarian cancer, liver cancer, and/or testicular cancer. In some embodiments, the cancer is non-small cell lung cancer (NSCLC), small cell lung cancer (SCLC), renal cell carcinoma (RCC), squamous cell carcinoma of the head and neck (SCCHN), papillary thyroid cancer, classical Hodgkin lymphoma (cHL) primary mediastinal large B-cell lymphoma (PMBCL), soft-tissue sarcoma, liposarcoma (e.g., a dedifferentiated liposarcoma), leiomyosarcoma, carcinoma or adenocarcinoma of the prostate or a prostatic intraepithelial neoplasia, squamous cell carcinoma, gastric adenocarcinoma, melanoma, triple-negative breast cancer (TNBC), and combinations thereof. In some embodiments, the cancer comprises progressive metastatic disease, or progressive locally advanced diseases not amenable to local therapy.

In some embodiments, administering of the antibody that specifically binds to hCD163 (i.e., on a myeloid cell) and the immune checkpoint inhibitor promotes proliferation of CD3+ T cells in the subject as demonstrated in one or more in vitro assays comprising cells of the subject. In some embodiments, the administering of the immunomodulatory CD163 antibody that specifically binds to hCD163 and the immune checkpoint inhibitor promotes cytokine secretion in the subject. In some embodiments, the administering of the immunomodulatory CD163 antibody that specifically binds to hCD163 and the immune checkpoint inhibitor promotes T cell-mediated killing of tumor cells in the subject. In some embodiments, the treatment promotes an immune cell function as measured by one or both of the following parameters: activation of a CD4+ T cell, CD8+ T cell, NK cell, or any combination thereof; and proliferation of a CD4+ T cell, CD8+ T cell, NK cell, or any combination thereof. In some embodiments, the activation of a CD4+ T cell, CD8+ T cell, NK cell, or any combination thereof is measured as an increased level of a cytokine and/or peforin. In some embodiments, the cytokine is selected from the group consisting of IFN-γ, TNF-α, IL2, and any combination thereof. In some embodiments, the increased level of cytokine or perforin is increased as compared to a level in the absence of both the immunomodulatory CD163 antibody and the immune checkpoint inhibitor.

In some embodiments, treatment with a combination of the immunomodulatory CD163 antibody and the immune checkpoint inhibitor results in increased immunostimulatory activity in a tumor microenvironment. In some embodiments, the immunostimulatory activity in the tumor microenvironment is assessed via biopsy or in situ scanning.

In some embodiments, a subject has been selected based on detection of a biomarker in a sample from the subject indicating susceptibility of the cancer to treatment with an immune checkpoint inhibitor. In some embodiments, the biomarker is PD-L1. In some embodiments, cancer has been detected in the subject. In some embodiments, cancer cells from the subject have been characterized as expressing PD-L1 and, optionally, at a level higher than non-cancer cells of the subject or a control subject. In some such embodiments, a step comprising confirming PD-L1 expression by the cancer cells before commencing administration of the combination of the immunomodulatory CD163 antibody and the immune checkpoint inhibitor is performed.

Disclosed herein, in certain embodiments, are methods of treating a PD-L1-expressing cancer in a subject in need thereof, comprising administering to the subject an immunomodulatory CD163 antibody or fragment thereof that specifically binds to hCD163 expressed on a myeloid cell of the subject and (b) a PD-1 antagonist or a PD-L1 antagonist. In some embodiments, the cancer is renal cell carcinoma (RCC), breast cancer, colorectal cancer, gastric cancer, non-small cell lung cancer (NSCLC), papillary thyroid cancer, or testicular cancer.

Disclosed herein, in certain embodiments, are methods of treating a cancer in a subject in need thereof, wherein PD-L1 is expressed by cells of the cancer or immunosuppressive cells of the subject and wherein the treatment comprises administering to the subject an immunomodulatory CD163 antibody that specifically binds to hCD163 expressed on a myeloid cell of the subject and (b) a PD-1 antagonist or a PD-L1 antagonist.

In some embodiments, the immunosuppressive cells comprise antigen-presenting cells, dendritic cells, macrophages, fibroblasts, or T cells. In some embodiments, a step comprising detecting a genomic alteration in cells of the cancer, which genomic alteration is associated with increased expression of PD-L1 by the cancer cells is performed.

In some embodiments, the cancer is classical Hodgkin lymphoma (cHL) primary mediastinal large B-cell lymphoma (PMBCL), non-small cell lung cancer (NSCLC), squamous cell carcinoma, or gastric adenocarcinoma. In some embodiments, a predictive biomarker for: increased expression of PD-L1 in cells of the cancer has been detected and/or susceptibility to a PD-1 or PD-L1 antagonist has been detected, wherein the biomarker is selected from increased histone acetylation, increased methylation of histone H3 on lysine 4, expression of zeste homolog 2 (EZH2), overactivity or overexpression of MYC, upregulation of ALK, loss of p53 function, post-translational N-linked glycosylation, serine/threonine or tyrosine phosphorylation, polyubiquitination, palmitoylation of PD-L1, exosomal PD-L1, soluble PD-L1 or splicing variants thereof, or mutation or hyperactivation of HIF1/2α, NF-κB, MAPK, PTEN/PI3K and/or EGFR pathways.

In some embodiments, a MEK inhibitor is administered to the subject in an amount effective to decrease expression of PD-L1 by the cancer.

In some embodiments, the cancer is pancreatic cancer.

In some embodiments, the subject is not known to be BRAF inhibitor refractory and the method further comprises administering an effective amount of a BRAF inhibitor. In some embodiments, the subject has been selected by assessing a relevant biological sample from the subject for one or more of the following predictive biomarkers: high number of tumor-associated macrophages, high number of M2-like macrophages, high number of myeloid-derived suppressor cells, high expression of CD163 by macrophages, high ratio of M2 (CD206+) to M1 (CD11c+) macrophages, and high ratio of M2 (CD163+) to M1 (CD163−) macrophages among tumor-associated (CD68+) macrophages.

Disclosed herein, in certain embodiments, are methods of characterizing an agent or a combination of agents comprising an in vitro assay which comprises steps of (a) contacting a cell preparation with a combination of (i) an immunomodulatory CD163 antibody that specifically binds to hCD163 and (ii) an immune checkpoint inhibitor; and (b) measuring expression or production of IL2, TNF-α, perforin, and/or IFN-γ, and comparing expression or production thereof to that by cells contacted with the immunomodulatory CD163 antibody or the immune checkpoint inhibitor alone. In some embodiments, the immunomodulatory CD163 antibody binding to the human myeloid cell potentiates an immune response in the subject as measured by at least one in vitro assay using cells from the subject. In some embodiments, the potentiated immune response in the subject is greater than when the immunomodulatory CD163 antibody binds in the absence of the immune checkpoint inhibitor. In some embodiments, the immunomodulatory CD163 antibody binding to the myeloid cell promotes an immunostimulatory function of the cell. In some embodiments, an immune response in the subject as measured by at least one in vitro assay using cells from the subject. In some embodiments, the immunostimulatory function of the cell is greater than when the immunomodulatory CD163 antibody binds in the absence of the immune checkpoint inhibitor.

Disclosed herein, in certain embodiments, are methods of treating a cancer using immunotherapy in a subject in need thereof, comprising administering to the subject a therapeutically effective amount of a combination comprising (a) an immunomodulatory CD163 antibody comprising a hCD163-binding domain and (b) a PD-L1 antagonist.

Disclosed herein, in certain embodiments, are methods of treating a cancer in a subject in need thereof, comprising administering to the subject a therapeutically effective amount of a combination comprising (a) an immunomodulatory CD163 antibody comprising a hCD163-binding domain and (b) a PD-L1 antagonist.

Disclosed herein, in certain embodiments, are methods of treating a cancer using immunotherapy in a subject in need thereof, comprising administering to the subject a therapeutically effective amount of a combination comprising (a) an immunomodulatory CD163 antibody that specifically binds to CD163 expressed on a human macrophage and (b) a PD-L1 antagonist.

Disclosed herein, in certain embodiments, are methods of treating a cancer in a subject in need thereof, comprising administering to the subject a therapeutically effective amount of a combination comprising (a) an immunomodulatory CD163 antibody that specifically binds to CD163 expressed on a human macrophage and (b) a PD-L1 antagonist. In some embodiments, the immunomodulatory CD163 antibody binding alters expression of at least one marker on the macrophage selected from CD16, CD64, TLR2, and Siglec-15.

Disclosed herein, in certain embodiments, are methods of treating a cancer in a subject in need thereof, comprising administering to the subject (i) an immunomodulatory CD163 antibody that specifically binds to hCD163; and (ii) an immune checkpoint inhibitor selected from a PD-1 antagonist or a PD-L1 antagonist.

Disclosed herein, in certain embodiments, are methods of treating a cancer using immunotherapy in a subject in need thereof, comprising administering to the subject a therapeutically effective amount of a combination comprising (a) an immunomodulatory CD163 antibody comprising a hCD163-binding domain and (b) a PD-1 antagonist.

Disclosed herein, in certain embodiments, are methods of treating a cancer using immunotherapy in a subject in need thereof, comprising administering to the subject a therapeutically effective amount of a combination comprising (a) an immunomodulatory CD163 antibody that specifically binds to CD163 expressed on a human macrophage and (b) a PD-1 antagonist.

Disclosed herein, in certain embodiments, are methods of treating a cancer using immunotherapy in a subject in need thereof wherein the subject has previously had to stop treatment with a PD-1 antagonist due to toxicity from the treatment and wherein the subject is treated by administering to the subject a therapeutically effective amount of a combination comprising (a) an immunomodulatory CD163 antibody that specifically binds to human CD163 expressed on a human macrophage and (b) a PD-1 antagonist, wherein the dose of the PD-1 antagonist in the combination treatment is lower than the dose that the patient received and had to stop.

Disclosed herein, in certain embodiments, are combination products comprising an immunomodulatory CD163 antibody and an immune checkpoint inhibitor. In some embodiments, a combination product comprises an immunomodulatory CD163 antibody and an immune checkpoint inhibitor selected from a PD-1 antagonist and a PD-L1 antagonist for use as a medicament, wherein the immunomodulatory CD163 antibody comprises a light chain variable domain (V_{L}) having a sequence at least 80% identical to the amino acid sequence selected from the group consisting of: SEQ ID NO: 28, SEQ ID NO: 30, SEQ ID NO: 32, SEQ ID NO: 34, SEQ ID NO: 36, SEQ ID NO: 38, and SEQ ID NO: 40, and a heavy chain variable domain (V_{H}) having a sequence at least 80% identical to the amino acid sequence selected from the group consisting of: SEQ ID NO: 29, SEQ ID NO: 31, SEQ ID NO: 33, SEQ ID NO: 35, SEQ ID NO: 37, SEQ ID NO: 39, and SEQ ID NO: 41. In some embodiments, the product is for use in treatment of cancer.

Disclosed herein, in certain embodiments, are combinations comprising an immunomodulatory CD163 antibody and an immune checkpoint inhibitor. In some embodiments, the present disclosure provides a combination comprising (i) an immunomodulatory CD163 antibody comprising a light chain variable domain (V_{L}) having a sequence at least 80% identical to the amino acid sequence selected from the group consisting of: SEQ ID NO: 28, SEQ ID NO: 30, SEQ ID NO: 32, SEQ ID NO: 34, SEQ ID NO: 36, SEQ ID NO: 38, and SEQ ID NO: 40, and a heavy chain variable domain (V_{H}) having a sequence at least 80% identical to the amino acid sequence selected from the group consisting of: SEQ ID NO: 29, SEQ ID NO: 31, SEQ ID NO: 33, SEQ ID NO: 35, SEQ ID NO: 37, SEQ ID NO: 39, and SEQ ID NO: 41; and (ii) an immune checkpoint inhibitor selected from a PD-1 antagonist and a PD-L1 antagonist, wherein the combination is used in the preparation of a medicament for the treatment of cancer.

Disclosed herein, in certain embodiments, are methods of relieving T cell suppression in a tumor microenvironment comprising contacting a tumor microenvironment with (i) an immunomodulatory CD163 antibody comprising: a light chain variable domain (V_{L}) having a sequence at least 80% identical to the amino acid sequence selected from the group consisting of: SEQ ID NO: 28, SEQ ID NO: 30, SEQ ID NO: 32, SEQ ID NO: 34, SEQ ID NO: 36, SEQ ID NO: 38, and SEQ ID NO: 40, and a heavy chain variable domain (V_{H}) having a sequence at least 80% identical to the amino acid sequence selected from the group consisting of: SEQ ID NO: 29, SEQ ID NO: 31, SEQ ID NO: 33, SEQ ID NO: 35, SEQ ID NO: 37, SEQ ID NO: 39, and SEQ ID NO: 41; and (ii) an immune checkpoint inhibitor selected from a PD-1 antagonist and a PD-L1 antagonist. In some embodiments, the T cell suppression is measured by an increase in IFN-γ, TNF-α, perforin, or IL2. In some embodiments, the increase is relative to levels prior to the administration of the immunomodulatory CD163 antibody and the immune checkpoint inhibitor.

Disclosed herein, in certain embodiments, are methods of promoting an immune cell function in a subject in need thereof, the method comprising: administering to the subject a combination comprising (a) an immunomodulatory CD163 antibody comprising a light chain variable domain (V_{L}) having a sequence at least 80% identical to the amino acid sequence selected from the group consisting of: SEQ ID NO: 28, SEQ ID NO: 30, SEQ ID NO: 32, SEQ ID NO: 34, SEQ ID NO: 36, SEQ ID NO: 38, and SEQ ID NO: 40, and a heavy chain variable domain (V_{H}) having a sequence at least 80% identical to the amino acid sequence selected from the group consisting of: SEQ ID NO: 29, SEQ ID NO: 31, SEQ ID NO: 33, SEQ ID NO: 35, SEQ ID NO: 37, SEQ ID NO: 39, and SEQ ID NO: 41; and (b) an immune checkpoint inhibitor, in which the combination is effective to promote an immune cell function measured by: (i) activation of a CD4+ T cell, CD8+ T cell, NK cell, or any combination thereof; (ii) proliferation of a CD4+ T cell, CD8+ T cell, NK cell, or any combination thereof; or (iii) a greater proportion of Th1 cells as compared to Th2 cells, and wherein the measurement of (i), (ii), and/or (iii) is performed using an in vitro assay using cells from the subject.

In some embodiments, the activation of a CD4+ T cell, CD8+ T cell, NK cell, or any combination thereof is measured as an increased level of IL2, IFN-γ, TNF-α, or perforin, or any combination thereof as measured after administration of the immunomodulatory CD163 antibody and the immune checkpoint inhibitor as compared to prior to administration of the immunomodulatory CD163 antibody and the immune checkpoint inhibitor. In some embodiments, the cell is an immunosuppressive cell. In some embodiments, the immunosuppressive human myeloid cell is a macrophage. In some embodiments, the immunosuppressive human myeloid cell is a myeloid-derived suppressor cell.

Disclosed herein, in certain embodiments, are methods of treating a cancer in an individual in need thereof, the method comprising administering to the individual a therapeutically effective amount of an immunomodulatory CD163 antibody comprising a light chain variable domain (V_{L}) having a sequence at least 80% identical to the amino acid sequence selected from the group consisting of: SEQ ID NO: 28, SEQ ID NO: 30, SEQ ID NO: 32, SEQ ID NO: 34, SEQ ID NO: 36, SEQ ID NO: 38, and SEQ ID NO: 40, and a heavy chain variable domain (V_{H}) having a sequence at least 80% identical to the amino acid sequence selected from the group consisting of: SEQ ID NO: 29, SEQ ID NO: 31, SEQ ID NO: 33, SEQ ID NO: 35, SEQ ID NO: 37, SEQ ID NO: 39, and SEQ ID NO: 41; and an immune checkpoint inhibitor selected from a PD-1 antagonist and a PD-L1 antagonist, whereby immunosuppression by a tumor-associated macrophage in the individual is reduced. In some embodiments, T cell-mediated tumor cell killing in the individual is increased.

Disclosed herein, in certain embodiments, are methods of reducing a tumor promoting activity of a tumor-associated macrophage in an individual in need thereof, the method comprising administering to the individual a therapeutically effective amount of each of an immunomodulatory CD163 antibody comprising a light chain variable domain (V_{L}) having a sequence at least 80% identical to the amino acid sequence selected from the group consisting of: SEQ ID NO: 28, SEQ ID NO: 30, SEQ ID NO: 32, SEQ ID NO: 34, SEQ ID NO: 36, SEQ ID NO: 38, and SEQ ID NO: 40, and a heavy chain variable domain (V_{H}) having a sequence at least 80% identical to the amino acid sequence selected from the group consisting of: SEQ ID NO: 29, SEQ ID NO: 31, SEQ ID NO: 33, SEQ ID NO: 35, SEQ ID NO: 37, SEQ ID NO: 39, and SEQ ID NO: 41; and an immune checkpoint inhibitor selected from a PD-1 antagonist and a PD-L1 antagonist, wherein the administration results in modulation of a CD4+ T cell activation, CD4+ T cell proliferation, CD8+ T cell activation, CD8+ T cell proliferation, or any combination. In some embodiments, the immunomodulatory CD163 antibody binds to a macrophage in a tumor microenvironment.

Disclosed herein, in certain embodiments, are methods of modulating an activity of a tumor-associated macrophage in a tumor microenvironment, the method comprising contacting the tumor-associated macrophage with an immunomodulatory CD163 antibody comprising a light chain variable domain (V_{L}) having a sequence at least 80% identical to the amino acid sequence selected from the group consisting of: SEQ ID NO: 28, SEQ ID NO: 30, SEQ ID NO: 32, SEQ ID NO: 34, SEQ ID NO: 36, SEQ ID NO: 38, and SEQ ID NO: 40, and a heavy chain variable domain (V_{H}) having a sequence at least 80% identical to the amino acid sequence selected from the group consisting of: SEQ ID NO: 29, SEQ ID NO: 31, SEQ ID NO: 33, SEQ ID NO: 35, SEQ ID NO: 37, SEQ ID NO: 39, and SEQ ID NO: 41; and a PD-L1 antagonist, wherein the contacting results in at least one of the following effects: (a) reduced expression of at least one marker on the tumor-associated macrophage, wherein the at least one marker is CD16, CD64, TLR2, or Siglec-15; (b) the immunomodulatory CD163 antibody is internalized by the tumor-associated macrophage; (c) the contacting of the tumor and/or binding of the immunomodulatory CD163 antibody is not cytotoxic to the tumor-associated macrophage; (d) increase in levels of IFN-γ, TNF-α, and/or perforin; (e) activation of a CD4+ T cell, CD8+ T cell, NK cell, or any combination thereof; (f) proliferation of a CD4+ T cell, CD8+ T cell, NK cell, or any combination thereof; and (g) promotion of tumor cell killing in the tumor microenvironment. In some embodiments, the contacting with the immunomodulatory CD163 antibody results in binding of the immunomodulatory CD163 antibody and, wherein the binding results in: two or more of (a) through (g); three or more of (a) through (g); four or more of (a) through (g); five or more of (a) through (g); six or more of (a) through (g); or all of (a) through (g), and the binding is additively or synergistically increased in the presence of an immune checkpoint inhibitor.

In some embodiments, the immunomodulatory CD163 antibody and the immune checkpoint inhibitor are additive or synergize. In some embodiments, each of the immunomodulatory CD163 antibody and the immune checkpoint inhibitor are present in subtherapeutic amounts relative to therapeutic amounts as a monotherapy. In some embodiments, each of the immunomodulatory CD163 antibody and the immune checkpoint inhibitor are present in amounts that would have therapeutic effect if given as a monotherapy.

In some embodiments, an immunomodulatory CD163 antibody specifically binds to a human CD163 protein expressed on an immunosuppressive human myeloid cell wherein binding of the immunomodulatory CD163 antibody to the myeloid cell occurs in the presence of an immune checkpoint inhibitor selected from a PD-1 antagonist and a PD-L1 antagonist and promotes an immune cell function as measured by one or both of the following parameters: (i) activation of a CD4+ T cell, CD8+ T cell, NK cell, or any combination thereof; and (ii) proliferation of a CD4+ T cell, CD8+ T cell, NK cell, or any combination thereof, wherein the activation and/or proliferation is increased as compared to binding of the immunomodulatory CD163 antibody in the absence of the PD-1 antagonist or PD-L1 antagonist.

In some embodiments, an immunomodulatory CD163 antibody specifically binds to a human CD163 protein expressed on a human myeloid cell, wherein binding of the immunomodulatory CD163 antibody to the myeloid cell occurs in the presence of an immune checkpoint inhibitor selected from a PD-1 antagonist and a PD-L1 antagonist and promotes an immune cell function as measured by one or both of the following parameters: (i) activation of a CD4+ T cell, CD8+ T cell, NK cell, or any combination thereof; and (ii) proliferation of a CD4+ T cell, CD8+ T cell, NK cell, or any combination thereof, wherein the activation and/or proliferation is increased as compared to binding of the immunomodulatory CD163 antibody in the absence of the PD-1 antagonist or PD-L1 antagonist. In some embodiments, the immune cell function is in a tumor microenvironment. In some embodiments, the immune cell function is in vivo.

In some embodiments, an immunomodulatory CD163 antibody specifically binds to a human CD163 protein expressed on a human myeloid cell, wherein binding of the immunomodulatory CD163 antibody to the myeloid cell occurs in the presence of an immune checkpoint inhibitor selected from a PD-1 antagonist and a PD-L1 antagonist and increases an immunostimulatory activity in a tumor microenvironment. In some embodiments, the tumor microenvironment is in vivo. In some embodiments, the binding of the immunomodulatory CD163 antibody to the myeloid cell reduces an immunosuppression activity of the myeloid cell. In some embodiments, the binding of the immunomodulatory CD163 antibody to the myeloid cell reduces a tumor promoting activity of the myeloid cell. In some embodiments, the myeloid cell is a macrophage. In some such embodiments, macrophage is a tumor associated macrophage or an M2 or M2-like macrophage. In some embodiments, the M2 or M2-like macrophage is an M2a, M2b, M2c, or M2d macrophage. In some embodiments, the CD163 antibody alters expression of at least one marker on the myeloid cell.

In some embodiments, binding to the hCD163 protein in the presence of the PD-1 antagonist or PD-L1 antagonist results in additivity or synergy that achieves greater expression of CD69, ICOS, OX40, PD-1, LAG3, CTLA-4, or any combination thereof by CD4+ T cells as compared to binding of the immunomodulatory CD163 antibody in the absence of the immune checkpoint inhibitor. In some embodiments, binding to the hCD163 protein in the presence of the PD-1 antagonist or PD-L1 antagonist promotes greater CD8+ T cell activation, greater CD8+ T cell proliferation, or both greater CD8+ T cell activation and proliferation as compared to the binding of the immunomodulatory CD163 antibody in the absence of the immune checkpoint inhibitor.

In some embodiments, binding to the hCD163 protein in the presence of the PD-1 antagonist or PD-L1 antagonist promotes greater expression of ICOS, OX40, PD1, LAG3, CTLA-4, or any combination thereof by CD8+ T cells as compared to the binding of the immunomodulatory CD163 antibody in the absence of the immune checkpoint inhibitor. In some embodiments, binding to the hCD163 protein in the presence of the PD-1 antagonist or PD-L1 antagonist reduces immunosuppression in a tumor microenvironment more than binding to the hCD163 protein in the absence of the PD-1 antagonist or PD-L1 antagonist. In some embodiments, binding to the hCD163 protein in the presence of the PD-1 antagonist or PD-L1 antagonist promotes increased expression of IFN-γ or IL-2 by T cells.

In some embodiments, binding to the hCD163 protein in the presence of the immune checkpoint inhibitor promotes skewing of Th1 cells to increase a proportion of Th1 cells as compared to Th2 cells as compared to binding to the hCD163 protein in the absence of the immune checkpoint inhibitor.

In some embodiments, the PD-1 antagonist is itself an antibody. In some embodiments, the PD-1 antibody is selected from the group consisting of nivolumab, pembrolizumab, cemiplimab, dostarlimab, JTX-4014, spartalizumab, camrelizumab, sintilimab, tislelizumab, toripalimab, INCMGA00012, AMP-224, AMP-514, zimberelimab, and fragments or combinations thereof. In some embodiments, the PD-1 antagonist comprises a PD-1 binding domain comprising CDRs of an antibody selected from the group consisting of nivolumab, pembrolizumab, cemiplimab, JTX-4014, spartalizumab, camrelizumab, sintilimab, tislelizumab, toripalimab, INCMGA00012, AMP-224, or AMP-514, zimberelimab, and fragments or combinations thereof. In some embodiments, the PD-1 antagonist is a small molecule. In some embodiments, the PD-1 antagonist is a rationally designed peptide antagonist of PD-1.

In some embodiments, the PD-L1 antagonist is an antibody. In some embodiments, the PD-L1 antibody is selected from the group consisting of avelumab, durvalumab, atezolizumab, envafolimab, cosibelimab (CK-301), LY3300054, CA-170, BMS-936559, and fragments or combinations thereof. In some embodiments, the PD-L1 antagonist comprises a PD-L1 binding domain comprising CDRs of an antibody selected from the group consisting of avelumab, durvalumab, atezolizumab, envafolimab, cosibelimab (CK-301), LY3300054, CA-170, BMS-936559, and fragments or combinations thereof. In some embodiments, the PD-L1 antagonist is a rationally-designed peptide antagonist of PD-L1, such as AUNP-12 or BMS-986189.

Disclosed herein, in certain embodiments, are methods of promoting an immune cell function, the method comprising: specifically binding an antibody to a CD163 protein expressed on an immunosuppressive human myeloid cell in the presence of an immune checkpoint inhibitor selected from a PD-1 antagonist and a PD-L1 antagonist; and promoting an immune cell function as measured by one or both of the following parameters: (i) activation of a CD4+ T cell, CD8+ T cell, NK cell, or any combination thereof; and (ii) proliferation of a CD4+ T cell, CD8+ T cell, NK cell, or any combination thereof, wherein the activation and/or proliferation is increased as compared to binding of the immunomodulatory CD163 antibody in the absence of the PD-1 antagonist or PD-L1 antagonist.

Disclosed herein, in certain embodiments, are methods of treating a cancer in an individual in need thereof, the method comprising administering to the individual a therapeutically effective amount of an immunomodulatory CD163 antibody that binds to a human myeloid cell in the presence of an immune checkpoint inhibitor according to an immunomodulatory CD163 antibody for use with the method disclosed herein, whereby immunosuppression by a tumor-associated macrophage in the individual is reduced. In some embodiments, the immunomodulatory CD163 antibody that binds to hCD163 comprises a pair of variable domains consisting of a light chain variable domain and a heavy chain variable domain, wherein the pair is represented by a pair of amino acid sequences selected from the group consisting of: (a) SEQ ID NOs: 28 and 29; (b) SEQ ID NOs: 30 and 31; (c) SEQ ID NOs: 32 and 33; (d) SEQ ID NOs: 34 and 35; (e) SEQ ID NOs: 36 and 37; (f) SEQ ID NOs: 38 and 39; and (g) SEQ ID NOs: 40 and 41.

Disclosed herein, in certain embodiments, are methods of treating a cancer in an individual in need thereof, the method comprising administering to the individual a therapeutically effective amount of an immunomodulatory CD163 antibody in the presence of an immune checkpoint inhibitor selected from a PD-1 antagonist and a PD-L1 antagonist, whereby T cell-mediated tumor cell killing in the individual is increased and/or T cell exhaustion is relieved.

In some embodiments, the immunomodulatory CD163 antibody comprises a heavy chain variable domain and a light chain variable domain, together comprising six CDRs as set forth in Tables 2 and 3, wherein the amino acid sequences of the six CDRs are selected from the group consisting of: (a) SEQ ID NOs: 1, 2, 3, 4, 5, and 6; (b) SEQ ID NOs: 7, 2, 8, 16, 17, and 18; (c) SEQ ID NOs: 7, 9, 10, 19, 20, and 21; (d) SEQ ID NOs: 7, 2, 11, 22, 23, and 24; (e) SEQ ID NOs: 7, 2, 8, 22, 17, and 18; (f) SEQ ID NOs: 7, 2, 10, 16, 17, and 24; and (g) SEQ ID NOs: 7, 2, 12, 19, 17, and 18.

In some embodiments, the immunomodulatory CD163 antibody comprises a heavy chain variable domain and a light chain variable domain, together comprising six CDRs, wherein the amino acid sequences of the six CDRs are set forth as follows:
(a) RASQSISX8YLN (SEQ ID NO: 13), wherein X8=S, R, K, H;
(b) AASSLQX9 (SEQ ID NO: 14), wherein X9=S, N, Q, T;
(c) QQSYSTX10X11GX12 (SEQ ID NO: 15), wherein X10=P, Q, T, S, N, A, G; X11=R, G, A, S; and X12=T, S, A, G, N;
(d) SX₁X₂MH, wherein X1=Y, E, Q, D; and X2=A, D, T, V, S, G, E;
(e) VISX3DGSNKYX4ADSVKG (SEQ ID NO: 26), wherein X3=Y, E, Q, D; and X4=Y, N, H, E, D, K, Q, R; and
(f) ENVRPYYDFWX5GYX6SEYYYYGX7DV (SEQ ID NO: 27), wherein X5=S, R, K, H; X6=Y, S, N, T, A, Q; and X7=M, L, I, V.

In some embodiments, an immune checkpoint inhibitor is a PD-1 antagonist.

In some embodiments, the PD-1 antagonist is a PD-1 antibody. In some embodiments, the PD-1 antibody is an IgG1 antibody or IgG4 antibody. In some embodiments, the PD-1 antibody is selected from the group consisting of nivolumab, pembrolizumab, cemiplimab, dostarlimab, JTX-4014, spartalizumab, camrelizumab, sintilimab, tislelizumab, toripalimab, INCMGA00012, AMP-224, or AMP-514, zimberelimab, and fragments or combinations thereof. In some embodiments, the PD-1 antagonist comprises a PD-1 binding domain comprising CDRs of an antibody selected from the group consisting of nivolumab, pembrolizumab, cemiplimab, dostarlimab, JTX-4014, spartalizumab, camrelizumab, sintilimab, tislelizumab, toripalimab, INCMGA00012, AMP-224, AMP-514, zimberelimab, and fragments or combinations thereof.

In some embodiments, the PD-1 antagonist is a small molecule.

In some embodiments, the PD-1 antagonist is a rationally designed peptide antagonist of PD-1.

In some embodiments, the PD-L1 antagonist is an PD-L1 antibody. In some embodiments, the PD-L1 antibody is an IgG1 antibody or IgG4 antibody. In some embodiments, the PD-L1 antibody is selected from the group consisting of avelumab, durvalumab, atezolizumab, envafolimab, cosibelimab, LY3300054, CA-170, BMS-936559, and fragments or combinations thereof. In some embodiments, the PD-L1 antagonist comprises a PD-L1-binding domain comprising CDRs of an antibody selected from the group consisting of avelumab, durvalumab, or atezolizumab, envafolimab, cosibelimab (CK-301), LY3300054, CA-170, BMS-936559, and fragments or combinations thereof. In some embodiments, the PD-L1 antagonist is a peptide (e.g., AUNP-12 or BMS-986189).

In some embodiments, an antagonist inhibits the interaction between PD-1 and PDL-1. In some embodiments, the antagonist is a macrocyclic compound (e.g., gramicidin S and derivatives thereof). In some embodiments, the antagonist is an antibiotic such as an ansamycin type antibiotic (e.g., rifabutin). In some embodiments, the antagonist is a phenolic compound (e.g., kaempferol, kaempferol-7-O-rhamnoside, caffeoylquinic acid, 3-O-caffeoylquinic acid, 4-O-caffeoylquinic acid, 5-O-caffeoylquinic acid, ellagic acid). In some embodiments, the antagonist is a heterocyclic compound (e.g., ZINC 67,902,090, ZINC 12,529,904). In some embodiments, the antagonist is a small molecule (e.g., CA-170, ARB-272572, INCB086550). In some embodiments, the antagonist is actinomycin D, amphotericin B, bacitracin, bryostatin, candicidin, clarithromycin, cyclosporin A, cyanocobalamin, erythromycin, everolimus, geldanamycin, ivermectin B1a, macbecin, metocurine, monocrotaline, nystatin, plerixafor, rifampin, sirolimus, troleandomycin, rifabutin, rifapentine, rifamycin SV, formyl rifamycin, rifaximin, gramicidin S, ZINC 67,902,090, ZINC 12,529,904, or derivatives thereof. In some embodiments, the antagonist is cyclo(-Leu-DTrp-Pro-Thr-Asp-Leu-DPhe-Lys(Dde)-Val-Arg) (SEQ ID NO: 46), rifabutin, kaempferol, kaempferol-7-O-rhamnoside, eriodictyol, fisetin, glyasperin C, cosmosiin, ellagic acid, caffeoylquinic acids, or derivatives thereof.

In some embodiments, the immunomodulatory CD163 antibody does not bind to a murine CD163 or any non-human primate CD163. In some embodiments, the immunomodulatory CD163 antibody binds selectively to human CD163, such CD163 expressed on human myeloid cells such as immunosuppressive macrophages. In such cases the immunomodulatory CD163 antibodies may be referred to as “hCD163 antibodies.”

In some embodiments, the immunomodulatory CD163 antibody comprises a pair of variable domains consisting of a light chain variable domain and a heavy chain variable domain, wherein the pair is represented by a pair of amino acid sequences selected from the group consisting of: (a) SEQ ID NOs: 28 and 29; (b) SEQ ID NOs: 30 and 31; (c) SEQ ID NOs: 32 and 33; (d) SEQ ID NOs: 34 and 35; (e) SEQ ID NOs: 36 and 37; (f) SEQ ID NOS: 38 and 39; and (g) SEQ ID NOs: 40 and 41.

In some embodiments, the immunomodulatory CD163 antibody comprises a heavy chain variable domain and a light chain variable domain, together comprising six CDRs as set forth in Tables 2 and 3, as follows, wherein the sequences of the six CDRs are selected from the group consisting of: (a) SEQ ID NOs: 1, 2, 3, 4, 5, and 6; (b) SEQ ID NOs: 7, 2, 8, 16, 17, and 18; (c) SEQ ID NOs: 7, 9, 10, 19, 20, and 21; (d) SEQ ID NOs: 7, 2, 11, 22, 23, and 24; (e) SEQ ID NOs: 7, 2, 8, 22, 17, and 18; (f) SEQ ID NOs: 7, 2, 10, 16, 17, and 24; and (g) SEQ ID NOs: 7, 2, 12, 19, 17, and 18.

Disclosed herein, in certain embodiments, are methods of treating a cancer in a subject in need thereof, comprising administering to the subject: an immunomodulatory CD163 antibody that binds at a localized region on a surface of human CD163 (hCD163) comprising the amino acid sequence IGRVNASKGFGHIWLDSVSCQGHEPAI (SEQ ID NO: 43), VVCRQLGCGSA (SEQ ID NO: 44), and WDCKNWQWGGLTCD (SEQ ID NO: 45); and an immune checkpoint inhibitor.

In some embodiments, the immunomodulatory CD163 antibody comprises: a light chain variable domain (V_{L}) having a sequence at least 80% identical to the amino acid sequence selected from the group consisting of: SEQ ID NO: 28, SEQ ID NO: 30, SEQ ID NO: 32, SEQ ID NO: 34, SEQ ID NO: 36, SEQ ID NO: 38, and SEQ ID NO: 40, and a heavy chain variable domain (V_{H}) having a sequence at least 80% identical to the amino acid sequence selected from the group consisting of: SEQ ID NO: 29, SEQ ID NO: 31, SEQ ID NO: 33, SEQ ID NO: 35, SEQ ID NO: 37, SEQ ID NO: 39, and SEQ ID NO: 41. In some embodiments, the immunomodulatory CD163 antibody specifically binds to an epitope of hCD163, wherein the immunomodulatory CD163 antibody comprises: (i) a light chain variable domain (V_{L}) and a heavy chain variable domain (V_{H}) with amino acid sequences as set forth in SEQ ID NOs: 40 and 41; and/or (ii) six CDRs with amino acid sequences as set forth in SEQ ID NOS: 1, 2, 3, 4, 5, and 6.

In some embodiments, the immune checkpoint inhibitor is selected from the group consisting of an antagonist to an immune checkpoint protein or ligand thereof, wherein the immune checkpoint protein is selected from the group consisting of CTLA-4, PD-1, TIM3, TIGIT, and any combination thereof.

In some embodiments, the immune checkpoint inhibitor is a PD-1 antagonist.

In some embodiments, the PD-1 antagonist is a PD-1 antibody. In some embodiments, the PD-1 antibody is selected from the group consisting of nivolumab, pembrolizumab, cemiplimab, dostarlimab, JTX-4014, spartalizumab, camrelizumab, sintilimab, tislelizumab, toripalimab, INCMGA00012, AMP-224, AMP-514, zimberelimab, and fragments or combinations thereof. In some embodiments, the PD-1 antagonist comprises a PD-1 binding domain comprising CDRs of an antibody selected from the group consisting of nivolumab, pembrolizumab, cemiplimab, dostarlimab, JTX-4014, spartalizumab, camrelizumab, sintilimab, tislelizumab, toripalimab, INCMGA00012, AMP-224, or AMP-514, zimberelimab, and fragments or combinations thereof. In some embodiments, the PD-1 antagonist is a rationally designed peptide antagonist of PD-1.

Disclosed herein, in some embodiments, are methods of treating a cancer in a subject in need thereof, comprising: administering to the subject: a) a therapeutic amount of an immunomodulatory CD163 antibody or antigen-binding fragment thereof comprising: a light chain variable domain (VL) having a sequence at least 80% identical to the amino acid sequence selected from the group consisting of: SEQ ID NO: 28, SEQ ID NO: 30, SEQ ID NO: 32, SEQ ID NO: 34, SEQ ID NO: 36, SEQ ID NO: 38, and SEQ ID NO: 40, and a heavy chain variable domain (VH) having a sequence at least 80% identical to the amino acid sequence selected from the group consisting of: SEQ ID NO: 29, SEQ ID NO: 31, SEQ ID NO: 33, SEQ ID NO: 35, SEQ ID NO: 37, SEQ ID NO: 39, and SEQ ID NO: 41; and b) a therapeutic amount of an immune checkpoint inhibitor. In some embodiments, the therapeutic amount of the CD163 antibody is about 150 milligrams (mg) to about 1200 mg administered intravenously from about once per week to about once per 3 weeks. In some embodiments, the immunomodulatory CD163 antibody comprises a light chain variable domain (VL) having a sequence 100% identical to the amino acid sequence selected from the group consisting of: SEQ ID NO: 28, SEQ ID NO: 30, SEQ ID NO: 32, SEQ ID NO: 34, SEQ ID NO: 36, SEQ ID NO: 38, and SEQ ID NO: 40. In some embodiments, the immunomodulatory CD163 antibody comprises a heavy chain variable domain (VH) having a sequence 100% identical to the amino acid sequence selected from the group consisting of: SEQ ID NO: 29, SEQ ID NO: 31, SEQ ID NO: 33, SEQ ID NO: 35, SEQ ID NO: 37, SEQ ID NO: 39, and SEQ ID NO: 41. In some embodiments, the immunomodulatory CD163 antibody comprises a sequence 100% identical to the amino acid sequence selected from the group consisting of: SEQ ID NO: 4, SEQ ID NO: 16, SEQ ID NO: 19, SEQ ID NO: 22 and SX₁X₂MH, wherein X1=Y, E, Q, D; and X2=A, D, T, V, S, G, E In some embodiments, the immunomodulatory CD163 antibody comprises a sequence 100% identical to the amino acid sequence selected from the group consisting of: SEQ ID NO: 5, SEQ ID NO: 17, SEQ ID NO: 20, SEQ ID NO: 23, and SEQ ID NO: 26. In some embodiments, the immunomodulatory CD163 antibody comprises a sequence 100% identical to the amino acid sequence selected from the group consisting of: SEQ ID NO: 6, SEQ ID NO: 18, SEQ ID NO: 21, SEQ ID NO: 24, and SEQ ID NO: 27. In some embodiments, the immunomodulatory CD163 antibody comprises a sequence 100% identical to the amino acid sequence selected from the group consisting of: SEQ ID NO: 1, SEQ ID NO: 7, and SEQ ID NO: 13. In some embodiments, the immunomodulatory CD163 antibody comprises a sequence 100% identical to the amino acid sequence selected from the group consisting of: SEQ ID NO: 2, SEQ ID NO: 9, and SEQ ID NO: 14. In some embodiments, the immunomodulatory CD163 antibody comprises a sequence 100% identical to the amino acid sequence selected from the group consisting of: SEQ ID NO: 3, SEQ ID NO: 8, SEQ ID NO: 10, SEQ ID NO: 11, SEQ ID NO: 12, and SEQ ID NO: 15 In some embodiments, the immunomodulatory CD163 comprises a sequence 100% identical to the amino acid sequence selected from the group consisting of: (a) SEQ ID NOs: 1, 2, 3, 4, 5, and 6; (b) SEQ ID NOs: 7, 2, 8, 16, 17, and 18; (c) SEQ ID NOs: 7, 9, 10, 19, 20, and 21; (d) SEQ ID NOs: 7, 2, 11, 22, 23, and 24; (e) SEQ ID NOs: 7, 2, 8, 22, 17, and 18; (f) SEQ ID NOs: 7, 2, 10, 16, 17, and 24; and (g) SEQ ID NOs: 7, 2, 12, 19, 17, and 18. In some embodiments, the immunomodulatory CD163 antibody comprises a VL and VH domains having sequences selected from the group consisting of: (a) SEQ ID NOs: 28 and 29; (b) SEQ ID NOs: 30 and 31; (c) SEQ ID NOs: 32 and 33; (d) SEQ ID NOs: 34 and 35; (e) SEQ ID NOs: 36 and 37; (f) SEQ ID NOs: 38 and 39; and (g) SEQ ID NOs: 40 and 41. In some embodiments, the immunomodulatory CD163 antibody comprises sequences selected from the group consisting of: (a) SEQ ID NOs: 1, 2, 3, 4, 5, and 6; (b) SEQ ID NOs: 7, 2, 8, 16, 17, and 18; (c) SEQ ID NOs: 7, 9, 10, 19, 20, and 21; (d) SEQ ID NOs: 7, 2, 11, 22, 23, and 24; (e) SEQ ID NOs: 7, 2, 8, 22, 17, and 18; (f) SEQ ID NOs: 7, 2, 10, 16, 17, and 24; and (g) SEQ ID NOs: 7, 2, 12, 19, 17, and 18. In some embodiments, the administering of the immunomodulatory CD163 antibody and the immune checkpoint inhibitor yields an additive or synergistic effect as compared to administration of either the immunomodulatory CD163 antibody or the immune checkpoint inhibitor alone. In some embodiments, the immunomodulatory CD163 antibody is an IgG1 antibody or IgG4 antibody. In some embodiments, the immunomodulatory CD163 antibody comprises a constant domain that interacts with a macrophage. In some embodiments, the therapeutic amount of the immunomodulatory CD163 antibody and/or the immune checkpoint inhibitor is less than the therapeutic amount required when the immunomodulatory CD163 antibody and/or the immune checkpoint inhibitor is administered as a monotherapy. In some embodiments, the immunomodulatory CD163 antibody binds to a myeloid cell. In some embodiments, the myeloid cell is an immunosuppressive macrophage, a myeloid-derived suppressor cell, or a tumor-associated macrophage. In some embodiments, the immunomodulatory CD163 antibody antagonizes an immunosuppressive function mediated by immune cells (i) directly, through cancer cell-immune cell interactions; and/or (ii) through secreted products of the cancer cells or the immune cells. In some embodiments, the antagonism of the immunosuppressive function is greater than when the immunomodulatory CD163 antibody is administered in the absence of the immune checkpoint inhibitor. In some embodiments, the immunomodulatory CD163 antibody binding promotes proliferation of CD3+ T cells in the subject. In some embodiments, the immunomodulatory CD163 antibody binding promotes proliferation of CD4+ T cells in the subject. In some embodiments, the immunomodulatory CD163 antibody binding promotes proliferation of CD8+ T cells in the subject. In some embodiments, the immunomodulatory CD163 antibody binding promotes proliferation of CD4+ and CD8+ T cells in the subject. In some embodiments, the proliferation of CD3+ T cells in the subject is greater than when the immunomodulatory CD163 antibody binds in the absence of the immune checkpoint inhibitor. In some embodiments, the binding of the immunomodulatory CD163 antibody to the myeloid cell promotes T-cell-mediated killing of cancer cells in the subject. In some embodiments, the immune checkpoint inhibitor inhibits binding of a receptor on a T-cell to its ligand. In some embodiments, the immune checkpoint inhibitor preventing the binding modulates immunosuppression by cancer cells in the subject, wherein at least a portion of the cancer cells express the receptor or its ligand. In some embodiments, the immune checkpoint inhibitor inhibits the inhibitory receptor-mediated or ligand-mediated immunosuppression by the cancer cell or other immunosuppressive cell in a tumor microenvironment (TME), and the immunomodulatory CD163 antibody promotes an immunostimulatory response against the cancer cell. In some embodiments, the immune checkpoint inhibitor is selected from the group consisting of an antagonist to an immune checkpoint protein or ligand thereof, wherein the immune checkpoint protein is selected from the group consisting of CTLA-4, PD-1, PD-L1, TIM3, LAG3, TIGIT, and any combination thereof. In some embodiments, the immune checkpoint inhibitor is a PD-1 antagonist. In some embodiments, the PD-1 antagonist is a PD-1 antibody. In some embodiments, the PD-1 antibody is an IgG1 antibody or IgG4 antibody. In some embodiments, the PD-1 antibody is selected from the group consisting of nivolumab, pembrolizumab, cemiplimab, dostarlimab, JTX-4014, spartalizumab, camrelizumab, sintilimab, tislelizumab, toripalimab, INCMGA00012, AMP-224, AMP-514, zimberelimab, and fragments or combinations thereof. In some embodiments, the PD-1 antagonist comprises a PD-1 binding domain comprising CDRs of an antibody selected from the group consisting of nivolumab, pembrolizumab, cemiplimab, dostarlimab, JTX-4014, spartalizumab, camrelizumab, sintilimab, tislelizumab, toripalimab, INCMGA00012, AMP-224, AMP-514, zimberelimab, and fragments or combinations thereof. In some embodiments, the PD-1 antagonist is a small molecule. In some embodiments, the PD-1 antagonist is a rationally-designed peptide antagonist of PD-1. In some embodiments, (a) the binding of the immunomodulatory CD163 antibody to the myeloid cell potentiates an immune response disinhibited by the PD-1 antagonist; (b) wherein the PD-1 antagonist disinhibits immunosuppression by cancer cells in the subject and the immunomodulatory CD163 antibody binding promotes immunostimulatory responses against the cancer cells; and/or (c) wherein the PD-1 antagonist inhibits PD-1 mediated immunosuppression by cancer cells in the subject and the immunomodulatory CD163 antibody promotes an immunostimulatory response against the cancer cells. In some embodiments, the immune checkpoint inhibitor is a PD-L1 antagonist. In some embodiments, the PD-L1 antagonist is a PD-L1 antibody. In some embodiments, the PD-L1 antibody is an IgG1 antibody or IgG4 antibody. In some embodiments, the PD-L1 antagonist is selected from the group consisting of avelumab, durvalumab, atezolizumab, envafolimab, cosibelimab (CK-301), LY3300054, CA-170, BMS-936559, and combinations and active fragments thereof. In some embodiments, the PD-L1 antagonist comprises AUNP-12, BMS-986189, a PD-L1 binding domain comprising CDRs of an antibody selected from the group consisting of avelumab, durvalumab, atezolizumab, envafolimab, cosibelimab (CK-301), LY3300054, CA-170, and BMS-936559, and active fragments thereof, or combinations thereof. In some embodiments, (a) the binding of the immunomodulatory CD163 antibody to the myeloid cell potentiates an immune response disinhibited by the PD-L1 antagonist; (b) wherein the PD-L1 antagonist disinhibits immunosuppression by cancer cells in the subject and the immunomodulatory CD163 antibody binding promotes immunostimulatory responses against the cancer cells; and/or (c) wherein the PD-L1 antagonist inhibits PD-L1 mediated immunosuppression by cancer cells in the subject and the immunomodulatory CD163 antibody promotes an immunostimulatory response against the cancer cells. In some embodiments, the immunomodulatory CD163 antibody and the immune checkpoint inhibitor are administered concomitantly or sequentially. In some embodiments, the sequential administration comprises administering the immunomodulatory CD163 antibody prior to administering the immune checkpoint inhibitor. In some embodiments, the sequential administration comprises administering the immune checkpoint inhibitor prior to administering the immunomodulatory CD163 antibody. In some embodiments, when administration is sequential, an interval between administration of the immunomodulatory CD163 antibody and administration of the immune checkpoint inhibitor is between one hour and thirty days. In some embodiments, the interval is about three weeks. In some embodiments, the therapeutic amount of the CD163 antibody is about 150 milligrams (mg) to about 1200 milligrams. In some embodiments, the immunomodulatory CD163 antibody is administered intravenously or subcutaneously. In some embodiments, more than one dose of the immunomodulatory CD163 antibody is administered. In some embodiments, the administration of the immunomodulatory CD163 antibody is over a 30-minute period. In some embodiments, administration of the immunomodulatory CD163 antibody occurs once per week. In some embodiments, the weekly administration of the immunomodulatory CD163 antibody is repeated for at least two, three, four, five, six, seven, eight, nine, ten, eleven, or twelve weeks. In some embodiments, the immune checkpoint inhibitor is administered intravenously or subcutaneously. In some embodiments, more than one dose of the immune checkpoint inhibitor is administered. In some embodiments, the administration of the immune checkpoint inhibitor is over a 30-minute period. In some embodiments, the 30-minute period is the same 30-minute period In some embodiments, the 30-minute period is a different 30-minute period. In some embodiments, a therapeutic amount of pembrolizumab is about 200 mg or about 400 mg. In some embodiments, the therapeutic amount of pembrolizumab is administered more than once. In some embodiments, when the therapeutic amount of pembrolizumab is about 200 mg, it is administered once every three weeks, and when the therapeutic amount of pembrolizumab is about 400 mg, it is administered once every six weeks. In some embodiments, the cemiplimab is administered at a therapeutic dose of about 350 mg. In some embodiments, the therapeutic amount of cemiplimab is administered more than once. In some embodiments, the therapeutic amount of cemiplimab is administered once every three weeks. In some embodiments, the nivolumab is administered at a therapeutic amount of about 240 mg or about 480 mg. In some embodiments, the therapeutic amount of nivolumab is administered more than once. In some embodiments, when the therapeutic amount of nivolumab is about 240 mg it is administered once every two weeks, and when the therapeutic amount of nivolumab is about 480 mg it is administered once every four weeks. In some embodiments, the therapeutic amount is not administered if disease progression or unacceptable toxicity occurs.

Disclosed herein, in some embodiments, are methods of treating a cancer in a subject in need thereof, comprising: administering to the subject: A) a therapeutic amount of an immunomodulatory CD163 antibody or antigen-binding fragment thereof comprising: (i) a light chain variable domain (VL) having amino acid sequences: (a) RASQSISX8YLN (SEQ ID NO: 13), wherein X8=S, R, K, H; (b) AASSLQX9 (SEQ ID NO: 14), wherein X9=S, N, Q, T; (c) QQSYSTX10X11GX12 (SEQ ID NO: 15), wherein X10=P, Q, T, S, N, A, G; X11=R, G, A, S; and X12=T, S, A, G, N; and (ii) a heavy chain variable domain (VH) having amino acid sequences: (a) SX₁X₂MH, wherein X1=Y, E, Q, D; and X2=A, D, T, V, S, G, E; (b) VISX3DGSNKYX4ADSVKG (SEQ ID NO: 26), wherein X3=Y, E, Q, D; and X4=Y, N, H, E, D, K, Q, R; and (c) ENVRPYYDFWX5GYX6SEYYYYGX7DV (SEQ ID NO: 27), wherein X5=S, R, K, H; X6=Y, S, N, T, A, Q; and X7=M, L, I, V; and B) a therapeutic amount of an immune checkpoint inhibitor.

Disclosed herein, in some embodiments, are methods of providing a cancer immunotherapy to a subject in need thereof, wherein the cancer is associated with a presence of immunosuppressive macrophages, the method comprising administering to the subject (a) a therapeutic amount of an immunomodulatory CD163 antibody and (b) a therapeutic amount of an immune checkpoint inhibitor. In some embodiments, the administering of the immunomodulatory CD163 antibody and the immune checkpoint inhibitor additively or synergistically exert a therapeutic effect as compared to administration of either the immunomodulatory CD163 antibody or the immune checkpoint inhibitor alone. In some embodiments, the immunomodulatory CD163 antibody comprises a light chain variable domain and a heavy chain variable domain, wherein the sequences of the light chain and heavy chain variable domains are selected from the group consisting of: (a) SEQ ID NOs: 28 and 29; (b) SEQ ID NOs: 30 and 31; (c) SEQ ID NOs: 32 and 33; (d) SEQ ID NOs: 34 and 35; (e) SEQ ID NOs: 36 and 37; (f) SEQ ID NOs: 38 and 39; and (g) SEQ ID NOs: 40 and 41. In some embodiments, the immunomodulatory CD163 antibody comprises sequences as set forth in Tables 2 and 3, wherein the sequences are selected from the group consisting of: (a) SEQ ID NOs: 1, 2, 3, 4, 5, and 6; (b) SEQ ID NOs: 7, 2, 8, 16, 17, and 18; (c) SEQ ID NOs: 7, 9, 10, 19, 20, and 21; (d) SEQ ID NOs: 7, 2, 11, 22, 23, and 24; (e) SEQ ID NOs: 7, 2, 8, 22, 17, and 18; (f) SEQ ID NOs: 7, 2, 10, 16, 17, and 24; and (g) SEQ ID NOs: 7, 2, 12, 19, 17, and 18. In some embodiments, the immunomodulatory CD163 antibody comprises sequences set forth as follows: (a) RASQSISX8YLN (SEQ ID NO: 13), wherein X8=S, R, K, H; (b) AASSLQX9 (SEQ ID NO: 14), wherein X9=S, N, Q, T; (c) QQSYSTX10X11GX12 (SEQ ID NO: 15), wherein X10=P, Q, T, S, N, A, G; X11=R, G, A, S; and X12=T, S, A, G, N; (d) SX₁X₂MH, wherein X1=Y, E, Q, D; and X2=A, D, T, V, S, G, E; (e) VISX3DGSNKYX4ADSVKG (SEQ ID NO: 26), wherein X3=Y, E, Q, D; and X4=Y, N, H, E, D, K, Q, R; and (f) ENVRPYYDFWX5GYX6SEYYYYGX7DV (SEQ ID NO: 27), wherein X5=S, R, K, H; X6=Y, S, N, T, A, Q; and X7=M, L, I, V. In some embodiments, the administering of the immunomodulatory CD163 antibody and the immune checkpoint inhibitor promotes (i) proliferation of CD3+ T cells in the subject as demonstrated in one or more in vitro assays comprising cells of the subject; and/or (ii) cytokine secretion in the subject. In some embodiments, the administering of the immunomodulatory CD163 antibody and the immune checkpoint inhibitor promotes T cell-mediated killing of tumor cells in the subject. In some embodiments, the administering of the immunomodulatory CD163 antibody and the immune checkpoint inhibitor promotes an immune cell function as measured by one or both of the following parameters: (a) activation of a CD4+ T cell, CD8+ T cell, NK cell, or any combination thereof; and (b) proliferation of a CD4+ T cell, CD8+ T cell, NK cell, or any combination thereof. In some embodiments, the administering of the immunomodulatory CD163 antibody and the immune checkpoint inhibitor results in increased immunostimulatory activity in a tumor microenvironment. In some embodiments, the immunostimulatory activity in the tumor microenvironment is assessed via biopsy or in situ scanning. In some embodiments, the subject has been selected based on detection of a biomarker in a sample from the subject indicating susceptibility of the cancer to treatment with an immune checkpoint inhibitor. In some embodiments, the biomarker comprises detecting PD-L1 expression by cells of the tumor. In some embodiments, the biomarker comprises detecting PD-L1 expression by tumor-associated macrophages. In some embodiments, cancer cells from the subject have been characterized as expressing PD-L1 and, optionally, at a level higher than non-cancer cells of the subject or a control subject. In some embodiments, the methods further comprise a step of confirming PD-L1 expression by the cancer cells before commencing administration of the combination of the immunomodulatory CD163 antibody and the immune checkpoint inhibitor.

Disclosed herein, in some embodiments, are methods of treating a PD-L1-expressing cancer in a subject in need thereof, comprising administering to the subject (a) a therapeutic amount of an immunomodulatory hCD163 antibody or fragment thereof and (b) a therapeutic amount of a PD-1 antagonist or a PD-L1 antagonist. In some embodiments, the administering of (i) the immunomodulatory hCD163 antibody and the PD-1 antagonist or PD-L1 antagonist gives rise to an additive or synergistic effect as compared to administration of either the immunomodulatory CD163 antibody or the PD-1 antagonist or PD-L1 antagonist alone.

Disclosed herein, in some embodiments, are methods of treating a cancer in a subject in need thereof, wherein PD-L1 is expressed by cells of the cancer or immunosuppressive cells of the subject, the method comprising administering to the subject (a) a therapeutic amount of an immunomodulatory hCD163 antibody and (b) a therapeutic amount of a PD-1 antagonist or a PD-L1 antagonist. In some embodiments, the administering of (i) the immunomodulatory CD163 antibody and the PD-1 antagonist or PD-L1 antagonist results in an additive or synergistic effect as compared to administration of either the immunomodulatory CD163 antibody or the PD-1 antagonist or PD-L1 antagonist alone. In some embodiments, the immunosuppressive cells comprise antigen-presenting cells, dendritic cells, macrophages, fibroblasts, or T cells. In some embodiments, the methods further comprise a step of detecting a genomic alteration in cells of the cancer, which genomic alteration is associated with increased expression of PD-L1 by the cancer cells. In some embodiments, a predictive biomarker for: (a) increased expression of PD-L1 in cells of the cancer has been detected and/or (b) susceptibility to a PD-1 or PD-L1 antagonist has been detected, and wherein the biomarker is selected from increased histone acetylation, increased methylation of histone H3 on lysine 4, expression of zeste homolog 2 (EZH2), overactivity or overexpression of MYC, upregulation of ALK, loss of p53 function, post-translational N-linked glycosylation, serine/threonine or tyrosine phosphorylation, polyubiquitination, palmitoylation of PD-L1, exosomal PD-L1, soluble PD-L1 or splicing variants thereof, or mutation or hyperactivation of HIF1/2α, NF-κB, MAPK, PTEN/PI3K and/or EGFR pathways. In some embodiments, the methods further comprise administering a MEK inhibitor to the subject in an amount effective to decrease expression of PD-L1 by the cancer. In some embodiments, the cancer is pancreatic cancer. In some embodiments, the subject is not known to be BRAF inhibitor refractory and the method further comprises administering an effective amount of a BRAF inhibitor. In some embodiments, the subject has been selected by assessing a relevant biological sample from the subject for one or more of the following predictive biomarkers: high number of tumor-associated macrophages, high number of M2-like macrophages, high number of myeloid-derived suppressor cells, high expression of CD163 by macrophages, high ratio of M2 (CD206+) to M1 (CD11c+) macrophages, and high ratio of M2 (CD163+) to M1 (CD163−) macrophages among tumor-associated (CD68+) macrophages.

Disclosed herein, in some embodiments, are methods of characterizing an agent or a combination of agents comprising an in vitro assay which comprises steps of (a) contacting a myeloid cell preparation with a combination of (i) an immunomodulatory CD163 antibody and (ii) an immune checkpoint inhibitor; and (b) measuring expression or production of IL2, TNF-α, perforin, and/or IFN-γ as compared to cells contacted with the immunomodulatory CD163 antibody or the immune checkpoint inhibitor alone. In some embodiments, the immunomodulatory CD163 antibody binding to the myeloid cell potentiates an immune response in the subject as measured by at least one in vitro assay using cells from the subject. In some embodiments, the potentiated immune response in the subject is greater than when the immunomodulatory CD163 antibody binds in the absence of the immune checkpoint inhibitor. In some embodiments, the immunomodulatory CD163 antibody binding to the myeloid cell promotes an immunostimulatory function of the cell an immune response in the subject as measured by at least one in vitro assay using cells from the subject. In some embodiments, the immunostimulatory function of the cell is greater than when the immunomodulatory CD163 antibody binds in the absence of the immune checkpoint inhibitor.

Disclosed herein, in some embodiments, are methods of treating a cancer using immunotherapy in a subject in need thereof, comprising administering to the subject a therapeutic amount of each of (a) an immunomodulatory CD163 antibody comprising a hCD163-binding domain and (b) a PD-L1 antagonist.

Disclosed herein, in some embodiments, are methods of treating a cancer in a subject in need thereof, comprising administering to the subject (a) a therapeutic amount of an immunomodulatory CD163 antibody comprising an hCD163-binding domain and (b) a therapeutic amount of a PD-L1 antagonist.

Disclosed herein, in some embodiments, are methods of treating a cancer using immunotherapy in a subject in need thereof, comprising administering to the subject (a) a therapeutic amount of an immunomodulatory CD163 antibody and (b) a therapeutic amount of a PD-L1 antagonist.

Disclosed herein, in some embodiments, are methods of treating a cancer in a subject in need thereof, comprising administering to the subject (a) a therapeutic amount of an immunomodulatory CD163 and (b) a therapeutic amount of a PD-L1 antagonist. In some embodiments, the administering of the immunomodulatory CD163 antibody and PD-L1 antagonist results in an additive or synergistic effect as compared to administration of either the immunomodulatory CD163 antibody or the PD-L1 antagonist alone. In some embodiments, the immunomodulatory CD163 antibody binding alters expression of at least one marker on the macrophage selected from CD16, CD64, TLR2, and Siglec-15.

Disclosed herein, in some embodiments, are methods of treating a cancer in a subject in need thereof, comprising administering to the subject (i) a therapeutic amount of an immunomodulatory CD163 antibody that specifically binds to hCD163; and (ii) a therapeutic amount of an immune checkpoint inhibitor selected from a PD-1 antagonist or a PD-L1 antagonist.

Disclosed herein, in some embodiments, are methods of providing a cancer immunotherapy in a subject in need thereof, comprising administering to the subject (a) a therapeutic amount of an immunomodulatory CD163 antibody comprising an hCD163-binding domain and (b) a therapeutic amount of a PD-1 antagonist.

Disclosed herein, in some embodiments, are methods of providing a cancer immunotherapy in a subject in need thereof, comprising administering to the subject (a) a therapeutic amount of an immunomodulatory CD163 antibody that specifically binds to hCD163 and (b) a therapeutic amount of a PD-1 antagonist.

Disclosed herein, in some embodiments, are methods of providing a cancer immunotherapy in a subject in need thereof wherein the subject has previously had to stop treatment with a PD-1 antagonist due to toxicity from the treatment and wherein the subject is treated by administering to the subject a combination comprising (a) a therapeutic amount of an immunomodulatory CD163 antibody that specifically binds to hCD163 and (b) a therapeutic amount of a PD-1 antagonist, wherein the dose of the PD-1 antagonist in the combination treatment is lower than the dose that the patient received and had to stop. In some embodiments, the administering of the immunomodulatory CD163 antibody and PD-1 antagonist results in an additive or synergistic effect as compared to administration of either the immunomodulatory CD163 antibody or the PD-1 antagonist alone.

Disclosed herein, in some embodiments, are methods of relieving T cell suppression in a tumor microenvironment comprising contacting a tumor microenvironment with (i) an immunomodulatory CD163 antibody comprising: a light chain variable domain (VL) having a sequence at least 80% identical to the amino acid sequence selected from the group consisting of: SEQ ID NO: 28, SEQ ID NO: 30, SEQ ID NO: 32, SEQ ID NO: 34, SEQ ID NO: 36, SEQ ID NO: 38, and SEQ ID NO: 40, and a heavy chain variable domain (VH) having a sequence at least 80% identical to the amino acid sequence selected from the group consisting of: SEQ ID NO: 29, SEQ ID NO: 31, SEQ ID NO: 33, SEQ ID NO: 35, SEQ ID NO: 37, SEQ ID NO: 39, and SEQ ID NO: 41; and (ii) an immune checkpoint inhibitor selected from a PD-1 antagonist and a PD-L1 antagonist. In some embodiments, the T cell suppression is measured by an increase in IFN-γ, TNF-α, perforin, or IL2. In some embodiments, the increase is relative to levels prior to the administration of the immunomodulatory CD163 antibody and the immune checkpoint inhibitor.

Disclosed herein, in some embodiments, are methods of promoting an immune cell function in a subject in need thereof, the method comprising: administering to the subject a combination comprising (1) a therapeutic amount of an antibody comprising a light chain variable domain (VL) having a sequence at least 80% identical to the amino acid sequence selected from the group consisting of: SEQ ID NO: 28, SEQ ID NO: 30, SEQ ID NO: 32, SEQ ID NO: 34, SEQ ID NO: 36, SEQ ID NO: 38, and SEQ ID NO: 40, and a heavy chain variable domain (VH) having a sequence at least 80% identical to the amino acid sequence selected from the group consisting of: SEQ ID NO: 29, SEQ ID NO: 31, SEQ ID NO: 33, SEQ ID NO: 35, SEQ ID NO: 37, SEQ ID NO: 39, and SEQ ID NO: 41; and (2) a therapeutic amount of an immune checkpoint inhibitor, in which the combination is effective to promote an immune cell function measured by: (i) activation of a CD4+ T cell, CD8+ T cell, NK cell, or any combination thereof; and/or (ii) proliferation of a CD4+ T cell, CD8+ T cell, NK cell, or any combination thereof.

Disclosed herein, in some embodiments, are methods of promoting an immune cell function in a subject in need thereof, the method comprising: (a) administering to the subject (i) a therapeutic amount of an immunomodulatory CD163 antibody comprising a light chain variable domain (VL) having a sequence at least 80% identical to the amino acid sequence selected from the group consisting of: SEQ ID NO: 28, SEQ ID NO: 30, SEQ ID NO: 32, SEQ ID NO: 34, SEQ ID NO: 36, SEQ ID NO: 38, and SEQ ID NO: 40, and a heavy chain variable domain (VH) having a sequence at least 80% identical to the amino acid sequence selected from the group consisting of: SEQ ID NO: 29, SEQ ID NO: 31, SEQ ID NO: 33, SEQ ID NO: 35, SEQ ID NO: 37, SEQ ID NO: 39, and SEQ ID NO: 41; and (ii) a therapeutic amount of an immune checkpoint inhibitor, in which the combination is effective to promote an immune cell function in the subject; and (b) measuring a parameter selected from: (i) activation of a CD4+ T cell, CD8+ T cell, NK cell, or any combination thereof; (ii) proliferation of a CD4+ T cell, CD8+ T cell, NK cell, or any combination thereof; and/or (iii) a greater proportion of Th1 cells as compared to Th2 cells, using an in vitro assay comprising cells from the subject. In some embodiments, a first measuring step measuring the parameter is performed prior to administering the immunomodulatory CD163 antibody and the immune checkpoint inhibitor and a second measuring step measuring the parameter is performed after administering the immunomodulatory CD163 antibody and the immune checkpoint inhibitor, and the method further comprises comparing a value of the parameter measured in the first measuring step to a value measured in the second measuring step to confirm promotion of the immune cell function in the subject. In some embodiments, the activation of a CD4+ T cell, CD8+ T cell, NK cell, or any combination thereof is measured as an increased level of IL2, IFN-γ, TNF-α, or perforin, or any combination thereof as measured after administration of the immunomodulatory CD163 antibody and the immune checkpoint inhibitor as compared to prior to administration of the immunomodulatory CD163 antibody and the immune checkpoint inhibitor. In some embodiments, the immune cell is an immunosuppressive myeloid cell. In some embodiments, the immunosuppressive myeloid cell is a macrophage. In some embodiments, the immunosuppressive myeloid cell is a myeloid-derived suppressor cell.

Disclosed herein, in some embodiments, are methods of treating a cancer in a subject in need thereof, the method comprising administering to the subject (a) a therapeutic amount of an immunomodulatory CD163 antibody comprising a light chain variable domain (VL) having a sequence at least 80% identical to the amino acid sequence selected from the group consisting of: SEQ ID NO: 28, SEQ ID NO: 30, SEQ ID NO: 32, SEQ ID NO: 34, SEQ ID NO: 36, SEQ ID NO: 38, and SEQ ID NO: 40, and a heavy chain variable domain (VH) having a sequence at least 80% identical to the amino acid sequence selected from the group consisting of: SEQ ID NO: 29, SEQ ID NO: 31, SEQ ID NO: 33, SEQ ID NO: 35, SEQ ID NO: 37, SEQ ID NO: 39, and SEQ ID NO: 41; and; and (b) a therapeutic amount of a PD-1 antagonist or a PD-L1 antagonist, whereby immunosuppression by a tumor-associated macrophage in the subject is reduced. In some embodiments, T cell-mediated tumor cell killing in the subject is increased.

Disclosed herein, in some embodiments, are methods of reducing a tumor promoting activity of a tumor-associated macrophage in a subject in need thereof, the method comprising administering to the subject (i) a therapeutic amount of an immunomodulatory CD163 antibody comprising a light chain variable domain (VL) having a sequence at least 80% identical to the amino acid sequence selected from the group consisting of: SEQ ID NO: 28, SEQ ID NO: 30, SEQ ID NO: 32, SEQ ID NO: 34, SEQ ID NO: 36, SEQ ID NO: 38, and SEQ ID NO: 40, and a heavy chain variable domain (VH) having a sequence at least 80% identical to the amino acid sequence selected from the group consisting of: SEQ ID NO: 29, SEQ ID NO: 31, SEQ ID NO: 33, SEQ ID NO: 35, SEQ ID NO: 37, SEQ ID NO: 39, and SEQ ID NO: 41; and (ii) a therapeutic amount of a PD-1 antagonist or a PD-L1 antagonist, wherein the administration results in modulation of a CD4+ T cell activation, CD4+ T cell proliferation, CD8+ T cell activation, CD8+ T cell proliferation, or any combination. In some embodiments, the immunomodulatory CD163 antibody binds to a macrophage in a tumor microenvironment.

Disclosed herein, in some embodiments, are methods of modulating an activity of a tumor-associated macrophage in a tumor microenvironment, the method comprising contacting the tumor-associated macrophage with (i) an immunomodulatory CD163 antibody comprising a light chain variable domain (VL) having a sequence at least 80% identical to the amino acid sequence selected from the group consisting of: SEQ ID NO: 28, SEQ ID NO: 30, SEQ ID NO: 32, SEQ ID NO: 34, SEQ ID NO: 36, SEQ ID NO: 38, and SEQ ID NO: 40, and a heavy chain variable domain (VH) having a sequence at least 80% identical to the amino acid sequence selected from the group consisting of: SEQ ID NO: 29, SEQ ID NO: 31, SEQ ID NO: 33, SEQ ID NO: 35, SEQ ID NO: 37, SEQ ID NO: 39, and SEQ ID NO: 41; and (ii) a PD-L1 antagonist, wherein the contacting results in at least one of the following effects: (a) reduced expression of at least one marker on the tumor-associated macrophage, wherein the at least one marker is CD16, CD64, TLR2, or Siglec-15; (b) the immunomodulatory CD163 antibody is internalized by the tumor-associated macrophage; (c) the contacting of the tumor and/or binding of the immunomodulatory CD163 antibody is not cytotoxic to the tumor-associated macrophage; (d) increase in levels of IFN-γ, TNF-α and/or perforin; (e) activation of a CD4+ T cell, CD8+ T cell, NK cell, or any combination thereof; (f) proliferation of a CD4+ T cell, CD8+ T cell, NK cell, or any combination thereof; and (g) promotion of tumor cell killing in the tumor microenvironment. In some embodiments, the contacting with the immunomodulatory CD163 antibody results in: two or more of (a) through (g); three or more of (a) through (g); four or more of (a) through (g); five or more of (a) through (g); six or more of (a) through (g); or all of (a) through (g). In some embodiments, administering the immunomodulatory CD163 antibody and the immune checkpoint inhibitor results in an additive or synergistic effect as compared to administration of either the immunomodulatory CD163 antibody or the immune checkpoint inhibitor alone. In some embodiments, each of the immunomodulatory CD163 antibody and the immune checkpoint inhibitor are administered in submaximal or subtherapeutic amounts relative to amounts when administered as a monotherapy.

Disclosed herein, in some embodiments, are methods of promoting an immune cell function, the method comprising: specifically binding an antibody to a CD163 protein expressed on an immunosuppressive human myeloid cell in the presence of an immune checkpoint inhibitor selected from a PD-1 antagonist and a PD-L1 antagonist; and promoting an immune cell function as measured by one or both of the following parameters: (i) activation of a CD4+ T cell, CD8+ T cell, NK cell, or any combination thereof; and (ii) proliferation of a CD4+ T cell, CD8+ T cell, NK cell, or any combination thereof, wherein the activation and/or proliferation is increased as compared to binding of the antibody in the absence of the PD-1 antagonist or PD-L1 antagonist. In some embodiments, the method is performed in vitro or in vivo.

Disclosed herein, in some embodiments, are methods of treating a cancer characterized by the expression of an immune checkpoint protein in a subject in need thereof, the method comprising administering to the subject a therapeutic amount of an immunomodulatory CD163 antibody that binds to a human myeloid cell in the presence of an immune checkpoint inhibitor, whereby immunosuppression by a tumor-associated macrophage in the subject is reduced.

Disclosed herein, in some embodiments, are methods of treating a cancer in a subject treated with an immune checkpoint inhibitor therapy, the method comprising administering to the subject a therapeutic amount of an immunomodulatory CD163 antibody that binds to a human myeloid cell, whereby immunosuppression by a tumor-associated macrophage in the subject is reduced. In some embodiments, the immune checkpoint inhibitor inhibits an immune checkpoint protein selected from PD-1, CTLA-4, LAG3, TIGIT, TIM-3, or combinations thereof.

Disclosed herein, in some embodiments, are methods of treating a cancer in a subject in need thereof, the method comprising administering to the subject a therapeutic amount of an immunomodulatory CD163 antibody in the presence of an immune checkpoint inhibitor, wherein the immune checkpoint inhibitor is selected from a PD-1 antagonist and a PD-L1 antagonist, and whereby T cell-mediated tumor cell killing in the subject is increased and/or T cell exhaustion is relieved. In some embodiments, the immunomodulatory CD163 antibody comprises a light chain variable domain and a heavy chain variable domain, having amino acid sequences selected from the group consisting of: (a) SEQ ID NOs: 28 and 29; (b) SEQ ID NOs: 30 and 31; (c) SEQ ID NOs: 32 and 33; (d) SEQ ID NOs: 34 and 35; (e) SEQ ID NOs: 36 and 37; (f) SEQ ID NOs: 38 and 39; and (g) SEQ ID NOs: 40 and 41. In some embodiments, the immunomodulatory CD163 antibody comprises amino acid sequences selected from the group consisting of: (a) SEQ ID NOs: 1, 2, 3, 4, 5, and 6; (b) SEQ ID NOs: 7, 2, 8, 16, 17, and 18; (c) SEQ ID NOs: 7, 9, 10, 19, 20, and 21; (d) SEQ ID NOs: 7, 2, 11, 22, 23, and 24; (e) SEQ ID NOs: 7, 2, 8, 22, 17, and 18; (f) SEQ ID NOs: 7, 2, 10, 16, 17, and 24; and (g) SEQ ID NOs: 7, 2, 12, 19, 17, and 18. In some embodiments, the immunomodulatory hCD163 antibody comprises sequences as set forth as follows: (a) RASQSISX8YLN (SEQ ID NO: 13), wherein X8=S, R, K, H; (b) AASSLQX9 (SEQ ID NO: 14), wherein X9=S, N, Q, T; (c) QQSYSTX10X11GX12 (SEQ ID NO: 15), wherein X10=P, Q, T, S, N, A, G; X11=R, G, A, S; and X12=T, S, A, G, N; (d) SX₁X₂MH wherein X1=Y, E, Q, D; and X2=A, D, T, V, S, G, E; (e) VISX3DGSNKYX4ADSVKG (SEQ ID NO: 26), wherein X3=Y, E, Q, D; and X4=Y, N, H, E, D, K, Q, R; and (f) ENVRPYYDFWX5GYX6SEYYYYGX7DV (SEQ ID NO: 27), wherein X5=S, R, K, H; X6=Y, S, N, T, A, Q; and X7=M, L, I, V. In some embodiments, the PD-1 antagonist is a PD-1 antibody. In some embodiments, the PD-1 antibody is an IgG1 antibody or IgG4 antibody. In some embodiments, the PD-1 antibody is selected from the group consisting of nivolumab, pembrolizumab, cemiplimab, dostarlimab, JTX-4014, spartalizumab, camrelizumab, sintilimab, tislelizumab, toripalimab, INCMGA00012, AMP-224, AMP-514, zimberelimab, and fragments or combinations thereof. In some embodiments, the PD-1 antagonist comprises a PD-1 binding domain comprising CDRs of an antibody selected from the group consisting of nivolumab, pembrolizumab, cemiplimab, dostarlimab, JTX-4014, spartalizumab, camrelizumab, sintilimab, tislelizumab, toripalimab, INCMGA00012, AMP-224, AMP-514, zimberelimab, and fragments or combinations thereof. In some embodiments, the PD-1 antagonist is a small molecule. In some embodiments, the PD-1 antagonist is a rationally-designed peptide antagonist of PD-1. In some embodiments, the PD-L1 antagonist is an PD-L1 antibody. In some embodiments, the PD-L1 antibody is an IgG1 antibody or IgG4 antibody. In some embodiments, the PD-L1 antibody is selected from the group consisting of avelumab, durvalumab, atezolizumab, envafolimab, cosibelimab, LY3300054 CA-170, and fragments thereof combinations thereof. In some embodiments, the PD-L1 antagonist comprises AUNP-12, BMS-986189, a PD-L1 binding domain comprising CDRs of an antibody selected from the group consisting of avelumab, durvalumab, atezolizumab, envafolimab, cosibelimab (CK-301), LY3300054, CA-170, and BMS-936559, and active fragments thereof, or combinations thereof. In some embodiments, the immunomodulatory CD163 antibody does not bind to a murine CD163 or any non-human primate CD163.

Disclosed herein, in some embodiments, are methods of treating a cancer in a subject in need thereof, comprising administering to the subject: (a) a therapeutic amount of an immunomodulatory CD163 antibody that binds to an epitope of human CD163 (hCD163) comprising amino acid sequence IGRVNASKGFGHIWLDSVSCQGHEPAI (SEQ ID NO: 43), VVCRQLGCGSA (SEQ ID NO: 44), and WDCKNWQWGGLTCD (SEQ ID NO: 45); and (b) a therapeutic amount of an immune checkpoint inhibitor. In some embodiments, the immunomodulatory CD163 antibody comprises: a light chain variable domain (VL) having a sequence at least 80% identical to the amino acid sequence selected from the group consisting of: SEQ ID NO: 28, SEQ ID NO: 30, SEQ ID NO: 32, SEQ ID NO: 34, SEQ ID NO: 36, SEQ ID NO: 38, and SEQ ID NO: 40, and a heavy chain variable domain (VH) having a sequence at least 80% identical to the amino acid sequence selected from the group consisting of: SEQ ID NO: 29, SEQ ID NO: 31, SEQ ID NO: 33, SEQ ID NO: 35, SEQ ID NO: 37, SEQ ID NO: 39, and SEQ ID NO: 41. In some embodiments, the immunomodulatory CD163 antibody specifically binds to an epitope of hCD163 and comprises: (i) a light chain variable domain (VL) and a heavy chain variable domain (VH) with amino acid sequences as set forth in SEQ ID NOs: 40 and 41; and/or (ii) amino acid sequences as set forth in SEQ ID NOS: 1, 2, 3, 4, 5, and 6. In some embodiments, the immune checkpoint inhibitor is selected from the group consisting of an antagonist to an immune checkpoint protein or ligand thereof, wherein the immune checkpoint protein is selected from the group consisting of CTLA-4, PD-1, TIM3, TIGIT, and any combination thereof. In some embodiments, the immune checkpoint inhibitor is a PD-1 antagonist or PD-L1 antagonist. In some embodiments, the PD-1 antagonist is a PD-1 antibody. In some embodiments, the PD-1 antibody is selected from the group consisting of nivolumab, pembrolizumab, cemiplimab, dostarlimab, JTX-4014, spartalizumab, camrelizumab, sintilimab, tislelizumab, toripalimab, INCMGA00012, AMP-224, AMP-514, zimberelimab, and fragments or combinations thereof. In some embodiments, the PD-1 antagonist comprises a PD-1 binding domain comprising CDRs of an antibody selected from the group consisting of nivolumab, pembrolizumab, cemiplimab, dostarlimab, JTX-4014, spartalizumab, camrelizumab, sintilimab, tislelizumab, toripalimab, INCMGA00012, AMP-224, or AMP-514, zimberelimab, and fragments or combinations thereof. In some embodiments, the PD-1 antagonist is a rationally-designed peptide antagonist of PD-1. In some embodiments, the PD-L1 antagonist is a PD-L1 antibody. In some embodiments, the PD-L1 antibody is selected from the group consisting of avelumab, durvalumab, atezolizumab, envafolimab, cosibelimab, LY3300054 CA-170, BMS-936559, and PD-L1-binding fragments or combinations thereof. In some embodiments, the PD-L1 antagonist comprises AUNP-12, BMS-986189, a PD-L1 binding domain comprising CDRs of an antibody selected from the group consisting of avelumab, durvalumab, atezolizumab, envafolimab, cosibelimab (CK-301), LY3300054, CA-170, and BMS-936559, and active fragments thereof, or combinations thereof. In some embodiments, the immunomodulatory CD163 antibody and the immune checkpoint inhibitor are co-formulated. In some embodiments, the immunomodulatory CD163 antibody and the immune checkpoint inhibitor are in separate formulations.

Disclosed herein, in some embodiments, are methods of treating a cancer in a subject in need thereof, comprising administering to the subject a therapeutic amount of (a) a means for binding human CD163 (hCD163); and (b) a means for inhibiting an immune checkpoint protein or a ligand thereof. In some embodiments, the means for binding hCD163 binds hCD163 expressed on a myeloid cell or soluble hCD163. In some embodiments, the means for binding hCD163 binds hCD163 expressed on a myeloid cell. In some embodiments, the myeloid cell is a tumor-associated macrophage. In some embodiments, the means for binding hCD163 binds domain 3 of hCD163.

Disclosed herein, in some embodiments, are methods wherein the means for binding hCD163 binds at a localized region of the hCD163 comprising an amino acid sequence IGRVNASKGFGHIWLDSVSCQGHEPAI (SEQ ID NO: 43), VVCRQLGCGSA (SEQ ID NO: 44), WDCKNWQWGGLTCD (SEQ ID NO: 45), or any combination thereof. In some embodiments, the means for binding hCD163 specifically binds hCD163. In some embodiments, the means for binding hCD163 comprises an antibody or antigen-binding fragment thereof. In some embodiments, the antibody is an IgG1 antibody. In some embodiments, the immune checkpoint protein is PD-1. In some embodiments, the ligand is PD-L1. In some embodiments, the means for inhibiting an immune checkpoint protein or a ligand thereof comprises an antibody or antigen-binding fragment thereof.

Disclosed herein, in some embodiments, are methods of treating a cancer in a subject in need thereof, comprising administering to the subject a therapeutic amount of (a) a means for binding a human myeloid cell expressing human CD163 (hCD163) protein at a localized region on a surface of the hCD163 protein comprising an amino acid sequence IGRVNASKGFGHIWLDSVSCQGHEPAI (SEQ ID NO: 43), VVCRQLGCGSA (SEQ ID NO: 44), WDCKNWQWGGLTCD (SEQ ID NO: 45), or any combination thereof; and (b) a means for inhibiting an immune checkpoint protein or a ligand thereof. In some embodiments, the means for binding a human myeloid cell comprises an antibody or antigen-binding fragment thereof. In some embodiments, the antibody is an IgG1 antibody.

Disclosed herein, in some embodiments, are methods of treating a cancer in a subject in need thereof, comprising administering to the subject a therapeutic amount of: (a) means for modulating an immune function of a tumor-associated macrophage expressing human CD163 protein (hCD163) by binding the hCD163 protein at a localized region on a surface of the hCD163 protein comprising an amino acid sequence IGRVNASKGFGHIWLDSVSCQGHEPAI (SEQ ID NO: 43), VVCRQLGCGSA (SEQ ID NO: 44), WDCKNWQWGGLTCD (SEQ ID NO: 45), or any combination thereof; and (b) a means for inhibiting an immune checkpoint protein or a ligand thereof. In some embodiments, the means for modulating an immune function comprises an antibody or antigen-binding fragment thereof. In some embodiments, the antibody is an IgG1 antibody. In some embodiments, the modulating of an immune function comprises: (i) inducing enhanced function of CD3+ T cells, CD4+ T cells, CD8+ T cells, and/or CD4+ T cells and CD8+ T cells; (ii) relieving T cell suppression or T cell exhaustion that may be induced by cancer cells; (iii) increasing T cell-mediated killing of cancer cell; (iv) or stimulating proliferation of T cells. In some embodiments, the modulating of an immune function comprises: (a) reducing expression of at least one marker on the macrophages selected from the group consisting of CD16, CD64, TLR2, and Siglec-15; (b) internalizing of bound antibody by the macrophages; (c) increasing IFN-γ, TNF-α, or perforin in the subject; (d) promoting activation of CD4+ T cells, CD8+ T cells, or NK cells; (e) promoting proliferation of CD4+ T cells, CD8+ T cells, or NK cells; or (f) promoting tumor cell killing in the tumor microenvironment

Disclosed herein, in some embodiments, are methods of treating a cancer in a subject in need thereof, comprising administering to the subject a therapeutic amount of: (a) a modulator of tumor-associated macrophages comprising a means for binding human CD163 (hCD163) comprising at least 90% sequence identity to SEQ ID NO: 42; and (b) a means for inhibiting an immune checkpoint protein or a ligand thereof.

Disclosed herein, in some embodiments, are methods of treating a cancer in a subject in need thereof, comprising administering to the subject a therapeutic amount of a pharmaceutical acceptable composition comprising (a) a means for binding human CD163 (hCD163); (b) a means for inhibiting an immune checkpoint protein or a ligand thereof; and (c) a pharmaceutically acceptable excipient. In a method of treating a cancer in a subject in need thereof, comprising: administering to the subject an immune checkpoint inhibitor, the improvement comprising administering an immunomodulatory CD163 antibody or antigen-binding fragment thereof comprising: a light chain variable domain (VL) having a sequence at least 80% identical to the amino acid sequence selected from the group consisting of: SEQ ID NO: 28, SEQ ID NO: 30, SEQ ID NO: 32, SEQ ID NO: 34, SEQ ID NO: 36, SEQ ID NO: 38, and SEQ ID NO: 40, and a heavy chain variable domain (VH) having a sequence at least 80% identical to the amino acid sequence selected from the group consisting of: SEQ ID NO: 29, SEQ ID NO: 31, SEQ ID NO: 33, SEQ ID NO: 35, SEQ ID NO: 37, SEQ ID NO: 39, and SEQ ID NO: 41. In a method of treating a cancer in a subject in need thereof, comprising: administering to the subject an immune checkpoint inhibitor, the improvement comprising administering an immunomodulatory CD163 antibody comprising amino acid sequences as forth as follows: (a) RASQSISX8YLN (SEQ ID NO: 13), wherein X8=S, R, K, H; (b) AASSLQX9 (SEQ ID NO: 14), wherein X9=S, N, Q, T; (c) QQSYSTX10X11GX12 (SEQ ID NO: 15), wherein X10=P, Q, T, S, N, A, G; X11=R, G, A, S; and X12=T, S, A, G, N; (d) SX₁X₂MH, wherein X1=Y, E, Q, D; and X2=A, D, T, V, S, G, E; (e) VISX3DGSNKYX4ADSVKG (SEQ ID NO: 26), wherein X3=Y, E, Q, D; and X4=Y, N, H, E, D, K, Q, R; and (f) ENVRPYYDFWX5GYX6SEYYYYGX7DV (SEQ ID NO: 27), wherein X5=S, R, K, H; X6=Y, S, N, T, A, Q; and X7=M, L, I, V. In a method of treating a cancer in a subject in need thereof, comprising: administering to the subject an immune checkpoint inhibitor, the improvement comprising administering an immunomodulatory CD163 antibody comprising amino acid sequences selected from the group consisting of: (a) SEQ ID NOs: 1, 2, 3, 4, 5, and 6; (b) SEQ ID NOs: 7, 2, 8, 16, 17, and 18; (c) SEQ ID NOs: 7, 9, 10, 19, 20, and 21; (d) SEQ ID NOs: 7, 2, 11, 22, 23, and 24; (e) SEQ ID NOs: 7, 2, 8, 22, 17, and 18; (f) SEQ ID NOs: 7, 2, 10, 16, 17, and 24; and (g) SEQ ID NOs: 7, 2, 12, 19, 17, and 18. In some embodiments, the cancer is or comprises a carcinoma, sarcoma, or melanoma. In some embodiments, the cancer is lung cancer, skin cancer, head and neck cancer, hematological cancer, breast cancer, pancreatic cancer, colorectal cancer, gastrointestinal cancer, gastric cancer, thyroid cancer, brain cancer, prostate cancer, uterine cancer, cervical cancer, ovarian cancer, liver cancer, testicular cancer, or combinations thereof. In some embodiments, the cancer is non-small cell lung cancer (NSCLC), small cell lung cancer (SCLC), papillary thyroid cancer, classical Hodgkin lymphoma (cHL) primary mediastinal large B-Cell lymphoma (PMBCL), non-small cell lung cancer (NSCLC), soft-tissue sarcoma, liposarcoma, leiomyosarcoma, carcinoma, adenocarcinoma of the prostate or a prostatic intraepithelial neoplasia, squamous cell carcinoma, gastric adenocarcinoma, melanoma, triple-negative breast cancer, or combinations thereof. In some embodiments, the cancer comprises progressive metastatic disease or progressive locally advanced diseases not amenable to local therapy. In some embodiments, the prostate cancer is or comprises a carcinoma that is or comprises an adenocarcinoma of the prostate or a prostatic intraepithelial neoplasia (PIN). In some embodiments, the sarcoma is or comprises a soft tissue sarcoma. In some embodiments, the sarcoma is or comprises a liposarcoma or leiomyosarcoma. In some embodiments, the liposarcoma is dedifferentiated liposarcoma. In some embodiments, the lung cancer is a lung carcinoma or a lung sarcoma. In some embodiments, the lung carcinoma is or comprises a non-small cell lung carcinoma (NSCLC). In some embodiments, the skin cancer is or comprises melanoma. In some embodiments, the head and neck cancer is or comprises a squamous cell carcinoma of the head and neck (SCCHN). In some embodiments, the breast cancer is or comprises triple negative breast cancer.

Disclosed herein, in some embodiments, are combination products, comprising (i) a therapeutic amount of an immunomodulatory CD163 antibody and (ii) a therapeutic amount of an immune checkpoint inhibitor selected from a PD-1 antagonist and a PD-L1 antagonist, for use in the manufacture of a medicament, wherein the immunomodulatory CD163 antibody comprises a light chain variable domain (VL) having a sequence at least 80% identical to the amino acid sequence selected from the group consisting of: SEQ ID NO: 28, SEQ ID NO: 30, SEQ ID NO: 32, SEQ ID NO: 34, SEQ ID NO: 36, SEQ ID NO: 38, and SEQ ID NO: 40, and a heavy chain variable domain (VH) having a sequence at least 80% identical to the amino acid sequence selected from the group consisting of: SEQ ID NO: 29, SEQ ID NO: 31, SEQ ID NO: 33, SEQ ID NO: 35, SEQ ID NO: 37, SEQ ID NO: 39, and SEQ ID NO: 41. In some embodiments, the combination product is for use in treatment of cancer.

Disclosed herein, in some embodiments, are combinations comprising (i) a therapeutic amount of an immunomodulatory CD163 antibody comprising a light chain variable domain (VL) having a sequence at least 80% identical to the amino acid sequence selected from the group consisting of: SEQ ID NO: 28, SEQ ID NO: 30, SEQ ID NO: 32, SEQ ID NO: 34, SEQ ID NO: 36, SEQ ID NO: 38, and SEQ ID NO: 40, and a heavy chain variable domain (VH) having a sequence at least 80% identical to the amino acid sequence selected from the group consisting of: SEQ ID NO: 29, SEQ ID NO: 31, SEQ ID NO: 33, SEQ ID NO: 35, SEQ ID NO: 37, SEQ ID NO: 39, and SEQ ID NO: 41, and (ii) a therapeutic amount of an immune checkpoint inhibitor selected from a PD-1 antagonist and a PD-L1 antagonist, wherein the combination is used in the preparation of a medicament for the treatment of a cancer.

Disclosed herein, in some embodiments, are combinations comprising (i) a therapeutic amount of an immunomodulatory CD163 antibody comprising a light chain variable domain (VL) having a sequence at least 80% identical to the amino acid sequence selected from the group consisting of: SEQ ID NO: 28, SEQ ID NO: 30, SEQ ID NO: 32, SEQ ID NO: 34, SEQ ID NO: 36, SEQ ID NO: 38, and SEQ ID NO: 40, and a heavy chain variable domain (VH) having a sequence at least 80% identical to the amino acid sequence selected from the group consisting of: SEQ ID NO: 29, SEQ ID NO: 31, SEQ ID NO: 33, SEQ ID NO: 35, SEQ ID NO: 37, SEQ ID NO: 39, and SEQ ID NO: 41, and (ii) a therapeutic amount of an immune checkpoint inhibitor, wherein the combination is for use in treating a cancer. In some embodiments, the immunomodulatory CD163 antibody and the immune checkpoint inhibitor are co-formulated. In some embodiments, the immunomodulatory CD163 antibody and the immune checkpoint inhibitor are in separate formulations. In some embodiments, the immune checkpoint inhibitor is selected from the group consisting of an antagonist to an immune checkpoint protein or ligand thereof, wherein the immune checkpoint protein is selected from the group consisting of CTLA-4, PD-1, PD-L1, TIM3, LAG3, TIGIT, and any combination thereof. In some embodiments, the immune checkpoint inhibitor is a PD-1 antagonist. In some embodiments, the PD-1 antagonist is a PD-1 antibody. In some embodiments, the PD-1 antibody is an IgG1 antibody or IgG4 antibody. In some embodiments, the PD-1 antibody is selected from the group consisting of nivolumab, pembrolizumab, cemiplimab, dostarlimab, JTX-4014, spartalizumab, camrelizumab, sintilimab, tislelizumab, toripalimab, INCMGA00012, AMP-224, AMP-514, zimberelimab, and fragments or combinations thereof. In some embodiments, the PD-1 antagonist comprises a PD-1 binding domain comprising CDRs of an antibody selected from the group consisting of nivolumab, pembrolizumab, cemiplimab, dostarlimab, JTX-4014, spartalizumab, camrelizumab, sintilimab, tislelizumab, toripalimab, INCMGA00012, AMP-224, or AMP-514, zimberelimab, and fragments or combinations thereof. In some embodiments, the PD-1 antagonist is a small molecule. In some embodiments, the PD-1 antagonist is a rationally-designed peptide antagonist of PD-1. In some embodiments, the therapeutic amount of the immunomodulatory CD163 antibody is about 150 milligrams (mg) to about 1200 mg. In some embodiments, the therapeutic amount of the immune checkpoint inhibitor is about 150 milligrams (mg) to about 600 mg.

Disclosed herein, in some embodiments, are combinations comprising (i) a therapeutic amount of an immunomodulatory CD163 antibody comprising amino acid sequences set forth as follows: (a) RASQSISX8YLN (SEQ ID NO: 13), wherein X8=S, R, K, H; (b) AASSLQX9 (SEQ ID NO: 14), wherein X9=S, N, Q, T; (c) QQSYSTX10X11GX12 (SEQ ID NO: 15), wherein X10=P, Q, T, S, N, A, G; X11=R, G, A, S; and X12=T, S, A, G, N; (d) SX₁X₂MH, wherein X1=Y, E, Q, D; and X2=A, D, T, V, S, G, E; (e) VISX3DGSNKYX4ADSVKG (SEQ ID NO: 26), wherein X3=Y, E, Q, D; and X4=Y, N, H, E, D, K, Q, R; and (f) ENVRPYYDFWX5GYX6SEYYYYGX7DV (SEQ ID NO: 27), wherein X5=S, R, K, H; X6=Y, S, N, T, A, Q; and X7=M, L, I, V, and (ii) a therapeutic amount of an immune checkpoint inhibitor, wherein the combination is for use in treating a cancer.

Disclosed herein, in some embodiments, are combinations, comprising: (i) a therapeutic amount of an immunomodulatory CD163 antibody comprising amino acid sequences selected from the group consisting of: (a) SEQ ID NOs: 1, 2, 3, 4, 5, and 6; (b) SEQ ID NOs: 7, 2, 8, 16, 17, and 18; (c) SEQ ID NOs: 7, 9, 10, 19, 20, and 21; (d) SEQ ID NOs: 7, 2, 11, 22, 23, and 24; (e) SEQ ID NOs: 7, 2, 8, 22, 17, and 18; (f) SEQ ID NOs: 7, 2, 10, 16, 17, and 24; and (g) SEQ ID NOs: 7, 2, 12, 19, 17, and 18; and (ii) a therapeutic amount of an immune checkpoint inhibitor, wherein the combination is for use in treating a cancer.

Disclosed herein, in some embodiments, are combinations, comprising (i) about 150 milligrams (mg) to about 1200 mg of an immunomodulatory CD163 antibody comprising a light chain variable domain (VL) having a sequence at least 80% identical to the amino acid sequence selected from the group consisting of: SEQ ID NO: 28, SEQ ID NO: 30, SEQ ID NO: 32, SEQ ID NO: 34, SEQ ID NO: 36, SEQ ID NO: 38, and SEQ ID NO: 40, and a heavy chain variable domain (VH) having a sequence at least 80% identical to the amino acid sequence selected from the group consisting of: SEQ ID NO: 29, SEQ ID NO: 31, SEQ ID NO: 33, SEQ ID NO: 35, SEQ ID NO: 37, SEQ ID NO: 39, and SEQ ID NO: 41, and (ii) a therapeutic amount of an immune checkpoint inhibitor, wherein the combination is for use in treating a cancer. In some embodiments, the immune checkpoint inhibitor is selected from the group consisting of an antagonist to an immune checkpoint protein or ligand thereof, wherein the immune checkpoint protein is selected from the group consisting of CTLA-4, PD-1, PD-L1, TIM3, LAG3, TIGIT, and any combination thereof. In some embodiments, the immune checkpoint inhibitor is a PD-1 antagonist. In some embodiments, the PD-1 antagonist is a PD-1 antibody. In some embodiments, the PD-1 antibody is an IgG1 antibody or IgG4 antibody. In some embodiments, the PD-1 antibody is selected from the group consisting of nivolumab, pembrolizumab, cemiplimab, dostarlimab, JTX-4014, spartalizumab, camrelizumab, sintilimab, tislelizumab, toripalimab, INCMGA00012, AMP-224, AMP-514, zimberelimab, and fragments or combinations thereof. In some embodiments, the PD-1 antagonist comprises a PD-1 binding domain comprising CDRs of an antibody selected from the group consisting of nivolumab, pembrolizumab, cemiplimab, dostarlimab, JTX-4014, spartalizumab, camrelizumab, sintilimab, tislelizumab, toripalimab, INCMGA00012, AMP-224, or AMP-514, zimberelimab, and fragments or combinations thereof. In some embodiments, the PD-1 antagonist is a small molecule. In some embodiments, the PD-1 antagonist is a rationally-designed peptide antagonist of PD-1. In some embodiments, the therapeutic amount of the immunomodulatory CD163 antibody is about 150 milligrams (mg) to about 1200 mg. In some embodiments, the therapeutic amount of the immune checkpoint inhibitor is about 150 milligrams (mg) to about 600 mg. In some embodiments, the immunomodulatory CD163 antibody and the immune checkpoint inhibitor are co-formulated. In some embodiments, the immunomodulatory CD163 antibody and the immune checkpoint inhibitor are in separate formulations. In some embodiments, the cancer is or comprises a carcinoma, sarcoma, or melanoma. In some embodiments, the cancer is lung cancer, skin cancer, head and neck cancer, hematological cancer, breast cancer, pancreatic cancer, colorectal cancer, gastrointestinal cancer, gastric cancer, thyroid cancer, brain cancer, prostate cancer, uterine cancer, cervical cancer, ovarian cancer, liver cancer, testicular cancer, or combinations thereof. In some embodiments, the cancer is non-small cell lung cancer (NSCLC), small cell lung cancer (SCLC), papillary thyroid cancer, classical Hodgkin lymphoma (cHL) primary mediastinal large B-Cell lymphoma (PMBCL), non-small cell lung cancer (NSCLC), soft-tissue sarcoma, liposarcoma, leiomyosarcoma, carcinoma, adenocarcinoma of the prostate or a prostatic intraepithelial neoplasia, squamous cell carcinoma, gastric adenocarcinoma, melanoma, triple-negative breast cancer, or combinations thereof. In some embodiments, the cancer comprises progressive metastatic disease or progressive locally advanced diseases not amenable to local therapy. In some embodiments, the prostate cancer is or comprises a carcinoma that is or comprises an adenocarcinoma of the prostate or a prostatic intraepithelial neoplasia (PIN). In some embodiments, the sarcoma is or comprises a soft tissue sarcoma. In some embodiments, the sarcoma is or comprises a liposarcoma or leiomyosarcoma. In some embodiments, the liposarcoma is dedifferentiated liposarcoma. In some embodiments, the lung cancer is a lung carcinoma or a lung sarcoma. In some embodiments, the lung carcinoma is or comprises a non-small cell lung carcinoma (NSCLC). In some embodiments, the skin cancer is or comprises melanoma. In some embodiments, the head and neck cancer is or comprises a squamous cell carcinoma of the head and neck (SCCHN). In some embodiments, the breast cancer is or comprises triple negative breast cancer.

Disclosed herein, in some embodiments, are methods of providing a cancer immunotherapy to a subject in need thereof, comprising: administering to the subject: a) a therapeutic amount of an immunomodulatory CD163 antibody or antigen-binding fragment thereof comprising sequences 100% identical to the amino acid sequences according to SEQ ID NOs: 1, 2, 3, 4, 5, and 6 and b) a therapeutic amount of an immune checkpoint inhibitor selected from the group consisting of an antagonist to an immune checkpoint protein or ligand thereof, wherein the immune checkpoint protein is selected from the group consisting of CTLA-4, PD-1, PD-L1, TIM3, LAG3, TIGIT, and any combination thereof. In some embodiments, the immunomodulatory CD163 antibody is an IgG1 antibody or IgG4 antibody. In some embodiments, the therapeutic amount of the CD163 antibody is about 150 milligrams (mg) to about 1200 mg administered intravenously from about once per week to about once per 3 weeks. In some embodiments, the immunomodulatory CD163 antibody comprises a light chain variable domain (V_{L}) having a sequence at least 80% identical to the amino acid sequence according to SEQ ID NO: 40 and a heavy chain variable domain (V_{H}) having a sequence at least 80% identical to the amino acid sequence according to SEQ ID NO: 41. In some embodiments, the immunomodulatory CD163 antibody comprises a light chain variable domain (V_{L}) having a sequence at least 100% identical to the amino acid sequence according to SEQ ID NO: 40 and a heavy chain variable domain (V_{H}) having a sequence at least 100% identical to the amino acid sequence according to SEQ ID NO: 41. In some embodiments, the administering of the immunomodulatory CD163 antibody and the immune checkpoint inhibitor yields an additive or synergistic effect as compared to administration of either the immunomodulatory CD163 antibody or the immune checkpoint inhibitor alone. In some embodiments, the immune checkpoint inhibitor is a PD-1 antagonist. In some embodiments, the PD-1 antagonist is a PD-1 antibody, small molecule, or a rationally-designed peptide antagonist of PD-1. In some embodiments, the PD-1 antibody is an IgG1 antibody or IgG4 antibody. In some embodiments, the PD-1 antibody is selected from the group consisting of nivolumab, pembrolizumab, cemiplimab, dostarlimab, JTX-4014, spartalizumab, camrelizumab, sintilimab, tislelizumab, toripalimab, INCMGA00012, AMP-224, AMP-514, zimberelimab, and fragments or combinations thereof. In some embodiments, the immune checkpoint inhibitor is a PD-L1 antagonist. In some embodiments, the PD-L1 antagonist is a PD-L1 antibody. In some embodiments, the PD-L1 antibody is an IgG1 antibody or IgG4 antibody. In some embodiments, the PD-L1 antagonist is selected from the group consisting of avelumab, durvalumab, atezolizumab, envafolimab, cosibelimab (CK-301), LY3300054, CA-170, BMS-936559, and combinations and active fragments thereof. In some embodiments, the immunomodulatory CD163 antibody and the immune checkpoint inhibitor are administered concomitantly or sequentially. In some embodiments, when administration is sequential, an interval between administration of the immunomodulatory CD163 antibody and administration of the immune checkpoint inhibitor is between one hour and thirty days. In some embodiments, the interval is about three weeks. In some embodiments, the therapeutic amount of the CD163 antibody is about 150 milligrams (mg) to about 1200 milligrams. In some embodiments, the immunomodulatory CD163 antibody is administered intravenously or subcutaneously. In some embodiments, the cancer is non-small cell lung cancer (NSCLC), small cell lung cancer (SCLC), papillary thyroid cancer, classical Hodgkin lymphoma (cHL) primary mediastinal large B-Cell lymphoma (PMBCL), soft-tissue sarcoma, liposarcoma, leiomyosarcoma, carcinoma, adenocarcinoma of the prostate or a prostatic intraepithelial neoplasia, squamous cell carcinoma, gastric adenocarcinoma, melanoma, triple-negative breast cancer, or combinations thereof.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A-1B show IFNγ production in M2c/exhausted T cell cocultures after treatment with AB101, anti-PD-1, or a combination of AB101 and anti-PD-1.

FIGS. 2A-2D show IFNγ production in M2c/exhausted T cell cocultures after treatment with AB101, anti-PD-L1, or a combination of AB101 and anti-PD-L1.

FIG. 3A is a schematic showing an exemplary protocol for an M2c/T cell coculture followed by measurement of IL-2 production by activated T cells.

FIGS. 3B-3D show IL-2 production by activated T cells after treatment with AB101 (closed circles), a combination of AB101 and anti-PD-1 (closed squares), a combination of AB101 and hIgG4 (closed triangles), and a combination of hIgG1 and anti-PD-1 (closed diamonds). hIgG1 and hIgG4 served as isotype controls for AB101 and anti-PD-1 antibody, respectively. Significance and p-values (as calculated using a multiple t test) are shown in each graph, as applicable.

FIG. 4A is a schematic showing an exemplary protocol for an M2c/T cell coculture followed by measurement of CD8+ and CD4+ T cell proliferation.

FIGS. 4B-4C show percentages of divided CD8+ T cells in M2c/T cell cocultures after treatment with AB101 (closed circles), a combination of AB101 and anti-PD-1 (closed squares), a combination of AB101 and hIgG4 (closed triangles), and a combination of hIgG1 and anti-PD-1 (closed diamonds).

FIGS. 4D-4F show percentages of divided CD4+ T cells in M2c/T cell cocultures after treatment with AB101 (closed circles), a combination of AB101 and anti-PD-1 (closed squares), a combination of AB101 and hIgG4 (closed triangles), and a combination of hIgG1 and anti-PD-1 (closed diamonds). Significance and p-values (as calculated using a one-way ANOVA) are shown in each graph, as applicable.

FIGS. 5A-5D show tumor volume over time for A549 (FIG. 5A) and H1975 (FIG. 5C) tumors following treatment with an isotype control (circles), AB101 (squares) or anti-PD-1 antibody (triangles). FIGS. 5B and 5D show tumor weight for A549 (FIG. 5B) and H1975 (FIG. 5D) tumors following treatment with an isotype control (circles), AB101 (squares), or anti-PD-1 antibody (triangles).

### DETAILED DESCRIPTION

The present disclosure provides, among other things, technologies (e.g., combinations, methods of manufacturing, articles of manufacture, methods of use, etc.) comprising an immunomodulatory CD163 antibody and an immune checkpoint inhibitor. In some embodiments, a method of administering the immunomodulatory CD163 antibody and the immune checkpoint inhibitor to a subject having cancer exerts a therapeutic effect such that there is a greater therapeutic effect than one would see if either the immunomodulatory CD163 antibody or the immune checkpoint inhibitor were administered alone. In some embodiments, a method of administering the immunomodulatory CD163 antibody and the immune checkpoint inhibitor to a subject having cancer additively or synergistically exerts a therapeutic effect such that there is a greater than additive therapeutic effect than one would see if either the immunomodulatory CD163 antibody or the immune checkpoint inhibitor were administered alone.

Disclosed herein, in certain embodiments, are uses of an immunomodulatory CD163 antibody together with an immune checkpoint inhibitor to treat cancer in a subject in need thereof. An “immunomodulatory CD163 antibody” is an antibody that binds CD163 expressed on an immunosuppressive myeloid cell and modulates the cell's activity or phenotype. An “immunomodulatory CD163 antibody” is an antibody that promotes or enhances an immune response, for example, induces inflammation.

CD163 expression is commonly recognized as a marker for immunosuppressive myeloid cells, such as, for example, myeloid-derived suppressor cells, immunosuppressive macrophages, M2-like macrophages, M2c macrophages, and tumor-associated macrophages. Without being bound by any particular theory, it appears that an immunomodulatory CD163 antibody useful as described herein exerts an effect through binding to hCD163 expressed on a myeloid cell to alter the immunologic status of the cell, thus promoting or enhancing an immune response.

Immunomodulatory CD163 antibodies useful according to the present disclosure include those that relieve or mitigate one or more immunosuppressive effects of such an immunosuppressive myeloid cell.

Again, without being bound by any particular theory, it appears that an immunomodulatory CD163 antibody useful in accordance with the present disclosure causes the immunosuppressive myeloid cell to be “re-oriented” or “re-educated” such that it takes on a functional status that promotes or restores immune responses to cells, such as cancer cells, that may otherwise be suppressed through factors secreted by or interactions with cells such as cancer cells. In some cases, the immunosuppressive myeloid cell may be an M2-like, “anti-inflammatory,” macrophage which may be said to be repolarized by the antibody to an M1-like or “pro-inflammatory” character.

In experimental studies, immunomodulatory CD163 antibodies have been observed to: (i) induce enhanced T cell function, notably function by CD3+ T cells, CD4+ T cells, CD8+ T cells, and/or CD4+ T cells and CD8+ T cells; (ii) relieve T cell suppression or T cell exhaustion that may be induced by cancer cells, such as when an immunomodulatory CD163 antibody useful in accordance with the present disclosure and described herein, acts to “de-suppress” an immune response; (iii) enhance or promote T cell-mediated killing of cancer cells; and/or (iv) stimulate proliferation of T cells, such as those mentioned above and herein. Such observed effects may be termed “immunostimulatory.”

Certain cancer cells overexpress immune checkpoint proteins (e.g., PD-1). These proteins bind to their ligands and this binding results in the immune cell not recognizing the cancer cell as cancerous, thus stopping the immune cell from attacking the cancer cell. Checkpoint inhibitors block the ability of the immune checkpoint protein to bind to its ligand, thus inhibiting the ability of the cancer cell to evade an immune response. In the case of the PD-1/PD-L1 checkpoint, a cancer cell may express PD-L1, which binds to PD-1 expressed on a T cell to suppress the T cell's activity.

Disclosed herein, in certain embodiments, are methods of treating a cancer in a subject in need thereof comprising administering to the subject an immunomodulatory CD163 antibody and an immune checkpoint inhibitor, wherein the administration of the immunomodulatory CD163 antibody and the immune checkpoint inhibitor additively or synergistically exerts a therapeutic effect compared to administration of either the immunomodulatory CD163 antibody or the immune checkpoint inhibitor alone. Without being bound by any theory, the additive or synergistic effect appears to result from an additive or synergistic increase in activity of anti-cancer immune cells (e.g., macrophages, T cells). For example, the present disclosure contemplates that de-suppression or dis-inhibition of immunological anticancer mechanisms in a cancer patient may occur by additive or synergistic activity of an immunomodulatory CD163 antibody and an immune checkpoint inhibitor, such as an antagonist of the PD-1/PD-L1 axis.

Without being bound by any theory, it appears that the binding of the immunomodulatory CD163 antibody to the myeloid cell potentiates an immune response disinhibited by the immune checkpoint inhibitor. Experimental data suggest that the immune checkpoint inhibitor disinhibits immunosuppression by cancer cells in the subject and the immunomodulatory CD163 antibody binding promotes immunostimulatory responses against the cancer cells. More specifically, it appears that the PD-1 antagonist inhibits PD-1-mediated immunosuppression by PD-L1-expressing cancer cells in the subject and the immunomodulatory CD163 antibody promotes an immunostimulatory response against the cancer cells.

In some embodiments, the immunomodulatory CD163 antibody specifically binds to hCD163. In some embodiments, the immunomodulatory CD163 antibody reduces immune suppression by immunosuppressive macrophages (e.g., tumor-associated macrophages, M2 macrophages, M2-like macrophages). For example, in some embodiments, binding of an immunomodulatory CD163 antibody to an immunosuppressive macrophage alters function of the immunosuppressive macrophage and reeducates or converts the immunosuppressive macrophage to be less immunosuppressive (e.g., more M1 or M1-like) causing inhibition of certain immunosuppressive activities.

In some such embodiments, reduction of immunosuppressive activity results in an increase in T cell activity and/or proliferation. Accordingly, in some embodiments, reduction of immunosuppressive activity of immunosuppressive macrophages corresponds with an increase in immunologic response to tumors.

In some embodiments, the immunomodulatory effect of immunomodulatory CD163 antibodies, is assessed by measuring changes in the expression or secretion of soluble factors, or its interactions with other cells, its responses to soluble factors and cellular interactions in the environment, and other such parameters. In some embodiments, the immunomodulatory effect is assessed by measuring activities or phenotypes of cells with which the myeloid cell interacts. In some embodiments, the immunomodulatory effect is assessed by measuring holistic parameters such as an index of overall immune function, e.g., response to a cancer. In some embodiments, the immunomodulatory CD163 antibody is an antibody that binds CD163 expressed on an immunosuppressive macrophage and alters its activity or phenotype. In some embodiments, the immunomodulatory CD163 antibody directly modulates activity of an immunosuppressive myeloid cell and indirectly promotes or enhances an immune response (e.g., via T cells). In some embodiments, the immunomodulatory CD163 antibody reprograms M2-like tumor-associated macrophages (TAM) leading to increased adaptive T-cell activation and proliferation. In some embodiments, the immunomodulatory CD163 antibody specifically binds to a CD163 protein expressed on a human TAM and reduces expression of CD16, CD64, TLR2, Siglec-15, or a combination thereof by the macrophage.

In some embodiments, the immunomodulatory CD163 antibody is an antibody that induces at least one of the following effects when administered to a subject for treatment of a cancerous tumor:
(a) the binding of the antibody to tumor-associated macrophages reduces expression of at least one marker on the macrophages, e.g., CD16, CD64, TLR2, or Siglec-15, or combinations thereof;
(b) upon binding of the antibody with the CD163 protein on tumor-associated macrophages, the antibody is internalized by the macrophages;
(c) the binding of the antibody on tumor-associated macrophages is not cytotoxic to the tumor-associated macrophages;
(d) the binding of the antibody increases the levels of IFN-γ, TNF-α, and/or perforin in the subject;
(e) the binding of the antibody promotes activation of CD4+ T cells, CD8+ T cells, NK cells, or any combination thereof;
(f) the binding of the antibody promotes proliferation of CD4+ T cells, CD8+ T cells, NK cells, or any combination thereof; and
(g) the binding of the antibody promotes tumor cell killing in the tumor microenvironment.

Examples of immunostimulatory CD163 antibodies useful in the methods and combinations of the invention are provided herein, including one such antibody currently being evaluated in a human clinical trial.

### Certain Terminology

Unless defined otherwise, all technical and scientific terms used herein have the same meaning as is commonly understood by one of skill in the art to which the claimed subject matter belongs. Generally, nomenclatures utilized in connection with, and techniques of, immunology, oncology, cell and tissue culture, molecular biology, and protein and oligo- or polynucleotide chemistry and hybridization described herein are those well-known and commonly used in the art. Units of measure not otherwise defined accord with The International System of Units (SI), NIST Special Publication 330, 2019 edition.

It is to be understood that the foregoing general description and the following detailed description are exemplary and explanatory only and are not restrictive of any subject matter claimed. The section headings used herein are for organizational purposes only and are not to be construed as limiting the subject matter described.

As used herein, singular forms “a,” “and,” and “the” include plural referents unless the context clearly indicates otherwise. Thus, for example, reference to “an antibody” includes a plurality of antibodies and reference to “an antibody” in some embodiments includes multiple antibodies, and so forth.

As used herein, all numerical values or numerical ranges include whole integers within or encompassing such ranges and fractions of the values or the integers within or encompassing ranges unless the context clearly indicates otherwise. Thus, for example, reference to a range of 90-100%, includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth. In another example, reference to a range of 1-5,000-fold includes 1-, 2-, 3—, 4-, 5-, 6-, 7-, 8-, 9-, 10-, 11-, 12-, 13-, 14-, 15-, 16-, 17-, 18-, 19-, or 20-fold, etc., as well as 1.1-, 1.2-, 1.3-, 1.4-, or 1.5-fold, etc., 2.1-, 2.2-, 2.3-, 2.4-, or 2.5-fold, etc., and so forth.

“About” a number, as used herein, refers to range including the number and ranging from 10% below that number to 10% above that number. “About” a range refers to 10% below the lower limit of the range, spanning to 10% above the upper limit of the range.

“Percent identity” and “% identity” refers to the extent to which two sequences (nucleotide or amino acid) have the same residue at the same positions in an alignment. For example, “an amino acid sequence is X % identical to SEQ ID NO: Y” refers to % identity of the amino acid sequence to SEQ ID NO: Y and is elaborated as X % of residues in the amino acid sequence are identical to the corresponding residues of sequence disclosed in SEQ ID NO: Y. A sequence said to be X % identical to a reference sequence may contain more nucleotide or amino acid residues than specified in the reference sequence, but must contain sequence corresponding to the reference sequence. In most cases, the sequence in question will contain sequence that corresponds to all of the specified reference sequence. Generally, computer programs are employed for such calculations. Exemplary programs that compare and align pairs of sequences, include ALIGN (Myers and Miller, Comput Appl Biosci. 1988 March; 4(1):11-7), FASTA (Pearson and Lipman, Proc Natl Acad Sci USA. 1988 April; 85(8):2444-8; Pearson, Methods Enzymol. 1990; 183:63-98) and gapped BLAST (Altschul et al., Nucleic Acids Res. 1997 Sep. 1; 25(17):3389-40), BLASTP, BLASTN, or GCG (Devereux et al., Nucleic Acids Res. 1984 Jan. 11; 12(1 Pt 1):387-95).

As used herein “antibody” refers to a protein that binds an antigen. An antibody often comprises a variable domain and a constant domain in each of a heavy chain and a light chain. Accordingly, most antibodies have a heavy chain variable domain (V_{H}) and a light chain variable domain (V_{L}) that together form the portion of the antibody that binds to the antigen, sometimes referred to as the variable region or the “antigen receptor.” Within each variable domain are three complementarity-determining domains (CDR), which form loops in the heavy chain variable domain (V_{H}) and light chain variable domain (V_{L}) and contact the surface of the antigen. “Antibody” includes, but is not limited to, polyclonal, monoclonal, monospecific, multispecific (e.g., bispecific antibodies), natural, humanized, human, chimeric, synthetic, recombinant, hybrid, mutated, grafted, antibody fragments (e.g., a portion of a full-length antibody, generally the antigen-binding or variable region thereof, e.g., Fab, Fab′, F(ab′)₂, and Fv fragments), and in vitro-generated antibodies having the antigen-binding activity. The term also includes single chain antibodies, e.g., single chain Fv (sFv or scFv) antibodies, in which a variable heavy and a variable light chain are joined together (directly or through a peptide linker) to form a continuous polypeptide. “Antibody” also includes cell surface receptor forms in which the constant region comprises hydrophobic portions that permit cell surface expression, in contrast to the hydrophilic portion of the constant region characteristic of circulating or secreted antibodies. Such cell surface forms include, e.g., B-cell receptor and T-cell receptor forms, which can be manufactured using recombinant techniques known in the art. Cell surface receptor forms can be engineered to associate with accessory proteins such as antigen-nonspecific signaling molecules for purposes of signaling when the receptor is bound by antigen.

As used herein “antagonist” refers to an agent that is capable of downwardly modulating activity of a biological target such as a protein. An antagonist may modulate by, fully or partially, blocking, inhibiting, neutralizing, reducing, decreasing, and/or eliminating activity of the target by direct or indirect action (e.g., such as by modulating one or more pathways of the target that results in antagonism of the target).

As used herein “complementarity-determining regions” or “CDRs” or “hypervariable regions” refer to the parts of the variable domains in antibodies that determine the binding specificities of the antibodies to their specific antigen. As noted, a single variable domain of an antibody polypeptide will typically comprise three CDRs, usually designated CDR1, CDR2, and CDR3. More particularly, a heavy chain variable domain may contain CDRs designated H1, H2, and H3; likewise, a light chain variable domain may contain CDRs designated L1, L2, and L3. Multiple methods may be used to define a CDR. The current art utilizes various numbering schemes with different definitions of CDR lengths and positions. For example, the Kabat numbering scheme is based on sequence alignment and uses “variability parameter” of a given amino acid position (the number of different amino acids at a given position divided by the frequency of the most occurring amino acid at that position) to predict CDRs. The Chothia numbering scheme, on the other hand, is a structure-based numbering scheme where antibody crystal structures are aligned as define the loop structures as CDRs. The Martin numbering scheme focuses on the structure alignment of different framework regions of unconventional lengths. IMGT numbering scheme is a standardized numbering system based on alignments of sequences from a complete reference gene database including the whole immunoglobulin superfamily. Honneger's numbering scheme (AHo) is based on structural alignments of the 3D structure of the variable regions and uses structurally conserved Cα positions to deduce framework and CDR lengths. One of skill in the art will note that the definition of a CDR will vary based on the method used. Any method of defining a CDR is contemplated with the sequences disclosed herein.

The terms “recipient,” “individual,” “subject,” “host,” and “patient,” are used interchangeably herein and refer to any mammal for whom diagnosis, treatment, or therapy is desired, particularly humans. None of these terms requires supervision of any medical personnel. “Mammal” for purposes of treatment refers to any animal classified as a mammal, including humans, domestic and farm animals, and laboratory, zoo, sports, or pet animals, such as dogs, horses, cats, cows, sheep, goats, pigs, mice, rats, rabbits, guinea pigs, monkeys, etc. In some embodiments, the mammal is a human.

As used herein, the terms “treatment,” “treating,” and the like, in some cases refer to administering an agent or carrying out a procedure, for the purposes of obtaining an effect. In some embodiments, the effect is prophylactic in terms of completely or partially preventing a disease or symptom thereof and/or is therapeutic in terms of effecting a partial or complete cure for a disease and/or symptoms of the disease. “Treatment,” as used herein, includes treatment of a disease or disorder (e.g., cancer) in a mammal, particularly in a human, and includes: (a) preventing the disease or a symptom of a disease from occurring in a subject which is predisposed to the disease but has not yet been diagnosed as having it (e.g., including diseases that is associated with or caused by a primary disease; (b) inhibiting the disease, i.e., arresting its development; and (c) relieving the disease, i.e., causing regression of the disease. In some embodiments, treating refers to any indicia of success in the treatment or amelioration or prevention of cancer, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms is based on one or more objective or subjective parameters, including the results of an examination by a physician. Accordingly, the term “treating” includes the administration of the compounds or agents of the present disclosure to prevent or delay, to alleviate, or to arrest or inhibit development of the symptoms or conditions associated with diseases (e.g., cancer). The term “therapeutic effect” refers to the reduction, elimination, or prevention of the disease, symptoms of the disease, or side effects of the disease in the subject. For example, in some embodiments, a subject is “treated” for a disease or disorder if, after receiving a therapeutic amount of an immunomodulatory CD163 antibody and an immune checkpoint inhibitor, the patient shows one or more observable and/or measurable changes in a parameter or symptom of the disease or disorder.

In some embodiments, “inducing a response” refers to the alleviation or reduction of signs or symptoms of illness in a subject, and specifically includes, without limitation, prolongation of survival.

As used herein “modulate” refers to a change or an alteration that includes but is not limited to a change in or to a target, pathway, cell, cell population, subject, etc. Modulation may include any change, such as, without limitation, an increase or decrease, in activity, binding, structure, function or other characteristic that can be impacted and an outcome measured.

The term “avidity” refers to the resistance of a complex of two or more agents to dissociation after dilution.

In some embodiments, antibody “effector functions” refers to those biological activities attributable to the Fc region (a native sequence Fc region or amino acid sequence variant Fc region) of an antibody and vary with the antibody isotype.

“Fc receptor” or “FcR” refers to a receptor that binds to the Fc region of an antibody.

“Human effector cells” as used herein refers to leukocytes that express one or more FcRs and perform effector functions. For example, the cells express at least FcγRIII and perform an antibody dependent cell-mediated cytotoxicity (ADCC) effector function. Examples of human leukocytes that mediate ADCC include, but are not limited to, peripheral blood mononuclear cells (PBMC), NK cells, monocytes, macrophages, cytotoxic T cells, and neutrophils.

“Complement-dependent cytotoxicity” or “CDC” refers to the lysis of a target cell in the presence of complement. Activation of the classical complement pathway is initiated by the binding of the first component of the complement system (C1q) to antibodies (of the appropriate subclass) that are bound to their cognate antigen. To assess complement activation, a CDC assay, for example, is performed.

An antibody that “internalizes” is one that is taken up by (i.e., enters) the cell upon binding to an antigen on a mammalian cell (e.g., a cell surface polypeptide or receptor). The internalizing antibody comprises antibody fragments, human or chimeric antibody, and antibody conjugates. In some cases, internalization of an antibody (e.g., such as disclosed herein) alter the biology of the cell, causing it to change its function.

An “antigen-binding domain,” “antigen-binding region,” or “antigen-binding site” is a portion of an antibody that contains amino acid residues (or other moieties) that interact with an antigen and contribute to the antibody's specificity and affinity for the antigen. For an antibody that specifically binds to its antigen, this will include at least part of at least one of its CDR domains.

The antigen-binding region of an antibody is referred to as a “paratope,” which binds to an antigenic determinant, the “epitope” of an antigen, that is, a portion of the antigen molecule that can be bound by an antibody. In some embodiments, an antigen substance has one or more portions that are recognizable by antibodies, i.e., more than one epitope, and thus a single antigen substance is specifically bound by different antibodies each having specificity for a different epitope. In some embodiments, an epitope comprises noncontiguous portions of the antigen. For example, in a polypeptide, amino acid residues that are not contiguous in the polypeptide's primary sequence but that, in the context of the polypeptide's tertiary and quaternary structure, are near enough to each other to be bound by an antigen-binding protein, constitutes an epitope.

An “antibody fragment” comprises a portion of an intact antibody. In some embodiments, the antibody fragment comprises an antigen-binding or variable region of the intact antibody.

The terms “antigen-binding portion of an antibody,” “antigen-binding fragment,” “antigen-binding domain,” “antibody fragment” are used interchangeably herein to refer to any fragment of an antibody that retains the ability to specifically bind to the antigen. Non-limiting examples of antibody fragments included within such terms include, but are not limited to, (i) a Fab fragment, a monovalent fragment consisting of the V_{L}, V_{H}, C_{L}, and C_{H1} domains; (ii) a F(ab′)₂ fragment, a bivalent fragment containing two Fab fragments linked by a disulfide bridge at the hinge region; (iii) a Fd fragment consisting of the V_{H} and C_{H} domains; (iv) a Fv fragment containing the V_{L} and V_{H} domains of a single arm of an antibody, (v) a dAb fragment (Ward et al., Nature 341(6242):544-6 (1989)), containing a V_{H} domain; and (vi) an isolated CDR. Also included are “one-half” antibodies comprising a single heavy chain and a single light chain. Other forms of single chain antibodies, such as diabodies are also encompassed herein.

A “functional antibody fragment” as used herein refers in context to an antibody fragment that not only binds the antibody's antigen, but also possesses a functional attribute that characterizes the intact antibody. For example, if an antibody depends for a function on possessing a Fc domain that enables an effector function, such as ADCC, a functional fragment would possess such function. It is hypothesized that CD163 antibodies useful in accordance with the present disclosure are effective in modulating the functional state of macrophages, such as tissue-resident or infiltrating macrophages, or reorienting, reeducating, or dampening the immunosuppressive/M2-status macrophages (e.g., to or towards non-immunosuppressive/M1 or M1-like status), when they comprise an Fc portion that binds to a macrophage Fc receptor, such as CD16 (FcγRIIIa) or CD64 (FcγRI) in some embodiments; it is further hypothesized that when such an antibody is used in combination with an immune checkpoint inhibitor, the efficacy of the antibody, the immune checkpoint inhibitor, or both, is improved beyond more than an additive effect of each.

The phrase “functional fragment or analog” of an antibody is a compound having qualitative biological activity in common with a full-length antibody. For example, a functional fragment or analog of an anti-IgE antibody is one that binds to an IgE immunoglobulin to prevent or substantially reduce the ability of such molecule from having the ability to bind to the high affinity receptor, FcγRI.

An “antigen-binding protein” is a protein comprising a portion that comprises an antigen-binding portion of an antibody, optionally also including a scaffold or framework portion that allows the antigen-binding portion to adopt a conformation that promotes binding of the antigen-binding protein to the antigen.

An “intact” antibody is one that comprises an antigen-binding site as well as a C_{L} and at least heavy chain constant domains, C_{H}1, C_{H}2, and C_{H}3. In some embodiments, the constant domains are native sequence constant domains (e.g., human native sequence constant domains) or amino acid sequence variant thereof.

The term “recombinant antibody” as used herein refers to an antibody comprising an antigen-binding domain of a first antibody, such as, for example, a CDR, a V_{H} domain, or an intact light chain, and a domain from one or more other antibodies or proteins. Chimeric, hybrid, and humanized antibodies are examples of recombinant antibodies.

A “CDR-grafted antibody” is an antibody comprising one or more CDRs derived from an antibody of one species or isotype and the framework of another antibody of the same or different species or isotype.

The term “human antibody” includes all antibodies that have one or more variable and constant domains derived from human immunoglobulin sequences. In one embodiment, all of the variable and constant domains of the antibody are derived from human immunoglobulin sequences (referred to as a “fully human antibody”).

As used herein, the term “affinity” refers to the equilibrium constant for the reversible binding of two agents and is expressed as the equilibrium dissociation constant, K_{D}. In one embodiment, the antibodies or antigen-binding fragments thereof exhibit binding affinity as measured by K_{D} for CD163 in the range of 10⁻⁶ M or less, or ranging down to 10⁻¹⁶ M or lower, (e.g., about 10⁻⁷, 10⁻⁸, 10⁻⁹, 10⁻¹⁰, 10⁻¹¹, 10⁻¹², 10⁻¹³, 10⁻¹⁴, 10⁻¹⁵, 10⁻¹⁶ M or less). In certain embodiments, antibodies as describe herein specifically bind to a human CD163 (hCD163) polypeptide with a K_{D} of less than or equal to 10⁻⁴ M, less than or equal to about 10⁻⁵ M, less than or equal to about 10⁻⁶ M, less than or equal to 10⁻⁷ M, or less than or equal to 10⁻⁸ M.

The terms “preferentially binds” or “specifically binds” mean that the antibodies or fragments thereof bind to an epitope with greater affinity than it binds unrelated amino acid sequences, and, if cross-reactive to other polypeptides containing the epitope, are not toxic at the levels at which they are formulated for administration to human use. In some embodiments, such affinity is at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater than the affinity of the antibody or fragment thereof for unrelated amino acid sequences.

The term “specific” refers to a situation in which an antibody will preferentially bind to molecules other than the antigen containing the epitope recognized by the antibody. The term is also applicable where for example, an antigen-binding domain is specific for a particular epitope which is carried by a number of antigens, in which case the antibody or antigen-binding fragment thereof carrying the antigen-binding domain will be able to bind to the various antigens carrying the epitope.

As used herein, an antibody is said to be “immunospecific” or “specific” for, or to “specifically bind” to, an antigen if that antibody reacts at a detectable level with the antigen, preferably with an affinity constant (association constant), K_{A}, of greater than or equal to about 10⁴ M⁻¹, or greater than or equal to about 10⁵ M⁻¹, greater than or equal to about 10⁶ M⁻¹, greater than or equal to about 10⁷ M⁻¹, or greater than or equal to 10⁹ M⁻¹.

The term “monospecific,” as used herein, refers to an antibody composition that contains an antibody that displays a preferential affinity for one particular epitope. In some embodiments, monospecific antibody preparations are made up of about 10%, 20%, 30%, 40%, 50%, 60%, 70%, 75%, 80%, 85%, 90%, 95%, 97%, 99%, or 99.9% antibody having specific binding activity for the antigen.

The term “polypeptide” is used in its conventional meaning, i.e., as a sequence of amino acids. The polypeptides are not limited to a specific length of the product. Peptides, oligopeptides, and proteins are included within the definition of polypeptide, and such terms are used interchangeably herein unless specifically indicated otherwise. This term also does not refer to or exclude post-expression modifications of the polypeptide, for example, glycosylations, acetylations, phosphorylations, and the like, as well as other modifications known in the art, both naturally occurring and non-naturally occurring. In some embodiments, a polypeptide is an entire protein, or a subsequence thereof. Polypeptides of interest in the context of the CD163 antibodies of this disclosure include amino acid subsequences comprising CDRs and are capable of binding human immunosuppressive macrophages (e.g., tumor-associated macrophages, e.g., M2 or M2-like macrophages) or CD163 protein expressed by such cells.

As used herein, “substantially pure,” and “substantially free” refer to a solution or suspension containing less than, for example, about 20% or less extraneous material, about 10% or less extraneous material, about 5% or less extraneous material, about 4% or less extraneous material, about 3% or less extraneous material, about 2% or less extraneous material, or about 1% or less extraneous material.

The term “isolated” refers to a protein (e.g., an antibody), nucleic acid, or other substance that is substantially free of other cellular material and/or chemicals. In some embodiments, the antibodies, or antigen-binding fragments thereof, and nucleic acids of the disclosure are isolated. In some embodiments, the antibodies, or antigen-binding fragments thereof, and nucleic acids of the disclosure are substantially pure.

When applied to polypeptides, “isolated” generally means a polypeptide that has been separated from other proteins and nucleic acids with which it naturally occurs. Preferably, the polypeptide is also separated from substances such as antibodies or gel matrices (polyacrylamide) which are used to purify it. In some cases, the term means a polypeptide or a portion thereof which, by virtue of its origin or manipulation: (i) is present in a host cell as the expression product of a portion of an expression vector; or (ii) is linked to a protein or other chemical moiety other than that to which it is linked in nature; or (iii) does not occur in nature, for example, a protein that is chemically manipulated by appending, or adding at least one hydrophobic moiety to the protein so that the protein is in a form not found in nature. By “isolated” it is further meant a protein that is: (i) synthesized chemically; or (ii) expressed in a host cell and purified away from associated and contaminating proteins.

The term “effective amount” as used herein, refers to that amount of an antibody, or an antigen-binding portion thereof as described herein, that is sufficient to induce a response, e.g., to effect treatment, prognosis, or diagnosis of a disease associated with macrophage activity, as described herein, when administered to a subject. Therapeutically effective amounts of antibodies provided herein, when used alone or with an immune checkpoint inhibitor, will vary depending upon the relative activity of the antibodies and combinations (e.g., in treating/reducing/ameliorating cancer) and depending upon the subject and disease condition being treated, the weight and age of the subject, the severity of the disease condition, the manner of administration, and the like, which, in some cases, are readily determined by one of ordinary skill in the art.

The term “therapeutically effective amount” or “therapeutic amount” generally refers to an amount of an antibody or a drug effective to “treat” a disease or disorder in a subject or mammal. In some embodiments, a composition described herein is administered to a subject in an amount that is effective for producing some desired therapeutic effect by inhibiting a disease or disorder as described herein at a reasonable benefit/risk ratio applicable to any medical treatment. A therapeutically effective amount is an amount that achieves at least partially a desired therapeutic or prophylactic effect in an organ or tissue. The amount of an antibody or drug necessary to bring about prevention and/or therapeutic treatment of a disease or disorder is not fixed per se. In some embodiments, the amount of an antibody or drug administered varies with the type of disease, extensiveness of the disease, and size of the mammal suffering from the disease or disorder. When used in conjunction with therapeutic methods involving administration of a therapeutic agent after the subject presents symptoms of a disease or disorder, the term “therapeutically effective” means that, after treatment, one or more signs or symptoms of the disease or disorder is ameliorated or eliminated.

When a combination of treatment modalities (e.g., an immunomodulatory CD163 antibody and an immune checkpoint inhibitor) is employed in the treatment of disease, each treatment modality in the combination may be used in an amount that would itself be therapeutically effective (when used as a monotherapy) or in an amount that would be considered subtherapeutic (e.g., insufficient relative to the monotherapy), but each is considered administered in a therapeutically effective amount because when administered together, the result is therapeutic effect. Thus, in a combination of the present disclosure, an agent that would be expected to be effective at a dose or range of doses when used alone may be administered in a subtherapeutic amount, i.e., an amount that is below that normally associated with a treatment effect. The therapeutically effective amount of the combination may thus rely on the effects of each modality in the combination exerting complementary effects that might not be otherwise observable in the subject were the modalities administered individually. However, an advantage of the method of the present disclosure is that, in some embodiments, an immunomodulatory CD163 antibody and an immune checkpoint inhibitor may each be administered at doses that are, in some embodiments, individually effective, but together their effect in the patient may be observed to be more than merely additive. That is, in some embodiments, administration of an immunomodulatory CD163 antibody and an immune checkpoint inhibitor may result in an additive or synergistic benefit, measured, e.g., as an improved outcome for or response in the subject.

An “effective response” in accordance with the present disclosure is achieved when the subject experiences partial or total alleviation or reduction of signs or symptoms of illness and, in the case of the treatment of cancer, specifically includes, without limitation, amelioration of symptoms, inhibition of progression, cure, remission, prolongation of survival, reduction of tumor burden, or other meaningful responses. In some embodiments, the expected progression-free survival times are measured in months to years, depending on prognostic factors including the number of relapses, stage of disease, and other factors. Prolonging survival includes without limitation times of at least 1 month (mo.), about at least 2 mos., about at least 3 mos., about at least 4 mos., about at least 6 mos., about at least 1 year, about at least 2 years, about at least 3 years, etc. Overall survival is also measured, for example, in months to years. Alternatively, an effective response, in some embodiments, is that a subject's symptoms or disease burden remain static. Further indications of treatment are described in more detail below.

In some embodiments, administration of a therapeutic agent in a prophylactic method occurs prior to the manifestation of symptoms of an undesired disease or disorder, such that the disease or disorder is prevented or, alternatively, delayed in its progression. Thus, when used in conjunction with prophylactic methods, the term “therapeutically effective” means that, after treatment, a smaller number of subjects (on average) develop the undesired disease or disorder or progress in severity of symptoms or disease burden.

The term “checkpoint inhibitor” or “immune checkpoint inhibitor” refers to an agent that modulates an immune checkpoint protein (a “checkpoint protein”). An immune checkpoint inhibitor may achieve total or partial reduction, inhibition, interference to an activity of the immune checkpoint protein, or produce other changes to structure of the immune checkpoint protein that change binding of an immune checkpoint protein to a ligand, and/or impact a pathway related to activity of the immune checkpoint protein such as, for example, by acting as an antagonist to an immune checkpoint protein or a ligand of the immune checkpoint protein. In some embodiments, the immune checkpoint is a T-cell immune checkpoint, and the immune checkpoint inhibitor, in some embodiments, is a modulator of the immune checkpoint protein itself or of a ligand, e.g., a natural ligand thereof. For example, an immune checkpoint inhibitor may bind to a cell expressing a particular immune checkpoint protein or may bind to another cell that expresses a ligand of the immune checkpoint protein.

The phrase “pharmaceutically acceptable” refers to molecular entities and compositions that are physiologically tolerable and do not typically produce intolerable allergic or similar untoward reaction, when administered to a human. In some embodiments, a composition is pharmaceutically acceptable if, when administered to the patient, any unexpected or undesirable side effects caused by or attributable to the active ingredient and/or any excipient in the composition are deemed by the patient or the clinician staff to be minimal or acceptable given the overall treatment status and condition of the patient. A pharmaceutical composition, or its route of administration, in some embodiments, induces side effects considered to be mild or tolerable by the patient or the physician and be considered pharmaceutically acceptable.

The term “contacting” is defined herein as a means of bringing a composition as provided herein in physical proximity with a cell, organ, tissue, or fluid as described herein.

The term “combination” (and related phrases such as “in combination with”) refers to administration of one treatment modality in addition to another treatment modality. Accordingly, “in combination with” refers to administration of one treatment modality before, during, or after administration of the other treatment modality to the subject. In some embodiments, “combination” may relate to articles of manufacture that comprise treatment modalities for combination use.

### Methods of Treatment and CD163 and Checkpoint Combinations

Described herein, in some embodiments, are methods of treating a cancer in a subject in need thereof, comprising: administering to the subject an effective amount of: a) a means for binding human CD163 (hCD163); and b) a means for inhibiting an immune checkpoint protein or a ligand thereof. Further described herein, in some embodiments, are methods of treating a cancer in a subject in need thereof, comprising administering to the subject an effective amount of (a) means for modulating an immune function of a tumor-associated macrophage expressing human CD163 protein (hCD163) by binding the hCD163 protein at a localized region on a surface of the hCD163 protein comprising an amino acid sequence IGRVNASKGFGHIWLDSVSCQGHEPAI (SEQ ID NO: 43), VVCRQLGCGSA (SEQ ID NO: 44), WDCKNWQWGGLTCD (SEQ ID NO: 45), or any combination thereof; and (b) a means for inhibiting an immune checkpoint protein or a ligand thereof. Further described herein, in some embodiments, are methods of treating a cancer in a subject in need thereof, comprising administering to the subject an effective amount of: (a) means for modulating an immune function of a tumor-associated macrophage that expresses human CD163 protein (hCD163) by binding the hCD163 protein at a localized region on a surface of the hCD163 protein comprising an amino acid sequence IGRVNASKGFGHIWLDSVSCQGHEPAI (SEQ ID NO: 43), VVCRQLGCGSA (SEQ ID NO: 44), WDCKNWQWGGLTCD (SEQ ID NO: 45), or any combination thereof; and (b) a means for inhibiting an immune checkpoint protein or a ligand thereof. Further described herein, in some embodiments, are methods of treating a cancer in a subject in need thereof, comprising administering to the subject an effective amount of: (a) a modulator of tumor-associated macrophages comprising a means for binding human CD163 (hCD163) comprising at least 90% sequence identity to SEQ ID NO: 42; and (b) a means for inhibiting an immune checkpoint protein or a ligand thereof. Further described herein, in some embodiments, are methods of treating a cancer in a subject in need thereof, comprising administering to the subject an effective amount of a pharmaceutical acceptable composition comprising (a) a means for binding human CD163 (hCD163); (b) a means for inhibiting an immune checkpoint protein or a ligand thereof; and (c) a pharmaceutically acceptable excipient.

In some embodiments, the means for binding hCD163 binds hCD163 expressed on a myeloid cell or soluble hCD163. In some embodiments, the means for binding hCD163 binds hCD163 expressed on a myeloid cell. In some embodiments, the means for binding the human myeloid cell is a tumor-associated macrophage. In some embodiments, the means for binding the means for binding hCD163 binds domain 3 of hCD163. In some embodiments, the means for binding hCD163 binds the hCD163 protein at a localized region on a surface of the hCD163 protein comprising an amino acid sequence IGRVNASKGFGHIWLDSVSCQGHEPAI (SEQ ID NO: 43), VVCRQLGCGSA (SEQ ID NO: 44), WDCKNWQWGGLTCD (SEQ ID NO: 45), or any combination thereof. In some embodiments, the means for binding the means for binding hCD163 specifically binds hCD163. In some embodiments, the means for binding hCD163 comprises an antibody or antigen-binding fragment thereof. In some embodiments, the means for binding the antibody is an IgG1 antibody.

In some embodiments, the immune checkpoint protein is PD-1, CD28, CTLA-4, ICOS, TMIGD2, 4-1BB, BTLA, CD160, LIGHT, LAG3, OX40, CD27, CD40L, GITR, DNAM-1, TIGIT, CD96, 2B4, TIM-3, CEACAM1, SIRP alpha, DC-SIGN, CD200R, DR3, CDCHK1, CHK2, A2aR, or B-7 family proteins. In some embodiments, the immune checkpoint protein is PD-1. In some embodiments, the ligand is PD-L1 (B7-H1), PD-L2 (B7-DC), ICOS ligand, VISTA, 4-1BBL, Herpesvirus Entry Mediator (HVEM), tumor necrosis factor receptor superfamily member 14 or TNFRSF14, MHC class I, MHC class II, OX-40L, CD70, CD40, GITRL, CD155, CD48, GAL9; HMGB1, CEASAM-1, Phosphatidyl serine (PtdSer), IDO, TDO, CD47, BTN2A1, CD200, TL1A, CD112, CD155, MHCII, LSECtin, CHK1, CHK2, A2aR, or a B-7 family ligand (e.g., CD80 (B7-1), CD86 (B7-2), B7-H3, B7-H4, B7-H7 (HHLA2), etc.). In some embodiments, the ligand is PD-L1. In some embodiments, the means for inhibiting an immune checkpoint protein or a ligand thereof is an immune checkpoint inhibitor. In some embodiments, the means for inhibiting an immune checkpoint protein or a ligand thereof comprises an antibody or antigen-binding fragment thereof.

Described herein, in some embodiments, are methods of treating a cancer in a subject in need thereof, comprising administering to the subject an effective amount of: (a) means for modulating an immune function of a tumor-associated macrophage that expresses human CD163 protein (hCD163) by the hCD163 protein at a localized region on a surface of the hCD163 protein comprising an amino acid sequence IGRVNASKGFGHIWLDSVSCQGHEPAI (SEQ ID NO: 43), VVCRQLGCGSA (SEQ ID NO: 44), WDCKNWQWGGLTCD (SEQ ID NO: 45), or any combination thereof; and (b) a means for inhibiting an immune checkpoint protein or a ligand thereof. In some embodiments, the means for modulating an immune function comprises an antibody or antigen-binding fragment thereof. In some embodiments, the antibody is an IgG1 antibody. In some embodiments, the modulating of an immune function comprises: (i) inducing enhanced function of CD3+ T cells, CD4+ T cells, CD8+ T cells, and/or CD4+ T cells and CD8+ T cells; (ii) relieving T cell suppression or T cell exhaustion that may be induced by cancer cells; (iii) increasing T cell-mediated killing of cancer cell; (iv) or stimulating proliferation of T cells. In some embodiments, the means for modulating of an immune function comprises: (a) reducing expression of at least one marker on the macrophages selected from the group consisting of CD16, CD64, TLR2, and Siglec-15; (b) internalizing of bound antibody by the macrophages; (c) increasing IFN-γ, TNF-α, or perforin in the subject; (d) promoting activation of CD4+ T cells, CD8+ T cells, or NK cells; (e) promoting proliferation of CD4+ T cells, CD8+ T cells, or NK cells; or (f) promoting tumor cell killing in the tumor microenvironment.

Disclosed herein, in certain embodiments, are methods of treating a cancer in a subject in need thereof, comprising: administering to the subject: a) an immunomodulatory CD163 antibody that binds to hCD163 on a myeloid cell; and b) an immune checkpoint inhibitor, wherein the immune checkpoint inhibitor inhibits the activity of an immune checkpoint protein expressed on a cell of the cancer. In some embodiments, administering the immunomodulatory CD163 antibody that binds to hCD163 on the myeloid cell and the immune checkpoint inhibitor is additive or synergizes to result in improved treatment of the cancer. In some embodiments, administering the immunomodulatory CD163 antibody and the immune checkpoint inhibitor to a subject additively or synergistically exerts a therapeutic effect such that there is a greater than additive effect than achieved with administration of either the immunomodulatory CD163 antibody or the immune checkpoint inhibitor were administered alone.

Further disclosed herein, in certain embodiments, is an additive or synergistic combination of a) an immunomodulatory CD163 antibody that binds to hCD163 on a myeloid cell; and b) an immune checkpoint inhibitor. In some embodiments, the myeloid cell is an immunosuppressive macrophage (e.g., a tumor associated macrophage, M2 or M2-like macrophage). In some embodiments, presence or administration of (i) an immunomodulatory CD163 antibody (i.e., an hCD163 antibody) that specifically binds to human myeloid cells and (ii) an immune checkpoint inhibitor results in additive or synergistic immunomodulation. For example, without being bound by any particular theory, in some such embodiments, the immunomodulatory antibody results in a decrease in immunosuppressive activity by an immunosuppressive macrophage and, along with the immune checkpoint inhibitor, results in additive or synergistic impact on relief of immunosuppression and increase in T-cell activity such as, for example, T-cell mediated activity towards a cancer cell.

In some embodiments, a subject is in need of modulation of immune activity. In some embodiments, a subject has a pathologically or inappropriately elevated level of immunosuppressive macrophages (e.g., tumor-associated macrophages, e.g., M2-like macrophages). In some embodiments, a subject is experiencing immunosuppressive macrophage-mediated T cell suppression. That is, in some embodiments, immunosuppressive macrophages (e.g., tumor-associated macrophages, M2 or M2-like macrophages) are causing suppression of an immune response by T cells. In some embodiments, a subject has cancer. In some embodiments, a subject has a tumor. In some such embodiments, a tumor is non-responsive to treatment with another therapy (e.g., an antibody, including an immunomodulatory CD163 antibody; an immune checkpoint inhibitor; radiotherapy, chemotherapy, etc.). In some such embodiments, a tumor is responsive to treatment with an immunomodulatory CD163 antibody and an immune checkpoint inhibitor. In some embodiments, the tumor responds to an additive or synergistic effect of a treatment with an immunomodulatory CD163 antibody and an immune checkpoint inhibitor.

Without being bound by any particular theory, the present disclosure contemplates that a use of an immunomodulatory hCD163 antibody and an immune checkpoint inhibitor (i.e., to prevent an immune checkpoint protein-ligand interaction) results in additivity or synergy that relieves macrophage-mediated T cell suppression more effectively, and more than as would be expected from additive impact, than use of either the CD163 antibody or the immune checkpoint inhibitor alone. In some embodiments, “unblocking” an immune checkpoint protein using an immune checkpoint inhibitor allows a T cell to act on (e.g., kill) a cancer cell. In some such embodiments, an immune checkpoint inhibitor and an immunomodulatory CD163 antibody are additive or synergize to more effectively relieve macrophage-mediated immune suppression and activate T cells such that they are more functional and effective at responding, to, for example, a tumor (e.g., as measured by increased secretion of cytokines and proliferation of CD3+ T cells and subtypes therein).

In some embodiments, an immunomodulatory CD163 antibody for use with a method disclosed herein comprises or consists of one or more variable domains comprising or consisting of one or more sequences set forth in Table 1. In some embodiments, a variable domain sequence comprises or consists of a sequence that has a particular percent identity to any one of SEQ ID NOs: 28-41.

In some embodiments, an immunomodulatory CD163 antibody for use with a method disclosed herein binds to particular epitope of CD163 on a myeloid cell. In some embodiments, the sequence of the epitope comprises or consists of a sequence that has a particular percent identity to any of SEQ ID NOs 43-45. In some embodiments, the binding of the immunomodulatory CD163 antibody in the presence of an immune checkpoint inhibitor (e.g., prior to, concomitantly with, and/or subsequent to presence of an immune checkpoint inhibitor) results in an additive or synergistic outcome that is not observed or achieved with the immunomodulatory CD163 antibody or the immune checkpoint inhibitor alone.

In some embodiments, the immune checkpoint protein expressed on a cell of the cancer is selected from the group consisting of: PD-1, CD28, CTLA-4, ICOS, TMIGD2, 4-1BB, BTLA, CD160, LIGHT, LAG3, OX40, CD27, CD40L, GITR, DNAM-1, TIGIT, CD96, 2B4, TIM-3, CEACAM1, SIRP alpha, DC-SIGN, CD200R, DR3, CDCHK1, CHK2, A2aR, or B-7 family proteins, and ligands thereof that enable the immune checkpoint function.

In some embodiments, the immune checkpoint inhibitor is an antagonist of an immune checkpoint protein. In some embodiments, the immune checkpoint inhibitor is an antagonist of an immune checkpoint protein ligand. In some embodiments, the antagonist is an antibody.

In some embodiments, the cancer is characterized by insufficient T cell activity, which may be due to insufficient numbers of T cells (e.g., tumor-infiltrating lymphocytes), possibly due to the cancer expressing few antigens, or to the T cells that are resident in the tumor or the stroma being relatively inactive, possibly through immunosuppression mediated by the cancer.

In some embodiments, the cancer is associated with a presence of immunosuppressive macrophages (e.g., tumor-associated macrophages, e.g., M2 or M2-like macrophages, etc.).

In some embodiments, cancer cells (e.g., of a subject) express PD-1 or PD-L1. In some embodiments, cancer cells express PD-1.

In some embodiments, the present disclosure provides methods of treating a cancer in an individual in need thereof, comprising administering to the individual an immunomodulatory CD163 antibody as provided herein and an immune checkpoint inhibitor. In some embodiments, the CD163 antibody is an immunomodulatory CD163 antibody. In some such embodiments, the immunomodulatory CD163 antibody binds to human myeloid cells. In some such embodiments, the human myeloid cells are macrophages. In some embodiments, the macrophages are immunosuppressive macrophages (e.g., tumor-associated macrophages, M2 or M2-like macrophages, etc.).

In some such embodiments, the disclosure provides a use of a combination of the CD163 antibody and an immune checkpoint inhibitor, for the manufacture of a medicament for treating a cancer in a human subject. In some embodiments, the immunomodulatory CD163 antibody specifically binds to a CD163 protein expressed on human tumor associated macrophages and, when administered with an immune checkpoint inhibitor, the macrophages express reduced levels of at least one of CD16, CD64, TLR2, or Siglec-15 than is or would be expected with either the immunomodulatory CD163 antibody or checkpoint inhibitor alone.

Disclosed herein, in certain embodiments, are methods of modulating immune activity in a subject in need thereof, comprising administering to said subject an immunomodulatory CD163 antibody (e.g., as disclosed herein) and an immune checkpoint inhibitor. In some embodiments, treatment with an immune checkpoint inhibitor and an immunomodulatory CD163 antibody that specifically binds to a CD163 protein expressed on human tumor associated macrophages (i.e., hCD163), is additive or synergizes and reduces expression of at least one of CD16, CD64, TLR2, or Siglec-15 by the macrophages to a greater degree than with the CD163 antibody or checkpoint inhibitor alone and/or as would be expected from additive effect of the CD163 antibody and checkpoint inhibitor.

Disclosed herein, in certain embodiments, are methods of treating a subject with pathologically or inappropriately elevated levels of immunosuppressive macrophages (e.g., tumor-associated, e.g., M2-like macrophages). In some such embodiments, inappropriately elevated means relative to the level useful for promoting immune-mediated tumor cell killing in the subject.

In some embodiments, the present disclosure provides a method of treating a subject with pathologically or inappropriately elevated levels of tumor-associated macrophages comprising administering to said subject an immunomodulatory CD163 antibody described herein and an immune checkpoint inhibitor. In some embodiments, treatment with the CD163 antibody and the immune checkpoint inhibitor additively or synergistically reduces expression of at least one of CD16, CD64, TLR2, or Siglec-15 by the macrophages to a greater degree than with the CD163 antibody or checkpoint inhibitor alone.

Disclosed herein, in certain embodiments, are methods of modulating an activity of a tumor-associated macrophage in a tumor microenvironment, the method comprising contacting the tumor-associated macrophage with a combination of an immunomodulatory CD163 antibody and an immune checkpoint inhibitor, wherein the method results in at least one of the following effects: (a) reduced expression of at least one marker by the human macrophage, wherein the at least one marker is CD16, CD64, TLR2, or Siglec-15; (b) internalization of the immunomodulatory CD163 antibody by the human macrophage; (c) activation of a CD4⁺ T cell, CD8⁺ T cell, NK cell, or any combination thereof; (d) proliferation of a CD4⁺ T cell, CD8⁺ T cell, NK cell, or any combination thereof; and (e) promotion of tumor cell killing in a tumor microenvironment, wherein the results of one or more of (a)-(e) is more pronounced with the combination than with the CD163 antibody or an immune checkpoint inhibitor alone.

Disclosed herein, in certain embodiments, are methods of modulating an activity of a tumor-associated macrophage in a tumor microenvironment, the method comprising contacting the tumor-associated macrophage with an immunomodulatory CD163 antibody and an immune checkpoint inhibitor, wherein the method results in at least two of the following effects: (a) reduced expression of at least one marker by the human macrophage, wherein the at least one marker is CD16, CD64, TLR2, or Siglec-15; (b) internalization of the immunomodulatory CD163 antibody by the human macrophage; (c) activation of a CD4⁺ T cell, CD8⁺ T cell, NK cell, or any combination thereof; (d) proliferation of a CD4⁺ T cell, CD8⁺ T cell, NK cell, or any combination thereof; and (e) promotion of tumor cell killing in a tumor microenvironment, wherein the results of one or more of (a)-(e) is more pronounced with the combination than with CD163 antibody or an immune checkpoint inhibitor alone.

Disclosed herein, in certain embodiments, are methods of modulating an activity of a tumor-associated macrophage in a tumor microenvironment, the method comprising contacting the tumor-associated macrophage with an immunomodulatory CD163 antibody and an immune checkpoint inhibitor, wherein the method results in at least three of the following effects: (a) reduced expression of at least one marker by the human macrophage, wherein the at least one marker is CD16, CD64, TLR2, or Siglec-15; (b) internalization of the immunomodulatory CD163 antibody by the human macrophage; (c) activation of a CD4⁺ T cell, CD8⁺ T cell, NK cell, or any combination thereof; (d) proliferation of a CD4⁺ T cell, CD8⁺ T cell, NK cell, or any combination thereof; and (e) promotion of tumor cell killing in a tumor microenvironment, wherein the results of one or more of (a)-(e) is more pronounced with the combination than with CD163 antibody or an immune checkpoint inhibitor alone . . .

Disclosed herein, in certain embodiments, are methods of modulating an activity of a tumor-associated macrophage in a tumor microenvironment, the method comprising contacting the tumor-associated macrophage with an immunomodulatory CD163 antibody and an immune checkpoint inhibitor, wherein the method results in at least four of the following effects: (a) reduced expression of at least one marker by the human macrophage, wherein the at least one marker is CD16, CD64, TLR2, or Siglec-15; (b) internalization of the immunomodulatory CD163 antibody by the human macrophage; (c) activation of a CD4⁺ T cell, CD8⁺ T cell, NK cell, or any combination thereof; (d) proliferation of a CD4⁺ T cell, CD8⁺ T cell, NK cell, or any combination thereof; and (e) promotion of tumor cell killing in a tumor microenvironment, wherein the results of one or more of (a)-(e) is more pronounced with the combination than with CD163 antibody or an immune checkpoint inhibitor alone.

Disclosed herein, in certain embodiments, are methods of modulating an activity of a tumor-associated macrophage in a tumor microenvironment, the method comprising contacting the tumor-associated macrophage with an immunomodulatory CD163 antibody and an immune checkpoint inhibitor, wherein the method results in at least five of the following effects: (a) reduced expression of at least one marker by the human macrophage, wherein the at least one marker is CD16, CD64, TLR2, or Siglec-15; (b) internalization of the immunomodulatory CD163 antibody by the human macrophage; (c) activation of a CD4⁺ T cell, CD8⁺ T cell, NK cell, or any combination thereof; (d) proliferation of a CD4⁺ T cell, CD8⁺ T cell, NK cell, or any combination thereof; and (e) promotion of tumor cell killing in a tumor microenvironment, wherein the results of one or more of (a)-(e) is more pronounced with the combination than with CD163 antibody or an immune checkpoint inhibitor alone.

Disclosed herein, in certain embodiments, are methods of functionally reorienting tumor-associated macrophages to reduce immunosuppression in a patient having cancer, comprising administering to the patient an amount of a pharmaceutical composition comprising an immunomodulatory CD163 antibody and an immune checkpoint inhibitor such that the improvement on CD4⁺ or CD8⁺ T cell activity or proliferation in the tumor microenvironment is additive or synergistic, resulting in greater efficacy than administration of either the immunomodulatory CD163 antibody or checkpoint inhibitor. In some such embodiments, a pharmaceutical composition may include more than one (e.g., two or more) individual pharmaceutical compositions. For instance, in some embodiments, a pharmaceutical composition may comprise an immunomodulatory CD163 antibody and an immune checkpoint inhibitor each supplied and administered as their own composition, but part of the same pharmaceutical composition. In some embodiments, a pharmaceutical composition may comprise one, or more than one active agents (e.g., anti-hCD163 antibody, e.g., checkpoint inhibitor, e.g., anti-hCD163 antibody and checkpoint inhibitor).

Disclosed herein, in certain embodiments, are methods of promoting lymphocyte-mediated tumor cell killing in a human subject in need thereof, comprising administering to the subject an effective amount of a pharmaceutical composition comprising an immunomodulatory CD163 antibody and an immune checkpoint inhibitor.

In some embodiments, the disclosure provides a use of an immunomodulatory CD163 antibody for the manufacture of a medicament that reduces immunosuppression by tumor-associated macrophages in a human subject having a cancer, which can be combined with use of an immune checkpoint inhibitor for the manufacture of a medicament for use in treatment of a human subject having cancer, which, when combined, is additive or synergizes to provide an improved medicament relative to a medicament comprising either the an immunomodulatory CD163 antibody or checkpoint inhibitor alone.

In some embodiments, the disclosure provides a use of an immunomodulatory CD163 antibody for the manufacture of a medicament that promotes T cell-mediated tumor cell killing in a human subject having a cancer, which can be combined with use of an immune checkpoint inhibitor for the manufacture of a medicament for use in treatment of a human subject having cancer. In some embodiments, treatment with the immunomodulatory CD163 antibody and checkpoint inhibitor results in an additive or synergistic effect as compared to treatment with either the immunomodulatory CD163 antibody or checkpoint inhibitor alone.

Any of the methods disclosed herein, in some instances, further comprise administering to said subject an additional anticancer therapy, in addition to the immunomodulatory CD163 antibody and an immune checkpoint inhibitor. Anticancer therapies include, but are not limited to, surgical therapy, chemotherapy, radiation therapy, cryotherapy, hormonal therapy, immunotherapy, and cytokine therapy, and combinations thereof. In one embodiment, the immunomodulatory CD163 antibody or antigen-binding fragment thereof and the anticancer therapy are administered concurrently or sequentially.

Described herein, in some embodiments, are combinations for use in treating a cancer. In some embodiments, the combination (e.g., a combination product) comprises (i) an immunomodulatory CD163 antibody and (ii) an immune checkpoint inhibitor selected from a PD-1 antagonist and a PD-L1 antagonist, for use in the manufacture of a medicament, wherein the immunomodulatory CD163 antibody comprises a light chain variable domain (V_{L}) having a sequence at least 80% identical to the amino acid sequence selected from the group consisting of: SEQ ID NO: 28, SEQ ID NO: 30, SEQ ID NO: 32, SEQ ID NO: 34, SEQ ID NO: 36, SEQ ID NO: 38, and SEQ ID NO: 40, and a heavy chain variable domain (V_{H}) having a sequence at least 80% identical to the amino acid sequence selected from the group consisting of: SEQ ID NO: 29, SEQ ID NO: 31, SEQ ID NO: 33, SEQ ID NO: 35, SEQ ID NO: 37, SEQ ID NO: 39, and SEQ ID NO: 41. In some embodiments, the combination comprises (i) an immunomodulatory CD163 antibody comprising a light chain variable domain (V_{L}) having a sequence at least 80% identical to the amino acid sequence selected from the group consisting of: SEQ ID NO: 28, SEQ ID NO: 30, SEQ ID NO: 32, SEQ ID NO: 34, SEQ ID NO: 36, SEQ ID NO: 38, and SEQ ID NO: 40, and a heavy chain variable domain (V_{H}) having a sequence at least 80% identical to the amino acid sequence selected from the group consisting of: SEQ ID NO: 29, SEQ ID NO: 31, SEQ ID NO: 33, SEQ ID NO: 35, SEQ ID NO: 37, SEQ ID NO: 39, and SEQ ID NO: 41, and (ii) an immune checkpoint inhibitor selected from a PD-1 antagonist and a PD-L1 antagonist, wherein the combination is used in the preparation of a medicament for the treatment of cancer. In some embodiments, the combination comprises (i) an immunomodulatory CD163 antibody comprising a light chain variable domain (V_{L}) having a sequence at least 80% identical to the amino acid sequence selected from the group consisting of: SEQ ID NO: 28, SEQ ID NO: 30, SEQ ID NO: 32, SEQ ID NO: 34, SEQ ID NO: 36, SEQ ID NO: 38, and SEQ ID NO: 40, and a heavy chain variable domain (V_{H}) having a sequence at least 80% identical to the amino acid sequence selected from the group consisting of: SEQ ID NO: 29, SEQ ID NO: 31, SEQ ID NO: 33, SEQ ID NO: 35, SEQ ID NO: 37, SEQ ID NO: 39, and SEQ ID NO: 41, and (ii) an immune checkpoint inhibitor, wherein the combination is for use in treating a cancer.

Described herein, in some embodiments, are combinations for use in treating a cancer, wherein the combination comprises (i) an immunomodulatory CD163 antibody comprising a six CDRs set forth as follows: (a) RASQSISX₈YLN (SEQ ID NO: 13), wherein X₈=S, R, K, H; (b) AASSLQX₉ (SEQ ID NO: 14), wherein X₉=S, N, Q, T; (c) QQSYSTX₁₀X₁₁GX₁₂ (SEQ ID NO: 15), wherein X₁₀=P, Q, T, S, N, A, G; X₁₁=R, G, A, S; and X₁₂=T, S, A, G, N; (d) SX₁X₂MH, wherein X₁=Y, E, Q, D; and X₂=A, D, T, V, S, G, E; (e) VISX₃DGSNKYX₄ADSVKG (SEQ ID NO: 26), wherein X₃=Y, E, Q, D; and X₄=Y, N, H, E, D, K, Q, R; and (f) ENVRPYYDFWX₅GYX₆SEYYYYGX₇DV (SEQ ID NO: 27), wherein X₅=S, R, K, H; X₆=Y, S, N, T, A, Q; and X₇=M, L, I, V, and (ii) an immune checkpoint inhibitor. Further described herein, in some embodiments, are combinations for use in treating a cancer, wherein the combination comprises: (i) an immunomodulatory CD163 antibody comprising CDR L1, CDR L2, CDR L3, CDR H1, CDR H2, and CDR H3 selected from the group consisting of: (a) SEQ ID NOs: 1, 2, 3, 4, 5, and 6; (b) SEQ ID NOs: 7, 2, 8, 16, 17, and 18; (c) SEQ ID NOs: 7, 9, 10, 19, 20, and 21; (d) SEQ ID NOs: 7, 2, 11, 22, 23, and 24; (e) SEQ ID NOs: 7, 2, 8, 22, 17, and 18; (f) SEQ ID NOs: 7, 2, 10, 16, 17, and 24; and (g) SEQ ID NOs: 7, 2, 12, 19, 17, and 18; and (ii) an immune checkpoint inhibitor, wherein the combination is for use in treating a cancer. Further described herein, in some embodiments, are combinations for use in treating a cancer, wherein the combination comprises (i) about 150 milligrams (mg) to about 1200 mg of an immunomodulatory CD163 antibody comprising a light chain variable domain (V_{L}) having a sequence at least 80% identical to the amino acid sequence selected from the group consisting of: SEQ ID NO: 28, SEQ ID NO: 30, SEQ ID NO: 32, SEQ ID NO: 34, SEQ ID NO: 36, SEQ ID NO: 38, and SEQ ID NO: 40, and a heavy chain variable domain (V_{H}) having a sequence at least 80% identical to the amino acid sequence selected from the group consisting of: SEQ ID NO: 29, SEQ ID NO: 31, SEQ ID NO: 33, SEQ ID NO: 35, SEQ ID NO: 37, SEQ ID NO: 39, and SEQ ID NO: 41, and (ii) an immune checkpoint inhibitor, wherein the combination is for use in treating a cancer.

In some embodiments, the immune checkpoint inhibitor is selected from the group consisting of an antagonist to an immune checkpoint protein or ligand thereof, wherein the immune checkpoint protein is selected from the group consisting of CTLA-4, PD-1, PD-L1, TIM3, LAG3, TIGIT, and any combination thereof. In some embodiments, the immune checkpoint inhibitor is a PD-1 antagonist. In some embodiments, the PD-1 antagonist is a PD-1 antibody. In some embodiments, the PD-1 antibody is an IgG1 antibody or IgG4 antibody. In some embodiments, the PD-1 antibody is selected from the group consisting of nivolumab, pembrolizumab, cemiplimab, dostarlimab, JTX-4014, spartalizumab, camrelizumab, sintilimab, tislelizumab, toripalimab, INCMGA00012, AMP-224, AMP-514, zimberelimab, and fragments or combinations thereof. In some embodiments, the PD-1 antagonist comprises a PD-1 binding domain comprising CDRs of an antibody selected from the group consisting of nivolumab, pembrolizumab, cemiplimab, dostarlimab, JTX-4014, spartalizumab, camrelizumab, sintilimab, tislelizumab, toripalimab, INCMGA00012, AMP-224, or AMP-514, zimberelimab, and fragments or combinations thereof. In some embodiments, the PD-1 antagonist is a small molecule. In some embodiments, the PD-1 antagonist is a rationally-designed peptide antagonist of PD-1. In some embodiments, an antagonist inhibits the interaction between PD-1 and PDL-1. In some embodiments, the antagonist is a macrocyclic compound (e.g., gramicidin S and derivatives thereof). In some embodiments, the antagonist is an antibiotic such as an ansamycin type antibiotic (e.g., rifabutin). In some embodiments, the antagonist is a phenolic compound (e.g., kaempferol, kaempferol-7-O-rhamnoside, caffeoylquinic acid, 3-O-caffeoylquinic acid, 4-O-caffeoylquinic acid, 5-O-caffeoylquinic acid, ellagic acid). In some embodiments, the antagonist is a heterocyclic compound (e.g., ZINC 67,902,090, ZINC 12,529,904). In some embodiments, the antagonist is a small molecule (e.g., CA-170, ARB-272572, INCB086550). In some embodiments, the antagonist is actinomycin D, amphotericin B, bacitracin, bryostatin, candicidin, clarithromycin, cyclosporin A, cyanocobalamin, erythromycin, everolimus, geldanamycin, ivermectin B1a, macbecin, metocurine, monocrotaline, nystatin, plerixafor, rifampin, sirolimus, troleandomycin, rifabutin, rifapentine, rifamycin SV, formyl rifamycin, rifaximin, gramicidin S, ZINC 67,902,090, ZINC 12,529,904, or derivatives thereof. In some embodiments, the antagonist is cyclo(-Leu-DTrp-Pro-Thr-Asp-Leu-DPhe-Lys(Dde)-Val-Arg) (SEQ ID NO: 46), rifabutin, kaempferol, kaempferol-7-O-rhamnoside, eriodictyol, fisetin, glyasperin C, cosmosiin, ellagic acid, caffeoylquinic acids, or derivatives thereof.

In some embodiments, the combination comprises about 150 milligrams (mg) to about 1200 mg of the immunomodulatory CD163 antibody. In some embodiments, the combination comprises about 150 milligrams (mg) to about 600 mg of the immune checkpoint inhibitor.

### Human CD163

Human CD163 (scavenger receptor cysteine-rich type 1 protein M130; hemoglobin scavenger receptor) is a cell surface protein which acts as a scavenger receptor for the hemoglobin-haptoglobin complex and protects tissues from free hemoglobin-mediated oxidative damage. Four isoforms of CD163 protein, with molecular weights of 125,451, 125,982, 121,609 and 124,958 Da have been reported. Isoform 1 is the most prevalent isoform of CD163, with a molecular weight of 125,451 Da, and consisting of 1115 amino acid-residue polypeptide comprising an extracellular domain, a transmembrane segment, and a cytoplasmic tail. The extracellular domain comprises nine cysteine-rich repeat domains. Isoform 1 of CD163 protein has four N-linked glycosylation sites, and, in M2 macrophages, CD163 protein shows two distinct bands, at ˜150 kDa and ˜130 kDa, in SDS-PAGE under reducing conditions.

Human CD163 protein is encoded by the CD163 gene. The amino acid sequence of human CD163 is:

(SEQ ID NO: 42)
MSKLRMVLLEDSGSADFRRHFVNLSPFTITVVLLLSACFVTSSLGGTD
KELRLVDGENKCSGRVEVKVQEEWGTVCNNGWSMEAVSVICNQLGCPT
AIKAPGWANSSAGSGRIWMDHVSCRGNESALWDCKHDGWGKHSNCTHQ
QDAGVTCSDGSNLEMRLTRGGNMCSGRIEIKFQGRWGTVCDDNFNIDH
ASVICRQLECGSAVSFSGSSNFGEGSGPIWFDDLICNGNESALWNCKH
QGWGKHNCDHAEDAGVICSKGADLSLRLVDGVTECSGRLEVRFQGEWG
TICDDGWDSYDAAVACKQLGCPTAVTAIGRVNASKGFGHIWLDSVSCQ
GHEPAIWQCKHHEWGKHYCNHNEDAGVTCSDGSDLELRLRGGGSRCAG
TVEVEIQRLLGKVCDRGWGLKEADVVCRQLGCGSALKTSYQVYSKIQA
TNTWLFLSSCNGNETSLWDCKNWQWGGLTCDHYEEAKITCSAHREPRL
VGGDIPCSGRVEVKHGDTWGSICDSDFSLEAASVLCRELQCGTVVSIL
GGAHFGEGNGQIWAEEFQCEGHESHLSLCPVAPRPEGTCSHSRDVGVV
CSRYTEIRLVNGKTPCEGRVELKTLGAWGSLCNSHWDIEDAHVLCQQL
KCGVALSTPGGARFGKGNGQIWRHMFHCTGTEQHMGDCPVTALGASLC
PSEQVASVICSGNQSQTLSSCNSSSLGPTRPTIPEESAVACIESGQLR
LVNGGGRCAGRVEIYHEGSWGTICDDSWDLSDAHVVCRQLGCGEAINA
TGSAHFGEGTGPIWLDEMKCNGKESRIWQCHSHGWGQQNCRHKEDAGV
ICSEFMSLRLTSEASREACAGRLEVFYNGAWGTVGKSSMSETTVGVVC
RQLGCADKGKINPASLDKAMSIPMWVDNVQCPKGPDTLWQCPSSPWEK
RLASPSEETWITCDNKIRLQEGPTSCSGRVEIWHGGSWGTVCDDSWDL
DDAQVVCQQLGCGPALKAFKEAEFGQGTGPIWLNEVKCKGNESSLWDC
PARRWGHSECGHKEDAAVNCTDISVQKTPQKATTGRSSRQSSFIAVGI
LGVVLLAIFVALFFLTKKRRQRQRLAVSSRGENLVHQIQYREMNSCLN
ADDLDLMNSSGGHSEPH.

CD163 mRNA expression is generally restricted to myeloid cells but is also expressed by certain human cancers. CD163 has also been reported to be a macrophage scavenger receptor and promote immunosuppression. In some embodiments, the interaction of the hemoglobin-haptoglobin complex with CD163 induces the secretion of the immunosuppressive cytokine IL-10 and the expression heme-oxygenase-1 (HO-1). HO-1 produces the anti-inflammatory metabolites Fe²⁺, CO, and bilirubin.

Soluble CD163 occurs in humans via ectodomain shedding and is reported to have anti-inflammatory properties, such as downregulating T-cell responses, including lymphocyte proliferation stimulated by phytohemagglutinin (PHA) or 12-O-tetradecanoylphorbol-13-acetate (TPA).

In some embodiments, CD163 is expressed on a myeloid lineage cell. In some embodiments, the myeloid lineage cell is a myeloid cell such as a macrophage. In some embodiments, CD163 is expressed on a myeloid-derived suppressor cell.

### CD163 and Myeloid Cells

As noted, CD163 is a cell surface protein that is normally expressed in certain cells, including myeloid cells such as certain macrophages. Without being bound by any particular theory, macrophages that express CD163 appear to have an immunosuppressive phenotype, such as tumor-associated macrophages and macrophages that have been activated by the alternative pathway or so-called M2 or M2-like macrophages. Binding of immunomodulatory CD163 antibodies such as, for example, those disclosed in WO 2021/016128 A1 to such macrophages can alter the function of the macrophages, appearing to reeducate or reorient these cells, causing inhibition of certain immunosuppressive activities. It some embodiments, reduction of immunosuppressive activity of such macrophages can induce increases in T cell activity and proliferation, which can result in greater immunologic response to tumors and hence be useful in treatment of patients in need thereof, such as those with cancer. Antibodies that bind to CD163 expressed on macrophages are termed “CD163 antibodies” Or “hCD163 antibodies.” In some embodiments, the CD163 antibodies are immunomodulatory and induce changes in the immunologic activity of the macrophages.

In some embodiments, CD163 is expressed on myeloid cells. In some embodiments, the cells are immunosuppressive myeloid cells. In some embodiments, the CD163⁺ cells are human CD163 expressing myeloid cells. In some embodiments, the CD163⁺ immunosuppressive myeloid cells are human macrophages. In some embodiments, the human CD163⁺ immunosuppressive macrophages are M2 or M2-like macrophages. In some embodiments, the immunosuppressive myeloid cells are myeloid-derived suppressor cells (MDSC). In some embodiments, the human macrophages express high levels of CD163 (CD163^{Hi}). By contrast, other human hematopoietic cells or primary non-immune human cells do not express CD163. For example, M1 and M1-like macrophages do not express CD163. In some embodiments, the macrophage is a tumor associated macrophage. In some embodiments, the binding of the immunomodulatory CD163 antibody to CD163 results in the macrophage switching from an M2 phenotype to an M1 phenotype.

Monocytes and macrophages exposed to certain inflammatory cytokines or microbe-associated molecular patterns differentiate into pro-inflammatory (M1 or M1-like) or anti-inflammatory (M2 or M2-like) macrophages. M1 and M2 are classifications used to define macrophages activated in vitro as pro-inflammatory (when classically activated with IFN-γ and lipopolysaccharide) or anti-inflammatory (when alternatively activated with IL-4 or IL-10), respectively, whereas in vivo or ex vivo macrophages with M1 or M2 phenotypes are defined as M1-like or M2-like macrophages. In some embodiments, M2 macrophages are generated by their exposure to certain cytokines. In some embodiments, the M2 macrophages are differentiated by IL-4, IL-10, IL-13, or a combination thereof.

M2-like macrophages have functions and phenotypes corresponding to M2 macrophages and their subtypes. An M2-like macrophage is any in vivo or ex vivo macrophage having a subset of the functional or phenotypic characteristics of M2 macrophages.

In some embodiments, CD163 antibodies of the present disclosure have high avidity and specific binding for immunosuppressive myeloid cells, in particular, macrophages. For example, in some embodiments, such macrophages are tumor-associated macrophages. In some embodiments, such macrophages are M2 macrophages. In some embodiments, such macrophages are M2-like macrophages.

Macrophages generally fall into two categories, M1 or M1-like “proinflammatory” and M2 or M2-like “immunosuppressive” macrophages, based on their functional characteristics, including their relationships to T helper cell (CD4⁺) types Th1 and Th2. Proinflammatory macrophages are a model of “classical” and can be generated with IFN-γ with either innate immune activators such as pathogen associated molecular patters (PAMP) (e.g., lipopolysaccharide (LPS)) or damage-associated molecular patterns (DAMPs) as well as inflammatory cytokines (e.g., tumor necrosis factor-alpha (TNF-α). In addition, T cell dependent macrophage activation via the CD40-CD40 ligand pathway induces M1 differentiation. Proinflammatory macrophages have pro-inflammatory, bactericidal, and cytotoxic functions. These macrophages promote the antigen-dependent induction of Th1 cells and activation of Th1 and CD8⁺ T cells. In some embodiments, proinflammatory (e.g., M1-like) macrophages are characterized by surface marker expression measured by flow cytometry and have either a CD80⁺ CD86⁺ CD163^{Lo/-} or CD206^{Lo/-} phenotype. M1 macrophages secrete IL-12, and low level of IL-10 and/or TGF-β.

By contrast, immunosuppressive macrophages are a model of “alternative” or “non-classical” activation, which can be generated with IL-4 or IL-10 in vitro, are anti-inflammatory and promote wound healing and tissue repair. For example, in some embodiments, M2-like immunosuppressive macrophages are polarized from monocyte-derived macrophages and recruited by factors secreted to tissues in need of wound-healing and/or other forms of tissue repair. Such immunosuppressive macrophages are the principal macrophage cell type involved in tissue-regeneration, such as activating and stimulating proliferation of fibroblasts. M2-like macrophages express the surface markers CD15, CD23, CD64, CD68, CD163^{Hi}, CD204^{Hi}, CD206^{Hi}, and/or other M2 macrophage markers determined by flow cytometry. M2 macrophages secrete high levels of IL-10 and TGF-beta1, and low levels of IL-12.

Sub-types of M2 macrophages include M2a, M2b, M2c, and M2d subtypes. M2a macrophages are induced by IL-4 and IL-13, which evokes upregulated expression of CD163, arginase-1, mannose receptor MRC1 (CD206), antigen presentation by MHC II system, and production of IL-10 and TGF-β, leading to tissue regeneration and the inhibition of pro-inflammatory molecules to prevent an inflammatory response. M2b macrophages produce IL-1, IL-6, IL-10, and TNF-α as a response to immune complexes. M2c macrophages are induced by IL-10, transforming growth factor beta (TGF-β) and glucocorticoids exposure, and produce IL-10 and TGF-β, leading to suppression of inflammatory response. M2d subtypes are activated as a response to IL-6 and adenosines.

Macrophage populations can be plastic and differentiate into either proinflammatory (M1) or immunosuppressive (M2) phenotypes depending on the environment (e.g., tissue environment), such as the cytokine environments described above. Macrophage populations can also shift phenotypes during a response. For example, an initial tissue injury or insult (e.g., pathogen, auto-immune, or mechanical mediated injury) can first lead to a pro-inflammatory environment promoting an M1 phenotype then switch to an M2 phenotype during a resolution/rehabilitation phase that can include wound-healing and/or tissue regeneration.

In some embodiments, macrophages are tissue macrophages. In some embodiments, the tissue macrophage results in a lung, a kidney, a heart, or a liver. In some embodiments, macrophages are pulmonary macrophages. In some embodiments, macrophages are alveolar macrophages (AMs). In some embodiments, the macrophage is a dermal macrophage. In some embodiments, the macrophage is a breast tissue resident. In some embodiments, macrophages are interstitial macrophages. In some embodiments, the macrophages are infiltrating macrophages. In some embodiments, the macrophages are tumor-associated macrophages or a macrophages in a tumor microenvironment, such as resident in the tumor itself or in the stroma.

### Immunomodulatory CD163 Antibodies

In some embodiments, an immunomodulatory CD163 antibody for use as disclosed herein specifically binds to a CD163 protein expressed on human CD163+ cell (e.g., an hCD163 antibody). In some embodiments, such immunomodulatory CD163 antibodies are used, administered, or otherwise combined with an immune checkpoint inhibitor. In some such embodiments, an immunomodulatory CD163 antibody and an immune checkpoint inhibitor are administered to a subject and result in an additive or synergistic effect. In some embodiments, such an immunomodulatory CD163 antibody does not bind to a murine or non-human primate CD163. In some embodiments, such an immunomodulatory CD163 antibody does not bind to CD163 expressed on non-myeloid cells.

In some embodiments, the CD163⁺ cell is an immunosuppressive myeloid cell. In some embodiments, the immunosuppressive myeloid cell is human macrophage. In some embodiments, the human macrophage is an immunosuppressive macrophage. In some embodiments, the immunosuppressive is an M2 or M2-like macrophage. In some embodiments, the binding of an immunomodulatory CD163 antibody alters expression of at least one marker on the human macrophage. In some embodiments, the binding of the immunomodulatory CD163 antibody to CD163 results in the macrophage switching from an M2 phenotype to an M1 phenotype. In some embodiments, the combination of the immunomodulatory CD163 antibody and the immune checkpoint inhibitor results in an additive synergistic impacts on macrophage phenotype switching as compared to either the immunomodulatory CD163 antibody or checkpoint inhibitor alone.

In some embodiments, an immunomodulatory CD163 antibody used in a method disclosed herein does not have appreciable binding to M1 or M1-like macrophages. M1-activated macrophages express transcription factors such as Interferon-Regulatory Factor (IRF5), Nuclear Factor of kappa light polypeptide gene enhancer (NF-κB), Activator-Protein (AP-1) and STAT1. M1 macrophages secrete pro-inflammatory cytokines such as IFN-γ, IL-1, IL-6, IL-12, IL-23 and TNF-α. M1 macrophages have functions and phenotypes corresponding to M1 macrophages. An M1-like macrophage is any in vivo or ex vivo macrophage having a subset of the functional or phenotypic characteristics of M1 macrophages.

In some embodiments, an immunomodulatory CD163 antibody for use with a method disclosed herein does not bind to primary human cells. In some embodiments, the antibodies of the present disclosure do not bind to hematopoietic stem cells, leukocytes, T cells, B cells, NK cells, and granulocytes. In some embodiments, an immunomodulatory CD163 antibody does not bind to murine CD163 or to any non-human primate CD163.

In some embodiments, an immunomodulatory CD163 antibody for use with a method disclosed herein binds to a hCD163 protein expressed on a human M2 or M2-like immunosuppressive macrophage. In some embodiments, the immunomodulatory CD163 antibody specifically binds to a CD163 protein that is an approximately 140 kDa glycoform of hCD163. In some embodiments, the CD163 antibody specifically binds to extracellular domain 3 of hCD163. In some embodiments, the CD163 antibody specifically binds to extracellular domain 4 of hCD163. In some embodiments, the antibody specifically binds to extracellular domain 3 and extracellular domain 4 of hCD163. In some embodiments, the immunomodulatory CD163 antibody specifically binds to hCD163, resulting in a conformational change of hCD163. In some embodiments, the conformational change to hCD163 exposes extracellular domains 2, 5, and 9 of hCD163. In some embodiments, the immunomodulatory CD163 antibody does not specifically bind a lower molecular weight (˜115 kDa) glycoform of hCD163.

In some embodiments, an immunomodulatory CD163 antibody for use with a method disclosed herein binds to a human CD163⁺ immunosuppressive myeloid cell and causes an alteration in the expression of certain cell markers that characterize a M2 or M2-like immunosuppressive macrophage (such as a M2c macrophage), indicating a functional differentiation of the macrophages to a non- or less immunosuppressive state. In some embodiments, an immunomodulatory CD163 antibody for use with a method disclosed herein binds to a M2 or M2-like immunosuppressive macrophage and causes a decrease in the expression of certain cell markers that characterize a M2 or M2-like macrophage, indicating a functional differentiation of the macrophages to an altered differentiation state. In some embodiments, an immunomodulatory CD163 antibody for use with a method disclosed herein reduces expression of one or more of CD16, CD64, TLR2, and Siglec-15 by the CD163⁺ immunosuppressive myeloid cell.

In some embodiments, the binding of a CD163 antibody for use with a method disclosed herein to a CD163⁺ immunosuppressive myeloid cells induces a functional change in the CD163⁺ immunosuppressive myeloid cell, such that the CD163 antibody is an immunomodulatory CD163 antibody. In some embodiments, the binding of the immunomodulatory CD163 antibody to the CD163⁺ immunosuppressive myeloid cell induces changes in marker expression in the myeloid cell itself or in the T cells with which the myeloid cell interacts.

In some embodiments, an immunomodulatory CD163 antibody for use with a method disclosed herein reduces immunosuppression caused by tumor-associated macrophages in tumor microenvironments. In some embodiments, a reduction in immunosuppression by tumor-associated macrophages in the tumor microenvironment corresponds to an increase in immunostimulation, e.g., production of promotion of T cell activation, T cell proliferation, NK cell activation, NK cell proliferation, or any combination thereof. In some embodiments, T cell activation and/or NK cell activation results in increased production of IFN-γ, TNF-α, perforin, or a combination thereof by T cells and/or NK cells. In some embodiments, the antibodies of the present disclosure increase immunostimulation, e.g., production of promotion of T cell activation, T cell proliferation, NK cell activation, NK cell proliferation, or any combination thereof. In some embodiments, T cell activation and/or NK cell activation results in increased production of IFN-γ, TNF-α, perforin, or a combination thereof by T cells and/or NK cells.

In some embodiments, an immunomodulatory CD163 antibody for use with a method disclosed herein specifically binds to a CD163 protein expressed on a human macrophage, wherein the human macrophage has a first immunosuppression activity before binding of the immunomodulatory CD163 antibody and a second immunosuppression activity after binding of the immunomodulatory CD163 antibody, and wherein the second immunosuppression activity lower than the first immunosuppression activity. In various embodiments, the first and second immunosuppression activities are each non-zero.

In some embodiments, an immunomodulatory CD163 antibody for use with a method disclosed herein promotes T cell activation and proliferation. In some embodiments, the immunomodulatory CD163 antibody skews a T cell population towards an anti-tumor T cell phenotype. In some embodiments, the immunomodulatory CD163 antibody reduces or blocks myeloid cell suppression of T cell activation. In some embodiments, the immunomodulatory CD163 antibody reduces the ability of TAMs to suppress T-cell activation. In some such embodiments, such reduction leads to greater T-cell stimulation and IL-2 production. In some embodiments, the immunomodulatory CD163 antibody blocks the ability of TAMs to suppress T-cell activation; in some such embodiments, such blocking leads to greater T-cell stimulation and IL-2 production.

In some embodiments, an immunomodulatory CD163 antibody for use with a method disclosed herein reduces myeloid suppression of T cell proliferation. For instance, in some embodiments, the immunomodulatory CD163 antibody reduces the ability of TAMs to suppress CD3+ T cell activation. Thus, in some embodiments, the immunomodulatory CD163 antibody enhances both CD4⁺ and CD8⁺ T cell activation and proliferation. In some embodiments, the immunomodulatory CD163 antibody reduces TAM suppression of Th1 cell proliferation. Proliferated T cells show enhanced expression of activation markers on CD4⁺ T cells.

In some embodiments, an immunomodulatory CD163 antibody for use with a method disclosed herein alters an immunosuppressive macrophage such that the macrophage exhibits a phenotype that is associated with alleviation of immunosuppressive effects by the immunosuppressive macrophages. For example, in some embodiments, binding of an immunomodulatory CD163 antibody to an M2-polarized macrophage alters the macrophage such that it exhibits an M1-like phenotype that alleviates immunosuppressive effects of M2 macrophages. In some embodiments, an immunomodulatory CD163 antibody for use with a method described herein influences monocyte-derived macrophages to differentiate to a less immunosuppressive and more anti-tumor differentiation state.

In some embodiments, an immunomodulatory CD163 antibody for use with a method disclosed herein specifically binds to CD163 that is expressed on a human myeloid cell, such as a macrophage, and reduces expression of at least one of CD16, CD64, TLR2, or Siglec-15 by the myeloid cell. In some embodiments, the human macrophage is an immunosuppressive myeloid cell. In some embodiments, the human myeloid cell is an M2-like immunosuppressive myeloid cell g. In some embodiments, the human myeloid cell is a tissue-resident macrophage. In some embodiments, the tissue-resident myeloid cell e resides in a lung, a kidney, a heart, or a liver. In some embodiments, the human myeloid cell is a pulmonary macrophage. In some embodiments, the human myeloid cell is an alveolar macrophage (AM). In some embodiments, the human myeloid cell is a dermal macrophage. In some embodiments, the human myeloid cell is a breast tissue resident. In some embodiments, the human myeloid cell is an interstitial macrophage. In some embodiments, the human myeloid cell is an infiltrating macrophage. In some embodiments, the human myeloid cell (e.g., macrophage) is a tumor-associated macrophage or a macrophage in a tumor microenvironment, such as resident in the tumor itself or in the stroma.

In some embodiments, an immunomodulatory CD163 antibody for use with a method disclosed herein binds to a CD163 protein that is expressed by a macrophage as a component of a complex comprising at least one other protein expressed by the macrophages. In some embodiments, the complex is a cell surface complex. In some embodiments, the complex comprises at least one other protein selected from a galectin-1 protein, a LILRB2 protein, and a casein kinase II protein.

In some embodiments, an immunomodulatory CD163 antibody for use with a method disclosed herein promotes CD3⁺ T cell activity or proliferation. In some embodiments, the immunomodulatory CD163 antibody promotes expression of CD69, ICOS, OX40, PD1, LAG3, or CTLA-4 by CD3⁺ T cells.

In some embodiments, an immunomodulatory CD163 antibody for use with a method disclosed herein promotes CD4⁺ T cell activity or proliferation. In some embodiments, the immunomodulatory CD163 antibody promotes expression of CD69, ICOS, OX40, PD1, LAG3, or CTLA-4 by CD4⁺ T cells.

In some embodiments, an immunomodulatory CD163 antibody for use with a method disclosed herein promotes CD8⁺ T cell activity or proliferation. In some embodiments, the immunomodulatory CD163 antibody promotes expression of ICOS, OX40, PD1, LAG3, or CTLA-4 by CD8⁺ T cells.

In some embodiments, an immunomodulatory CD163 antibody for use with a method disclosed herein promotes tumor cell killing in a tumor microenvironment by promoting CD8⁺ T cell activity or proliferation. In some embodiments, the immunomodulatory CD163 antibody promotes cytotoxic lymphocyte-mediated killing of cancer cells. In some embodiments, the immunomodulatory CD163 antibody promotes NK cell-mediated tumor cell killing.

In some embodiments, an immunomodulatory CD163 antibody for use with a method disclosed herein promotes expression of IL-2 by T cells. In some embodiments, the binding of antibodies of the present disclosure to CD163 protein increases CD4⁺ T cells, CD196⁻ T cells, CXCR3⁺ T cells, CCR4⁻ T cells, or any combination thereof. In some embodiments, the immunomodulatory CD163 antibody increases CD4⁺ CD196⁻ CXCR3⁺ CCR4⁻ T cells.

In some embodiments, an immunomodulatory CD163 antibody for use with a method disclosed herein has a constant domain that binds to an Fc receptor expressed on a macrophage. In some embodiments, the immunomodulatory CD163 antibody specifically binds hCD163 and has a constant domain that binds to an Fc receptor. In some embodiments, the immunomodulatory CD163 antibody has a constant domain that binds to an Fc receptor expressed on CD163⁺ immunosuppressive myeloid cells such as CD16 (FcγRIIIa), CD32 (FcγRII), or CD64 (FcγRI). In some embodiments, the hCD163 and Fc receptor are expressed on the same cell. In some embodiments, the hCD163 and Fc receptor are expressed on different cells. In some embodiments, the immunomodulatory CD163 antibody variable domain specifically binds hCD163 and the antibody constant domain binds to an Fc receptor simultaneously.

In some embodiments, an immunomodulatory CD163 antibody for use with a method disclosed herein binds to a CD163 protein on a macrophage and is internalized by the macrophage.

In some embodiments, an immunomodulatory CD163 antibody for use with a method disclosed herein is not cytotoxic to a macrophage to which it is bound.

In some embodiments, an immunomodulatory CD163 antibody for use with a method disclosed herein has a constant domain that binds to an Fc receptor expressed on a macrophage. In some embodiments, the immunomodulatory CD163 antibody specifically binds hCD163 and has a constant domain that binds to an Fc receptor. In some embodiments, the immunomodulatory CD163 antibody has a constant domain that binds to an Fc receptor expressed on CD163⁺ immunosuppressive myeloid cells such as CD16 (FcγRIIIa), CD32 (FcγRII), or CD64 (FcγRI). In some embodiments, the hCD163 and Fc receptor are expressed on the same cell. In some embodiments, the hCD163 and Fc receptor are expressed on different cells. In some embodiments, the immunomodulatory CD163 antibody variable domain specifically binds hCD163 and the antibody constant domain binds to an Fc receptor simultaneously.

In some embodiments, an immunomodulatory CD163 antibody for use with a method disclosed herein specifically binds to a CD163 protein expressed on human M2 and M2-like macrophages, wherein said binding results in at least one of the following effects:
(a) reduced expression of at least one marker by the human macrophage, wherein the at least one marker is CD16, CD64, TLR2, or Siglec-15;
(b) internalization of the immunomodulatory CD163 antibody by the human macrophage;
(c) reduces activation and/or proliferation of fibroblasts; and
(d) reduces secretion by the macrophage of TGF-β, PDGF, VEGF, IGF-1, Galectin-3, IL-10, or combinations thereof.

In some embodiments, an immunomodulatory CD163 antibody for use with a method disclosed herein selectively binds to human CD163⁺ immunosuppressive myeloid cells in a tissue-resident macrophage population, in which the immunomodulatory CD163 antibody specifically binds to a CD163 protein expressed on the immunosuppressive macrophages (e.g., M2) of the tissue-resident population. In some embodiments, an immunomodulatory CD163 antibody for use with a method disclosed herein selectively binds to human CD163⁺ immunosuppressive myeloid cells in an infiltrating macrophage population, in which the immunomodulatory CD163 antibody specifically binds to a CD163 protein expressed on the immunosuppressive (e.g., M2) macrophages and reduces an immunosuppressive activity of the infiltrating population.

In some embodiments, an immunomodulatory CD163 antibody for use with a method disclosed herein selectively binds to human CD163⁺ immunosuppressive myeloid cells in a tumor microenvironment, in which the immunomodulatory CD163 antibody specifically binds to a CD163 protein expressed on the immunosuppressive (e.g., M2) macrophages and reduces macrophage-mediated immunosuppression. In some embodiments, an immunomodulatory CD163 antibody for use with a method disclosed herein is human, humanized, or chimeric. In some embodiments, an immunomodulatory CD163 antibody for use with a method disclosed herein is an antigen-binding fragments thereof that bind as described.

In some embodiments, an immunomodulatory CD163 antibody for use with a method disclosed herein is an intact immunoglobulin molecule, such as, for example, a human antibody, as well as those portions of a humanized Ig molecule that contain the antigen-binding site (i.e., paratope) or a single heavy chain and a single light chain, including those portions known in the art as Fab, Fab′, F(ab)′, F(ab′)₂, Fd, scFv, a variable heavy domain, a variable light domain, a variable NAR domain, bi-specific scFv, a bi-specific Fab₂, a tri-specific Fab₃ a single chain binding polypeptide, a dAb fragment, a diabody, and others also referred to as antigen-binding fragments. When constructing an immunoglobulin molecule or fragments thereof, variable domains or portions thereof are, in some embodiments, fused to, connected to, or otherwise joined to one or more constant domains or portions thereof to produce any of the antibodies or fragments thereof described herein. Thus, in some embodiments, the antigen-binding fragment of any one of the antibodies described above is a Fab, a Fab′, a Fd, a F(ab′)₂, a Fv, a scFv, a single chain binding polypeptide (e.g., a scFv with Fc portion) or any other functional fragment thereof as described herein.

In some embodiments, an immunomodulatory CD163 antibody for use with a method disclosed herein is any immunoglobulin class, and, therefore, in some embodiments, have a gamma, mu, alpha, delta, or epsilon heavy chain. In some embodiments, the gamma chain is gamma 1, gamma 2, gamma 3, or gamma 4. In some embodiments, the alpha chain is alpha 1 or alpha 2.

In some embodiments, an immunomodulatory CD163 antibody for use with a method disclosed herein is an IgG immunoglobulin. In some embodiments, an immunomodulatory CD163 antibody for use with a method disclosed herein is any IgG subclass. In some embodiments the immunomodulatory CD163 antibody is IgG1.

In some embodiments, an immunomodulatory CD163 antibody for use with a method disclosed herein comprises a variable light chain that is either kappa or lambda. In some embodiments, the lambda chain is of any of subtype, including, e.g., lambda 1, lambda 2, lambda 3, and lambda 4. In some embodiments, the light chain is kappa.

In some embodiments, an immunomodulatory CD163 antibody for use with a method disclosed herein comprises a human variable framework and a human constant. In some embodiments, an immunomodulatory CD163 antibody for use with a method disclosed herein comprises a human light chain variable framework and a human light chain constant. In some embodiments, an immunomodulatory CD163 antibody for use with a method disclosed herein comprises a human heavy chain variable framework and a human heavy chain constant. In some embodiments, an immunomodulatory CD163 antibody for use with a method disclosed herein comprises a human light chain variable framework, a human light chain constant, a human heavy chain variable framework, and a human heavy chain constant.

In some embodiments, an immunomodulatory CD163 antibody for use with a method disclosed herein comprises a human variable framework and a murine constant domain. In some embodiments, an immunomodulatory CD163 antibody for use with a method disclosed herein comprises a human heavy chain variable framework and a murine heavy chain constant domain. In some embodiments, an immunomodulatory CD163 antibody for use with a method disclosed herein comprises a human light chain variable framework, a murine light chain constant domain, a human heavy chain variable framework, and a murine heavy chain constant domain.

Binding of an antibody or antigen-binding fragment to a CD163 protein expressed on immunosuppressive macrophages (e.g., tumor-associated macrophages, e.g., M2 macrophages) or other myeloid cells partially (e.g., 5%, 10%, 20%, 30%, 40%, 50%, 60%, 70%, 80%, 90%, 95%, 98%, 99% or any number therein) or completely modulate a biological function of such cells in some embodiments. The activity of an antibody or antigen-binding fragment, for example, are determined using an in vitro assay and/or in vivo using art-recognized assays such as those described herein or otherwise known in the art.

In some embodiments, an immunomodulatory CD163 antibody for use with a method disclosed herein is further modified to alter the specific properties of the immunomodulatory CD163 antibody while retaining the desired functionality, if needed. For example, in one embodiment, an immunomodulatory CD163 antibody for use with a method disclosed herein is modified to alter a pharmacokinetic property of the immunomodulatory CD163 antibody, including, but not limited to, in vivo stability, solubility, bioavailability, or half-life.

In some embodiments, an immunomodulatory CD163 antibody for use with a method disclosed herein has a dissociation constant (K_{D}) of about 1 to about 10 pM, from about 10 to about 20 pM, from about 1 to about 30 pM, from about 30 to about 40 pM, from about 10 to about 100 pM, or from about 20 to about 500 pM.

In some embodiments, an immunomodulatory CD163 antibody for use with a method disclosed herein has a dissociation constant (K_{D}) of less than about 500 pM, less than about 400 pM, less than about 300 pM, less than about 200 pM, less than about 100 pM, less than about 75 pM, less than about 50 pM, less than about 30 pM, less than about 25 pM, less than about 20 pM, less than about 18 pM, less than about 15 pM, less than about 10 pM, less than about 75 pM, less than about 5 pM, less than about 2.5 pM, or less than about 1 pM.

In some embodiments, an immunomodulatory CD163 antibody for use with a method disclosed herein has an affinity for a hCD163 protein or peptide of from about 10⁻⁹ to about 10⁻¹⁴, from about 10⁻¹⁰ to about 10⁻¹⁴, from about 10⁻¹¹ to about 10⁻¹⁴, from about 10⁻¹² to about 10⁻¹⁴, from about 10⁻¹³ to about 10⁻¹⁴, from about 10⁻¹⁰ to about 10⁻¹¹, from about 10⁻¹¹ to about 10⁻¹², from about 10⁻¹² to about 10⁻¹³, or 10⁻¹³ to about 10⁻¹⁴ M.

In some embodiments, an immunomodulatory CD163 antibody for use with a method disclosed herein has more than one binding site. In some embodiments, the binding sites are identical to one another. In some embodiments, the binding sites are different from one another. A naturally occurring human immunoglobulin typically has two identical binding sites, while engineered antibodies, for example, have two or more different binding sites.

In some embodiments, an immunomodulatory CD163 antibody for use with a method disclosed herein is an SMIP or binding domain immunoglobulin fusion protein specific for the target protein. These constructs are single-chain polypeptides comprising antigen-binding domains fused to immunoglobulin domains necessary to carry out antibody effector functions.

In some embodiments, an immunomodulatory CD163 antibody for use with a method disclosed herein comprises a single chain binding polypeptide having a heavy chain variable domain, and/or a light chain variable domain which binds an epitope disclosed herein and has, optionally, an immunoglobulin Fc region. Such a molecule is a single chain variable fragment (scFv) optionally having effector function or increased half-life through the presence of the immunoglobulin Fc region.

### Anti-CD163 Antibodies

In some embodiments, an immunomodulatory CD163 antibody for use with a method disclosed herein specifically binds to CD163 proteins. In some embodiments, CD163-binding antibodies comprise at least one heavy chain and at least one light chain. In some embodiments, CD163-binding antibodies comprise at least one heavy chain comprising a heavy chain variable domain (V_{H}) and at least one light chain comprising a light chain variable domain (V_{L}). Each V_{H} and V_{L} comprises three complementarity determining regions (CDR). Amino acid sequences of the V_{H} and V_{L} and the CDRs determine the antigen binding specificity and antigen binding strength of the immunomodulatory CD163 antibody. V_{H} and V_{L} domains of antibodies of the present disclosure are provided in Table 1. Amino acid sequences of the CDRs of exemplary immunomodulatory CD163 antibodies useful according to the present disclosure are provided in Table 2 and Table 3.

In some embodiments, an immunomodulatory CD163 antibody for use with a method disclosed herein binds to human CD163 (hCD163). In some embodiments, an immunomodulatory CD163 antibody for use with a method disclosed herein cannot bind to non-human CD163. In some embodiments, an immunomodulatory CD163 antibody for use with a method disclosed herein specifically binds to hCD163.

In some embodiments, an immunomodulatory CD163 antibody for use with a method disclosed herein a monoclonal antibody. In some embodiments, an immunomodulatory CD163 antibody for use with a method disclosed herein is an antigen binding fragment. In some embodiments, an immunomodulatory CD163 antibody for use with a method disclosed herein is selected from a whole immunoglobulin, an scFv, a Fab, a F(ab′)₂, or a disulfide linked Fv. In some embodiments, an immunomodulatory CD163 antibody for use with a method disclosed herein is an IgG or an IgM. In some embodiments, an immunomodulatory CD163 antibody for use with a method disclosed herein is humanized. In some embodiments, an immunomodulatory CD163 antibody for use with a method disclosed herein is chimeric.

In some embodiments, an immunomodulatory CD163 antibody for use with a method disclosed herein is RM3/1 antibody. RM3/1 antibody is a mouse monoclonal IgG1 (kappa light chain) that was raised against human monocytes. The RM3/1 antibody binds to the cysteine-rich domain 9 of human CD163, reduces LPS-induced TNFα, and enhances IL-10 secretion by macrophages.

In some embodiments, an immunomodulatory CD163 antibody for use with a method disclosed herein is Ki-M8. Ki-M8 antibody is a mouse monoclonal IgG1 that has specificity for human monocytes and macrophages.

In some embodiments, an immunomodulatory CD163 antibody for use with a method disclosed herein is Cymac-001. Cymac-001 is a human monoclonal IgG antibody.

In some embodiments, an immunomodulatory CD163 antibody for use with a method disclosed herein is MAC2-158. MAC2-158 is a mouse monoclonal antibody IgG1 that targets an epitope within the SRCR1 domain from the N-terminal region.

In some embodiments, an immunomodulatory CD163 antibody for use with a method disclosed herein is EdHu-1. EdHu-1 is a mouse monoclonal antibody IgG1.

In some embodiments, an immunomodulatory CD163 antibody for use with a method disclosed herein is TBI-304 (also known as TBI-304H or HRC-304) (Therapure Innovations). TBI-304 is a monoclonal antibody.

### Anti-CD163 Antibody Variable Domains

In some embodiments, an anti-CD163 antibody for use in a combination disclosed herein comprises or consists of at least one variable domain light chain sequence and at least one variable domain heavy chain sequence as set forth in Table 1. In some embodiments, a variable domain sequence comprises or consists of a sequence that has less than 100% percent identity to any one of SEQ ID NOs: 28-41.

TABLE 1
Exemplary Anti-CD163 Variable Domain Sequences.

| | | SEQ ID |
|---|---|---|
| | SEQUENCE | NO: |
| V1 Light | DIQMTQSPSSLSASVGDRVTITCRASQSISRYLNWYQQ | 28 |
| Chain | KPGKAPKLLIYAASSLQSGVPSRFSGSGSGTDFTLTISS | |
| | LQPEDFATYYCQQSYSTQRGSFGQGTKVEIKR | |
| V1 Heavy | EVQLVESGGGVVQPGRSLRLSCAASGFTFSSYDMHW | 29 |
| Chain | VRQAPGKGLEWVAVISEDGSNKYNADSVKGRFTISR | |
| | DNSKNTLYLQMNSLRAEDTAVYYCARENVRPYYDF | |
| | WSGYSSEYYYYGLDVWGQGTTVTVS | |
| V2 Light | DIQMTQSPSSLSASVGDRVTITCRASQSISRYLNWYQQ | 30 |
| Chain | KPGKAPKLLIYAASSLQNGVPSRFSGSGSGTDFTLTISS | |
| | LQPEDFATYYCQQSYSTTRGSFGQGTKVEIKR | |
| V2 Heavy | EVQLVESGGGVVQPGRSLRLSCAASGFTFSSETMHW | 31 |
| Chain | VRQAPGKGLEWVAVISEDGSNKYHADSVKGRFTISR | |
| | DNSKNTLYLQMNSLRAEDTAVYYCARENVRPYYDF | |
| | WSGYNSEYYYYGMDVWGQGTTVTVSS | |
| V3 Light | DIQMTQSPSSLSASVGDRVTITCRASQSISRYLNWYQQ | 32 |
| Chain | KPGKAPKLLIYAASSLQSGVPSRFSGSGSGTDFTLTISS | |
| | LQPEDFATYYCQQSYSTQRGAFGQGTKVEIKR | |
| V3 Heavy | EVQLVESGGGVVQPGRSLRLSCAASGFTFSSYVMHW | 33 |
| Chain | VRQAPGKGLEWVAVISEDGSNKYEADSVKGRFTISR | |
| | DNSKNTLYLQMNSLRAEDTAVYYCARENVRPYYDF | |
| | WRGYNSEYYYYGLDVWGQGTTVTVSS | |
| V4 Light | DIQMTQSPSSLSASVGDRVTITCRASQSISRYLNWYQQ | 34 |
| Chain | KPGKAPKLLIYAASSLQSGVPSRFSGSGSGTDFTLTISS | |
| | LQPEDFATYYCQQSYSTQRGSFGQGTKVEIKR | |
| V4 Heavy | EVQLVESGGGVVQPGRSLRLSCAASGFTFSSYVMHW | 35 |
| Chain | VRQAPGKGLEWVAVISEDGSNKYNADSVKGRFTISR | |
| | DNSKNTLYLQMNSLRAEDTAVYYCARENVRPYYDF | |
| | WSGYSSEYYYYGLDVWGQGTTVTVSS | |
| V5 Light | DIQMTQSPSSLSASVGDRVTITCRASQSISRYLNWYQQ | 36 |
| Chain | KPGKAPKLLIYAASSLQSGVPSRFSGSGSGTDFTLTISS | |
| | LQPEDFATYYCQQSYSTTRGSFGQGTKVEIKR | |
| V5 Heavy | EVQLVESGGGVVQPGRSLRLSCAASGFTFSSYDMHW | 37 |
| Chain | VRQAPGKGLEWVAVISEDGSNKYNADSVKGRFTISR | |
| | DNSKNTLYLQMNSLRAEDTAVYYCARENVRPYYDF | |
| | WRGYNSEYYYYGLDVWGQGTTVTVSS | |
| V6 Light | DIQMTQSPSSLSASVGDRVTITCRASQSISRYLNWYQQ | 38 |
| Chain | KPGKAPKLLIYAASSLQSGVPSRFSGSGSGTDFTLTISS | |
| | LQPEDFATYYCQQSYSTTGGTFGQGTKVEIKR | |
| V6 Heavy | EVQLVESGGGVVQPGRSLRLSCAASGFTFSSETMHW | 39 |
| Chain | VRQAPGKGLEWVAVISEDGSNKYNADSVKGRFTISR | |
| | DNSKNTLYLQMNSLRAEDTAVYYCARENVRPYYDF | |
| | WSGYSSEYYYYGLDVWGQGTTVTVSS | |
| AB101 | DIQMTQSPSSLSASVGDRVTITCRASQSISSYLNWYQQ | 40 |
| Light Chain | KPGKAPKLLIYAASSLQSGVPSRFSGSGSGTDFTLTISS | |
| | LQPEDFATYYCQQSYSTPRGTFGQGTKVEIKR | |
| AB101 | EVQLVESGGGVVQPGRSLRLSCAASGFTFSSYAMHW | 41 |
| Heavy Chain | VRQAPGKGLEWVAVISYDGSNKYYADSVKGRFTISR | |
| | DNSKNTLYLQMNSLRAEDTAVYYCARENVRPYYDF | |
| | WSGYYSEYYYYGMDVWGQGTTVTVSSASTKGPSVF | |
| | PLAPSSKSTSGGTAALGCLVKDYF | |

Underlined text in Table 1 indicates CDRs, with domain boundary annotations based on the IMGT database and the CDR region annotations based on the Honegger (AHo) numbering scheme.

In some embodiments, an immunomodulatory CD163 antibody for use with a method disclosed herein comprises a light chain variable domain (V_{L}) having an amino acid sequence at least about 70% identical to the amino acid sequence set forth as SEQ ID NO: 28. In some such embodiments, the V_{L} of the immunomodulatory CD163 antibody has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence set forth as SEQ ID NO: 28. In some embodiments, the V_{L} has an amino acid sequence 100% identical to that of SEQ ID NO: 28.

In some embodiments, an immunomodulatory CD163 antibody for use with a method disclosed herein comprises a light chain variable domain (V_{L}) having an amino acid sequence at least about 70% identical to the amino acid sequence set forth as SEQ ID NO: 30. In some such embodiments the V_{L} of the immunomodulatory CD163 antibody has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence set forth as SEQ ID NO: 30. In some embodiments, the V_{L} has an amino acid sequence 100% identical to that of SEQ ID NO: 30.

In some embodiments, an immunomodulatory CD163 antibody for use with a method disclosed herein comprises a light chain variable domain (V_{L}) having an amino acid sequence at least about 70% identical to the amino acid sequence set forth as SEQ ID NO: 32. In some embodiments the V_{L} has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence set forth as SEQ ID NO: 32. In some embodiments, the V_{L} has an amino acid sequence 100% identical to the amino acid sequence set forth as SEQ ID NO: 32.

In some embodiments, an immunomodulatory CD163 antibody for use with a method disclosed herein comprises a light chain variable domain (V_{L}) having an amino acid sequence at least about 70% identical to the amino acid sequence set forth as SEQ ID NO: 34. In some embodiments the V_{L} has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence set forth as SEQ ID NO: 34. In some embodiments, the V_{L} has an amino acid sequence 100% identical to the amino acid sequence set forth as SEQ ID NO: 34.

In some embodiments, an immunomodulatory CD163 antibody for use with a method disclosed herein comprises a light chain variable domain (V_{L}) having an amino acid sequence at least about 70% identical to the amino acid sequence set forth as SEQ ID NO: 36. In some embodiments the V_{L} has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence set forth as SEQ ID NO: 36. In some embodiments, the V_{L} has an amino acid sequence 100% identical to the amino acid sequence set forth as SEQ ID NO: 36.

In some embodiments, an immunomodulatory CD163 antibody for use with a method disclosed herein comprises a light chain variable domain (V_{L}) having an amino acid sequence at least about 70% identical to the amino acid sequence set forth as SEQ ID NO: 38. In some embodiments the V_{L} has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence set forth as SEQ ID NO: 38. In some embodiments, the V_{L} has an amino acid sequence 100% identical to the amino acid sequence set forth as SEQ ID NO: 38.

In some embodiments, an immunomodulatory CD163 antibody for use with a method disclosed herein comprises a light chain variable domain (V_{L}) having an amino acid sequence at least about 70% identical to the amino acid sequence set forth as SEQ ID NO: 40. In some embodiments the V_{L} has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence set forth as SEQ ID NO: 40. In some embodiments, the V_{L} has an amino acid sequence 100% identical to the amino acid sequence set forth as SEQ ID NO: 40.

In some embodiments, an immunomodulatory CD163 antibody for use with a method disclosed herein comprises a heavy chain variable domain (V_{H}) having an amino acid sequence at least about 70% identical to the amino acid sequence set forth as SEQ ID NO: 29. In some embodiments the V_{H} has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence set forth as SEQ ID NO: 29. In some embodiments, the V_{H} has an amino acid sequence 100% identical to the amino acid sequence set forth as SEQ ID NO: 29.

In some embodiments, an immunomodulatory CD163 antibody for use with a method disclosed herein comprises a heavy chain variable domain (V_{H}) having an amino acid sequence at least about 70% identical to the amino acid sequence set forth as SEQ ID NO: 31. In some embodiments the V_{H} has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence set forth as SEQ ID NO: 31. In some embodiments, the V_{H} has an amino acid sequence 100% identical to the amino acid sequence set forth as SEQ ID NO: 31.

In some embodiments, an immunomodulatory CD163 antibody for use with a method disclosed herein comprises a heavy chain variable domain (V_{H}) having an amino acid sequence at least about 70% identical to the amino acid sequence set forth as SEQ ID NO: 33. In some embodiments the V_{H} has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence set forth as SEQ ID NO: 33. In some embodiments, the V_{H} has an amino acid sequence 100% identical to the amino acid sequence set forth as SEQ ID NO: 33.

In some embodiments, an immunomodulatory CD163 antibody for use with a method disclosed herein comprises a heavy chain variable domain (V_{H}) having an amino acid sequence at least about 70% identical to the amino acid sequence set forth as SEQ ID NO: 35. In some embodiments the V_{H} has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence set forth as SEQ ID NO: 35. In some embodiments, the V_{H} has an amino acid sequence 100% identical to the amino acid sequence set forth as SEQ ID NO: 35.

In some embodiments, an immunomodulatory CD163 antibody for use with a method disclosed herein comprises a heavy chain variable domain (V_{H}) having an amino acid sequence at least about 70% identical to the amino acid sequence set forth as SEQ ID NO: 37. In some embodiments the V_{H} has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence set forth as SEQ ID NO: 37. In some embodiments, the V_{H} has an amino acid sequence 100% identical to the amino acid sequence set forth as SEQ ID NO: 37.

In some embodiments, an immunomodulatory CD163 antibody for use with a method disclosed herein comprises a heavy chain variable domain (V_{H}) having an amino acid sequence at least about 70% identical to the amino acid sequence set forth as SEQ ID NO: 39. In some embodiments the V_{H} has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence set forth as SEQ ID NO: 39. In some embodiments, the V_{H} has an amino acid sequence 100% identical to the amino acid sequence set forth as SEQ ID NO: 39.

In some embodiments, an immunomodulatory CD163 antibody for use with a method disclosed herein comprises a heavy chain variable domain (V_{H}) having an amino acid sequence at least about 70% identical to the amino acid sequence set forth as SEQ ID NO: 41. In some embodiments the V_{H} has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence set forth as SEQ ID NO: 41. In some embodiments, the V_{H} has an amino acid sequence 100% identical to the amino acid sequence set forth as SEQ ID NO: 41.

In some embodiments, an immunomodulatory CD163 antibody for use with a method disclosed herein comprises a light chain variable domain (V_{L}) having an amino acid sequence at least about at least about 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% identical to the amino acid sequence set forth in the group consisting of: SEQ ID NO: 28, SEQ ID NO: 30, SEQ ID NO: 32, SEQ ID NO: 34, SEQ ID NO: 36, SEQ ID NO: 38, and SEQ ID NO: 40; and a heavy chain variable domain (V_{H}) having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% identical to the amino acid sequence set forth as in the group consisting of: SEQ ID NO: 29, SEQ ID NO: 31, SEQ ID NO: 33, SEQ ID NO: 35, SEQ ID NO: 37, SEQ ID NO: 39, and SEQ ID NO: 41. In some embodiments, the sequence of the immunomodulatory CD163 antibody is 100% identical at each of CDR H1, CDR H2, and CDR H3 to sequences as set forth in any one of SEQ ID NOs: 29, 31, 33, 35, 37, 39, or 41; and 100% identical at each of CDR L1, CDR L2, and CDR L3 to sequences as set forth in any one of SEQ ID NOs: 28, 30, 32, 34, 36, 38, and 40.

In some embodiments, an immunomodulatory CD163 antibody for use with a method disclosed herein comprises a light chain variable domain (V_{L}) having an amino acid sequence at least about at least about 80% identical to the amino acid sequence set forth in the group consisting of: SEQ ID NO: 28, SEQ ID NO: 30, SEQ ID NO: 32, SEQ ID NO: 34, SEQ ID NO: 36, SEQ ID NO: 38, and SEQ ID NO: 40; and a heavy chain variable domain (V_{H}) having an amino acid sequence at least about 80% identical to the amino acid sequence set forth as in the group consisting of: SEQ ID NO: 29, SEQ ID NO: 31, SEQ ID NO: 33, SEQ ID NO: 35, SEQ ID NO: 37, SEQ ID NO: 39, and SEQ ID NO: 41. In some embodiments, the sequence of the immunomodulatory CD163 antibody is 100% identical at each of CDR H1, CDR H2, and CDR H3 to sequences as set forth in any one of SEQ ID NOs: 29, 31, 33, 35, 37, 39, or 41; and 100% identical at each of CDR L1, CDR L2, and CDR L3 to sequences as set forth in any one of SEQ ID NOs: 28 30, 32, 34, 36, 38, and 40. In some embodiments, an immunomodulatory CD163 antibody for use with a method disclosed herein comprises a pair of variable domains, which variable domains comprise or consist of a light chain and a heavy chain variable domain and wherein the sequences of the light and heavy chains comprise: SEQ ID NOs: 28 and 29; SEQ ID NOs: 30 and 31; SEQ ID NOs: 32 and 33; SEQ ID NOs: 34 and 35; SEQ ID NOs: 36 and 37; SEQ ID NOs: 38 and 39; and SEQ ID NOs: 40 and 41.

### Exemplary Anti-CD163 Complementarity Determining Regions

In some embodiments, an immunomodulatory CD163 antibody for use with a method disclosed herein comprises or consists of one or more complementarity determining regions (CDRs) set forth in Tables 2 (light chain CDR sequences) and 3 (heavy chain CDR sequences). In some embodiments, a CDR sequence may comprise or consist of a sequence that has a particular percent identity to any one of SEQ ID NOs: 1-27.

TABLE 2
Anti-CD163 Light Chain CDR Sequences

| Name | CDR L1 | CDR L2 | CDR L3 |
|---|---|---|---|
| AB101 | RASQSISSYLN | AASSLQS | QQSYSTPRGT |
| | (SEQ ID NO: 1) | (SEQ ID NO: 2) | (SEQ ID NO: 3) |
| V1 | RASQSISRYLN | AASSLQS | QQSYSTQRGS |
| | (SEQ ID NO: 7) | (SEQ ID NO: 2) | (SEQ ID NO: 8) |
| V2 | RASQSISRYLN | AASSLQN | QQSYSTTRGS |
| | (SEQ ID NO: 7) | (SEQ ID NO: 9) | (SEQ ID NO: 10) |
| V3 | RASQSISRYLN | AASSLQS | QQSYSTQRGA |
| | (SEQ ID NO: 7) | (SEQ ID NO: 2) | (SEQ ID NO: 11) |
| V4 | RASQSISRYLN | AASSLQS | QQSYSTQRGS |
| | (SEQ ID NO: 7) | (SEQ ID NO: 2) | (SEQ ID NO: 8) |
| V5 | RASQSISRYLN | AASSLQS | QQSYSTTRGS |
| | (SEQ ID NO: 7) | (SEQ ID NO: 2) | (SEQ ID NO: 10) |
| V6 | RASQSISRYLN | AASSLQS | QQSYSTTGGT |
| | (SEQ ID NO: 7) | (SEQ ID NO: 2) | (SEQ ID NO: 12) |
| | RASQSISX₈YLN | AASSLQX₉ | QQSYSTX₁₀X₁₁GX₁₂ |
| | (SEQ ID NO: 13) | (SEQ ID NO: 14) | (SEQ ID NO: 15) |
| | X₈ = S, R, K, H | X₉ = S, N, Q, T | X₁₀ = P, Q, T, S, N, A, G |
| | | | X₁₁ = R, G, A, S |
| | | | X₁₂ = T, S, A, G, N |

TABLE 3
Anti-CD163 Heavy Chain CDR Sequences

| Name | CDRH1 | CDRH2 | CDR H3 |
|---|---|---|---|
| AB101 | SYAMH | VISYDGSNKYYADSV | ENVRPYYDFWSGYYSEYYYYG |
| | (SEQ ID NO: 4) | KG | MDV |
| | | (SEQ ID NO: 5) | (SEQ ID NO: 6) |
| V1 | SYDMH | VISEDGSNKYNADSV | ENVRPYYDFWSGYSSEYYYYGL |
| | (SEQ ID NO: 16) | KG | DV |
| | | (SEQ ID NO: 17) | (SEQ ID NO: 18) |
| V2 | SETMH | VISEDGSNKYHADSV | ENVRPYYDFWSGYNSEYYYYG |
| | (SEQ ID NO: 19) | KG | MDV |
| | | (SEQ ID NO: 20) | (SEQ ID NO: 21) |
| V3 | SYVMH | VISEDGSNKYEADSV | ENVRPYYDFWRGYNSEYYYYGL |
| | (SEQ ID NO: 22) | KG | DV |
| | | (SEQ ID NO: 23) | (SEQ ID NO: 24) |
| V4 | SYVMH | VISEDGSNKYNADSV | ENVRPYYDFWSGYSSEYYYYGL |
| | (SEQ ID NO: 22) | KG | DV |
| | | (SEQ ID NO: 17) | (SEQ ID NO: 18) |
| V5 | SYDMH | VISEDGSNKYNADSV | ENVRPYYDFWRGYNSEYYYYGL |
| | (SEQ ID NO: 16) | KG | DV |
| | | (SEQ ID NO: 17) | (SEQ ID NO: 24) |
| V6 | SETMH | VISEDGSNKYNADSV | ENVRPYYDFWSGYSSEYYYYGL |
| | (SEQ ID NO: 19) | KG | DV |
| | | (SEQ ID NO: 17) | (SEQ ID NO: 18) |
| | SX₁X₂MH | VISX₃DGSNKYX₄ADS | ENVRPYYDFWX₅GYX₆SEYYYY |
| | X₁ = Y, E, Q, D | VKG | GX₇DV |
| | X₂ = A, D, T, V, S, | (SEQ ID NO: 26) | (SEQ ID NO: 27) |
| | G, E | X₃ = Y, E, Q, D | X₅ = S, R, K, H |
| | | X₄ = Y, N, H, E, D, K, | X₆ = Y, S, N, T, A, Q |
| | | Q, R | X₇ = M, L, I, V |

In some embodiments, an immunomodulatory CD163 antibody for use with a method disclosed herein comprises a light chain CDR1 (CDR L1) having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence set forth in the group consisting of: SEQ ID NO: 1, SEQ ID NO: 7, and SEQ ID NO: 13; a light chain CDR2 (CDR L2) having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence set forth in the group consisting of: SEQ ID NO: 2, SEQ ID NO: 9, and SEQ ID NO: 14; and a light chain CDR3 (CDR L3) having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence set forth in the group consisting of: SEQ ID NO: 3, SEQ ID NO: 8, SEQ ID NO: 10, SEQ ID NO: 11, SEQ ID NO: 12, and SEQ ID NO: 15.

In some embodiments, an immunomodulatory CD163 antibody for use with a method disclosed herein comprises a light chain CDR1 having an amino acid sequence 100% identical to the amino acid sequence set forth in the group consisting of: SEQ ID NO: 1, SEQ ID NO: 7, and SEQ ID NO: 13; a light chain CDR2 having an amino acid sequence 100% identical to the amino acid sequence set forth in the group consisting of: SEQ ID NO: 2, SEQ ID NO: 9, and SEQ ID NO: 14; and a light chain CDR3 having an amino acid sequence 100% identical to the amino acid sequence set forth in the group consisting of: SEQ ID NO: 3, SEQ ID NO: 8, SEQ ID NO: 10, SEQ ID NO: 11, SEQ ID NO: 12, and SEQ ID NO: 15.

In some embodiments, an immunomodulatory CD163 antibody for use with a method disclosed herein comprises a heavy chain CDR1 (CDR H1) having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence set forth in the group consisting of: SEQ ID NO: 4, SEQ ID NO: 16, SEQ ID NO: 19, SEQ ID NO: 22 and SX₁X₂MH, wherein X1=Y, E, Q, D; and X2=A, D, T, V, S, G, E; a heavy chain CDR2 (CDR H2) having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence set forth in the group consisting of: SEQ ID NO: 5, SEQ ID NO: 17, SEQ ID NO: 20, SEQ ID NO: 23, and SEQ ID NO: 26; a heavy chain CDR3 (CDR H3) having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence set forth in the group consisting of: SEQ ID NO: 6, SEQ ID NO: 18, SEQ ID NO: 21, SEQ ID NO: 24, and SEQ ID NO: 27.

In some embodiments, an immunomodulatory CD163 antibody for use with a method disclosed herein comprises a heavy chain CDR1 having an amino acid sequence 100% identical to the amino acid sequence set forth in the group consisting of: SEQ ID NO: 4, SEQ ID NO: 16, SEQ ID NO: 19, SEQ ID NO: 22 and SX₁X₂MH, wherein X1=Y, E, Q, D; and X2=A, D, T, V, S, G, E; a heavy chain CDR2 having an amino acid sequence 100% identical to the amino acid sequence set forth in the group consisting of: SEQ ID NO: 5, SEQ ID NO: 17, SEQ ID NO: 20, SEQ ID NO: 23, and SEQ ID NO: 26; a heavy chain CDR3 having an amino acid sequence 100% identical to the amino acid sequence set forth in the group consisting of: SEQ ID NO: 6, SEQ ID NO: 18, SEQ ID NO: 21, SEQ ID NO: 24, and SEQ ID NO: 27.

In some embodiments, an immunomodulatory CD163 antibody for use with a method disclosed herein comprises (a) a light chain CDR1 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence set forth in the group consisting of: SEQ ID NO: 1, SEQ ID NO: 7, and SEQ ID NO: 13; a light chain CDR2 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence set forth in the group consisting of: SEQ ID NO: 2, SEQ ID NO: 9, and SEQ ID NO: 14; and a light chain CDR3 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence set forth in the group consisting of: SEQ ID NO: 3, SEQ ID NO: 8, SEQ ID NO: 10, SEQ ID NO: 11, SEQ ID NO: 12, and SEQ ID NO: 15; and (b) a heavy chain CDR1 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence set forth in the group consisting of: SEQ ID NO: 4, SEQ ID NO: 16, SEQ ID NO: 19, SEQ ID NO: 22 and SX₁X₂MH, wherein X1=Y, E, Q, D; and X2=A, D, T, V, S, G, E; a heavy chain CDR2 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence set forth in the group consisting of: SEQ ID NO: 5, SEQ ID NO: 17, SEQ ID NO: 20, SEQ ID NO: 23, and SEQ ID NO: 26; a heavy chain CDR3 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence set forth in the group consisting of: SEQ ID NO: 6, SEQ ID NO: 18, SEQ ID NO: 21, SEQ ID NO: 24, and SEQ ID NO: 27.

In some embodiments, an immunomodulatory CD163 antibody for use with a method disclosed herein comprises (a) a light chain CDR1 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence set forth in the group consisting of: SEQ ID NO: 1, SEQ ID NO: 7, and SEQ ID NO: 13; a light chain CDR2 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence set forth in the group consisting of: SEQ ID NO: 2, SEQ ID NO: 9, and SEQ ID NO: 14; and a light chain CDR3 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence set forth in the group consisting of: SEQ ID NO: 3, SEQ ID NO: 8, SEQ ID NO: 10, SEQ ID NO: 11, SEQ ID NO: 12, and SEQ ID NO: 15; and (b) a heavy chain CDR1 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence set forth in the group consisting of: SEQ ID NO: 4, SEQ ID NO: 16, SEQ ID NO: 19, SEQ ID NO: 22 and SX₁X₂MH, wherein X1=Y, E, Q, D; and X2=A, D, T, V, S, G, E; a heavy chain CDR2 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence set forth in the group consisting of: SEQ ID NO: 5, SEQ ID NO: 17, SEQ ID NO: 20, SEQ ID NO: 23, and SEQ ID NO: 26; a heavy chain CDR3 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence set forth in the group consisting of: SEQ ID NO: 6, SEQ ID NO: 18, SEQ ID NO: 21, SEQ ID NO: 24, and SEQ ID NO: 27.

In some embodiments, an immunomodulatory CD163 antibody for use with a method disclosed herein comprises six CDRs set forth as follows: (a) a light chain CDR1 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 1; a light chain CDR2 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 2; and a light chain CDR3 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 3; and (b) a heavy chain CDR1 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 4; a heavy chain CDR2 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical SEQ ID NO: 5; a heavy chain CDR3 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 6.

In some embodiments, an immunomodulatory CD163 antibody for use with a method disclosed herein comprises six CDRs as set forth in Tables 2 and 3, wherein the sequences of the six CDRs are selected from the group consisting of: (a) SEQ ID NOs: 1, 2, 3, 4, 5, and 6; (b) SEQ ID NOs: 7, 2, 8, 16, 17, and 18; (c) SEQ ID NOs: 7, 9, 10, 19, 20, and 21; (d) SEQ ID NOs: 7, 2, 11, 22, 23, and 24; (e) SEQ ID NOs: 7, 2, 8, 22, 17, and 18; (f) SEQ ID NOs: 7, 2, 10, 16, 17, and 24; and (g) SEQ ID NOs: 7, 2, 12, 19, 17, and 18.

In some embodiments, an immunomodulatory CD163 antibody for use with a method disclosed herein comprises six CDRs in which the light chain CDRs are:
(a) RASQSISX8YLN (SEQ ID NO: 13), wherein X8=S, R, K, H;
(b) AASSLQX9 (SEQ ID NO: 14), wherein X9=S, N, Q, T;
(c) QQSYSTX10X11GX12 (SEQ ID NO: 15), wherein X10=P, Q, T, S, N, A, G; X11=R, G, A, S; and X12=T, S, A, G, N; and the heavy chain CDRs are:
(d) SX₁X₂MH, wherein X1=Y, E, Q, D; and X2=A, D, T, V, S, G, E;
(e) VISX3DGSNKYX4ADSVKG (SEQ ID NO: 26), wherein X3=Y, E, Q, D; and X4=Y, N, H, E, D, K, Q, R; and
(f) ENVRPYYDFWX5GYX6SEYYYYGX7DV (SEQ ID NO: 27), wherein X5=S, R, K, H; X6=Y, S, N, T, A, Q; and X7=M, L, I, V.

### Exemplary Antibodies

In some embodiments, the immunomodulatory CD163 antibody comprises amino acid sequences comprising a light chain variable domain and a heavy chain variable domain are selected from the group consisting of: (a) SEQ ID NOs: 28 and 29; (b) SEQ ID NOs: 30 and 31; (c) SEQ ID NOs: 32 and 33; (d) SEQ ID NOs: 34 and 35; (e) SEQ ID NOs: 36 and 37; (f) SEQ ID NOs: 38 and 39; and (g) SEQ ID NOs: 40 and 41.

In some embodiments, the immunomodulatory CD163 antibody is V1 and comprises a light chain variable domain (V_{L}) having an amino acid sequence as set forth as SEQ ID NO: 28, and a heavy chain variable domain (V_{H}) having an amino acid sequence as set forth as SEQ ID NO: 29.

In some embodiments, the immunomodulatory CD163 antibody is V2 and comprises a light chain variable domain (V_{L}) having an amino acid sequence as set forth as SEQ ID NO: 30, and a heavy chain variable domain (V_{H}) having an amino acid sequence as set forth as SEQ ID NO: 31.

In some embodiments, the immunomodulatory CD163 antibody is V3 and comprises a light chain variable domain (V_{L}) having an amino acid sequence as set forth as SEQ ID NO: 32, and a heavy chain variable domain (V_{H}) having an amino acid sequence as set forth as SEQ ID NO: 33.

In some embodiments, the immunomodulatory CD163 antibody is V4 and comprises a light chain variable domain (V_{L}) having an amino acid sequence as set forth as SEQ ID NO: 34, and a heavy chain variable domain (V_{H}) having an amino acid sequence as set forth as SEQ ID NO: 35.

In some embodiments, the immunomodulatory CD163 antibody is V5 and comprises a light chain variable domain (V_{L}) having an amino acid sequence as set forth as SEQ ID NO: 36, and a heavy chain variable domain (V_{H}) having an amino acid sequence as set forth as SEQ ID NO: 37.

In some embodiments, the immunomodulatory CD163 antibody is V6 and comprises a light chain variable domain (V_{L}) having an amino acid sequence as set forth as SEQ ID NO: 38, and a heavy chain variable domain (V_{H}) having an amino acid sequence as set forth as SEQ ID NO: 39.

In some embodiments, the immunomodulatory CD163 antibody comprises a light chain variable domain (V_{L}) having an amino acid sequence as set forth as SEQ ID NO: 40, and a heavy chain variable domain (V_{H}) having an amino acid sequence as set forth as SEQ ID NO: 41.

In some embodiments, the immunomodulatory CD163 antibody comprises amino acid sequences comprising six CDRs as set forth in Tables 2 and 3, wherein the six CDRs are selected from the group consisting of: (a) SEQ ID NOs: 1, 2, 3, 4, 5, and 6; (b) SEQ ID NOs: 7, 2, 8, 16, 17, and 18; (c) SEQ ID NOs: 7, 9, 10, 19, 20, and 21; (d) SEQ ID NOs: 7, 2, 11, 22, 23, and 24; (e) SEQ ID NOs: 7, 2, 8, 22, 17, and 18; (f) SEQ ID NOs: 7, 2, 10, 16, 17, and 24; and (g) SEQ ID NOs: 7, 2, 12, 19, 17, and 18.

In some embodiments, the immunomodulatory CD163 antibody is V1 and comprises a CDR L1 having an amino acid sequence as set forth as SEQ ID NO: 7, CDR L2 having an amino acid sequence as set forth as SEQ ID NO: 2, and CDR L3 having an amino acid sequence as set forth as SEQ ID NO: 8; and a CDR H1 having an amino acid sequence as set forth as SEQ ID NO: 16, CDR H2 having an amino acid sequence as set forth as SEQ ID NO: 17, and CDR H3 having an amino acid sequence as set forth as SEQ ID NO: 18.

In some embodiments, the immunomodulatory CD163 antibody is V2 and comprises a CDR L1 having an amino acid sequence as set forth as SEQ ID NO: 7, CDR L2 having an amino acid sequence as set forth as SEQ ID NO: 9, and CDR L3 having an amino acid sequence as set forth as SEQ ID NO: 10; and a CDR H1 having an amino acid sequence as set forth as SEQ ID NO: 19, CDR H2 having an amino acid sequence as set forth as SEQ ID NO: 20, and CDR H3 having an amino acid sequence as set forth as SEQ ID NO: 21.

In some embodiments, the immunomodulatory CD163 antibody is V3 and comprises a CDR L1 having an amino acid sequence as set forth as SEQ ID NO: 7, CDR L2 having an amino acid sequence as set forth as SEQ ID NO: 2, and CDR L3 having an amino acid sequence as set forth as SEQ ID NO: 11; and a CDR H1 having an amino acid sequence as set forth as SEQ ID NO: 22, CDR H2 having an amino acid sequence as set forth as SEQ ID NO: 23, and CDR H3 having an amino acid sequence as set forth as SEQ ID NO: 24.

In some embodiments, the immunomodulatory CD163 antibody is V4 and comprises a CDR L1 having an amino acid sequence as set forth as SEQ ID NO: 7, CDR L2 having an amino acid sequence as set forth as SEQ ID NO: 2, and CDR L3 having an amino acid sequence as set forth as SEQ ID NO: 8; and a CDR H1 having an amino acid sequence as set forth as SEQ ID NO: 22, CDR H2 having an amino acid sequence as set forth as SEQ ID NO: 17, and CDR H3 having an amino acid sequence as set forth as SEQ ID NO: 18.

In some embodiments, the immunomodulatory CD163 antibody is V5 and comprises a CDR L1 having an amino acid sequence as set forth as SEQ ID NO: 7, CDR L2 having an amino acid sequence as set forth as SEQ ID NO: 2, and CDR L3 having an amino acid sequence as set forth as SEQ ID NO: 10; and a CDR H1 having an amino acid sequence as set forth as SEQ ID NO: 16, CDR H2 having an amino acid sequence as set forth as SEQ ID NO: 17, and CDR H3 having an amino acid sequence as set forth as SEQ ID NO: 24.

In some embodiments, the immunomodulatory CD163 antibody is V6 and comprises a CDR L1 having an amino acid sequence as set forth as SEQ ID NO: 7, CDR L2 having an amino acid sequence as set forth as SEQ ID NO: 2, and CDR L3 having an amino acid sequence as set forth as SEQ ID NO: 12; and a CDR H1 having an amino acid sequence as set forth as SEQ ID NO: 19, CDR H2 having an amino acid sequence as set forth as SEQ ID NO: 17, and CDR H3 having an amino acid sequence as set forth as SEQ ID NO: 18.

In some embodiments, the immunomodulatory CD163 antibody comprises a CDR L1 having an amino acid sequence as set forth as SEQ ID NO: 1, CDR L2 having an amino acid sequence as set forth as SEQ ID NO: 2, and CDR L3 having an amino acid sequence as set forth as SEQ ID NO: 3; and a CDR H1 having an amino acid sequence as set forth as SEQ ID NO: 4, CDR H2 having an amino acid sequence as set forth as SEQ ID NO: 5, and CDR H3 having an amino acid sequence as set forth as SEQ ID NO: 6.

In some embodiments, the immunomodulatory CD163 antibody comprises a CDR L1 having an amino acid sequence as set forth as SEQ ID NO: 13, CDR L2 having an amino acid sequence as set forth as SEQ ID NO: 14, and CDR L3 having an amino acid sequence as set forth as SEQ ID NO: 15; and a CDR H1 having an amino acid sequence as set forth as SX₁X₂MH, wherein X1=Y, E, Q, D; and X2=A, D, T, V, S, G, E, CDR H2 having an amino acid sequence as set forth as SEQ ID NO: 26, and CDR H3 having an amino acid sequence as set forth as SEQ ID NO: 27.

In some embodiments, the immunomodulatory CD163 antibody comprises a CDR L1 having an amino acid sequence as set forth as SEQ ID NO: 1, CDR L2 having an amino acid sequence as set forth as SEQ ID NO: 2, a CDR L3 having an amino acid sequence as set forth as SEQ ID NO: 3, CDR H1 having an amino acid sequence as set forth as SEQ ID NO: 4, CDR H2 having an amino acid sequence as set forth as SEQ ID NO: 5, and CDR H3 having an amino acid sequence as set forth as SEQ ID NO: 6.

### Binding Affinity and Immunoreactivity

Binding affinity and/or avidity of antibodies or antigen-binding fragments thereof are improved by modifying framework regions. Any suitable methods for modifications of framework regions are known in the art and are contemplated herein. Selection of one or more relevant framework amino acid positions to alter depends on a variety of criteria. One criterion for selecting relevant framework amino acids to change is, for example, the relative differences in amino acid framework residues between the donor and acceptor molecules. Selection of relevant framework positions to alter using this approach has the advantage of avoiding any subjective bias in residue determination or any bias in CDR binding affinity contribution by the residue.

Binding interactions are manifested as an intermolecular contact with one or more amino acid residues of one or more CDRs in some embodiments. Antigen-binding involves, for example, a CDR or a CDR pair or, in some cases, interactions of up to all six CDRs of the V_{H} and V_{L} chains.

Binding affinity and avidity of antibodies or antigen-binding fragments can be measured by surface plasmon resonance (SPR) measurements, AlphaLisa assays or flow cytometry of the equilibrium dissociation constant (K_{D}).

In some embodiments, an immunomodulatory CD163 antibody for use with a method disclosed herein specifically binds to human CD163 with a K_{D} from 0.1 nM to 1000 nM. In some embodiments, the antibodies specifically bind to human CD163 with a K_{D} from about 0.1 to about 500 nM, from about 0.1 to about 100 nM, from about 0.1 to about 50 nM, from about 0.1 to about 20 nM, from about 0.1 to about 10 nM, from about 0.1 to about 5 nM, from about 0.1 to about 2 nM, from about 0.1 to about 1 nM, from about 0.1 to about 0.5 nM, from about 0.5 to about 1000 nM, from about 0.5 to about 500 nM, from about 0.5 to about 100 nM, from about 0.5 to about 50 nM, from about 0.5 to about 20 nM, from about 0.5 to about 10 nM, from about 0.5 to about 5 nM, from about 0.5 to about 2 nM, from about 0.5 to about 1 nM, from about 1 to about 1000 nM, from about 1 to about 500 nM, from about 1 to about 100 nM, from about 1 to about 50 nM, from about 1 to about 20 nM, from about 1 to about 10 nM, from about 1 to about 5 nM, from about 1 to about 2 nM, from about 2 to about 1000 nM, from about 2 to about 500 nM, from about 2 to about 100 nM, from about 2 to about 50 nM, from about 2 to about 20 nM, from about 2 to about 10 nM, from about 2 to about 5 nM, from about 5 to about 1000 nM, from about 5 to about 500 nM, from about 5 to about 100 nM, from about 5 to about 50 nM, from about 5 to about 20 nM, from about 5 to about 10 nM, from about 10 to about 1000 nM, from about 10 to about 500 nM, from about 10 to about 100 nM, from about 10 to about 50 nM, from about 10 to about 20 nM, from about 20 to about 1000 nM, from about 20 to about 500 nM, from about 20 to about 100 nM, from about 20 to about 50 nM, from about 50 to about 1000 nM, from about 50 to about 500 nM, from about 50 to about 100 nM, from about 100 to about 500 nM, from about 100 to about 1000 nM, from about 500 to about 1000 nM. In some embodiments, the antibodies specifically bind to human CD163 with a K_{D} of 1.8 nM, 12 nM, 45 nM or 89 nM.

In some embodiments, an immunomodulatory CD163 antibody for use with a method disclosed herein specifically binds to human CD163 with a K_{D} of 0.824 nM. In some embodiments, an antibody disclosed herein specifically bind to human CD163 with a K_{D} of 0.937 nM. In some embodiments, an antibody disclosed herein specifically bind to human CD163 with a K_{D} of 0.964 nM. In some embodiments, an antibody disclosed herein specifically bind to human CD163 with a K_{D} of 0.991 nM. In some embodiments, an antibody disclosed herein specifically bind to human CD163 with a K_{D} of 1.03 nM. In some embodiments, an antibody disclosed herein specifically bind to human CD163 with a K_{D} of 1.25 nM.

In some embodiments, an immunomodulatory CD163 antibody for use with a method disclosed herein binds to the myeloid scavenger receptor CD163, which is highly expressed on immunosuppressive macrophages such as M2 macrophages. The binding affinity between the antibodies disclosed herein and IL-10 polarized M2c macrophages are measured by flow cytometry assays.

In some such embodiments, an immunomodulatory CD163 antibody for use with a method disclosed herein specifically binds to M2c macrophages with a K_{D} from 0.1 nM to 1000 nM. In some embodiments, the antibodies specifically bind to M2c macrophages with a K_{D} from about 0.1 to about 500 nM, from about 0.1 to about 100 nM, from about 0.1 to about 50 nM, from about 0.1 to about 20 nM, from about 0.1 to about 10 nM, from about 0.1 to about 5 nM, from about 0.1 to about 2 nM, from about 0.1 to about 1 nM, from about 0.1 to about 0.5 nM, from about 0.5 to about 1000 nM, from about 0.5 to about 500 nM, from about 0.5 to about 100 nM, from about 0.5 to about 50 nM, from about 0.5 to about 20 nM, from about 0.5 to about 10 nM, from about 0.5 to about 5 nM, from about 0.5 to about 2 nM, from about 0.5 to about 1 nM, from about 1 to about 1000 nM, from about 1 to about 500 nM, from about 1 to about 100 nM, from about 1 to about 50 nM, from about 1 to about 20 nM, from about 1 to about 10 nM, from about 1 to about 5 nM, from about 1 to about 2 nM, from about 2 to about 1000 nM, from about 2 to about 500 nM, from about 2 to about 100 nM, from about 2 to about 50 nM, from about 2 to about 20 nM, from about 2 to about 10 nM, from about 2 to about 5 nM, from about 5 to about 1000 nM, from about 5 to about 500 nM, from about 5 to about 100 nM, from about 5 to about 50 nM, from about 5 to about 20 nM, from about 5 to about 10 nM, from about 10 to about 1000 nM, from about 10 to about 500 nM, from about 10 to about 100 nM, from about 10 to about 50 nM, from about 10 to about 20 nM, from about 20 to about 1000 nM, from about 20 to about 500 nM, from about 20 to about 100 nM, from about 20 to about 50 nM, from about 50 to about 1000 nM, from about 50 to about 500 nM, from about 50 to about 100 nM, from about 100 to about 500 nM, from about 100 to about 1000 nM, from about 500 to about 1000 nM. In some embodiments, the antibodies specifically bind to M2c macrophages with a K_{D} of 7.7 nM.

### Binding Epitopes

Antibody epitopes may be a linear peptide sequence (i.e., “continuous”) or may be composed of noncontiguous amino acid sequences (i.e., “conformational” or “discontinuous”). In some embodiments, an antibody recognizes one or more amino acid sequences; therefore, an epitope defines more than one distinct amino acid sequence. Epitopes recognized by antibodies are determined, for example, by peptide mapping and sequence analysis techniques well known to one of skill in the art. Binding interactions are manifested as intermolecular contacts with one or more amino acid residues of a CDR.

In some embodiments, an immunomodulatory CD163 antibody for use with a method disclosed herein specifically binds to an epitope in human CD163. In some embodiments, an immunomodulatory CD163 antibody for use with a method disclosed herein binds to an epitope comprising noncontiguous amino acid sequences. In some embodiments, an immunomodulatory CD163 antibody for use with a method disclosed herein binds to an epitope of human CD163 comprising the amino acid sequence IGRVNASKGFGHIWLDSVSCQGHEPAI (SEQ ID NO: 43). In some embodiments, an immunomodulatory CD163 antibody for use with a method disclosed herein binds to an epitope of human CD163 comprising the amino acid sequence VVCRQLGCGSA (SEQ ID NO: 44). In some embodiments, an immunomodulatory CD163 antibody for use with a method disclosed herein binds at a localized region on a surface of human CD163 protein that comprises an amino acid sequence of human CD163 comprising the amino acid sequence WDCKNWQWGGLTCD (SEQ ID NO: 45). In some embodiments, an immunomodulatory CD163 antibody for use with a method disclosed herein binds to an epitope of human CD163 comprising the amino acid sequences of SEQ ID NOs: 43-45.

In some embodiments, an antibody comprising SEQ ID NOs: 1, 2, 3, 4, 5, and 6 binds to an epitope in human CD163. In some embodiments, an antibody comprising SEQ ID NOs: 40 and 41 binds to an epitope in human CD163.

Also disclosed herein are additional antibodies that specifically bind to the epitope disclosed herein. These additional antibodies, or antigen-binding fragments thereof that specifically bind to the epitope disclosed herein can be identified using techniques known in the art. For example, a computational approach is used to design epitope-specific antibodies. Nimrod et al., Cell Reports 25, 2121-2131 Nov. 20, 2018, (incorporated herein by reference). Another approach can be used to identify antibodies that bind to specific epitopes from a library of antibodies that bind to the antigen, such as the following: first incorporate noncanonical amino acids (ncAAs) p-benzoyl-L-phenylalanine (pBpa) and p-azido-L-phenylalanine (pAzF) into the target epitope and then select the antibodies that cross-link with the ncAA incorporated epitope after UV irradiation. Because cross-linking only occurs when the distance between the antibody and the epitope is close enough, this method can efficiently select antibodies that specifically bind to the target epitope. Chen et al. Science Advances, 6(14), eaaz7825, 1 Apr. 2020.

### Modifications of Antibodies

Antibodies, or antigen-binding fragments thereof, are modified, in some cases, using techniques known in the art for various purposes such as, for example, by addition of polyethylene glycol (PEG). In some embodiments, PEG modification (PEGylation) leads to one or more of improved circulation time, improved solubility, improved resistance to proteolysis, reduced antigenicity and immunogenicity, improved bioavailability, reduced toxicity, improved stability, and easier formulation.

In some cases when an antigen-binding fragment does not contain an Fc portion, an Fc portion is added to (e.g., recombinantly) the fragment, for example, to increase half-life of the antigen-binding fragment in circulation in blood when administered to a subject. Choice of an appropriate Fc region and methods of to incorporate such fragments are known in the art. Incorporating an Fc region of an IgG into a polypeptide of interest so as to increase its circulatory half-life, but so as not to lose its biological activity is accomplished, for example, by using conventional techniques known in the art. In some embodiments, Fc portions of antibodies are further modified to increase half-life of the antigen-binding fragment in circulation in blood when administered to a subject. Modifications are, for example, determined using conventional means in the art.

Additionally, in some embodiments, antibodies and antigen-binding fragments thereof are produced or expressed so that they do not contain fucose on their complex N-glycoside-linked sugar chains. The removal of the fucose from the complex N-glycoside-linked sugar chains is known to increase effector functions of the antibodies and antigen-binding fragments, including but not limited to ADCC and CDC. Similarly, antibodies or antigen-binding fragments thereof that bind an epitope are, in some cases, attached at their C-terminal end to all or part of an immunoglobulin heavy chain derived from any antibody isotype, e.g., IgG, IgA, IgE, IgD, and IgM, and any of the isotype sub-classes, particularly IgG1, IgG2, IgG3, and IgG4.

Additionally, the antibodies or antigen-binding fragments described herein are also modified so that they are able to cross the blood-brain barrier in some embodiments. Such modification of the antibodies or antigen-binding fragments described herein allows for the treatment of brain diseases such as glioblastoma multiforme (GBM). Exemplary modifications to allow proteins such as antibodies or antigen-binding fragments to cross the blood-brain barrier are described in US Pat. Publ. 2007/0082380.

Glycosylation of immunoglobulins has been shown to have significant effects on their effector functions, structural stability, and rate of secretion from antibody-producing cells. The carbohydrate groups responsible for these properties are generally attached to the constant (C) domains of the antibodies. For example, glycosylation of IgG at asparagine 297 in the C_{H}2 domain is required for full capacity of IgG to activate the classical pathway of complement-dependent cytolysis (Tao and Morrison, J Immunol 143:2595 (1989)). Glycosylation of IgM at asparagine 402 in the C_{H}3 domain is necessary for proper assembly and cytolytic activity of a given antibody (Muraoka and Shulman, J Immunol 142:695 (1989)). Removal of glycosylation sites as positions 162 and 419 in the C_{H}1 and C_{H}3 domains of an IgA antibody led to intracellular degradation and at least 90% inhibition of secretion (Taylor and Wall, Mol Cell Biol 8:4197 (1988)). Additionally, in some embodiments, antibodies and antigen-binding fragments thereof are produced or expressed so that they do not contain fucose on their complex N-glycoside-linked sugar chains. The removal of the fucose from the complex N-glycoside-linked sugar chains is known to increase effector functions of the antibodies and antigen-binding fragments, including but not limited to, ADCC and CDC. These “defucosylated” antibodies and antigen-binding fragments are produced, in some embodiments, through a variety of systems utilizing molecular cloning techniques known in the art, including but not limited to, transgenic animals, transgenic plants, or cell-lines that have been genetically engineered so that they no longer contain the enzymes and biochemical pathways necessary for the inclusion of a fucose in the complex N-glycoside-linked sugar chains (also known as fucosyltransferase knock-out animals, plants, or cells). Non-limiting examples of cells that are engineered to be fucosyltransferase knock-out cells include CHO cells, SP2/0 cells, NS0 cells, and YB2/0 cells.

Glycosylation of immunoglobulins in the variable (V) domain has also been observed. Sox and Hood reported that about 20% of human antibodies are glycosylated in the V domain (Proc Natl Acad Sci USA 66:975 (1970)). Glycosylation of the V domain is believed to arise from fortuitous occurrences of the N-linked glycosylation signal Asn-Xaa-Ser/Thr in the V domain sequence and has not been recognized in the art as playing a role in immunoglobulin function.

Glycosylation at a variable domain framework residue, in some cases, alters the binding interaction of an antibody with antigen. The present disclosure includes criteria by which a limited number of amino acids in the framework or CDRs of a humanized immunoglobulin chain are chosen to be mutated (e.g., by substitution, deletion, or addition of residues) to increase the affinity of an antibody.

In some embodiments, cysteine residue(s) are removed or introduced in the Fc region of an antibody or Fc-containing polypeptide, thereby eliminating or increasing interchain disulfide bond formation in this region. A homodimeric specific binding agent or antibody generated using such methods, in some embodiments, exhibit improved internalization capability and/or increased complement-mediated cell killing and ADCC.

It has been shown that sequences within the CDR cause an antibody to bind to MHC Class II and trigger an unwanted helper T-cell response in some cases. In some embodiments, a conservative substitution allows an antibody to retain binding activity yet reduce its ability to trigger an unwanted T-cell response. In one embodiment, one or more of the N-terminal 20 amino acids of the heavy or light chain is removed.

In some embodiments, antibody molecules are produced with altered carbohydrate structure resulting in altered effector activity, including antibody molecules with absent or reduced fucosylation that exhibit improved ADCC activity. A variety of ways are known in the art to accomplish this. For example, ADCC effector activity is mediated by binding of an antibody molecule to the FcγRIII receptor, which has been shown to be dependent on the carbohydrate structure of the N-linked glycosylation at the Asn-297 of the C_{H}2 domain. Non-fucosylated antibodies bind this receptor with increased affinity and trigger FcγRIII-mediated effector functions more efficiently than native, fucosylated antibodies. Some host cell strains, e.g., Lec13 or rat hybridoma YB2/0 cell line naturally produce antibodies with lower fucosylation levels. An increase in the level of bisected carbohydrate, e.g., through recombinantly producing antibody in cells that overexpress GnTIII enzyme, has also been determined to increase ADCC activity. In some embodiments, the absence of only one of the two fucose residues are sufficient to increase ADCC activity.

Covalent modifications of an antibody (e.g., an immunomodulatory CD163 antibody) are also included herein. In some embodiments, they are made by chemical synthesis or by enzymatic or chemical cleavage of an antibody, if applicable. In some embodiments, other types of covalent modifications are introduced by reacting targeted amino acid residues with an organic derivatizing agent that is capable of reacting with selected side chains or the N- or C-terminal residues.

Cysteinyl residues most commonly are reacted with alpha-haloacetates (and corresponding amines), such as chloroacetic acid or chloroacetamide, to give carboxymethyl or carboxyamidomethyl derivatives. Cysteinyl residues also are derivatized by reaction with bromotrifluoroacetone, alpha-bromo-beta-(5-imidozoyl) propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole.

In some embodiments, histidyl residues are derivatized by reaction with diethylpyrocarbonate at pH 5.5-7.0 because this agent is relatively specific for the histidyl side chain. In some embodiments, para-bromophenacyl bromide also is useful; the reaction, in some embodiments, is performed in 0.1 M sodium cacodylate at pH 6.0.

In some embodiments, lysinyl and amino-terminal residues are reacted with succinic or other carboxylic acid anhydrides. Derivatization with these agents has the effect of reversing the charge of the lysinyl residues. Other suitable reagents for derivatizing alpha-amino-containing residues include imidoesters such as methyl picolinimidate, pyridoxal phosphate, pyridoxal, chloroborohydride, trinitrobenzenesulfonic acid, O-methylisourea, 2,4-pentanedione, and transaminase-catalyzed reaction with glyoxylate.

In some embodiments, arginyl residues are modified by reaction with one or several conventional reagents, such as phenylglyoxal, 2,3-butanedione, 1,2-cyclohexanedione, and ninhydrin. Derivatization of arginine residues requires that the reaction be performed in alkaline conditions because of the high pKa of the guanidine functional group. Furthermore, these reagents, in some embodiments, react with the groups of lysine as well as the arginine epsilon-amino group.

In some embodiments, the specific modification of tyrosyl residues is made, with particular interest in introducing spectral labels into tyrosyl residues by reaction with aromatic diazonium compounds or tetranitromethane. Most commonly, N-acetylimidazole and tetranitromethane are used to form O-acetyl tyrosyl species and 3-nitro derivatives, respectively, in some embodiments. Tyrosyl residues are iodinated using ¹²⁵I or ¹³¹I to prepare labeled proteins for use in radioimmunoassay.

Carboxyl side groups (aspartyl or glutamyl) are specifically modified by reaction with carbodiimides (R—N═C═N—R′), where R and R′ are different alkyl groups, such as 1-cyclohexyl-3-(2-morpholinyl-4-ethyl)carbodiimide or 1-ethyl-3-(4-azonia-4,4-dimethylpentyl)carbodiimide. Furthermore, aspartyl and glutamyl residues are converted to asparaginyl and glutaminyl residues by reaction with ammonium ions.

In some embodiments, glutaminyl and asparaginyl residues are deamidated to the corresponding glutamyl and aspartyl residues, respectively. These residues are deamidated under neutral or basic conditions.

Other modifications include hydroxylation of proline and lysine, phosphorylation of hydroxyl groups of seryl or threonyl residues, methylation of the alpha-amino groups of lysine, arginine, and histidine side chains, acetylation of the N-terminal amine, and amidation of any C-terminal carboxyl group.

Another type of covalent modification involves chemically or enzymatically coupling glycosides to the specific binding agent or antibody. These procedures do not require production of the polypeptide or antibody in a host cell that has glycosylation capabilities for N- or O-linked glycosylation. Depending on the coupling mode used, in some embodiments, the sugar(s) are attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine.

Removal of any carbohydrate moieties present on the polypeptide or antibody are, in some embodiments, accomplished chemically or enzymatically. Chemical deglycosylation involves exposure of an antibody to the compound trifluoromethanesulfonic acid, or an equivalent compound. This treatment results in the cleavage of most or all sugars except the linking sugar (N-acetylglucosamine or N-acetylgalactosamine), while leaving the antibody intact. Enzymatic cleavage of carbohydrate moieties on an antibody is achieved by the use of a variety of endo- and exo-glycosidases in some embodiments.

Another type of covalent modification comprises linking an antibody to one of a variety of non-proteinaceous polymers, e.g., polyethylene glycol, polypropylene glycol, polyoxyethylated polyols, polyoxyethylated sorbitol, polyoxyethylated glucose, polyoxyethylated glycerol, polyoxyalkylenes, or polysaccharide polymers such as dextran. Such methods are known in the art.

Affinity for binding a pre-determined polypeptide antigen, generally, is modulated by introducing one or more mutations into the V domain framework, typically in areas adjacent to one or more CDRs and/or in one or more framework regions. Typically, such mutations involve the introduction of conservative amino acid substitutions that either destroy or create the glycosylation site sequences but do not substantially affect the hydropathic structural properties of the polypeptide. Typically, mutations that introduce a proline residue are avoided.

### Effector Functions

Examples of antibody effector functions include: C1q binding and complement dependent cytotoxicity; Fc receptor binding; ADCC; phagocytosis; down-regulation of cell surface receptors (e.g., B cell receptor); and B cell activation. Typically, the Fc-mediated functions involve binding of the Fc portion of an antibody by specialized receptor molecules, “Fc receptors” or “FcR,” expressed by the cell whose function is to be affected.

IgG is considered the most versatile immunoglobulin because it carries out all of the functions of immunoglobulin molecules in some embodiments. IgG is the major Ig in serum, and the only class of Ig that crosses the placenta. IgG also fixes complement, although the IgG4 subclass does not. Macrophages, monocytes, polymorphonuclear leukocytes (PMNs), and some lymphocytes have receptors for the Fc region of IgG. Not all subclasses bind equally well: IgG2 and IgG4 do not bind to Fc receptors. A consequence of binding to the Fc receptors on PMNs, monocytes, and macrophages is that the cell now internalizes the antigen better in some cases. IgG is an opsonin that enhances phagocytosis. Binding of IgG to Fc receptors on other types of cells results in the activation of other functions.

In certain embodiments, the FcR is a native sequence human FcR. Moreover, a preferred FcR is one that binds an IgG antibody (a gamma (“γ”) receptor) and includes receptors of the FcγRI (CD64), FcγRII (CD32), and FcγRIII (CD16) subclasses, including allelic variants and alternatively spliced forms of these receptors FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain.

“Antibody-dependent cell-mediated cytotoxicity” or “ADCC” refers to a form of cytotoxicity in which secreted Ig bound to Fc receptors (FcRs) present on certain cytotoxic cells (e.g., Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are required for such killing. The primary cells for mediating ADCC, NK cells, express FcγRIII only, whereas monocytes express FcγRI, FcγRII, and FcγRIII. To assess ADCC activity of a molecule of interest, an in vitro ADCC assay is performed in some embodiments. Useful effector cells for such assays include peripheral blood mononuclear cells (PBMC) and Natural Killer (NK) cells.

Alternatively, or additionally, in some embodiments, ADCC activity of the molecule of interest is assessed in vivo, e.g., in an animal model.

In some embodiments, the antibodies of the disclosure bind to a surface membrane protein of and are internalized by M2-like macrophages. This internalization process is believed to be involved in the observed alteration of the functional immunosuppressive characteristics of these cells, i.e., the differentiation of the cells from M2 status to subtly activated state, without killing them or inhibiting their proliferation. In some embodiments, upon internalization, the antibodies decrease the expression of immunosuppressive soluble factors while increasing expression of soluble factors that stimulate or promote the activity or proliferation of T cells, including CD4⁺ helper T cells and cytotoxic lymphocytes.

For certain therapeutic applications, the internalization process is employed for purposes of killing or decreasing the activity or proliferation of a target cell that expresses a CD163 protein. The number of antibody molecules internalized will be sufficient or adequate to kill a cell or inhibit its growth. Depending on the potency of an antibody or antibody conjugate, in some instances, the uptake of a single antibody molecule into the cell is sufficient to kill the target cell to which the antibody binds. For example, certain toxins are highly potent in killing such that internalization of one molecule of the toxin conjugated to the antibody is sufficient to kill the targeted cell.

In some embodiments, the immunomodulatory CD163 antibody or antigen-binding fragment provided herein is conjugated or linked to a therapeutic moiety, an imaging or detectable moiety, or an affinity tag. Methods for conjugating or linking polypeptides are well known in the art. Associations (binding) between compounds and labels include any means known in the art including, but not limited to, covalent and non-covalent interactions, chemical conjugation, as well as recombinant techniques. An antibody or antigen-binding fragment thereof is conjugated to, or recombinantly engineered with, an affinity tag (e.g., a purification tag), in some embodiments. Affinity tags such as, for example, poly-histidine (e.g., His6 (SEQ ID NO: 47)) tags are conventional in the art.

In some embodiments, the immunomodulatory CD163 antibody or antigen-binding fragment further comprises a detectable moiety. Detections accomplished, for example, in vitro, in vivo or ex vivo. In vitro assays for the detection and/or determination (quantification, qualification, etc.) of, e.g., hCD163 protein expressed by macrophages using the antibodies or antigen-binding fragments thereof include but are not limited to, for example, ELISAs, RIAs, and western blots. In some embodiments, in vitro detection, diagnosis, or monitoring of the antigen of the antibodies occurs by obtaining a sample (e.g., a blood sample) from a subject and testing the sample in, for example, a standard ELISA assay.

### Antibody Technology

As will be understood by the skilled artisan, general description of antibodies herein and methods of preparing and using the same also apply to individual antibody polypeptide constituents and antibody fragments.

The antibodies of the present disclosure are polyclonal or monoclonal antibodies. However, in preferred embodiments, they are monoclonal. In particular embodiments, antibodies of the present disclosure are human antibodies. Methods of producing polyclonal and monoclonal antibodies are known in the art.

Antibodies, antigen-binding fragments, and other proteins that bind hCD163 expressed by immunosuppressive macrophages (e.g., tumor-associated macrophages, e.g., M2 macrophages) are generated using such methods are tested for one or more of their binding affinity, avidity, and modulating capabilities in some embodiments.

Conventional methods, in some embodiments, are utilized to identify antibodies or antigen-binding fragments thereof that bind to a hCD163 protein. In some embodiments, antibodies and antigen-binding fragments are evaluated for one or more of binding affinity, association rates, disassociation rates, and avidity. Measurement of such parameters is, for example, accomplished using assays including, but not limited to, an enzyme-linked-immunosorbent assays (ELISA), ELISpot assays, Scatchard analysis, surface plasmon resonance (e.g., BIACORE) analysis, etc., competitive binding assays, and the like. In one non-limiting embodiment, an ELISA assay is used to measure the binding capability of specific antibodies or antigen-binding fragments that bind to a hCD163 protein. A surface plasmon resonance technique is described in Liljeblad et al., Glyco J 17:323-9 (2000).

In some embodiments, antibodies according to the disclosure are produced recombinantly, using vectors and methods available in the art, as described further below. In some embodiments, human antibodies are also be generated by in vitro activated B cells (see U.S. Pat. Nos. 5,567,610 and 5,229,275).

In some embodiments, human antibodies are produced in transgenic animals (e.g., mice) that are capable of producing a full repertoire of human antibodies in the absence of endogenous immunoglobulin production. For example, it has been described that the homozygous deletion of the antibody heavy-chain joining domain (JH) gene in chimeric and germ-line mutant mice results in complete inhibition of endogenous antibody production. Transfer of the human germ-line immunoglobulin gene array into such germ-line mutant mice results in the production of human antibodies upon antigen challenge. See, e.g., Jakobovits et al., Proc Natl Acad Sci USA, 90:2551 (1993); Jakobovits et al., Nature 362:255-58 (1993); Bruggemann et al., Year in Immunol., 7:33 (1993); U.S. Pat. Nos. 5,545,806, 5,569,825, 5,591,669; 5,545,807; and WO 97/17852. In some embodiments, such animals are genetically engineered to produce human antibodies comprising a polypeptide of the present disclosure.

The antibodies are, for example, isolated and purified from a culture supernatant or ascites (if produced in an animal) using methods known in the art, such as by saturated ammonium sulfate precipitation, euglobulin precipitation method, caproic acid method, caprylic acid method, ion exchange chromatography (DEAE or DE52), or affinity chromatography using anti-Ig column or a protein A, G, or L column.

As noted above, the disclosure further provides antibody fragments. In certain circumstances there are advantages of using antibody fragments, rather than whole antibodies. For example, the smaller size of the fragments allows for rapid clearance, and leads to improved access to certain tissues, such as organs (e.g., lung, kidney, liver, or heart), in some embodiments. Examples of antibody fragments include: Fab, Fab′, F(ab′)₂, and Fv fragments; diabodies; linear antibodies; single-chain antibodies; and multispecific antibodies formed from antibody fragments.

Various techniques have been developed to produce antibody fragments. Traditionally, these fragments were derived via proteolytic digestion of intact antibodies (see, e.g., Morimoto et al., J Biochem Biophys Methods 1992 24(1-2):107-17; and Brennan et al., Science 1985 229:81). However, these fragments are now be produced directly by recombinant host cells in some embodiments. In some embodiments, Fab, Fv, and ScFv antibody fragments all are expressed in and secreted from E. coli, thus allowing the facile production of large amounts of these fragments. In some embodiments, Fab′-SH fragments are directly recovered from E. coli and chemically coupled to form F(ab′)₂ fragments (Carter et al., Bio/Technology 10:163-167 (1992)). According to another approach, in some embodiments, F(ab′)₂ fragments are isolated directly from recombinant host cell culture. Fab and F(ab′)₂ fragment with increased in vivo half-life comprising a salvage receptor binding epitope taken from two loops of a C_{H}2 domain of an Fc region of an IgG are described in U.S. Pat. Nos. 5,869,046 and 6,121,022. Other techniques for producing antibody fragments will be apparent to the skilled practitioner.

In other embodiments, an antibody of choice is a single chain Fv fragment (scFv). See WO 93/16185; U.S. Pat. Nos. 5,571,894; and 5,587,458. Fv and sFv are the only species with intact combining sites that are devoid of constant regions. Thus, they are suitable for reduced nonspecific binding during in vivo use. In some embodiments, sFv fusion proteins are constructed to yield fusion of an effector protein at either the amino or the carboxy terminus of an sFv. See Antibody Engineering, Carl A. K. Borrebaeck (Ed.). In some embodiments, an antibody fragment is a “linear antibody,” e.g., as described in U.S. Pat. No. 5,641,870 for example. In some embodiments, such linear antibody fragments are monospecific or bispecific.

Methods for making bispecific or other multispecific antibodies are known in the art and include chemical cross-linking, use of leucine zippers (Kostelny et al., J Immunol 148:1547-53 (1992)); diabody technology (Hollinger et al., Proc Natl Acad Sci USA 90:6444-8 (1993)); scFv dimers (Gruber et al., J Immunol 152:5368 (1994)), linear antibodies (Zapata et al., Protein Eng 8:1057-62 (1995)); and chelating recombinant antibodies (Neri et al., J Mol Biol 246:367-73 (1995)).

Traditional production of full-length bispecific antibodies is based on the co-expression of two immunoglobulin heavy chain-light chain pairs, where the two chains have different specificities (Millstein et al., Nature 305:537-9 (1983)). Because of the random assortment of immunoglobulin heavy and light chains, these hybridomas (quadromas) produce a potential mixture of ten different antibody molecules, of which only one has the correct bispecific structure. Purification of the correct molecule are done, for example, by affinity chromatography. Similar procedures are disclosed in WO 93/08829, and in Traunecker et al., EMBO J 10:3655-9 (1991).

According to a different approach, antibody variable regions with the desired binding specificities (antibody-antigen combining sites) are fused to immunoglobulin constant domain sequences. Preferably, the fusion is with an Ig heavy chain constant domain, comprising at least part of the hinge, C_{H}2, and C_{H}3 domains. It is preferred that the first heavy-chain constant domain (C_{H}1) containing the site necessary for light chain bonding, be present in at least one of the fusions. DNAs encoding the immunoglobulin heavy chain fusions and, if desired, the immunoglobulin light chain, are inserted into separate expression vectors, and are co-transfected into a suitable host cell. This provides for greater flexibility in adjusting the mutual proportions of the four polypeptide fragments in embodiments when unequal ratios of the four polypeptide chains used in the construction provide the optimum yield of the desired bispecific antibody. It is, however, possible to insert the coding sequences for two or all four polypeptide chains into a single expression vector when the expression of at least two polypeptide chains in equal ratios results in high yields or when the ratios have no significant effect on the yield of the desired chain combination.

Bispecific antibodies are composed of, for example, a hybrid immunoglobulin heavy chain with a first binding specificity in one arm, and a hybrid immunoglobulin heavy chain-light chain pair (providing a second binding specificity) in the other arm. This asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods Enzymol 121:210 (1986).

According to another approach described in U.S. Pat. No. 5,731,168, the interfaces between a pair of antibody molecules are engineered to maximize the percentage of heterodimers that are recovered from recombinant cell culture in some embodiments. The preferred interface comprises at least a part of the C_{H}3 domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

### Identification and Preparation of Antibodies

Polynucleotide sequences encoding the antibodies (e.g., immunomodulatory CD163 antibodies), variable regions thereof, or antigen-binding fragments thereof are, in some embodiments, determined using conventional sequencing techniques, and subcloned into expression vectors for the recombinant production of the antibodies. This was accomplished by obtaining mononuclear cells from the blood of a subject; producing B cell clones from the mononuclear cells; inducing the B cells to become antibody-producing plasma cells; and screening the supernatants produced by the plasma cells to determine if it contains an antibody. Identification of other antibodies having the specificity of the antibodies of the disclosure are accomplished using a similar method in some embodiments. For example, once a B cell clone that produces an antibody is identified, reverse-transcription polymerase chain reaction (RT-PCR) is performed to clone the DNAs encoding the variable domains or portions thereof of the antibody. These sequences are then subcloned into expression vectors suitable for the recombinant production of human antibodies. The binding specificity is confirmed, in some embodiments, by determining the antibody's ability to bind M2 cells or other cells expressing a human CD163 polypeptide that is expressed by M2 cells.

In particular embodiments of the methods described herein, B cells isolated from peripheral blood or lymph nodes are sorted, e.g., based on their being CD19 positive, and plated, e.g., as low as a single cell specificity per well, e.g., in 96-, 384-, or 1536-well configurations. The cells are induced to differentiate into antibody-producing cells, e.g., plasma cells, and the culture supernatants are harvested and tested for binding to cells expressing the target polypeptide on their surface using, e.g., FMAT or FACS analysis. Positive wells are then subjected to whole well RT-PCR to amplify heavy and light chain variable domains of the IgG molecule expressed by the clonal daughter plasma cells. The resulting PCR products encoding the heavy and light chain variable domains, or portions thereof, are subcloned into human antibody expression vectors for recombinant expression. The resulting recombinant antibodies are then tested to confirm their original binding specificity and are further tested, in some embodiments, for cross-reactivity against other cells or proteins.

Thus, in one embodiment, a method of identifying antibodies is practiced as follows. First, full-length or approximately full-length CD163 cDNAs are transfected into a cell line for expression of CD163 polypeptides. Secondly, individual human plasma or sera samples are tested for antibodies that bind the cell-expressed polypeptides. And lastly, MAbs derived from plasma- or serum-positive individuals are characterized for binding to the same cell-expressed CD163 polypeptides. Further definition of the fine specificities of the MAbs are performed at this point in some embodiments.

Polynucleotides that encode the antibodies or portions thereof of the present disclosure are isolated from cells expressing the antibodies, according to methods available in the art and described herein, including amplification by polymerase chain reaction using primers specific for conserved domains of human antibody polypeptides, in some embodiments. For example, light chain and heavy chain variable domains is cloned from the B cell according to molecular biology techniques described in WO 92/02551; U.S. Pat. No. 5,627,052; or Babcook et al., Proc Natl Acad Sci USA 93:7843-48 (1996). In certain embodiments, polynucleotides encoding all or a region of both the heavy and light chain variable domains of the IgG molecule expressed by the clonal daughter plasma cells expressing the antibody are subcloned and sequenced. In some embodiments, the sequence of the encoded polypeptide is readily determined from the polynucleotide sequence.

Isolated polynucleotides encoding a polypeptide of the present disclosure is subcloned into an expression vector to recombinantly produce antibodies and polypeptides of the present disclosure, using procedures known in the art and described herein.

In some embodiments, binding properties of an antibody (or fragment thereof) to CD163 polypeptides or M2 cells are generally determined and assessed using immunodetection methods including, for example, immunofluorescence-based assays, such as immuno-histochemistry (IHC) and/or fluorescence-activated cell sorting (FACS). Immunoassay methods include, in some embodiments, controls and procedures to determine whether antibodies bind specifically to CD163 polypeptides or to immunosuppressive macrophages (e.g., M2 macrophages), and do not recognize or cross-react with control cells, e.g., M1 cells, or host cells transfected to express a control protein.

Following pre-screening of serum to identify patients that produce antibodies to a CD163 polypeptide or to immunosuppressive macrophages (e.g., M2 macrophages), the methods of the present disclosure typically include the isolation or purification of B cells from a biological sample previously obtained from a patient or subject. However, in some embodiments, it is understood that any biological sample comprising B cells is used for any of the embodiments of the present disclosure.

Once isolated, the B cells are induced to produce antibodies, e.g., by culturing the B cells under conditions that support B cell proliferation or development into a plasmacyte, plasmablast, or plasma cell. The antibodies are then screened, typically using high throughput techniques, to identify an antibody that specifically binds to a target antigen, e.g., a particular tissue, cell, or polypeptide. In certain embodiments, the specific antigen, e.g., cell surface polypeptide bound by the antibody is not known, while in other embodiments, the antigen specifically bound by the antibody is known.

According to the present disclosure, B cells are, in some embodiments, isolated from a biological sample, e.g., tissue, peripheral blood or lymph node sample by any means known and available in the art. B cells are typically sorted by FACS based on the presence on their surface of a B cell-specific marker, e.g., CD19, CD138, and/or surface IgG. However, other methods known in the art are employed in some embodiments, such as, e.g., column purification using CD19 magnetic beads or IgG-specific magnetic beads, followed by elution from the column. However, magnetic isolation of B cells utilizing any marker results in loss of certain B cells in some embodiments.

To identify B cells that produce an antibody, the B cells are typically plated at low density (e.g., a single cell specificity per well, 1-10 cells per well, 10⁻¹⁰⁰ cells per well, 1-100 cells per well, less than 10 cells per well, or less than 100 cells per well) in multi-well or microtiter plates, e.g., in 96, 384, or 1536 well configurations. When the B cells are initially plated at a density greater than one cell per well, then the methods of the present disclosure include the step of subsequently diluting cells in a well identified as producing an antigen-specific antibody, until a single cell specificity per well is achieved, thereby facilitating the identification of the B cell that produces the antigen-specific antibody in some embodiments. In some embodiments, cell supernatants or a portion thereof and/or cells are frozen and stored for future testing and later recovery of antibody polynucleotides.

In certain embodiments, the B cells are cultured under conditions that favor the production of antibodies by the B cells. For example, the B cells are cultured under conditions favorable for B cell proliferation and differentiation to yield antibody-producing plasmablasts, plasmacytes, or plasma cells. In particular embodiments, the B cells are cultured in the presence of a B cell mitogen, such as lipopolysaccharide (LPS) or CD40 ligand. In one specific embodiment, B cells are differentiated to antibody-producing cells by culturing them with feed cells and/or other B cell activators, such as CD40 ligand.

Cell culture supernatants or antibodies obtained therefrom are tested for their ability to bind to a target antigen, using routine methods available in the art, including those described herein, in some embodiments. In particular embodiments, culture supernatants are tested for the presence of antibodies that bind to a target antigen using high-throughput methods. For example, B cells are cultured in multi-well microtiter dishes, such that robotic plate handlers are used to simultaneously sample multiple cell supernatants and test for the presence of antibodies that bind to a target antigen. In particular embodiments, antigens are bound to beads, e.g., paramagnetic or latex beads) to facilitate the capture of antibody/antigen complexes. In other embodiments, antigens and antibodies are fluorescently labeled (with different labels) and FACS analysis is performed to identify the presence of antibodies that bind to target antigen. In one embodiment, antibody binding is determined using FMAT™ analysis and instrumentation (Applied Biosystems, Foster City, Calif.). FMAT is a fluorescence macro-confocal platform for high-throughput screening, which enables mix-and-read, non-radioactive assays using live cells or beads.

In comparing the binding of an antibody to a particular target antigen (e.g., a biological sample such as diseased tissue or cells, or infectious agents) to the antibody's binding to a control sample (e.g., a biological sample such as normal cells, comparator cells from another species, a different tissue or cell, or different infectious agent), in some embodiments, the antibody is considered to preferentially bind a particular target antigen if at least two-fold, at least three-fold, at least five-fold, or at least ten-fold more antibody binds to the particular target antigen as compared to the amount that binds a control sample.

Polynucleotides encoding antibody chains, variable domains thereof, or fragments thereof, are isolated from cells utilizing any means available in the art in some embodiments. In one embodiment, polynucleotides are isolated using polymerase chain reaction (PCR), e.g., reverse transcription-PCR (RT-PCR) using oligonucleotide primers that specifically bind to heavy or light chain encoding polynucleotide sequences or complements thereof using routine procedures available in the art. In one embodiment, positive wells are subjected to whole well RT-PCR to amplify the heavy and light chain variable domains of the IgG molecule expressed by the clonal daughter plasma cells. These PCR products, in some embodiments, are sequenced, and products encoding the heavy and light chain variable domains or portions thereof are then subcloned into human antibody expression vectors and recombinantly expressed according to routine procedures in the art (see, e.g., U.S. Pat. No. 7,112,439). The nucleic acid molecules encoding an immunosuppressive (e.g., M2) macrophage-specific antibody or fragment thereof as described herein are, in some embodiments, propagated and expressed according to any of a variety of well-known procedures for nucleic acid excision, ligation, transformation, and transfection. Thus, in certain embodiments expression of an antibody fragment are preferred in a prokaryotic host cell, such as E. coli (see, e.g., Pluckthun et al., Methods Enzymol 178:497-515 (1989)). In certain other embodiments, expression of the antibody or an antigen-binding fragment thereof are preferred in a eukaryotic host cell, such as yeast (e.g., Saccharomyces cerevisiae, S. pombe, Pichia pastoris); animal cells (including mammalian cells); or plant cells. Examples of suitable animal cells include, but are not limited to, myeloma, COS, CHO, or hybridoma cells. Examples of plant cells include tobacco, corn, soybean, and rice cells. By methods known to those having ordinary skill in the art and based on the present disclosure, a nucleic acid vector is designed for expressing foreign sequences in a particular host system, and then polynucleotide sequences encoding the immunosuppressive macrophage-specific antibody (or fragment thereof) is inserted, in some embodiments. The regulatory elements will vary according to the particular host.

One or more replicable expression vectors containing a polynucleotide encoding a variable and/or constant domain is, in some embodiments, prepared and used to transform an appropriate cell line, for example, a non-producing myeloma cell line, such as a mouse NSO line or a bacterium, such as E. coli, in which production of the antibody will occur. In order to obtain efficient transcription and translation, the polynucleotide sequence in each vector should include appropriate regulatory sequences, particularly a promoter and leader sequence operatively linked to the variable domain sequence.

Particular methods for producing antibodies in this way are generally well known and routinely used. For example, molecular biology procedures are described by Sambrook et al., Molecular Cloning, A Laboratory Manual, 2nd ed., Cold Spring Harbor Laboratory, New York, 1989; see also Sambrook et al., 3rd ed., Cold Spring Harbor Laboratory, New York, (2001)). While not required, in certain embodiments, domains of polynucleotides encoding the recombinant antibodies are sequenced. DNA sequencing are performed, for example, in any manner or using any systems known in the art. Basic sequencing technology is described for example, in Sanger et al., Proc Natl Acad Sci USA 74:5463 (1977)) and the Amersham International plc sequencing handbook and including improvements thereto.

In particular embodiments, the resulting recombinant antibodies or fragments thereof are then tested to confirm their original specificity, and are further tested for cross-reactivity, e.g., with related polypeptides, in some embodiments. In particular embodiments, an antibody identified or produced according to methods described herein is tested for ability to internalize or other effector function using conventional methods.

### Immune Checkpoint Proteins

Among other things, immune checkpoint proteins or checkpoint proteins play a role in regulating immune response. For instance, immune checkpoint proteins modulate immune response strength to guard against destruction of healthy cells. At the surface of cells, immune checkpoint proteins (e.g., on surfaces of T cells) bind to checkpoint ligands (e.g., on surfaces of myeloid cells, e.g., macrophages), and the binding of the immune checkpoint protein to its ligand suppresses or prevents any further T cell activity (such as T-cell mediated cell death). In some instances, however, such as cancer, immune checkpoint protein-ligand binding activity prevents T cells from killing cancer cells. T cell exhaustion occurs during periods of, for example, chronic infection and cancer and is characterized by poor effector function, sustained expression of inhibitory receptors and changes in transcriptional states (as compared to, e.g., functional effector cells). Relieving macrophage-mediated T cell exhaustion can allow an exhausted T-cell to regain its lost or suppressed effector function.

### Checkpoint Inhibitors

Checkpoint inhibitors have improved outcomes for many types of cancers; however, in some contexts checkpoint inhibitors are not effective. For example, in tumors where immune cells have not infiltrated and/or are not functioning effectively, checkpoint inhibitors are ineffective. The present disclosure provides the insight that a combination of an immunomodulatory CD163 antibody for use with a method disclosed herein and an immune checkpoint inhibitor not only relieves macrophage-mediated immunosuppression, allowing T-cells to kill cancer cells, but does so in an unexpectedly additive or synergistic way, achieving, among other things, relief of macrophage-mediated T-cell immunosuppression and improving T-cell effector function significantly more than would be expected from any additive effect of the antibody and checkpoint inhibitor in a method of the present disclosure. As disclosed herein, immune checkpoint proteins can, in certain contexts and states, interfere with T-cell mediated killing of cancer cells. Checkpoint inhibitors can reverse the interference of immune checkpoint proteins but interference with an immune checkpoint protein is not enough in certain types of cancers (e.g., certain solid tumors). The present disclosure contemplates that combining an immunomodulatory CD163 antibody for use with a method disclosed herein with an immune checkpoint inhibitor impacts macrophage-mediated T-cell suppression in an additive or synergistic way, relieving macrophage-mediated T-cell exhaustion and stimulating T-cell effector function better than with either an antibody or checkpoint inhibitor alone.

In some embodiments, an immune checkpoint inhibitor modulates an immune checkpoint protein. For example, in some embodiments, an immune checkpoint inhibitor reduces, inhibits, interferes with or otherwise changes binding and/or activity of an immune checkpoint protein. In some embodiments, an immune checkpoint inhibitor blocks receptor-ligand interactions. In some such embodiments, such blockage occurs by immune checkpoint protein antagonism. In some such embodiments, such blockage occurs by immune checkpoint protein ligand antagonism (i.e., preventing, in some way, an immune checkpoint protein from binding to its ligand); that is, in some embodiments, an immune checkpoint inhibitor that is an antagonist of an immune checkpoint protein may be or comprise an antagonist of the ligand of a given immune checkpoint protein. In some embodiments, an immune checkpoint inhibitor inhibits immune checkpoint protein and/or immune checkpoint protein ligand-induced signaling.

In some embodiments an immune checkpoint inhibitor inhibits one or more immune checkpoint proteins. Non-limiting examples of immune checkpoint proteins include: PD-1, CD28, CTLA-4, ICOS, TMIGD2, 4-1BB, BTLA, CD160, LIGHT, LAG3, OX40, CD27, CD40L, GITR, DNAM-1, TIGIT, CD96, 2B4, TIM-3, CEACAM1, SIRP alpha, DC-SIGN, CD200R, DR3, CDCHK1, CHK2, A2aR, or B-7 family proteins.

In some embodiments an immune checkpoint inhibitor interacts with a ligand of an immune checkpoint protein. For instance, by way of non-limiting example, a ligand of an immune checkpoint protein includes: PD-L1 (B7-H1), PD-L2 (B7-DC), ICOS ligand, VISTA, 4-1BBL, Herpesvirus Entry Mediator (HVEM), tumor necrosis factor receptor superfamily member 14 or TNFRSF14, MHC class I, MHC class II, OX-40L, CD70, CD40, GITRL, CD155, CD48, GAL9; HMGB1, CEASAM-1, Phosphatidyl serine (PtdSer), IDO, TDO, CD47, BTN2A1, CD200, TL1A, CD112, CD155, MHCII, LSECtin, CHK1, CHK2, A2aR, or a B-7 family ligand (e.g., CD80 (B7-1), CD86 (B7-2), B7-H3, B7-H4, B7-H7 (HHLA2), etc.).

In some embodiments, an immune checkpoint inhibitor is an antagonist. For example, in some embodiments, an immune checkpoint inhibitor antagonizes an immune checkpoint protein. In some embodiments, an immune checkpoint inhibitor is an antagonist to a ligand of an immune checkpoint protein. In some embodiments, the antagonist is a biological molecule such as a biological therapeutic. In some embodiments, an immune checkpoint inhibitor is an antibody or an antigen binding portion thereof. In some embodiments, an immune checkpoint inhibitor is a monoclonal antibody, a humanized antibody, a fully human antibody, a fusion protein, or a combination thereof. In some embodiments an immune checkpoint inhibitor is a small molecule. In some embodiments an immune checkpoint inhibitor is or comprises a rationally-designed peptide. In some embodiments an immune checkpoint inhibitor is or comprises a cell or cell preparation (e.g., cells that express an immune checkpoint inhibitor). In some embodiments, an antagonist inhibits the interaction between PD-1 and PDL-1. In some embodiments, the antagonist is a macrocyclic compound (e.g., gramicidin S and derivatives thereof). In some embodiments, the antagonist is an antibiotic such as an ansamycin type antibiotic (e.g., rifabutin). In some embodiments, the antagonist is a phenolic compound (e.g., kaempferol, kaempferol-7-O-rhamnoside, caffeoylquinic acid, 3-O-caffeoylquinic acid, 4-O-caffeoylquinic acid, 5-O-caffeoylquinic acid, ellagic acid). In some embodiments, the antagonist is a heterocyclic compound (e.g., ZINC 67,902,090, ZINC 12,529,904). In some embodiments, the antagonist is a small molecule (e.g., CA-170, ARB-272572, INCB086550). In some embodiments, the antagonist is actinomycin D, amphotericin B, bacitracin, bryostatin, candicidin, clarithromycin, cyclosporin A, cyanocobalamin, erythromycin, everolimus, geldanamycin, ivermectin B1a, macbecin, metocurine, monocrotaline, nystatin, plerixafor, rifampin, sirolimus, troleandomycin, rifabutin, rifapentine, rifamycin SV, formyl rifamycin, rifaximin, gramicidin S, ZINC 67,902,090, ZINC 12,529,904, or derivatives thereof. In some embodiments, the antagonist is cyclo(-Leu-DTrp-Pro-Thr-Asp-Leu-DPhe-Lys(Dde)-Val-Arg) (SEQ ID NO: 46), rifabutin, kaempferol, kaempferol-7-O-rhamnoside, eriodictyol, fisetin, glyasperin C, cosmosiin, ellagic acid, caffeoylquinic acids, or derivatives thereof.

In some embodiments, an immune checkpoint inhibitor inhibits PD-1. Programmed cell death 1 (PD-1) is a key checkpoint receptor expressed by activated T and B cells, and mediates immunosuppression. Among other things, PD-1 limits activity of T cells in peripheral tissues during an inflammatory response to infection. In addition, as an immune checkpoint protein, PD-1 blockade can enhance T-cell proliferation and cytokine production in response to a challenge by specific antigen targets or by allogeneic cells in mixed lymphocyte reactions.

The present disclosure contemplates that, in some embodiments, blockade of PD-1, in combination with an immunomodulatory hCD163 antibody as disclosed herein, is additive or synergizes to relieve macrophage-mediated T cell suppression/exhaustion, increase T cell proliferation and cytokine production and improve immune cell effector function. PD-1 blockade can be accomplished by a variety of mechanisms, usually by blocking or interrupting PD-1/PD-L1 interaction. In some embodiments, the immune checkpoint inhibitor blocks the PD-1/PD-L1 interaction by binding to PD-1 or to PD-L1. Herein, if an agent inhibits or antagonizes PD-1/PD-L1 interaction by preferentially or specifically binding to PD-1, then it is referred to as a PD-1 antagonist; if it does so by preferentially or specifically binding to PD-L1, then it is referred to as a PD-L1 antagonist.

In some embodiments, the immune checkpoint inhibitor is a PD-1 antagonist, and can be an anti-PD-1 antibody, a small molecule, a rationally-designed peptide, or a cell or cell preparation (e.g., cells that express a PD-1 binding agent, e.g., a PD-1 antibody).

In some embodiments, the PD-1 antagonist is or comprises an antibody or an antigen-binding fragment thereof. In some embodiments the PD-1 antagonist is nivolumab (OPDIVO®) (Bristol-Myers Squibb), pembrolizumab (KEYTRUDA®) (Merck), cemipilimab (e.g., cemiplimab-rwlc, LIBTAYO®) (Regeneron), dostarlimab (e.g., dostarlimab-gxly, JEMPERLI®) (GSK), JTX-4014 (IgG4 antibody) (Jounce Therapeutics), spartalizumab (PDR001) (Novartis), camrelizumab (SHR1210) (Jiangsu HengRui Medicine Co., Ltd.), sintilimab (IBI308) (Innovent and Eli Lilly), tislelizumab (BGB-A317) (BeiGene), toripalimab (JS 001) (Shanghai Junshi Bioscience Co., Ltd), INCMGA00012 (MGA012) (Incyte and MacroGenics), AMP-224 (PD-L2/Ig fusion) (AstraZeneca/MedImmune and GSK), and/or AMP-514 (MEDI0680; IgG4k antibody) (AstraZeneca), zimberelimab (Arcus Bioscience), or a PD-1 binding domain thereof.

In some embodiments, a PD-1 antagonist is or comprises a rationally designed peptide such as, e.g., APi2568, which comprises a B-cell epitope (amino acids 92-110 from PD-1) linked to a promiscuous T-cell epitope (amino acid residues 288-302 from measles virus fusion protein) via a 4-amino acid linker (Gly-Pro-Ser-Leu) (SEQ ID NO: 48), and combined with Water for Injection (WFI) forms the drug product, IMU-201, which becomes PD1-Vaxx when emulsified with excipient Montanide ISA 720 VG.

In some embodiments, a PD-1 antagonist is or comprises cells expressing a PD-1 antibody, for example, PD-1 antibody-expressing-CAR-T cells (e.g., HerinCAR-PD1 cells).

Alternatively, or additionally, in some embodiments, PD-1 blockade is achieved by blocking a PD-1 ligand such as PD-L1. Non-limiting examples of PD-1 and PD-L1 blockers are described in U.S. Pat. Nos. 7,488,802; 7,943,743; 8,008,449; 8,168,757; 8,217,149, and PCT Published Patent Application Nos: WO 03/042402, WO 2008/156712, WO 2010/089411, WO 2010/036959, WO 2011/066342, WO 2011/159877, WO 2011/082400, and WO 2011/161699, each of which is herein incorporated by reference in its entirety.

In some embodiments, the immune checkpoint inhibitor is a PD-L1 inhibitor (e.g., an antibody, e.g., a small molecule, peptide, etc.). In some embodiments, a PD-L1 inhibitor is an antibody or antigen binding portion thereof.

In some embodiments, the PD-L1 antagonist is or comprises is a PD-L1 antibody, such as avelumab (BAVENCIO®) (Merck), durvalumab (IMFINZI®) (MedImmune), and atezolizumab (TECENTRIQ®) (Genentech), envafolimab (KN035; single chain antibody) (Tracon Pharma; Simcere Pharmaceutical), cosibelimab (CK-301) (Checkpoint Therapeutics), CA-170 (a PD-L1 and VISTA antagonist) (Aurigene/Curis), BMS-936559 (Bristol-Myers Squibb), or an PD-L1-binding fragment thereof, or a combination of any of them.

In some embodiments, a PD-L1 antagonist is or comprises a small molecule, e.g., INCB086550 (Incyte) or WP-1066 (MDACC).

In some embodiments, a PD-L1 antagonist is or comprises a peptide, e.g., AUNP-12 (29-mer peptide) (Aurigene and Laboratoires Pierre Fabre), BMS-189/BMS-986189 (Bristol Meyers Squibb), or MT-6402 (Molecular Templates).

In some embodiments, an antagonist inhibits the interaction between PD-1 and PDL-1. In some embodiments, the antagonist is a macrocyclic compound (e.g., gramicidin S and derivatives thereof). In some embodiments, the antagonist is an antibiotic such as an ansamycin type antibiotic (e.g., rifabutin). In some embodiments, the antagonist is a phenolic compound (e.g., kaempferol, kaempferol-7-O-rhamnoside, caffeoylquinic acid, 3-O-caffeoylquinic acid, 4-O-caffeoylquinic acid, 5-O-caffeoylquinic acid, ellagic acid). In some embodiments, the antagonist is a heterocyclic compound (e.g., ZINC 67,902,090, ZINC 12,529,904). In some embodiments, the antagonist is a small molecule (e.g., CA-170, ARB-272572, INCB086550). In some embodiments, the antagonist is actinomycin D, amphotericin B, bacitracin, bryostatin, candicidin, clarithromycin, cyclosporin A, cyanocobalamin, erythromycin, everolimus, geldanamycin, ivermectin B1a, macbecin, metocurine, monocrotaline, nystatin, plerixafor, rifampin, sirolimus, troleandomycin, rifabutin, rifapentine, rifamycin SV, formyl rifamycin, rifaximin, gramicidin S, ZINC 67,902,090, ZINC 12,529,904, or derivatives thereof. In some embodiments, the antagonist is cyclo(-Leu-DTrp-Pro-Thr-Asp-Leu-DPhe-Lys(Dde)-Val-Arg) (SEQ ID NO: 46), rifabutin, kaempferol, kaempferol-7-O-rhamnoside, eriodictyol, fisetin, glyasperin C, cosmosiin, ellagic acid, caffeoylquinic acids, or derivatives thereof.

In some embodiments, an immune checkpoint inhibitor inhibits cytotoxic T-lymphocyte-associated protein 4 (CTLA-4) or a ligand thereof. In some embodiments, anti-CTLA-4 antibodies bind to CTLA-4 and block the interaction of CTLA-4 with its ligands CD80/CD86, which ligands are expressed on antigen presenting cells. Accordingly, in some embodiments, a CTLA-4 inhibitor that blocks interaction of CTLA-4 and its ligands can block negative down regulation of the immune responses elicited by the interaction of these molecules.

In some embodiments, an immune checkpoint inhibitor is a CTLA-4 antagonist. In some such embodiments, a CTLA-4 antagonist is an antibody such as described in U.S. Pat. Nos. 5,811,097; 5,811,097; 5,855,887; 6,051,227; 6,207,157; 6,682,736; 6,984,720; and 7,605,238. For example, without limitation, in some embodiments CTLA-4 antibodies include: ipilimumab (10D1, MDX-D010; Yervoy®) (Bristol-Myers Squibb) and tremelimumab (ticilimumab, CP-675,206) (AstraZeneca). In some embodiments, a CTLA-4 antagonist comprises a CTLA-4-binding domain or fragment thereof of any CTLA-4 antibody. In some embodiments, a CTLA-4 antagonist is or comprises a small molecule (see, e.g., Wang et al (2019) Bioch Bioph Acta (BBA) 1871 (2): 199-224).

Lymphocyte-activation gene 3 (LAG-3, also known as CD223) is a CD4-related transmembrane protein that competitively binds MHC II and acts as a co-inhibitory checkpoint for T cell activation (see, e.g., Goldberg and Drake (2011) Curr. Top. Microbiol. Immunol. 344:269-78).

In some embodiments, an immune checkpoint inhibitor is a LAG3 antagonist. In some embodiments, a LAG3 antagonist is a LAG-3-binding protein (e.g., an antibody) or a protein that binds to a LAG3 ligand.

In some embodiments, non-limiting examples of LAG-3 antibodies include: LAG525 (IMP701, Novartis/Prima Biomed), MK-4280 (Merck Sharp & Dohme), REGN3767 (Regeneron Pharmaceuticals), relatlimab (BMS-986016, Bristol-Myers Squibb), and BI 754111 (Boehringer Ingelheim). In some embodiments, a LAG3 antagonist comprises a LAG3-binding domain or fragment thereof of any LAG3 antagonist.

T cell immunoglobulin mucin 3 (TIM-3, also known as Hepatitis A virus cellular receptor (HAVCR2)) is a type I glycoprotein receptor that binds to S-type lectin galectin-9 (Gal-9). TIM-3, is a widely expressed ligand on lymphocytes, liver, small intestine, thymus, kidney, spleen, lung, muscle, reticulocytes, and brain tissue. Binding of Gal-9 by the TIM-3 receptor triggers downstream signaling to negatively regulate T cell survival and function.

In some embodiments, an immune checkpoint inhibitor is an agent that inhibits TIM-3. In some embodiments, an immune checkpoint inhibitor is a TIM-3 antagonist, such as a TIM3 antibody or an antibody to a TIM3 ligand. In some embodiments, a TIM-3 antagonist comprises a TIM-3-binding domain or fragment thereof of any TIM-3 antagonist.

Non-limiting examples of TIM-3 antagonists include: TSR-022 (AnaptysBio/Tesaro) and MGB453 (Novartis). Additional TIM-3 binding proteins (e.g., antibodies) are known in the art and are disclosed, e.g., in U.S. Pat. Nos. 9,103,832, 8,552, 156, 8,647,623, 8,841,418; U.S. Patent Application Publication Nos. 2016/0200815, 2015/0284468, 2014/0134639, 2014/0044728, 2012/0189617, 2015/0086574, 2013/0022623; and PCT Publication Nos. WO 2016/068802, WO 2016/068803, WO 2016/071448, WO 2011/155607, and WO 2013/006490, each of which is herein incorporated by reference in its entirety.

T cell immunoglobulin and ITIM domain (TIGIT) is an inhibitory receptor expressed on lymphocytes. TIGIT interacts with CD155 expressed on antigen-presenting cells or tumor cells to down-regulate T cell and natural killer (NK) cell functions.

In some embodiments, an immune checkpoint inhibitor is an TIGIT antagonist. In some embodiments, a TIGIT antagonist binds to TIGIT or to a TIGIT ligand. In some embodiments, a TIGIT antagonist is a TIGIT antibody or an antibody to a TIGIT ligand. In some embodiments, a TIGIT antagonist comprises a TIGIT-binding domain or fragment thereof of any TIGIT antagonist.

Non-limiting examples of TIGIT antagonists include: tiragolumab (MTIG7192A; RG6058) (Genentech/Roche), AB154 (Arcus Bioscience), MK-7684 (Merck), BMS-985207 (Bristol-Myers Squibb), and ASP8374 (Astellas Pharma; Potenza Therapeutics).

In some embodiments, the immunotherapy comprises a targeted immunocytokine, e.g., cergutuzumab amunaleukin (CEA-IL2v).

### Tumor Phenotyping

In some embodiments, an immunomodulatory CD163 antibody for use with a method disclosed herein is tested for tumor growth inhibition using cells isolated from a subject alone or in combination with an immune checkpoint inhibitor according to methods of the present disclosure. In some embodiments, a subject is treatment naïve. In some embodiments, a subject has been treated with an immunomodulatory CD163 antibody for use with a method disclosed herein as a monotherapy or in combination with an immune checkpoint inhibitor. In some embodiments, tumor cells are tested in vitro and treated with an immunomodulatory CD163 antibody for use with a method disclosed herein, an immune checkpoint inhibitor, and a combination of an antibody and checkpoint inhibitor.

In some embodiments, immunostimulatory activity in the tumor microenvironment is assessed via biopsy or in situ scanning. In some embodiments, tissues from biopsy of a subject, may include samples from an organ affected with cancer such as, e.g., lungs, liver, skin, breast tissue, etc. In some embodiments, a sample includes a tumor. In some embodiments, a sample of a tumor is assayed using panels for tumor-associated macrophages (“TAM”, assayed using flow cytometry); and T cell type flow panels and tumor microenvironment (TME) cytokine panels, e.g., using flow cytometry. In some embodiments, the human tissue is obtained from biopsy from a subject treated with an immunomodulatory CD163 antibody for use with a method disclosed herein as a monotherapy or in combination with an immune checkpoint inhibitor.

### Culture Systems

In some embodiments, a combination of an immunomodulatory CD163 antibody for use with a method disclosed herein and an immune checkpoint inhibitor promotes expression of IFN-γ by T cells. In some embodiments, a combination of an immunomodulatory CD163 antibody for use with a method disclosed herein and an immune checkpoint inhibitor reduces suppression of T cell activation (e.g., reduces the ability of TAMs to suppress T-cell activation), leading to greater T-cell stimulation and IL-2 production. In some embodiments, a combination of an antibody disclosed herein and an immune checkpoint inhibitor increases IL-2 production. In some embodiments, IL-2 production is a marker of T-cell stimulation and proliferation. In some embodiments, a combination comprising an immunomodulatory CD163 antibody and an immune checkpoint inhibitor, as disclosed herein, blocks the ability of myeloid cells to suppress T-cell activation, as evidenced by increased IL-2 production.

### Exemplary Outcomes

In the case of treatment of a cancer, examples of observable and/or measurable change in a parameter or symptom of the disease or disorder includes increased tumor cell killing activity as assessed ex vivo, lower levels of immunosuppressive secreted factors in blood, lower tumor volume or mass, increased cytotoxic lymphocytes and Th1 like T cell numbers in tumor biopsy, reduced morbidity or mortality, improvement in quality of life factors, or improvement in any objective indicia related to a parameter or symptom of the disease or disorder. In some embodiments, the parameters include converting immune cold tumors into immune hot, e.g., by increasing cytotoxic lymphocyte cell number as well as markers of T cell activation (CD69, ICOS, OX40, etc.) in tumor biopsy, or decreasing expression of CD16, CD64, TLR2, Siglec-15 on TAMs in tumor biopsy.

A response is achieved when the subject experiences partial or total alleviation, or reduction of signs or symptoms of illness, and specifically includes, without limitation, prolongation of survival. The expected progression-free survival times are measured, for example, in months to years, depending on prognostic factors including the number of relapses, stage of disease, and other factors. Prolonging survival includes without limitation times of at least 1 month (mo.), about at least 2 months (mos.), about at least 3 mos., about at least 4 mos., about at least 6 mos., about at least 1 year, about at least 2 years, about at least 3 years, or more. Overall survival is also be measured in months to years in some embodiments. The subject's symptoms remain static or decrease in some embodiments.

Human subjects treated with both (i) AB101 or another immunomodulatory CD163 antibody as disclosed herein and (ii) an immune checkpoint inhibitor (e.g., anti-PD-1 antibody) are evaluated on several measures. In some embodiments, primary endpoints, e.g., safety and tolerability, as well as dose and regimen, are determined prior to or during the combination trial. Subjects are evaluated for responsiveness to treatment (complete response, partial response, and/or disease stability). In some embodiments, objective response rate for anti-tumor activity is conducted using known criteria including RECIST v 1.1 (Eisenhauer 2009 Eur J Cancer 45:228-247; see also Aykan 2020 World J Clin Oncol 11(2):53-73, each of which is incorporated herein by reference in its entirety) and, optionally, iRECIST v1.1 criteria. In some embodiments, results are reported by tumor type cohort as a percentage of the efficacy population in that cohort, with 95% confidence intervals; any other anti-tumor activity variables are reported by standard methods.

In some embodiments, anti-tumor activity is also measured as determined by CT or MRI at week 6, 12, 18, 24, 36, 48, and every six months thereafter. In some embodiments, immunogenicity is evaluated to determine presence and frequency of anti-drug antibodies, including neutralizing antibodies to either AB101 and/or an immune checkpoint inhibitor (e.g., anti-PD-1 antibody) on pre-dose Day 1 of each three-week cycle for five cycles. Objective response rate, progression-free survival, and overall survival are also measured.

In some embodiments, subjects treated with combinations of AB101 or other immunomodulatory CD163 antibody as disclosed herein and an immune checkpoint inhibitor (e.g., anti-PD-1 antibody) are found to have success in treatment of refractory or treatment-resistant tumors compared to prior to any previous treatments. In some embodiments, subjects have enhanced anti-tumor activity as determined by CT or MRI and prolonged clinical responses to immunotherapy as compared to prior clinical responses with prior or no treatments. In some embodiments, subjects treated with AB101 or the like in combination with an immune checkpoint inhibitor (e.g., anti-PD-1 antibody) do not exhibit anti-drug antibodies (ADAs) or significant increases in anti-drug antibodies to AB101 or the like and/or antibodies to an immune checkpoint inhibitor over the course of treatment. In some embodiments, subjects exhibits some anti-drug antibodies to AB101 or the like and/or antibodies to an immune checkpoint inhibitor over the course of treatment, but the antibodies are not neutralizing and are considered to be not clinically significant.

Disclosed herein are methods of treating a human subject having a cancer, comprising administering to a subject a therapeutically effective amount of an antibody as described herein and an immune checkpoint inhibitor.

In some embodiments, in a method disclosed herein, immunosuppression by tumor-associated macrophages in a subject is antagonized. In some embodiments, in a method disclosed herein, immunosuppression by tumor-associated macrophages in a subject is reduced. In some embodiments, in a method disclosed herein, immunosuppression by tumor-associated macrophages in the subject is antagonized by administration of a combination disclosed herein compared to the immune checkpoint inhibitor administered in the absence of the immunomodulatory CD163 antibody.

In some embodiments, a method disclosed herein antagonizes immunosuppressive function of tumor-associated macrophages in a subject. In some embodiments, a method disclosed herein potentiates one or more immune responses in a subject. In some embodiments, a method disclosed herein increases T cell-mediated tumor cell killing in a subject. In some embodiments, a method disclosed herein increases proliferation of CD3+ cells (e.g., CD4+ and/or CD8+ T cells). In some embodiments, a method disclosed herein promotes increased cytokine levels in the subject.

In some embodiments, a method disclosed herein promotes proliferation of CD8+ or CD4+ T cells in the subject. In some embodiments, a method disclosed herein promotes cytokine secretion in the subject. In some embodiments, a method disclosed herein promotes T cell-mediated killing of tumor cells in the subject.

In some embodiments, a method disclosed herein reduces myeloid cell suppression of CD8+ T cell activation and proliferation.

In some embodiments, the antibodies reduce myeloid cell suppression of CAR T-cell-mediated killing of cancer cells.

In some embodiments, the antibodies reduce myeloid cell suppression of NK cell-mediated killing of cancer cells by ADCC.

Disclosed herein, in certain embodiments, are methods of relieving T cells suppression. In some embodiments, the disclosure provides a method of decreasing suppression of signaling by receptors on T cells and/or increasing T cell activation. In some embodiments, the decreased suppression of receptor signaling in T cells and/or increased T cell activation is essential for mounting effective anti-tumor immunity.

In some embodiments, a method disclosed herein increases, enhances or prolongs anti-tumor activity by T cells.

In some embodiment, a clinical outcome is tumor regression, tumor shrinkage, tumor necrosis, increased anti-tumor response by the immune system, decreased tumor expansion, recurrence or spread, or a combination thereof.

### Pharmaceutical Compositions

Disclosed herein, in certain embodiments, are pharmaceutical compositions comprising an immunomodulatory CD163 antibody as disclosed herein and a pharmaceutically acceptable excipient or an immune checkpoint inhibitor and a pharmaceutically acceptable excipient.

In some embodiments, the CD163 antibody for use with a method disclosed herein can be formulated using commercially available technology. Many such technologies exist for formulating antibodies and other therapeutic proteins. For example, a survey of compositions used for commercialized antibodies is given in Strickley, J Pharm Sci, 2021 July; 110(7):2590-2608. In some embodiments, the immune checkpoint inhibitor can be formulated using commercially available technology known in the art.

Such compositions are useful for in vitro or in vivo analysis or, in the case of pharmaceutical compositions, for administration to a subject in vivo or ex vivo for treating a subject with the disclosed antibodies.

In some embodiments, the excipient is a carrier, buffer, stabilizer or other suitable materials known to those skilled in the art. Such materials should be non-toxic and should not interfere with the efficacy of the active ingredient. The precise nature of the carrier or other material will depend on the route of administration.

In some embodiments, a pharmaceutical composition comprises an immunomodulatory CD163 antibody for use with a method disclosed herein and a pharmaceutically acceptable excipient. In some embodiments, a pharmaceutical composition comprises an immune checkpoint inhibitor and a pharmaceutically acceptable excipient. In some embodiments, the immune checkpoint inhibitor blocks an immune checkpoint protein as disclosed herein.

Pharmaceutical formulations comprising an antibody or antigen-binding fragment, identified by the methods described herein are prepared for storage by mixing the protein having the desired degree of purity with optional physiologically acceptable carriers, excipients or stabilizers (see, e.g., Remington's Pharmaceutical Sciences, 16^{th} edition, Osol, A. Ed. (1980)), in the form of lyophilized formulations or aqueous solutions in some embodiments. Acceptable carriers, or stabilizers are those that are non-toxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN®, PLURONICS® or polyethylene glycol (PEG). In certain embodiments, the pharmaceutical composition comprises an immunomodulatory CD163 antibody at a concentration of between 5-200 mg/mL; preferably between 10⁻¹⁰⁰ mg/mL.

Acceptable carriers are physiologically acceptable to the administered subject and retain the therapeutic properties of the compounds with/in which it is administered. Acceptable carriers and their formulations are and generally described in, for example, Remington's Pharmaceutical Sciences, supra. One exemplary carrier is physiological saline. The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject compounds from the administration site of one organ, or portion of the body, to another organ, or portion of the body, or in an in vitro assay system. Each carrier is acceptable in the sense of being compatible with the other ingredients of the formulation and not injurious to a subject to whom it is administered. Nor should an acceptable carrier alter the specific activity of the subject compounds.

In another embodiment, a pharmaceutical composition disclosed herein further comprises an acceptable additive to improve the stability of the compounds in composition and/or to control the release rate of the composition. Acceptable additives do not alter the specific activity of the subject compounds. Exemplary acceptable additives include, but are not limited to, a sugar such as mannitol, sorbitol, glucose, xylitol, trehalose, sorbose, sucrose, galactose, dextran, dextrose, fructose, lactose, and mixtures thereof. Acceptable additives are combined with acceptable carriers and/or excipients such as dextrose in some embodiments. Alternatively, exemplary acceptable additives include, but are not limited to, a surfactant such as polysorbate 20 or polysorbate 80 to increase stability of the peptide and decrease gelling of the solution. In some embodiments, the surfactant is added to the composition in an amount of 0.01% to 5% of the solution. Addition of such acceptable additives increases the stability and half-life of the composition in storage.

In one embodiment, a pharmaceutical composition disclosed herein contains an isotonic buffer such as a phosphate, acetate, or TRIS buffer in combination with a tonicity agent such as a polyol, Sorbitol, sucrose or sodium chloride, which tonicifies and stabilizes. In some embodiments, a tonicity agent is present in the composition in an amount of about 5%.

In another embodiment, a pharmaceutical composition disclosed herein includes a surfactant such as to prevent aggregation and for stabilization at 0.01 to 0.02% wt/vol.

In another embodiment, the pH of a pharmaceutical composition disclosed herein ranges from 4.8-8.0, 4.5-6.5 or 4.5-5.5.

In some embodiments, a pharmaceutical composition disclosed herein also contains more than one active compound as necessary for the indication being treated, such as those with complementary activities that do not adversely affect each other. For example, a method of treatment further provides an immunosuppressive agent. Such molecules are suitably present in combination in amounts that are effective for the purpose intended.

In some embodiments, active ingredients are entrapped in microcapsule prepared, for example, by coacervation techniques or by interfacial polymerization, for example, hydroxy methylcellulose or gelatin-microcapsule and poly-(methylmethacrylate) microcapsule, respectively, in colloidal drug delivery systems (for example, liposomes, albumin microspheres, microemulsions, nanoparticles, and nanocapsules) or in macroemulsions. Such techniques are disclosed in Remington's Pharmaceutical Sciences, supra.

Suspensions and crystal forms of antibodies are also contemplated herein; methods to make suspensions and crystal forms are known to one of skill in the art.

In some embodiments, a pharmaceutical composition disclosed herein is sterile. In some embodiments, a pharmaceutical composition disclosed herein is sterilized by conventional, well known sterilization techniques. For example, sterilization is readily accomplished by filtration through sterile filtration membranes. In some embodiments, the resulting solutions is packaged for use or filtered under aseptic conditions and lyophilized, the lyophilized preparation being combined with a sterile solution prior to administration.

Freeze-drying is employed to stabilize polypeptides for long-term storage, such as when a polypeptide is relatively unstable in liquid compositions, in some embodiments.

In some embodiments, some excipients such as, for example, polyols (including mannitol, sorbitol, and glycerol); sugars (including glucose and sucrose); and amino acids (including alanine, glycine, and glutamic acid), act as stabilizers for freeze-dried products. Polyols and sugars are also used to protect polypeptides from freezing and drying-induced damage and to enhance the stability during storage in the dried state in some embodiments. Sugars are, in some embodiments, effective in both the freeze-drying process and during storage. Other classes of molecules, including mono- and disaccharides and polymers such as PVP, have also been reported as stabilizers of lyophilized products.

For injection, in some embodiments, a pharmaceutical composition disclosed herein is a powder suitable for reconstitution with an appropriate solution as described above. Examples of these include, but are not limited to, freeze dried, rotary dried or spray dried powders, amorphous powders, granules, precipitates, or particulates. For injection, the compositions optionally contain stabilizers, pH modifiers, surfactants, bioavailability modifiers and combinations of these.

Sustained-release preparations is prepared in some embodiments. Suitable examples of sustained-release preparations include semipermeable matrices of solid hydrophobic polymers containing an antibody (e.g., an immunomodulatory CD163 antibody), which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (see, e.g., U.S. Pat. No. 3,773,919), copolymers of L-glutamic acid and y ethyl-L-glutamate, non-degradable ethylene-vinyl acetate, degradable lactic acid-glycolic acid copolymers such as the Lupron Depot™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. In some embodiments, while encapsulated antibodies remain in the body for a long time, they denature or aggregate as a result of exposure to moisture at 37° C., resulting in a loss of biological activity and possible changes in immunogenicity. Rational strategies devised for stabilization are, in some cases, dependent on the mechanism involved. For example, if the aggregation mechanism is discovered to be intermolecular S-bond formation through thio-disulfide interchange, stabilization is achieved, in some cases, by modifying sulfhydryl residues, lyophilizing from acidic solutions, controlling moisture content, using appropriate additives, and developing specific polymer matrix compositions.

In some embodiments, a pharmaceutical composition disclosed herein is designed to be short-acting, fast-releasing, long-acting, or sustained-releasing as described herein. In one embodiment, a pharmaceutical composition disclosed herein is formulated for controlled release or for slow release.

The pharmaceutical composition is administered, for example, by injection, including, but not limited to, subcutaneous, intravitreal, intradermal, intravenous, intraarterial, intraperitoneal, intracerebrospinal, or intramuscular injection. Excipients and carriers for use in formulation of compositions for each type of injection are contemplated herein. The following descriptions are by example only and are not meant to limit the scope of the compositions. Compositions for injection include, but are not limited to, aqueous solutions (where water soluble) or dispersions, as well as sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In some embodiments, the carrier is a solvent or dispersion medium containing, for example, water, ethanol, polyol (for example, glycerol, propylene glycol, and liquid polyethylene glycol, and the like), and suitable mixtures thereof. Fluidity is maintained, for example, by the use of a coating such as lecithin, by the maintenance of the required particle size in the case of dispersion and by the use of surfactants. Antibacterial and antifungal agents include, for example, parabens, chlorobutanol, phenol, ascorbic acid, and thimerosal. Isotonic agents, for example, sugars, polyalcohols such as mannitol, sorbitol, and sodium chloride are included in the composition in some embodiments. In some embodiments, the resulting solutions are packaged for use as is, or lyophilized; the lyophilized preparation is later be combined with a sterile solution prior to administration in some embodiments. For intravenous, injection, or injection at the site of affliction, the active ingredient will be in the form of a parenterally acceptable aqueous solution which is pyrogen-free and has suitable pH, isotonicity, and stability. Those of relevant skill in the art are well able to prepare suitable solutions using, for example, isotonic vehicles such as Sodium Chloride Injection, Ringer's Injection, and Lactated Ringer's Injection. Preservatives, stabilizers, buffers, antioxidants, and/or other additives are included, as needed, in some embodiments. Sterile injectable solutions are prepared by incorporating an active ingredient in the required amount in an appropriate solvent with one or a combination of ingredients enumerated above, as required, followed by filtered sterilization, in some embodiments. Generally, dispersions are prepared by incorporating the active ingredient into a sterile vehicle which contains a basic dispersion medium and the required other ingredients from those enumerated above. In the case of sterile powders for the preparation of sterile injectable solutions, the preferred methods of preparation are vacuum drying and freeze drying which yields a powder of the active ingredient plus any additional desired ingredient from a previously sterile-filtered solution thereof.

Compositions are conventionally administered intravenously in some embodiments, such as by injection of a unit dose, for example. For injection, in some embodiments, an active ingredient is in the form of a parenterally acceptable aqueous solution which is substantially pyrogen-free and has suitable pH, isotonicity, and stability. In some embodiments, one prepares suitable solutions using, for example, isotonic vehicles such as Sodium Chloride Injection, Ringer's Injection, Lactated Ringer's Injection. Preservatives, stabilizers, buffers, antioxidants, and/or other additives are included, as required, in some embodiments. Additionally, compositions are administered via aerosolization in some embodiments. (Lahn et al., Int Arch Allergy Immunol 134:49-55 (2004)).

For parenteral administration, antibodies or checkpoint inhibitors are formulated in a unit dosage injectable form (solution, suspension, emulsion) in association with a pharmaceutically acceptable, parenteral vehicle. Examples of such vehicles are water, saline, Ringer's solution, dextrose solution, and 5% human serum albumin. Nonaqueous vehicles such as fixed oils and ethyl oleate are also used. Liposomes are used as carriers. The vehicle contains minor amounts of additives such as substances that enhance isotonicity and chemical stability, e.g., buffers and preservatives. The antibodies are typically formulated in such vehicles at concentrations of about 1 mg/mL to 10 mg/mL.

In one embodiment, a pharmaceutical composition disclosed herein is lyophilized, for example, to increase shelf-life in storage. When the compositions are considered for use in medicaments or any of the methods provided herein, in some embodiments, it is contemplated that the composition are substantially free of pyrogens such that the composition will not cause an inflammatory reaction or an unsafe allergic reaction when administered to a human subject. Testing compositions for pyrogens and preparing compositions substantially free of pyrogens are well understood to one or ordinary skill of the art and are accomplished using commercially available kits in some embodiments.

In some embodiments, acceptable carriers contain a compound that stabilizes, increases or delays absorption or clearance. Such compounds include, for example, carbohydrates, such as glucose, sucrose, or dextrans; low molecular weight proteins; compositions that reduce the clearance or hydrolysis of peptides; or excipients or other stabilizers and/or buffers. Agents that delay absorption include, for example, aluminum monostearate and gelatin. In some embodiments, detergents also be used to stabilize or to increase or decrease the absorption of the pharmaceutical composition, including liposomal carriers. To protect from digestion the compound, in some embodiments, is complexed with a composition to render it resistant to acidic and enzymatic hydrolysis, or the compound is, in some embodiments, complexed in an appropriately resistant carrier such as a liposome. Means of protecting compounds from digestion are known in the art.

The compositions are administered, in some embodiments, in a manner compatible with the dosage formulation, and in a therapeutically effective amount. The quantity to be administered depends on the subject to be treated, capacity of the subject's immune system to utilize the active ingredient, and degree of binding capacity desired. Precise amounts of active ingredient required to be administered depend on the judgment of the practitioner and are peculiar to each individual. Suitable regimes for initial administration and booster shots are also variable, but are typified by an initial administration followed by repeated doses at one- or more-hour intervals by a subsequent injection or other administration. Alternatively, continuous intravenous infusion that is sufficient to maintain concentrations in the blood are contemplated.

In some embodiments, the disclosure provides a use of the compositions described herein to make a medicament for treating a condition, disease, or disorder described herein. In some embodiments, medicaments are formulated based on the physical characteristics of the subject needing treatment, and are formulated in single or multiple formulations based on the stage of the condition, disease or disorder. Medicaments are packaged in a suitable package with appropriate labels for the distribution to hospitals and clinics in which the label is for the indication of treating a subject having a disease described herein in some embodiments. Medicaments are packaged as a single or multiple units in some embodiments. Instructions for the dosage and administration of the compositions are included with the packages as described below in some embodiments. The disclosure is further directed to medicaments comprising an antibody or antigen-binding fragment thereof described herein and a pharmaceutically acceptable carrier.

In some embodiments, amounts of the active ingredients in the compositions, the composition formulation, and the mode of administration, are among the factors that are varied to provide an amount of the active ingredient that is effective to achieve the desired therapeutic response for each subject, without being unduly toxic to the subject. The selected dosage level will depend upon a variety of factors including the activity of the particular compound employed, the route of administration, the time of administration, the rate of excretion or metabolism of the particular compound being employed, the duration of the treatment, other drugs, compounds and/or materials used in combination with the particular composition employed, the age, sex, weight, condition, general health, diet and prior medical history of the subject being treated, and like factors well known in the medical arts.

### Pharmaceutical Administration

In some embodiments, the antibodies and antigen-binding fragments described herein are administered to a subject in various dosing amounts and over various time frames. Non-limiting doses include about 0.01 mg/kg, about 0.05 mg/kg, about 0.1 mg/kg, about 0.5 mg/kg, about 1 mg/kg, about 5 mg/kg, about 10 mg/kg, about 20 mg/kg, about 30 mg/kg, about 40 mg/kg, about 50 mg/kg, about 60 mg/kg, about 70 mg/kg, about 80 mg/kg, about 90 mg/kg, about 100 mg/kg, about 125 mg/kg, about 150 mg/kg, about 175 mg/kg, about 200 mg/kg, or any integer in between.

Additionally, the dose(s) of an immunomodulatory CD163 antibody or antigen-binding fragment are administered, in some embodiments, about twice a week, about once per week, about every two weeks, about every three weeks, about every 4 weeks, about every 6 weeks, about every 8 weeks, about every 12 weeks, or any combination of numbers of weeks therein. Dosing cycles are also contemplated such as, for example, administering antibodies or antigen-binding fragments thereof once or twice a week for 4 weeks, followed by two weeks without therapy. In some embodiments, another such dosing regimen may include administering an immunomodulatory CD163 antibody or antigen-binding fragment thereof once a week on a continuing basis, such as for 8 weeks, 12 weeks, or longer.

Additional dosing cycles including, for example, different combinations of the doses and weekly cycles described herein are also contemplated within the disclosure.

Therapeutically effective amounts or therapeutic amounts of a composition, in some embodiments, varies and depends on the severity of the disease and the weight and general state of the subject being treated, but generally range from about 1.0 μg/kg to about 100 mg/kg body weight, or about 10 μg/kg to about 30 mg/kg, or about 0.1 mg/kg to about 10 mg/kg or about 1 mg/kg to about 10 mg/kg per application. Administration can be daily, on alternating days, weekly, twice a month, monthly or more or less frequently, as necessary depending on the response to the disorder or condition and the subject's tolerance of the therapy. In some embodiments, administration is continued for a period and/or depending upon occurrence or absence of one or more particular events or outcomes. In some embodiments, administration is discontinued after a period, and/or depending upon occurrence or absence of one or more particular events or outcomes. In some embodiments, administration of a composition of the present disclosure is continued as long as the disease is stable, as long as the patient experiences a partial response or a complete response, until disease progression, or until unacceptable toxicity occurs. In some embodiments, maintenance dosages over a longer period of time, such as 4, 5, 6, 7, 8, 10, or 12 weeks or longer is needed until a desired suppression of disorder symptoms occurs, and dosages are adjusted as necessary. The progress of this therapy is easily monitored by conventional techniques and assays.

In some embodiments, the amount and/or frequency of administration of the immunomodulatory CD163 antibody or fragment may be chosen or changed based on assessment of the concentration of the active moiety in a patient's blood, plasma, serum, or other sample. In some embodiments, the amount and/or frequency of administration is selected based on maintaining a trough level between doses above a level that is deemed to be effective for treatment of the patient. In some embodiments, the amount and frequency of administration is chosen to ensure that the measured level of the active moiety in the blood of a patient is maintained above 10 microgram/milliliter (μg/mL).

In some embodiments, an immunomodulatory CD163 antibody of the disclosure is administered intravenously in a physiological solution at a dose ranging between 0.01 mg/kg to 100 mg/kg at a frequency ranging from daily to weekly to monthly (e.g., every day, every other day, every third day, or 2, 3, 4, 5, or 6 times per week), preferably a dose ranging from 0.1 to 45 mg/kg, 0.1 to 15 mg/kg or 0.1 to 10 mg/kg at a frequency of 2 or 3 times per week, or up to 45 mg/kg once a month.

In some embodiments, an immunomodulatory CD163 antibody for use with a method disclosed herein is administered intravenously in a physiological solution at a flat dose ranging between 150 mg to 1200 mg. Non-limiting doses include about 150 mg, 300 mg, 450 mg, 600 mg, 900 mg, and 1200 mg.

In some embodiments, fixed doses are administered and unchanged in amount or frequency over time. In some embodiments, an escalating dose is administered. In some embodiments, the dose is escalated every one, two, or three weeks.

In some embodiments, the immunomodulatory CD163 antibody is administered in an escalating dose regimen, such as one that includes administration of 150 mg, 300 mg, 450 mg, 600 mg, 900 mg, and 1200 mg, with an escalation occurring every three weeks until maximum dosage is reached.

In some embodiments, the immunomodulatory CD163 antibody is administered in a decreasing dose regimen. In some embodiments, given clinical context, such as, safety considerations and/or changes in a subject's tolerance, weight, etc., a decreasing or dose regimen may be used to achieve therapeutic effect while mitigating or moderating undesirable side effects or ensuring patient safety.

In some embodiments, an immunomodulatory CD163 antibody for use with a method disclosed herein and an immune checkpoint inhibitor are administered simultaneously. In some embodiments, an immunomodulatory CD163 antibody for use with a method disclosed herein and an immune checkpoint inhibitor are administered sequentially. In some embodiments, the immunomodulatory CD163 antibody is administered prior to the immune checkpoint inhibitor. In some embodiments, the immune checkpoint inhibitor is administered prior to the immunomodulatory CD163 antibody. In some embodiments, the immunomodulatory CD163 antibody is administered one, two, three, four, five, six, or more times. In some embodiments, the immune checkpoint inhibitor is administered one, two, three, four, five, six, or more times.

In some embodiments, an immunomodulatory CD163 antibody for use with a method disclosed herein enhances and/or prolongs the effect of an immune checkpoint inhibitor. In another embodiment, an immunomodulatory CD163 antibody for use with a method disclosed herein allows a subject to respond to an immune checkpoint inhibitor. In another embodiment, an immunomodulatory CD163 antibody for use with a method disclosed herein enhances or prolongs the effect of an immune checkpoint inhibitor. In some embodiments, the immunomodulatory CD163 antibody and/or the immune checkpoint inhibitor are administered at a dose that is lower than a dose known to produce an efficacious effect when administered as a monotherapy, or at a does below the dose recommended for use in the monotherapy setting.

In some embodiments, an antibody described herein is administered to a subject in various dosing amounts and over various time frames. In some embodiments, an immune checkpoint inhibitor is administered to a subject in various dosing amounts and over various time frames. In some embodiments, the dose of an immunomodulatory CD163 antibody for use with a method disclosed herein is a submaximal dose. In some embodiments, a dose administered of an immune checkpoint inhibitor is a submaximal dose. In some embodiments, a dose administered of an immunomodulatory CD163 antibody for use with a method disclosed herein is a submaximal dose and the dose administered of an immune checkpoint inhibitor is a submaximal dose.

In an embodiment a “submaximal dose” may be below a recommended phase II dose (RP2D) dose for a given agent, as determined in a clinical trial. In some embodiments, the highest dose with acceptable toxicity is the dose of a particular agent (e.g., antibody, e.g., checkpoint inhibitor) that produces around 20% of dose-limiting toxicity.

In some embodiments, a submaximal dose of an agent is about 5-fold lower to about 50-fold lower than the ED₅₀ concentration of said agent. Non-limiting submaximal doses include about 5-fold, 10-fold, 15-fold, 20-fold, 25-fold, 30-fold, 35-fold, 40-fold, 45-fold, 50-fold or any integer in between, lower than the ED₅₀ concentration of an agent.

In some embodiments a submaximal dose may or may not be effective to treat a disease or disorder as described herein, depending on context; in some such embodiments, a submaximal dose refers to a dose when administered as a monotherapy. However, in some embodiments, when submaximal doses of an immune checkpoint inhibitor and/or an antibody are combined, a combination of the two agents may be effective to treat a disease.

In some embodiments, combination of an antibody and an immune checkpoint inhibitor allows the immunomodulatory CD163 antibody and/or the immune checkpoint inhibitor to be administered at lower doses in a combination therapy than in a monotherapy. Without being bound by any particular theory, the present disclosure contemplates, for example, that in some embodiments, where a dose of an immune checkpoint inhibitor antibody may be intolerable due to toxicity, combination of that checkpoint inhibitor with an immunomodulatory CD163 antibody or fragment thereof as disclosed herein allows the subject to receive treatment with both the immune checkpoint inhibitor and an immunomodulatory CD163 antibody for use with a method disclosed herein.

In some embodiments, the immunomodulatory CD163 antibody is administered in a regimen that comprises periodic doses. In some embodiments, an immunomodulatory CD163 antibody for use with a method disclosed herein is administered intravenously once every week, once every two weeks, once every three weeks, once every four weeks, once every five week, once every six weeks, once every seven weeks, or once every eight weeks. In some embodiments, the immunomodulatory CD163 antibody is administered intravenously in a physiological solution at a fixed dose ranging between 150 mg to 1200 mg at a frequency ranging from once per 4 weeks to once per week. In some embodiments, the immunomodulatory CD163 antibody is administered intravenously in a physiological solution at a fixed dose of 450 mg or 900 mg weekly, twice weekly, or three times weekly.

In some embodiments, an immunomodulatory CD163 antibody for use with a method disclosed herein is administered as a monotherapy or in combination with an immune checkpoint inhibitor. In some embodiments, an immunomodulatory CD163 antibody for use with a method disclosed herein is administered as a monotherapy. In some embodiments, an immunomodulatory CD163 antibody for use with a method disclosed herein is administered in combination with an immune checkpoint inhibitor.

In some embodiments, an immune checkpoint inhibitor is pembrolizumab. In some embodiments, pembrolizumab is administered intravenously in a physiological solution at a dose range in accordance with standard doses and administration protocols known in the art.

In some embodiments, pembrolizumab is administered intravenously in a physiological solution at a dose of 200 mg once every three weeks. In some embodiments, pembrolizumab is administered intravenously in a physiological solution at a dose 400 mg once every six weeks.

In some embodiments, an immune checkpoint inhibitor is nivolumab. In some embodiments, nivolumab is administered intravenously in a physiological solution at a dose range in accordance with standard doses and administration protocols known in the art.

In some embodiments, nivolumab is administered intravenously in a physiological solution at a dose of 240 mg once every two weeks. In some embodiments, nivolumab is administered intravenously in a physiological solution at a dose of 480 mg IV once every four weeks.

In some embodiments, an immune checkpoint inhibitor is durvalumab. In some embodiments, durvalumab is administered intravenously in a physiological solution at a dose range in accordance with standard doses and administration protocols known in the art.

A physician or veterinarian having ordinary skill in the art, in some cases, readily determines and prescribes the effective amount (ED₅₀) of the composition required. For example, the physician or veterinarian could start doses of the compounds employed in the composition at levels lower than that required to achieve the desired therapeutic effect and gradually increase the dosage until the desired effect is achieved. Alternatively, a dose remains constant in some embodiments.

In some embodiments, a composition (an immunomodulatory CD163 antibody or an antigen-binding fragment described herein) is administered alone or in combination with a second composition either simultaneously or sequentially dependent upon the condition to be treated. When two or more compositions are administered, the compositions are, for example, administered in combination (either sequentially or simultaneously). A composition is administered in a single dose or multiple doses in some embodiments.

In some embodiments, when formulated for administration to human subjects, the compositions are formulated to be free of pyrogens. Testing compositions for pyrogens and preparing pharmaceutical compositions free of pyrogens are well understood to one of ordinary skill in the art.

Antibodies, or antigen-binding fragments thereof, are formulated for any suitable route of administration to a subject including, but not limited to injection, in some embodiments. Injection includes, for example, subcutaneous, peritoneal, intravenous injection, intramuscular injection, or spinal injection into the cerebrospinal fluid (CSF). In some embodiments, administration is given in one, two, three, four, five, six, seven, or more injection sites. In one embodiment, administration is via six injection sites.

For in vivo applications, contacting occurs, for example, via administration of a composition (such as are described herein) to a subject by any suitable means. An immunomodulatory CD163 antibody described herein, in some embodiments, is administered by any suitable means, either systemically or locally, including via parenteral, subcutaneous, intraperitoneal, intracerebrospinal, intrapulmonary, and intranasal administration, and, if desired for local treatment, intralesional administration. Parenteral routes include, for example, intravenous, intraarterial, intraperitoneal, epidural, intramuscular, and intrathecal administration. Such administration, in some embodiments, is as a bolus, continuous infusion, or pulse infusion. In some embodiments, compositions are administered by injection depending in part on whether the administration is brief or chronic. Other modes of administration methods are contemplated, including topical, particularly transdermal, transmucosal, rectal, oral or local administration e.g., through a catheter placed close to the desired site.

Administration of a composition herein may be by any suitable means including, but not limited to, injection. In one embodiment, injection may be, for example, intravenous, subcutaneous, or intramuscular injection.

### Packages, Kits, and Pre-Filled Containers

Also provided herein are kits containing one or more compounds described above. The kit comprises, in some embodiments, an immunomodulatory CD163 antibody or antigen-binding fragment thereof as described herein in suitable container means. The kit comprises, in some embodiments, an immune checkpoint inhibitor as described herein in a suitable container means. In some embodiments, a kit comprises both an immunomodulatory CD163 antibody as provided herein and an immune checkpoint inhibitor, each with a suitable container means.

In some embodiments, there is provided is a container means comprising a composition described herein. In some embodiments, the container means is any suitable container which houses, for example, a liquid or lyophilized composition including, but not limited to, a vial, syringe, bottle, an in intravenous (IV) bag or ampoule. A syringe holds any volume of liquid suitable for injection into a subject, in some embodiments, including, but not limited to, 0.5 cc, 1 cc, 2 cc, 5 cc, 10 cc, or more.

Provided herein are kits, comprising a composition or compositions described herein. In some embodiments, provided herein is a kit for treating a subject having a cancer, comprising an immunomodulatory CD163 antibody as described herein and an immune checkpoint inhibitor.

In some embodiments, provided herein is a kit for treating a cancer, comprising an immunomodulatory CD163 antibody as described herein, and a label attached to or packaged with the container, the label describing use of the immunomodulatory CD163 antibody in combination with an immune checkpoint inhibitor.

In some embodiments, provided herein is a kit for treating a cancer, comprising an immune checkpoint inhibitor and a label attached to or packaged with the container, the label describing use of the immune checkpoint inhibitor with an immunomodulatory CD163 antibody as described herein.

In some embodiments, the container means of the kits will generally include at least one vial, test tube, flask, bottle, ampoule, syringe an intravenous (IV) bag, and/or other container means, into which the at least one polypeptide are placed, and/or preferably, suitably aliquoted. Provided herein is a container means comprising a composition described herein.

The kits, in some embodiments, include a means for containing at least one fusion protein, detectable moiety, reporter molecule, and/or any other reagent containers in close confinement for commercial sale. In some embodiments, such containers include injection and/or blow-molded plastic containers into which the desired vials are retained. In some embodiments, kits also include printed material for use of the materials in the kit.

Packages and kits additionally include a buffering agent, a preservative, and/or a stabilizing agent in a pharmaceutical formulation in some embodiments. In some embodiments, each component of the kit is enclosed within an individual container and all of the various containers can be within a single package. In some embodiments, disclosure kits are designed for cold storage or room temperature storage.

Additionally, in some embodiments, the preparations contain stabilizers to increase the shelf-life of the kits and include, for example, bovine serum albumin (BSA). Where the compositions are lyophilized, the kit contains, in some embodiments, further preparations of solutions to reconstitute the lyophilized preparations. Acceptable reconstitution solutions are well known in the art and include, for example, pharmaceutically acceptable phosphate buffered saline (PBS).

In some embodiments, packages and kits further include one or more components for an assay, such as, for example, an ELISA assay. Samples to be tested in this application include, for example, blood, plasma, tissue sections and secretions, urine, lymph, and products thereof. In some embodiments, packages and kits further include one or more components for collection of a sample (e.g., a syringe, a cup, a swab, etc.).

In some embodiments, packages and kits further include a label specifying information required by US FDA or similar regulatory authority, for example, a product description, amount and mode of administration, and/or indication of treatment. Packages provided herein can include any of the compositions as described herein.

The term “packaging material” refers to a physical structure housing the components of the kit. In some embodiments, the packaging material maintains the components sterilely and are made of material commonly used for such purposes (e.g., paper, corrugated fiber, glass, plastic, foil, ampules, etc.). In some embodiments, the label or packaging insert includes appropriate written instructions. Kits, therefore, additionally includes, in some embodiments, labels or instructions for using the kit components in any method of the disclosure. In some embodiments, a kit includes a compound in a pack, or dispenser together with instructions for administering the compound in a method described herein.

Instructions include instructions for practicing any of the methods described herein including treatment methods in some embodiments. Instructions additionally include indications of a satisfactory clinical endpoint or any adverse symptoms that occur, or additional information required by regulatory agencies such as the Food and Drug Administration for use on a human subject in some embodiments.

The instructions are, in some embodiments, on “printed matter,” e.g., on paper or cardboard within or affixed to the kit, or on a label affixed to the kit or packaging material, or attached to a vial or tube containing a component of the kit. Instructions are additionally included on a computer readable medium, such as, for example, CD-ROMs, DVDs, flash memory devices, solid state memory, magnetic disks and disk devices, magnetic tapes, cloud computing systems and services, and the like, in some embodiments. In some cases, the program and instructions are permanently, substantially permanently, semi-permanently, or non-transitorily encoded on the media.

Provided herein is a container means comprising a composition described herein. In some embodiments, the container means is any suitable container which houses a liquid or lyophilized composition including, but not limited to, a vial, syringe, bottle, intravenous (IV) bag, or ampoule. A syringe, in some embodiments, holds any volume of liquid suitable for injection into a subject including, but not limited to, 0.5 cc, 1 cc, 2 cc, 5 cc, 10 cc or more.

Provided herein are kits comprising a composition described herein. In some embodiments, provided herein is a kit for treating a cancer, comprising an immunomodulatory CD163 antibody as described herein in combination with an immune checkpoint inhibitor.

In some embodiments, provided herein is a kit for treating a cancer, comprising an immunomodulatory CD163 antibody for use with a method disclosed herein (i.e., an immunomodulatory CD163 antibody that binds to CD163 on human myeloid cells, i.e., hCD163), and a label attached to or packaged with the container, the label describing use of the immunomodulatory CD163 antibody, or an antigen-binding fragment thereof, with an immune checkpoint inhibitor. In some embodiments, the cancer is NSCLC, SCCHN, TNBC, or melanoma.

In some embodiments, provided herein is a kit for treating a cancer, comprising an immune checkpoint inhibitor and a label attached to or packaged with the container, the label describing use of the immune checkpoint inhibitor with an immunomodulatory CD163 antibody as described herein. In some embodiments, the cancer is NSCLC, SCCHN, TNBC, or melanoma.

In some embodiments, a kit comprises an immunomodulatory CD163 antibody for use with a method disclosed herein and an immune checkpoint inhibitor. In some such embodiments, each of the immunomodulatory CD163 antibody and the immune checkpoint inhibitor has a label attached to or packaged on a container comprising the immunomodulatory CD163 antibody or checkpoint inhibitor. In some such embodiments, the kit comprises at least one set of instructions; in some embodiments, the kit comprises two sets of instructions: one for the immunomodulatory CD163 antibody for use with a method disclosed herein and one for the immune checkpoint inhibitor.

### EXAMPLES

The present disclosure will be further illustrated in the following Examples which are given for illustration purposes only and are not intended to limit the disclosure in any way.

### Example 1: Isolation of Human Primary Cells

Apheresis products were collected from donors and autologous monocytes and T cells were isolated using techniques commonly used in the art. Briefly, human monocytes and T cells were isolated from white blood cells (WBCs) according to standard techniques. Here, WBCs were trapped within an integrated chamber (Trima LeukoReduction System (LRS) chambers No. 2490-08) during the collection process or LeukoPaks (No. 4510-01 Full Leuko Pack, purchased from BloodWorks Northwest, Seattle, WA). Peripheral blood mononuclear cells (PBMCs) were purified from the LRS chambers or LeukoPaks by standard density gradient centrifugation (FicollPaque® Premium 1.073, GE Healthcare No. 17-5449-52). The supernatant was discarded, and the pellet resuspended in 20 mL EasySep™ Buffer (StemCell Technologies No. 20144) for counting of PBMCs, and for further isolation of monocytes and T cells.

Monocytes were isolated using the EasySep™ Human Monocyte Isolation kit (Stemcell Technologies, No. 19359) following the manufacturer's instructions.

Total CD3+, CD4+, or CD8+ T cells were isolated using the EasySep™ Human CD3+, CD4+ and CD8+ T Cell Isolation kits (STEMCELL Technologies, Nos. 19051, 17952, 17953, respectively), following the manufacturer's instructions. These negative selection kits used antibodies to label undesired cell types for removal, allowing the desired target cells to be isolated from the sample.

### Example 2: M2c Macrophage Generation

M2c macrophages were generated from PBMC-derived monocytes using techniques commonly used in the art, such as those exemplified below.

At day 0, monocytes from individual donors (isolated as described in Example 1) were plated in 96-well culture plates (ThermoFisher (Costar), No. 09-761-145), at 25,000-50,000 cells/100 μL/well in M0 culture medium (90% X-VIVO™ 15+10% heat-inactivated FBS (Hyclone, No. SH30396.03)+100 ng/ml human M-CSF (PeproTech, No. 300-25)). Cells were incubated at 37° C. and 5% CO₂ for 5 to 6 days to produce differentiated “M0” macrophages.

At Day 5-6 of culture, the M0 macrophages were polarized to M2c macrophages by aspirating the medium gently from each plate and replacing it with 100 μL/well of M2c culture medium (M0 culture medium with 20 ng/mL huIL-10 (PeproTech, No. 200-10)). Cells were incubated at 37° C. and 5% CO₂ for 2 days.

At Day 7-8 of culture, the M2c macrophages were ready for coculture assay setup.

### Example 3: Generation of Exhausted T Cells

Exhausted T cells having a blast-like morphology were generated from human PBMCs by repeated (3×) phytohemagglutinin (PHA) stimulation. Cells were counted and incubated at 1×10⁶ cells/mL in T cell blast culture medium (90% IMDM (Thermo Fisher (Gibco), No. 12440053)+10% human serum+2 μg/mL PHA-L (Sigma-Aldrich (Roche), No. 11249738001)+4 ng/ml recombinant human IL-2, (R&D Systems, No. 202-IL)). Cells were split 1:2 or 1:3 every 3 to 4 days, and cultured for 10 days total (2 splits over 10 days; 3 total PHA stimulations). Fresh T cell blast culture medium was added to the cells at each split. The cells were harvested on day 10 and either set up for coculture assays or frozen down for future use. Exhausted T cell phenotype was confirmed by expression of, PD1, TIM3 and TIGIT (immune checkpoint proteins, which also serve as exhaustion markers), as well as the transcription factor Eomes (data not shown).

### Example 4A: Co-Culture of M2c Macrophages and T Cells

The effects on immune cell function of immunomodulatory CD163 antibody monotherapy and therapy of such an antibody with an immune checkpoint inhibitor were assessed using an in vitro coculture assay format in which suppressive M2c macrophages were cultured together with T cells. In this assay, the macrophages are presumed to be exerting a suppressive effect on the T cells, inhibiting their normal activity, as measured by accepted criteria, for example, IFNγ production, IL-2 production, or proliferation. Relief of the M2c-mediated suppression due to a treatment of the cocultured cells may be observed through measured changes in T cell activity.

In this example, M2c macrophages were cocultured with exhausted T cells. Activity of exhausted T cells was assessed by measuring interferon gamma (IFN03B3) production. Treatments included suboptimal concentrations of AB101 antibody, which is known to alter M2c macrophages, abating their immunosuppressive effect. Exemplary anti PD-1 and anti-PD-L1 antibodies were also tested, either alone or in combination with AB101 to assess relief of T cells immunosuppression.

To begin, M2c macrophages (˜50,000 cells/well in 96-well plate) were prepared as in Example 2. The polarization medium was gently aspirated from the 7-day old M2c macrophages, and assay medium (90% X-VIVO 15+10% FBS) 100 μL/well was added to wash once. The wash assay medium was aspirated and replaced with 50 μL/well of fresh assay medium. Plates were incubated at 37° C. for 1 hour prior to antibody treatment. T cells were washed with assay medium, treated with antibody (if required), and then 50,000 cells/well/50 μL added to the treated macrophage plates. The total volume of the assay was 200 μL. The coculture was maintained for 72 hours, and one or more parameters of interest, as further described in Examples 4B and 4C was measured.

In Examples 4B and 4C M2c macrophages were cocultured with exhausted T cells (chemically exhausted by repeated PHA stimulation) to examine effects of treatment on IFNγ production. In Example 6, the macrophages were cocultured with PBMC-derived T cells (i.e., non-chemically exhausted) to examine effects of treatment on IL-2 production and proliferation.

### Example 4B: Co-Culture Assays of M2c Macrophages and Exhausted T Cells with AB101/Anti-PD-1 Combination Studies

Prior to the addition of exhausted T cells, the macrophages were treated for two hours with 50 μL of AB101 or the isotype control for AB101, hIgG1. The final concentrations of AB101 or hIgG1 were either 0.16 or 0.08 μg/mL. After the initial two-hour pre-treatment, 50 μL/well of the anti-human CD3 clone OKT3 in assay medium (final assay concentration 0.25 μg/mL) was added and plates were incubated for 30 min at 37° C. before adding the exhausted T cells.

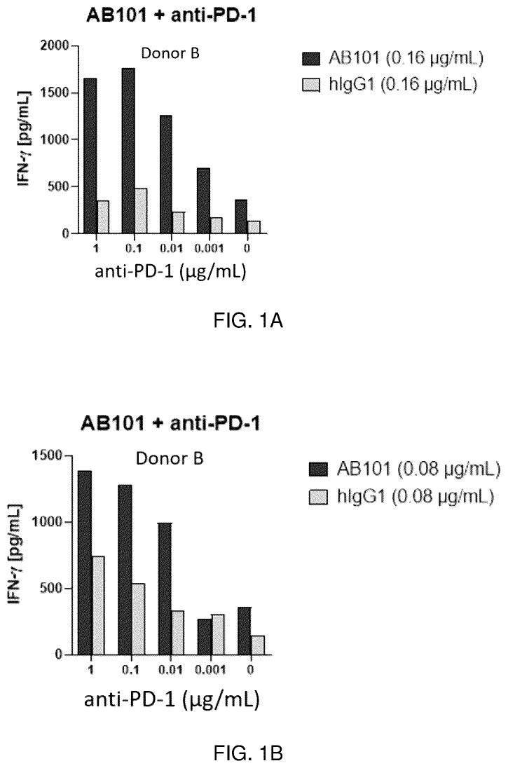

Exhausted T cells were pre-incubated with anti-PD-1 (Pem-hIgG4 S228P) antibody (InvivoGen, No. hpd1pe-mab14) or an isotype control (hIgG4) for 2 h, as indicated on FIGS. 1A-1B, 3A-3C, and 4A-4F. The final concentrations of anti-PD-1 antibody ranged from 0 μg/mL to 1 μg/mL (0, 0.001, 0.01, 0.1, and 1), as further described herein. The pre-incubated T cells combined with anti-PD-1 were then added to M2c macrophage plates treated with AB101 or hIgG1 and OKT3.

Suboptimal concentrations of AB101 were used (specifically, in these Examples, final concentrations of PD-1 were 0.16 μg/mL and 0.08 μg/mL, which are about 15- and 30-fold, respectively, lower than the EC₅₀ concentration of AB101 in this assay).

Supernatants from cocultures were harvested after 72 hours of treatment and IFNγ levels were measured using R&D ELISA kit (Human IFNγ DuoSet ELISA (R&D, No. DY285B) according to the manufacturer's instructions.

Treatment with a suboptimal concentration of AB101 and anti-PD-1 synergized to relieve M2c macrophage-mediated T-cell suppression better than the combination of anti-PD-1 and hIgG1, as shown by the enhanced IFNγ production in the presence of anti-PD-1 and AB101 at concentrations of 0.16 μg/mL (FIG. 1A) and 0.08 μg/mL (FIG. 1B). Data shown are representative data from a single subject.

### Example 4C: Co-Culture Assays of M2c Macrophages and Exhausted T Cells with Anti-PD-L1 Antibody

In this example, prior to the addition of exhausted T cells, the macrophages were treated for two hours with 50 μL of AB101 (or hIgG1) and anti-PD-L1 (OncoResponse; made using durvalumab variable domain sequences produced as an Fc-competent IgG1 antibody in accordance with the sequences disclosed in Lee et al. Scientific Reports 7:55532, 2017 which is herein incorporated herein by reference in its entirety). The final concentrations of AB101 or the isotype control was, as in Example 4A, either 0.16 or 0.08 μg/mL, while the final concentrations of anti-PD-L1 ranged from 0.001 μg/mL to 0.1 μg/mL (i.e., 0.001, 0.01, and 0.1). After the initial two-hour pre-treatment with the antibodies, 50 μL/well of the anti-human CD3 clone OKT3 in assay medium (final assay concentration 0.25 μg/mL) was added and plates were incubated for 30 min at 37° C. before adding the exhausted T cells.

Exhausted T cells were plated at ˜50,000 cells/well/50 μL in M2c macrophage plates to give final concentrations of either 0.16 μg/mL or 0.08 μg/mL+anti-PD-L1 antibody at final concentrations of concentrations of 0.1, 0.01, 0.001 μg/mL).

Supernatants from cocultures were harvested after 72 hours of treatment and IFNγ levels were measured using R&D ELISA kit (Human IFNγ DuoSet ELISA (R&D, No. DY285B) according to the manufacturer's instructions.

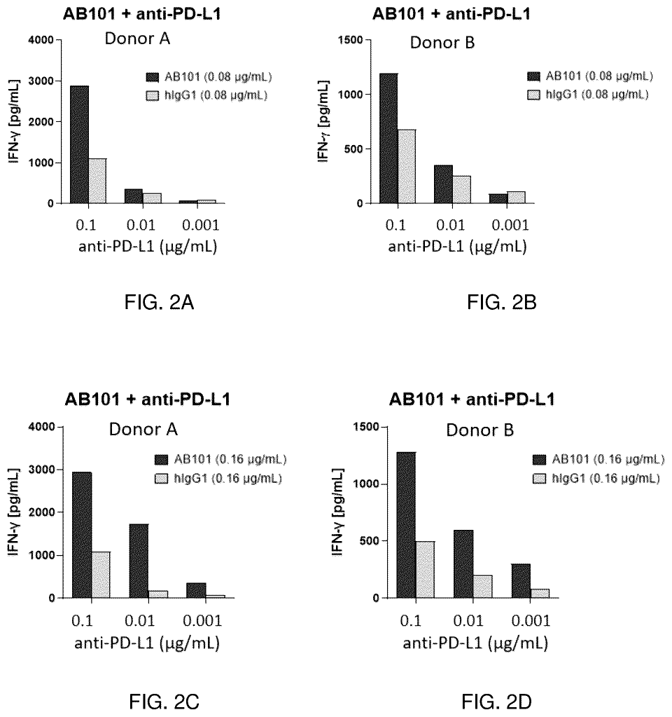

Overall, IFNγ production was synergistically increased in M2c/exhausted T cell cocultures treated with a combination of AB101 with anti-PD-L1 as compared to combination of AB101 isotype control with anti-PD-L1 at both concentrations of AB101 tested (FIGS. 2A-2D). Thus, AB101 enhanced the efficacy of the anti-PD-L1 antibody in the M2c/T cell coculture assay to rescue T-cell suppression by M2c macrophages (FIGS. 2A-2D). Treatment with suboptimal concentrations of AB101 and anti-PD-L1 synergized to relieve M2c macrophage-mediated T-cell suppression better than anti-PD-L1 alone as shown by the enhanced IFNγ production in the presence of anti-PD-L1 and AB101 at concentrations of 0.16 μg/mL (FIGS. 2C and 2D) and 0.08 μg/mL (FIGS. 2A and 2B). Data shown are for cells obtained from two donor subjects.

### Example 5: Co-Culture Assay of M2c Macrophages and Exhausted T Cells with a Panel of Checkpoint Inhibitors

The present example demonstrates effects of monotherapy or combination therapy on IFNγ production by anti-CD3-activated exhausted T cells in the presence of suppressive M2c macrophages.

An in vitro coculture assay using M2c macrophage and exhausted T cells is performed as described in Examples 4A-4C. Levels of IFN-γ secretion are measured after treatment with AB101, an anti-checkpoint antibody listed in Table 4, an anti-checkpoint ligand antibody as listed in Table 4 (and/or respective isotype controls), a combination of AB101 with anti-checkpoint antibody, or a combination of AB101 with anti-checkpoint ligand antibody.

TABLE 4
Antibodies to Exemplary Immune Checkpoint
Proteins and Their Ligands

| Checkpoint Antibody | Checkpoint Ligand Antibody |
|---|---|
| PD-1 | PD-L-1 |
| BTLA (CD272) | HVFM/TNFRSF14 |
| CTLA-4 | CD80 (B7-1); CD86 (B7-2) |
| TIM-3 | Galectin-9; HMGB1; Ceacam-1; |
| | Phosphatidyl serine (PtdSer) |
| TIGIT | CD112; CD155 |
| LAG-3 | MHCII; LSECtin |
| CD40 | CD40L |
| OX40 | OX40L |
| VISTA | VSIG-3/IGSF11 |
| B7-H3 (CD276) | TLT-2 |

Overall, IFNγ production is synergistically increased in M2c/exhausted T cell cocultures treated with a AB101 and an anti-checkpoint antibody or an anti-checkpoint ligand antibody as compared to isotype controls at both concentrations of AB101 tested. Thus, AB101 enhances the efficacy of the anti-checkpoint antibody or anti-checkpoint ligand antibody in the M2c/T cell coculture assay to rescue T-cell suppression by M2c macrophages. Treatment with suboptimal concentrations of AB101 and anti-checkpoint antibody or anti-checkpoint ligand antibody synergize to relieve M2c macrophage-mediated T-cell suppression better than anti-checkpoint antibody or anti-checkpoint ligand antibody alone as shown by the enhanced IFNγ production in the presence of the combination of AB101 and an anti-checkpoint or anti-checkpoint ligand antibody.

### Example 6: M2c Macrophage/PBMC-Derived T Cell Coculture Assay

Human M2c macrophages were generated as described in Example 2. Briefly, after polarization of M0 macrophages to M2c macrophages (Day 7), supernatants were removed from M2c macrophage cultures (2.5×10⁴ M2c/well of a 96-well flat bottom plate) and replaced with assay medium (100 μL of X-VIVO™ 15 media+10% FBS+0.5 μg/mL OKT3 (murine anti-human CD3 ab, BioLegend, No. 317302)).

Then, PBMC-derived CD3+ T cells, isolated and cultured for 18 h in X-VIVO™ 15 media+10% FBS, were added at 11.5×10⁴ T cells/well, or 2.5×10⁴ T cells/well, with a final OKT3 concentration of 0.25 μg/mL. IL-2 was quantified in supernatants taken 24 h after OKT3 stimulation using a HTRF IL-2 kit (CisBio, No. 62HIL02PEG). CD4+ and CD8+ T cell proliferation was determined 72 h after stimulation by flow cytometry using the CellTrace™ Violet Proliferation Dye kit (ThermoFisher, No. C34557).

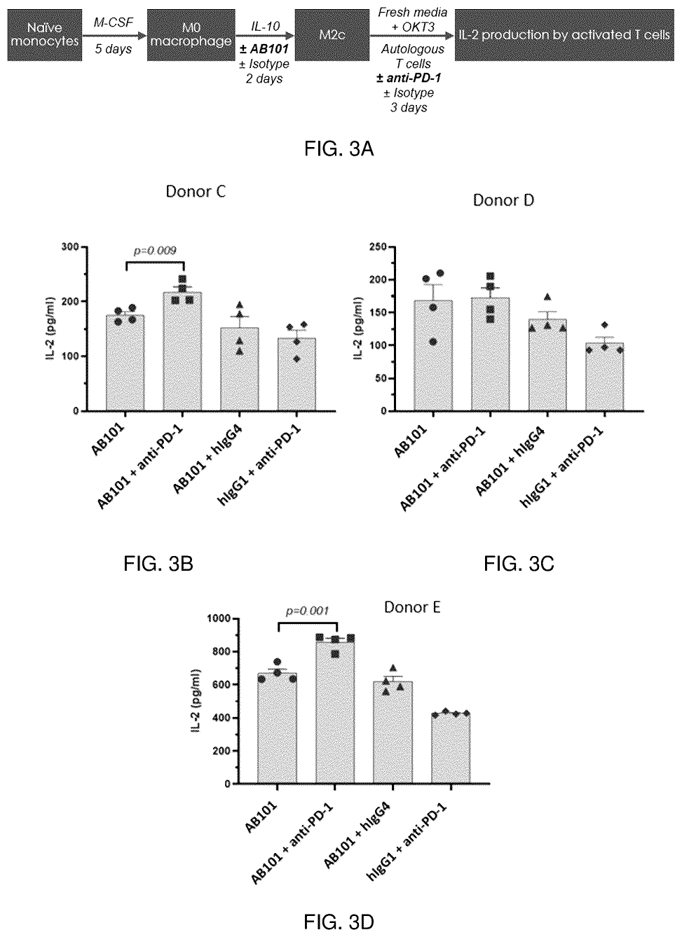

To assess the effect of a AB101 treatment on IL-2 secretion and/or CD4+ and CD8+ T-cell proliferation, M0 macrophages (“Pre”) and M2c/T cell cocultures (“Post”) were treated with antibodies or isotype controls (20 μg/mL) as follows: for the Pre regimen, antibodies or controls were added during M0 polarization to M2c and washed out for M2c/T cell coculture phase, while for the Pre/Post regimen, antibodies and controls were continuously present during polarization and coculture. (see FIGS. 3A (pre) and 4A (pre/post)).

### Coculture Assay Procedures

Cells were washed by removing medium from the plates by gently aspirating the medium, 50 μL/well of assay medium was added to M2c Macrophage and incubated at 37° C. Antibodies and isotype controls were diluted at 4× concentration (e.g., for 5 μg/mL final antibody concentration requires 20 μg/mL antibody) and 50 μL/well of assay medium was added to M2c macrophage plates and incubated at 37° C. for at least 2 hours. The final volume for cocultures including antibody treatment was 200 μL/well.

### Coculture Setup and Treatment

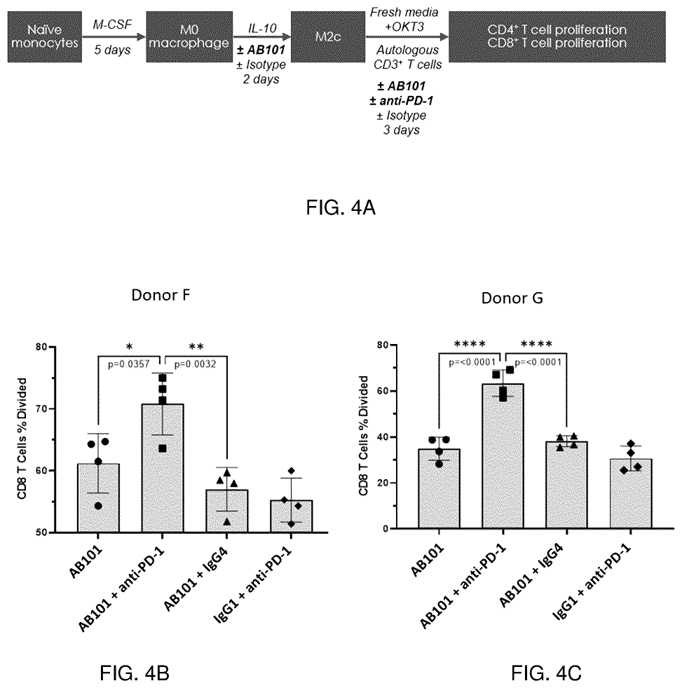

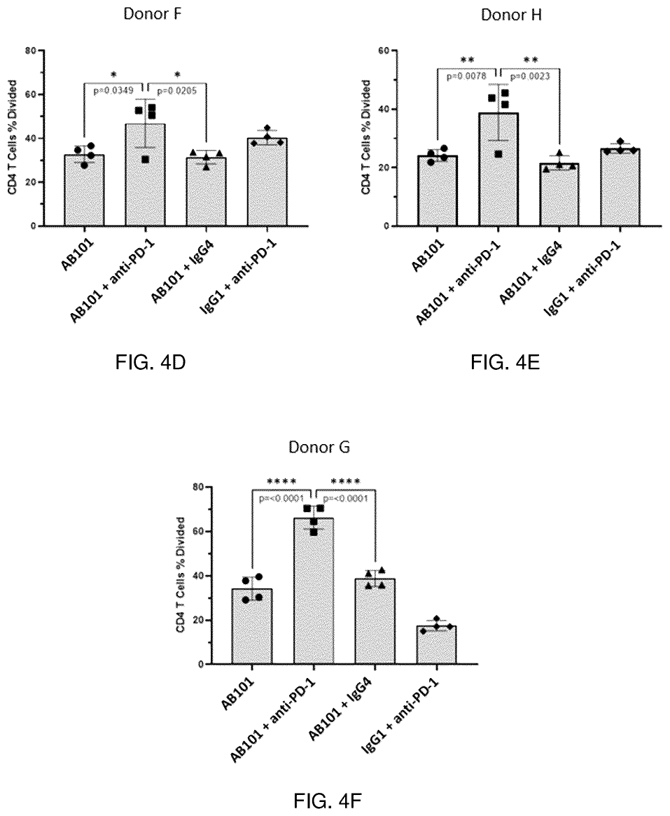

Cocultures were set up as described herein and/or as illustrated in schematics of FIG. 3A (Pre regimen), or FIG. 4A (Pre/Post regimen). Cocultures were treated with AB101, AB101 isotype control hIgG1, anti-PD-1, anti PD-1 isotype control hIgG4, or combinations thereof. IL-2 (pg/mL) (FIGS. 3B-3D), CD8+ T cell proliferation (FIGS. 4B-4C), and CD4+ T cell proliferation (FIGS. 4D-4F), were measured as described herein.

IL-2 Production after Combination of AB101 and Anti-PD-1 in M2c/Activated T Cell Coculture Assay.

IL-2 was measured in cocultures prepared and treated with AB101 Pre-regimen, anti-PD1, a combination of AB101 with anti-PD-1, or isotype control according to the schematic shown in FIG. 3A. To measure IL-2 production by T cells from M2c/T cell cocultures, IL-2 was quantified using supernatant harvested 24 h after OKT3 stimulation. As shown by increased IL-2 production by activated T cells (FIGS. 3B-3D), treatment with a combination of AB101 and anti-PD1 relieved M2c macrophage-mediated T-cell suppression better than either AB101 or anti-PD-1 alone (or in combination with isotype controls). Data shown are for cells obtained from three donor subjects.

Measurement of T Cell Proliferation after Treatment with Combination of AB101 and Anti-PD-1 in M2c/Activated T Cell Cocultures

M2c/T cell coculture assays were generated according to procedures described herein and the schematic of FIG. 4A, followed by measurement of CD8+ T cell proliferation or CD4+ T cell proliferation. Combination treatment with AB101 and anti-PD-1 significantly increased proliferation of activated T cells as shown by increases in CD8+ T cell proliferation (FIGS. 4B-4C) and CD4+ T cell proliferation (FIGS. 4D-4F) as compared to AB101 or PD-1 alone (or in combination with isotype controls). Data shown are for cells obtained from two and three donor subjects, respectively.

### Example 7: M2c Macrophage/Activated PBMC-Derived T Cell Coculture Assay with a Panel of Immune Checkpoint Inhibitors after Treatment with AB101 and an Immune Checkpoint Inhibitor

The present example demonstrates effects of monotherapy or combination therapy on IL-2 secretion and/or CD4+ and CD8+ T-cell proliferation on activated T cells in the presence of suppressive M2c macrophages.

An in vitro coculture assay using M2c macrophage and activated T cells is performed as described in Example 6. Levels of IL-2 secretion, CD4+ and/or CD8+ T-cell proliferation are measured after treatment with AB101, an antibody listed in Table 4 (and/or respective isotype controls), and at least one combination of AB101 and at least one antibody listed in Table 4.

IL-2 levels are increased in cells treated with a combination of AB101 and an antibody from Table 4 as compared to AB101 or the antibody in Table 4 alone (or in combination with isotype controls). Combination treatment with AB101 and an antibody in Table 4 synergizes to increase proliferation of activated T cells as shown by increases in CD8+ T cell proliferation and/or CD4+ T cell proliferation as compared to AB101 or an antibody in Table 4 alone (or in combination with isotype controls).

### Example 8: Immunophenotyping of M2c Macrophages and T Cells after Anti-CD3 Stimulation in M2c/Activated T Cell Cocultures Treated with a Combination of AB101 and an Immune Checkpoint Inhibitor

T cell surface marker expression is characterized 72 h after OKT3 stimulation by flow cytometry. Supernatants are collected from the M2c/T cell coculture 72 h after anti-CD3 stimulation in the coculture assay to assess the secretion of inflammatory (IL-6, TNF-α), immune suppressive (IL-10), and anti-cancer cytokines (IFN-γ) as well as the production of the cytolytic protein perforin. Cytokines are quantified using a Procardia Plex™ Magpix assay kit and measured as mean cytokine output in pg/mL of each cytokine of three biological replicates for each study subject.

Combination treatment of AB101 and an immune checkpoint inhibitor during M2c/T cell coculture relieves M2c mediated immunosuppression and induces a potent cytokine response by anti-CD3 activated CD8+ T cells. Combination of AB101 and an immune checkpoint inhibitor significantly enhances the IFN-γ and perforin, and IL-6 levels when compared to combination of AB101 isotype controls. In addition, treatment with combination of AB101 and an immune checkpoint inhibitor restores TNF-α secretion with significant increase over the corresponding combination with isotype control.

T cell phenotyping is also performed. Myeloid cells in the tumor microenvironment, tumor associated macrophages (TAMs), have been shown to orchestrate a dampened immune response which facilitates tumor grown. Often, this effect can be seen as skewing T cells to a lower ratio of Th1/Th2 (e.g., skewing T cells to a Th2 phenotype). Effect of treatment with a combination of AB101 and an immune checkpoint inhibitor is evaluated to determine effect on cross talk between the TAMs and tumor infiltrating lymphocytes (TILs) and whether the suppressive effect of the TAMs on the TILs is relieved.

The ratio of Th1 to Th2-helper cells is assessed by staining with cell surface marker antibody panels to determine ratio of Th1 to Th2 skewing. Following surface marker and cell viability staining, T cells are fixed and analyzed for presence of Th1 or Th2 markers by flow cytometry. Panel 1 is used to determine ratio of Th1/Th2, Th17, and Treg, while panel 2 is used to determine T cell activation and exhaustion. An antibody cocktail is made at 2× using remaining 50 μL/well of Blocking buffer, with Panel 1 antibodies at 1:50 (Final conc is 1:100), and Panel 2 antibodies at 1:50 (Final conc is 1:100).

TABLE 5
Antibody Cocktail Mixes for assessing Th1/Th2, Th17
(Panel 1) and Exhaustion/Activation (Panel 2) of T cells.

| | Antibody Panel 1: | Antibody Panel 2: |
|---|---|---|
| | Th1/Th2, Th17 | Exhaustion/Activation |
| Surface | CD4 - PE (BD Pharmingen No. | CD4 - PE (BD Pharmingen No. |
| markers | 55347) | 55347) |
| | CD69 - PE-Cy7 (BioLegend No. | CD8 - APC (BioLegend No. 344721) |
| | 104511) | LAG3 - BV421 (BioLegend No. |
| | CD25 - APC (BioLegend No. | 369313) |
| | 101909) | OX40 - BV510 (BioLegend No. |
| | CD127 - BV510 (BD BIOSCIENCE | 745040 |
| | NO. 563086) | PD-1 - PerCP-Cy5.5 (BioLegend No. |
| | CXCR3 - PerCP-Cy5.5 (BioLegend | 135207) |
| | No. 126513) | ICOS - PE-Cy7 (BioLegend No. |
| | CD194 (CCR4) - BV421 (BioLegend | 329805) |
| | No. 359413) | CTLA-4 - FITC (eBioscience 11- |
| | CD196 (CCR6) - BV510 (BioLegend | 1529-42) |
| | No. 353423) | |
| Marker | Th1: CD4⁺, CD69⁺, CD196⁻, | Activated: ICOS⁺, OX-40⁺ |
| Identification | CXCR3⁺, CCR4⁻ | Exhausted: LAG-3⁺, PD-1⁺, CTLA-4⁺ |
| | Th2: CD4⁺, CD196⁻, CXCR3⁻, | Th T cells: CD4⁺ |
| | CCR4⁺ | Tc T cells: CD8⁺ |
| | Treg: CD4⁺, CD25⁺, CD127⁻ | |
| | Th17: CD4⁻, CD196⁺, CXCR3⁻, | |
| | CCR4⁺ | |

The analysis shows that M2c cells have immunosuppressive effects on activated T cells in co-culture, inhibiting T cell proliferation.

Treatment with AB101 and an immune checkpoint inhibitor brings about a synergistic effect, relieving the suppressive effects of M2c cells shown by increased levels of IL2 and increased T cell proliferation at levels better than treatment with AB101 or checkpoint inhibitor alone (or in combination with isotype controls).

### Example 9: Immunophenotyping of M2c Macrophages after Treatment with Combination of AB101 and an Immune Checkpoint Inhibitor in M2c/Activated T Cell Cocultures

M2c macrophages express M2c markers CD163, CD206, and Mer-TK, as well as Fcγ receptors CD16 (FcγRIII), CD32 (FcγRII), CD64 (FcγRI), and at lower levels the pattern recognition receptor TLR2, the TNFR family member CD40. CD86, CD91, CD150, Calreticulin, Dectin-1, TIM4 and TLR4 are not expressed on M2c cells.

M2c macrophages are generated from PBMC-derived monocytes as described in Example 2 and/or using other methods commonly known in the art.

Briefly, at day 5-6 of culture, the M0 macrophages are polarized to M2c macrophages by aspirating the medium gently from each plate and replacing it with 100 μL/well of M2c culture medium (M0 culture medium with 20 ng/ml huIL-10 (PeproTech, No. 200-10)). Cells are incubated at 37° C. and 5% CO₂ for 2 days in the presence of AB101, isotype control antibody, a combination of AB101 with an immune checkpoint inhibitor, or a combination of AB101 isotype controls with an immune checkpoint inhibitor.

M2c macrophages are then stained for viability and for surface marker expression with different antibody panels (Table 6). Methods used for viability, staining, and FACS separation and analysis are performed using techniques commonly used in the art.

TABLE 6
Antibody panels to determine surface marker expression by M2c macrophages

| | Antibody M2c | Antibody M2c | Antibody M2c |
|---|---|---|---|
| | Phenotype Panel 1 | Phenotype Panel 2 | Phenotype Panel 3 |
| Surface | CD16 - PE | CD150 - PE | Dectin-1 - PE |
| Markers | CD32 - APC | PD-L1 - APC | TLR4 - APC |
| | CD64 - Pacific blue | CD40 - FITC | TLR2 - FITC |
| | LILRB2 - PE-Cy7 | CD86 - PE-Cy7 | Tim4 - PE-Cy7 |
| | | MHC Class II - PerCP-Cy5.5 | |
| | Antibody M2c | Antibody M2c Phenotype | |
| | Phenotype Panel 4 | Panel 5 | |
| Surface | CD91 - PE | CD163 - FITC | |
| Markers | Siglec-15 - AF647 | CD206 - PE | |
| | Calreticulin - AF488 | MHC Class II - PerCP-Cy5.5 | |
| | MERTK - PE-Cy7 | CD86 - PE-Cy7 | |

Changes in surface marker expression are shown using standard median fluorescence intensity measurements. Cells treated with a combination of AB101 and an immune checkpoint inhibitor show decreases in expression of TLR2 and Siglec-15, and an upregulation in HLA-Class II as compared to treatment with AB101 or an immune checkpoint inhibitor alone (or in combination with an isotype control). The combination treatment group also interferes with IL-10 induced upregulation of CD16 and CD64 M2c cells as compared to treatment with AB101 or an immune checkpoint inhibitor alone (or in combination with an isotype control).

### Example 10: Efficacy in Lung Cancer Xenograft Model

Lung cancer xenograft models were generated and analyzed as described herein. Xenograft models were generated using lung carcinoma or lung adenocarcinoma cells and NSG-SGM3 mice. In brief, NSG-SGM3 mice were used to generate xenograft models using methods known to those in the art using human lung cancer-derived cells with wild-type p53 “A549” cells (A-549, human lung carcinoma, p53 wild type; ATCC No. CCL-185); or mutated p53 “H1975” cells were used (NCI-H1975, human lung adenocarcinoma, p53 mutated, p.R273H; ATCC No. CRL-5908) cells. Xenografts were placed in flanks of each mouse and tumors were followed and measured using techniques known in the field. Mice were treated with either an isotype control, AB101, or an anti-PD-1 antibody.

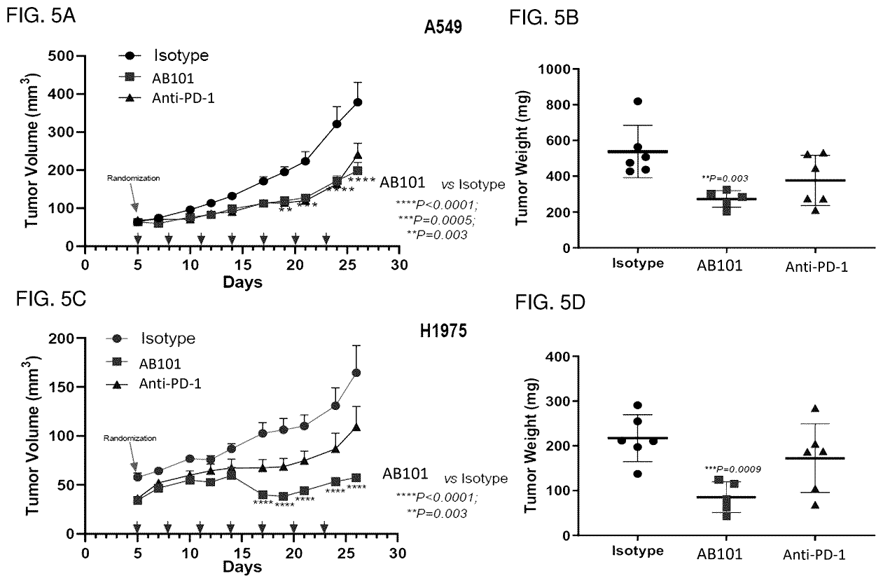

Both A549 (FIG. 5A) and H1975 (FIG. 5C) tumor volume was significantly reduced in treatment groups as compared to the group receiving isotype control antibody. Surprisingly, the AB101 group tumors had significantly smaller volumes than that of the isotype control group (FIG. 5A, **p=0.003; ***p=0.0005; ****p=0.0001 and FIG. 5C; **p=0.003; ****p=0.0001). Tumor volume was also significantly reduced in H1975 tumors for anti-PD-1 treated group as compared to isotype control (FIG. 5C; ****p=0.0001).

Interestingly, tumor weight was significantly reduced in the AB101 group compared to the isotype control group for both cancer types, while the tumor weights in the anti-PD-1-treated group was not significantly different from control for either cancer type (FIGS. 5B and 5D, A549 and H1975 tumors, respectively).

### Example 11: Clinical Combination Treatment of AB101 and a PD-1 Antagonist

A Phase 1/2 study is performed evaluating efficacy of AB101 alone or in combination with an anti-PD-1 therapy in human subjects (“subjects”) with advanced solid tumors (e.g., NSCLC, SCCHN, TNBC, or melanoma) who are eligible for anti-PD-1 monotherapy treatment. Subjects have advanced solid tumors for which no existing options are known to provide clinical benefit.

AB101 is supplied as a sterile, single-use, preservative-free solution for IV infusion in a vial containing a nominal fill volume of 10 mL (150 mg). The drug product is formulated at 15 mg/mL in 10 mM sodium acetate, 9% (w/v) sucrose, 0.015% (w/w) polysorbate 20, pH 5.2. The final container is a 20 mL USP Type I glass vial with a gray Flurotec®-coated plug and flip-off top seal. The recommended storage conditions are at −20° C. protected from light, and freeze-thaw should be avoided.

The AB101 product is diluted as outlined in the Pharmacy Manual prior to administration as IV infusion. The IV infusion is administered over at least 30 minutes through a dedicated line with an in-line filter and recommended product contact surfaces. The specific infusion time may be dose dependent.

Pembrolizumab is administered according to KEYTRUDA® (pembrolizumab) prescribing information, cemiplimab is administered according to LIBTAYO® (cemiplimab-rwlc) prescribing information, and nivolumab is administered according to OPDIVO®(nivolumab) prescribing information. Assessments of safety and tolerability as well as disease progression are made by the physician using conventional clinical criteria.

Primary outcome objectives evaluated during the trial are: safety and tolerability of AB101 in adult subjects with advanced malignancies; determination of dose and regimen of AB101 for further development; determination of recommended dose and regimen of AB101 when used in combination with PD-1 antagonists, nivolumab, cemiplimab, or pembrolizumab.

Secondary outcome objectives evaluated during the trial are: determination of serum PK of AB101 alone and in combination with a PD-1 antagonist; assessment of anti-tumor activity of AB101 alone and in combination with a PD-1 antagonist in subjects were certain malignancies enrolled in expansion cohorts; and determination of immunogenicity of AB101 alone and in combination with a PD-1 antagonist.

In addition to primary and secondary objectives, assessment of AB101 on tumor microenvironment and assessment of association between baseline pharmacodynamic markers and tumor response to AB101 are performed.

AB101 (150-1200 mg IV q3w) is administered as a monotherapy or in combination with pembrolizumab 200 mg administered IV q3w (or 400 mg IV q6w), cemipilimab 350 mg administered IV q3w, or nivolumab 240 mg IV q2w (or 480 mg IV q4w) as outlined, below.

The study is conducted in three parts: A, B, and C. Only part B is a combination study of AB101 and anti-PD-1 antibody. Part A is a monotherapy dose escalation study (e.g., 150, 300, 600, 1200 mg every 3 weeks intravenously or 450 mg and 900 mg weekly intravenously) with up to approximately 29 subjects designed to determine maximum tolerated dose (MTD) or recommended phase 2 dose (RP2D) of AB101. Part C is a biology cohort in which subjects not eligible for Parts A or B are enrolled in order to determine mechanisms of action and potential predictors of response to treatment. Subjects in Part C must have liposarcoma, leiomyosarcoma, squamous cell carcinoma of the head and neck, or another cancer (e.g, 10 subjects in each disease indication).

### Combination Treatment: AB101 and PD-1 Antagonist

A combination study (Part B) is conducted. Up to twelve subjects are included in a mini-dose escalation study at one of two doses of AB101 in combination with an anti-PD-1 antibody, in this case cemiplimab, or in combination with docetaxel followed by expansion of two cohorts B1, B2, and B3. Cohort B1 one has 20 subjects with NSCLC each treated with AB101 alone or the combination of AB101 and cemiplimab. Cohort B2 has 20 subjects with NSCLC treated with AB101 and docetaxel. Cohort B3 has 20 subjects with melanoma each treated with AB101 alone or the combination of AB101 and cemiplimab. Safety and preliminary anti-tumor activity of AB101 in combination with anti-PD-1 antibody is determined. Subjects in Cohorts B1 and B3 must have received prior treatment with an anti-PD-1 or anti-PD-L1 therapy. Subjects in cohort B2 are not required to have received anti-PD-1 or PD-L1 therapy but may have done so . . .

The RP2D is the highest dose with acceptable toxicity and presumed activity and is identified in Part A of the trial and is determined by dose limiting toxicity, overall safety assessment, PK parameters, available pharmacodynamic parameters, and evidence of clinical activity.

Subjects are treated with AB101 monotherapy or AB101 and either pembrolizumab, cemiplimab, or nivolumab at doses and intervals as described herein. Up to the first twenty subjects are treated. No dose-limiting toxicities (DLTs) are observed and combined safety data (including from monotherapy dosing of AB101 collected up to 100 days following exposure) demonstrate safety of the combination. Additional subjects are enrolled (a total of 20 subjects with melanoma and a total of 20 subjects with NSCLC are treated with AB101 and either pembrolizumab, cemiplimab, or nivolumab). AB101 is administered at the RP2D level. Doses are de-escalated to tolerability only if DLTs appear. Safety and preliminary anti-tumor activity of AB101 is determined in combination with a PD-1 antagonist.

Subjects treated with AB101 in combination with PD-1 antagonist therapy are evaluated on several measures. Primary endpoints, e.g., safety and tolerability, as well as dose and regimen and are determined prior to or during the combination trial. Subjects are evaluated for responsiveness to treatment (complete response, partial response, and/or disease stability).

Objective response rate for anti-tumor activity is conducted using known criteria including RECIST v 1.1 (Eisenhauer 2009 Eur J Cancer 45:228-247; see also Aykan 2020 World J Clin Oncol 11(2):53-73, each of which is incorporated herein by reference in its entirety) and, optionally, iRECIST v1.1 criteria. Results are reported by tumor type cohort as a percentage of the efficacy population in that cohort, with 95% confidence intervals; any other anti-tumor activity variables are reported by standard methods.

Anti-tumor activity is also measured as determined by CT or MRI at week 6, 12, 18, 24, 36, 48, and every six months thereafter. Immunogenicity is evaluated to determine presence and frequency of anti-drug antibodies, including neutralizing antibodies to either AB101 and/or anti-PD-1 on pre-dose Day 1 of each three-week cycle for five cycles. Objective response rate, progression-free survival, and overall survival are also measured. Subjects treated with AB101 and PD-1 antagonist therapy are found to have success in treatment of tumors known to be refractory or treatment-resistant. Treatment with AB101 and a PD-1 antagonist therapy delivers a synergistic effect on tumor treatment. Subjects have enhanced anti-tumor activity as determined by CT or MRI and prolonged clinical responses to immunotherapy as compared to prior clinical responses with prior or no treatments. Subjects treated with AB101 in combination with PD-1 antagonist therapy do not exhibit anti-drug antibodies (ADAs) or significant increases in anti-drug antibodies to AB101 and/or anti-PD-1 over the course of treatment.

What is claimed is:

1. A method of providing a cancer immunotherapy to a subject in need thereof, comprising:
administering to the subject:
a) a therapeutic amount of an immunomodulatory CD163 antibody or antigen-binding fragment thereof comprising sequences 100% identical to the amino acid sequences according to SEQ ID NOs: 1, 2, 3, 4, 5, and 6; and
b) a therapeutic amount of an immune checkpoint inhibitor selected from the group consisting of an antagonist to an immune checkpoint protein or ligand thereof, wherein the immune checkpoint protein is selected from the group consisting of CTLA-4, PD-1, PD-L1, TIM3, LAG3, TIGIT, and any combination thereof.

2. The method of claim 1, wherein the antibody comprises an Fc.

3. The method of claim 1, wherein the immunomodulatory CD163 antibody is an IgG1 antibody or IgG4 antibody.

4. The method of claim 1, wherein the therapeutic amount of the CD163 antibody is about 150 milligrams (mg) to about 1200 mg administered intravenously from about once per week to about once per 3 weeks.

5. The method of claim 1, wherein the immunomodulatory CD163 antibody comprises a light chain variable domain (V_{L}) having a sequence at least 80% identical to the amino acid sequence according to SEQ ID NO: 40 and a heavy chain variable domain (V_{H}) having a sequence at least 80% identical to the amino acid sequence according to SEQ ID NO: 41.

6. The method of claim 1, wherein the immunomodulatory CD163 antibody comprises a light chain variable domain (V_{L}) having a sequence at least 100% identical to the amino acid sequence according to SEQ ID NO: 40 and a heavy chain variable domain (V_{H}) having a sequence at least 100% identical to the amino acid sequence according to SEQ ID NO: 41.

7. The method of claim 1, wherein the administering of the immunomodulatory CD163 antibody and the immune checkpoint inhibitor yields an additive effect as compared to administration of either the immunomodulatory CD163 antibody or the immune checkpoint inhibitor alone.

8. The method of claim 1, wherein the administering of the immunomodulatory CD163 antibody and the immune checkpoint inhibitor yields a synergistic effect as compared to administration of either the immunomodulatory CD163 antibody or the immune checkpoint inhibitor alone.

9. The method of claim 1, wherein the immune checkpoint inhibitor is a PD-1 antagonist selected from the group consisting of: a PD-1 antibody, small molecule, or a rationally-designed peptide antagonist of PD-1.

10. The method of claim 8, wherein the PD-1 antibody is selected from the group consisting of nivolumab, pembrolizumab, cemiplimab, dostarlimab, JTX-4014, spartalizumab, camrelizumab, sintilimab, tislelizumab, toripalimab, INCMGA00012, AMP-224, AMP-514, zimberelimab, and fragments or combinations thereof.

11. The method of claim 1, wherein the immune checkpoint inhibitor is a PD-L1 antagonist.

12. The method of claim 11, wherein the PD-L1 antagonist is a PD-L1 antibody.

13. The method of claim 11, wherein the PD-L1 antagonist is selected from the group consisting of avelumab, durvalumab, atezolizumab, envafolimab, cosibelimab (CK-301), LY3300054, CA-170, BMS-936559, and combinations and active fragments thereof.

14. The method of claim 1, wherein the immunomodulatory CD163 antibody and the immune checkpoint inhibitor are administered concomitantly or sequentially.

15. The method of claim 14, wherein when administration is sequential, an interval between administration of the immunomodulatory CD163 antibody and administration of the immune checkpoint inhibitor is between one hour and thirty days.

16. The method of claim 15, wherein the interval is about three weeks.

17. The method of claim 1, wherein the therapeutic amount of the CD163 antibody is about 150 milligrams (mg) to about 1200 milligrams.

18. The method of claim 1, wherein the cancer is non-small cell lung cancer (NSCLC), small cell lung cancer (SCLC), papillary thyroid cancer, classical Hodgkin lymphoma (cHL) primary mediastinal large B-Cell lymphoma (PMBCL), soft-tissue sarcoma, liposarcoma, leiomyosarcoma, carcinoma, adenocarcinoma of the prostate or a prostatic intraepithelial neoplasia, squamous cell carcinoma, gastric adenocarcinoma, melanoma, triple-negative breast cancer, or combinations thereof.

19. The method of claim 1, wherein the immunomodulatory CD163 antibody is administered intravenously or subcutaneously.


SEQUENCE LISTING (48 sequences)

SEQ ID NO: 1 - protein, 11 aa, synthetic construct
RASQSISSYLN

SEQ ID NO: 2 - protein, 7 aa, synthetic construct
AASSLQS

SEQ ID NO: 3 - protein, 10 aa, synthetic construct
QQSYSTPRGT

SEQ ID NO: 4 - protein, 5 aa, synthetic construct
SYAMH

SEQ ID NO: 5 - protein, 17 aa, synthetic construct
VISYDGSNKYYADSVKG

SEQ ID NO: 6 - protein, 24 aa, synthetic construct
ENVRPYYDFWSGYYSEYYYYGMDV

SEQ ID NO: 7 - protein, 11 aa, synthetic construct
RASQSISRYLN

SEQ ID NO: 8 - protein, 10 aa, synthetic construct
QQSYSTQRGS

SEQ ID NO: 9 - protein, 7 aa, synthetic construct
AASSLQN

SEQ ID NO: 10 - protein, 10 aa, synthetic construct
QQSYSTTRGS

SEQ ID NO: 11 - protein, 10 aa, synthetic construct
QQSYSTQRGA

SEQ ID NO: 12 - protein, 10 aa, synthetic construct
QQSYSTTGGT

SEQ ID NO: 13 - protein, 11 aa, synthetic construct
  VARIANT 8: note = S or R or K or H
RASQSISXYLN

SEQ ID NO: 14 - protein, 7 aa, synthetic construct
  VARIANT 7: note = S or N or Q or T
AASSLQX

SEQ ID NO: 15 - protein, 10 aa, synthetic construct
  VARIANT 7: note = P or Q or T or S or N or A or G
  VARIANT 8: note = R or G or A or S
  VARIANT 10: note = T or S or A or G or N
QQSYSTXXGX

SEQ ID NO: 16 - protein, 5 aa, synthetic construct
SYDMH

SEQ ID NO: 17 - protein, 17 aa, synthetic construct
VISEDGSNKYNADSVKG

SEQ ID NO: 18 - protein, 24 aa, synthetic construct
ENVRPYYDFWSGYSSEYYYYGLDV

SEQ ID NO: 19 - protein, 5 aa, synthetic construct
SETMH

SEQ ID NO: 20 - protein, 17 aa, synthetic construct
VISEDGSNKYHADSVKG

SEQ ID NO: 21 - protein, 24 aa, synthetic construct
ENVRPYYDFWSGYNSEYYYYGMDV

SEQ ID NO: 22 - protein, 5 aa, synthetic construct
SYVMH

SEQ ID NO: 23 - protein, 17 aa, synthetic construct
VISEDGSNKYEADSVKG

SEQ ID NO: 24 - protein, 24 aa, synthetic construct
ENVRPYYDFWRGYNSEYYYYGLDV

SEQ ID NO: 25 - skipped

SEQ ID NO: 26 - protein, 17 aa, synthetic construct
  VARIANT 4: note = Y or E or Q or D
  VARIANT 11: note = Y or N or H or E or D or K or Q or R
VISXDGSNKYXADSVKG

SEQ ID NO: 27 - protein, 24 aa, synthetic construct
  VARIANT 11: note = S or R or K or H
  VARIANT 14: note = Y or S or N or T or A or Q
  VARIANT 22: note = M or L or I or V
ENVRPYYDFWXGYXSEYYYYGXDV

SEQ ID NO: 28 - protein, 109 aa, synthetic construct
DIQMTQSPSSLSASVGDRVTITCRASQSISRYLNWYQQKPGKAPKLLIYAASSLQSGVPSRFSGSGSGTDFTLTISSLQPEDFATYYCQQSYSTQRGSFGQGTKVEIKR

SEQ ID NO: 29 - protein, 132 aa, synthetic construct
EVQLVESGGGVVQPGRSLRLSCAASGFTFSSYDMHWVRQAPGKGLEWVAVISEDGSNKYNADSVKGRFTISRDNSKNTLYLQMNSLRAEDTAVYYCARENVRPYYDFWSGYSSEYYYYGLDVWGQGTTVTVS

SEQ ID NO: 30 - protein, 109 aa, synthetic construct
DIQMTQSPSSLSASVGDRVTITCRASQSISRYLNWYQQKPGKAPKLLIYAASSLQNGVPSRFSGSGSGTDFTLTISSLQPEDFATYYCQQSYSTTRGSFGQGTKVEIKR

SEQ ID NO: 31 - protein, 133 aa, synthetic construct
EVQLVESGGGVVQPGRSLRLSCAASGFTFSSETMHWVRQAPGKGLEWVAVISEDGSNKYHADSVKGRFTISRDNSKNTLYLQMNSLRAEDTAVYYCARENVRPYYDFWSGYNSEYYYYGMDVWGQGTTVTVSS

SEQ ID NO: 32 - protein, 109 aa, synthetic construct
DIQMTQSPSSLSASVGDRVTITCRASQSISRYLNWYQQKPGKAPKLLIYAASSLQSGVPSRFSGSGSGTDFTLTISSLQPEDFATYYCQQSYSTQRGAFGQGTKVEIKR

SEQ ID NO: 33 - protein, 133 aa, synthetic construct
EVQLVESGGGVVQPGRSLRLSCAASGFTFSSYVMHWVRQAPGKGLEWVAVISEDGSNKYEADSVKGRFTISRDNSKNTLYLQMNSLRAEDTAVYYCARENVRPYYDFWRGYNSEYYYYGLDVWGQGTTVTVSS

SEQ ID NO: 34 - protein, 109 aa, synthetic construct; identical to SEQ ID NO: 28

SEQ ID NO: 35 - protein, 133 aa, synthetic construct
EVQLVESGGGVVQPGRSLRLSCAASGFTFSSYVMHWVRQAPGKGLEWVAVISEDGSNKYNADSVKGRFTISRDNSKNTLYLQMNSLRAEDTAVYYCARENVRPYYDFWSGYSSEYYYYGLDVWGQGTTVTVSS

SEQ ID NO: 36 - protein, 109 aa, synthetic construct
DIQMTQSPSSLSASVGDRVTITCRASQSISRYLNWYQQKPGKAPKLLIYAASSLQSGVPSRFSGSGSGTDFTLTISSLQPEDFATYYCQQSYSTTRGSFGQGTKVEIKR

SEQ ID NO: 37 - protein, 133 aa, synthetic construct
EVQLVESGGGVVQPGRSLRLSCAASGFTFSSYDMHWVRQAPGKGLEWVAVISEDGSNKYNADSVKGRFTISRDNSKNTLYLQMNSLRAEDTAVYYCARENVRPYYDFWRGYNSEYYYYGLDVWGQGTTVTVSS

SEQ ID NO: 38 - protein, 109 aa, synthetic construct
DIQMTQSPSSLSASVGDRVTITCRASQSISRYLNWYQQKPGKAPKLLIYAASSLQSGVPSRFSGSGSGTDFTLTISSLQPEDFATYYCQQSYSTTGGTFGQGTKVEIKR

SEQ ID NO: 39 - protein, 133 aa, synthetic construct
EVQLVESGGGVVQPGRSLRLSCAASGFTFSSETMHWVRQAPGKGLEWVAVISEDGSNKYNADSVKGRFTISRDNSKNTLYLQMNSLRAEDTAVYYCARENVRPYYDFWSGYSSEYYYYGLDVWGQGTTVTVSS

SEQ ID NO: 40 - protein, 109 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPRGTFGQGTKVEIKR

SEQ ID NO: 41 - protein, 166 aa, synthetic construct
EVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARENVRPYYDFWSGYYSEYYYYGMDVWGQGTTVTVSSASTKGPSVFPLAPSSKSTSGGTAALGCLVKDYF

SEQ ID NO: 42 - protein, 1121 aa, Homo sapiens
MSKLRMVLLEDSGSADFRRHFVNLSPFTITVVLLLSACFVTSSLGGTDKELRLVDGENKCSGRVEVKVQEEWGTVCNNGWSMEAVSVICNQLGCPTAIKAPGWANSSAGSGRIWMDHVSCRGNESALWDCKHDGWGKHSNCTHQQDAGVTCSDGSNLEMRLTRGGNMCSGRIEIKFQGRWGTVCDDNFNIDHASVICRQLECGSAVSFSGSSNFGEGSGPIWFDDLICNGNESALWNCKHQGWGKHNCDHAEDAGVICSKGADLSLRLVDGVTECSGRLEVRFQGEWGTICDDGWDSYDAAVACKQLGCPTAVTAIGRVNASKGFGHIWLDSVSCQGHEPAIWQCKHHEWGKHYCNHNEDAGVTCSDGSDLELRLRGGGSRCAGTVEVEIQRLLGKVCDRGWGLKEADVVCRQLGCGSALKTSYQVYSKIQATNTWLFLSSCNGNETSLWDCKNWQWGGLTCDHYEEAKITCSAHREPRLVGGDIPCSGRVEVKHGDTWGSICDSDFSLEAASVLCRELQCGTVVSILGGAHFGEGNGQIWAEEFQCEGHESHLSLCPVAPRPEGTCSHSRDVGVVCSRYTEIRLVNGKTPCEGRVELKTLGAWGSLCNSHWDIEDAHVLCQQLKCGVALSTPGGARFGKGNGQIWRHMFHCTGTEQHMGDCPVTALGASLCPSEQVASVICSGNQSQTLSSCNSSSLGPTRPTIPEESAVACIESGQLRLVNGGGRCAGRVEIYHEGSWGTICDDSWDLSDAHVVCRQLGCGEAINATGSAHFGEGTGPIWLDEMKCNGKESRIWQCHSHGWGQQNCRHKEDAGVICSEFMSLRLTSEASREACAGRLEVFYNGAWGTVGKSSMSETTVGVVCRQLGCADKGKINPASLDKAMSIPMWVDNVQCPKGPDTLWQCPSSPWEKRLASPSEETWITCDNKIRLQEGPTSCSGRVEIWHGGSWGTVCDDSWDLDDAQVVCQQLGCGPALKAFKEAEFGQGTGPIWLNEVKCKGNESSLWDCPARRWGHSECGHKEDAAVNCTDISVQKTPQKATTGRSSRQSSFIAVGILGVVLLAIFVALFFLTKKRRQRQRLAVSSRGENLVHQIQYREMNSCLNADDLDLMNSSGGHSEPH

SEQ ID NO: 43 - protein, 27 aa, Homo sapiens
IGRVNASKGFGHIWLDSVSCQGHEPAI

SEQ ID NO: 44 - protein, 11 aa, Homo sapiens
VVCRQLGCGSA

SEQ ID NO: 45 - protein, 14 aa, Homo sapiens
WDCKNWQWGGLTCD

SEQ ID NO: 46 - protein, 10 aa, synthetic construct
  MOD_RES 2: note = D-Tryptophan
  MOD_RES 7: note = D-Phenylalanine
  MOD_RES 8: note = N-alpha-Fmoc-N-epsilon-1-(4,4-dimethyl-2-6-dioxocyc lohex-1-ylidene)ethyl-L-lysine
LWPTDLFKVR

SEQ ID NO: 47 - protein, 6 aa, synthetic construct
HHHHHH

SEQ ID NO: 48 - protein, 4 aa, synthetic construct
GPSL